(19) United States
(12) Patent Application Publication
(10) Pub. No.: US 2019/0091195 A1
(43) Pub. Date: Mar. 28, 2019

(54) USE OF DIANHYDROGALACTITOL AND DERIVATIVES THEREOF IN THE TREATMENT OF GLIOBLASTOMA, LUNG CANCER, AND OVARIAN CANCER

(71) Applicants: Jeffrey A. Bacha, Vancouver (CA); Beibei Zhai, Vancouver (CA); Anne Steinø, Vancouver (CA); Mads Daugaard, Vancouver (CA); Del Mar Pharmaceuticals, Vancouver (CA)

(72) Inventors: Jeffrey A. Bacha, Vancouver (CA); Beibei Zhai, Vancouver (CA); Anne Steinø, Vancouver (CA); Mads Daugaard, Vancouver (CA)

(73) Assignee: Del Mar Pharmaceuticals, Vancouver, BC (CA)

(21) Appl. No.: 15/759,104

(22) PCT Filed: Sep. 9, 2016
(86) PCT No.: PCT/IB16/01436

§ 371 Date: Mar. 9, 2018

Related U.S. Application Data
(60) Provisional application No. 62/320,155, filed on Apr. 8, 2016.
(60) Provisional application No. 62/252,143, filed on Nov. 6, 2015.
(60) Provisional application No. 62/216,860, filed on Sep. 10, 2015.

(51) Int. Cl. A61K 31/336; A61K 45/06; A61P 35/00

(52) U.S. Cl. CPC A61K 31/336; A61P 35/00; A61K 45/06

(57) ABSTRACT

Substituted hexitol derivatives such as dianhydrogalactitol are useful in the treatment of various neoplastic pathologies. Said pathologies include glioblastoma multiforme, non-small-cell lung carcinoma (NSCLC), ovarian cancer, and leptomeningeal carcinomatosis. The anti-neoplastic activity of dianhydrogalactitol is demonstrated to be due to its activity as an alkylating agent that creates N⁷ methylation and inter-strand DNA crosslinks. The hexitol derivatives may be used alone or in combination with other anti-neoplastic agents.

### CROSS-REFERENCE TO RELATED APPLICATION

This application claims the benefit of U.S. Provisional Patent Application Ser. No. 62/216,860 by B. Zhai et al., entitled “USE OF DIANHYDROGALACTITOL OR DERIVATIVES AND ANALOGS THEREOF FOR TREATMENT OF NON-SMALL-CELL LUNG CARCINOMA, GLIOBLASTOMA MULTIFORME, AND OVARIAN CARCINOMA BY INDUCTION OF DNA DAMAGE,” and filed on Sep. 10, 2015, the contents of which are hereby incorporated herein by this reference. This application also claims the benefit of U.S. Provisional Patent Application Ser. No. 62/252,143 by B. Zhai et al., entitled “USE OF DIANHYDROGALACTITOL OR DERIVATIVES AND ANALOGS THEREOF FOR TREATMENT OF NON-SMALL-CELL LUNG CARCINOMA, GLIOBLASTOMA MULTIFORME, AND OVARIAN CARCINOMA BY INDUCTION OF DNA DAMAGE,” and filed on Nov. 6, 2015, the contents of which are hereby incorporated herein by this reference. This application also claims the benefit of U.S. Provisional Application Ser. No. 62/320,155 by B. Zhai et al., entitled ““USE OF DIANHYDROGALACTITOL OR DERIVATIVES AND ANALOGS THEREOF FOR TREATMENT OF NON-SMALL-CELL LUNG CARCINOMA, GLIOBLASTOMA MULTIFORME, AND OVARIAN CARCINOMA BY INDUCTION OF DNA DAMAGE AND STALLING OF CELL CYCLE,” and filed on Apr. 8, 2016, the contents of which are hereby incorporated herein by this reference.

### FIELD OF THE INVENTION

This patent application is directed to the use of dianhydrogalactitol or derivatives and analogs thereof for treatment of non-small-cell lung carcinoma, glioblastoma, and ovarian carcinoma by induction of DNA damage, either as a single therapeutic agent or together with additional agents that can induce DNA damage, interrupt the replicative cell cycle, or block the cellular mechanisms that can repair DNA damage in malignant cells.

### BACKGROUND OF THE INVENTION

The search for and identification of cures for many life-threatening diseases that plague humans still remains an empirical and sometimes serendipitous process. While many advances have been made from basic scientific research to improvements in practical patient management, there still remains tremendous frustration in the rational and successful discovery of useful therapies particularly for life-threatening diseases such as cancer, inflammatory conditions, infection, and other conditions.

Since the “War on Cancer” began in the early 1970's by the United States National Cancer Institute (NCI) of the National Institutes of Health (NIH), a wide variety of strategies and programs have been created and implemented to prevent, diagnose, treat and cure cancer. One of the oldest and arguably most successful programs has been the synthesis and screening of small chemical entities (<1500 MW) for biological activity against cancer. This program was organized to improve and streamline the progression of events from chemical synthesis and biological screening to preclinical studies for the logical progression into human clinical trials with the hope of finding cures for the many types of life-threatening malignant tumors. The synthesis and screening of hundreds of thousands of chemical compounds from academic and industrial sources, in addition to the screening of natural products and extracts from prokaryotes, invertebrate animals, plant collections, and other sources from all over the world has been and continues to be a major approach for the identification of novel lead structures as potential new and useful medicines. This is in addition to other programs including biotherapeutics designed to stimulate the human immune system with vaccines, therapeutic antibodies, cytokines, lymphokines, inhibitors of tumor blood vessel development (angiogenesis) or gene and antisense therapies to alter the genetic make-up of cancer cells, and other biological response modifiers.

The work supported by the NCI, other governmental agencies both domestic and foreign in academic or industrial research and development laboratories has resulted in an extraordinary body of biological, chemical and clinical information. In addition, large chemical libraries have been created, as well as highly characterized in vitro and in vivo biological screening systems that have been successfully used. However, from the tens of billions of dollars spent over the past thirty years supporting these programs both preclinically and clinically, only a small number of compounds have been identified or discovered that have resulted in the successful development of useful therapeutic products. Nevertheless, the biological systems both in vitro and in vivo and the “decision trees” used to warrant further animal studies leading to clinical studies have been validated. These programs, biological models, clinical trial protocols, and other information developed by this work remain critical for the discovery and development of any new therapeutic agent.

Unfortunately, many of the compounds that have successfully met the preclinical testing and federal regulatory requirements for clinical evaluation were either unsuccessful or disappointing in human clinical trials. Many compounds were found to have untoward or idiosyncratic side-effects that were discovered during human clinical Phase I dose-escalation studies used to determine the maximum tolerated dose (MTD) and side-effect profile. In some cases, these toxicities or the magnitude of their toxicity were not identified or predicted in preclinical toxicology studies. In other cases, chemical agents where in vitro and in vivo studies suggested a potentially unique activity against a particular tumor type, molecular target or biological pathway were not successful in human Phase II clinical trials where specific examination of particular cancer indications/types were evaluated in government sanctioned (e.g., U.S. FDA), IRB approved clinical trials. In addition, there are those cases where potential new agents were evaluated in randomized Phase III clinical trials where a significant clinical benefit could not be demonstrated; such cases have also been the cause of great frustration and disappointment. Finally, a number of compounds have reached commercialization but their ultimate clinical utility has been limited by poor efficacy as monotherapy (<25% response rates) and untoward dose-limiting side-effects (Grade III and IV) (e.g., myelosuppression, neurotoxicity, cardiotoxicity, gastrointestinal toxicities, or other significant side effects).

In many cases, after the great time and expense of developing and moving an investigational compound into human clinical trials and where clinical failure has occurred, the tendency has been to return to the laboratory to create a better analog, look for agents with different structures but potentially related mechanisms of action, or try other modifications of the drug. In some cases, efforts have been made to try additional Phase I or II clinical trials in an attempt to make some improvement with the side-effect profile or therapeutic effect in selected patients or cancer indications. In many of those cases, the results did not realize a significant enough improvement to warrant further clinical development toward product registration. Even for commercialized products, their ultimate use is still limited by suboptimal performance.

With so few therapeutics approved for cancer patients and the realization that cancer is a collection of diseases with a multitude of etiologies and that a patient's response and survival from therapeutic intervention is complex with many factors playing a role in the success or failure of treatment including disease indication, stage of invasion and metastatic spread, patient gender, age, health conditions, previous therapies or other illnesses, genetic markers that can either promote or retard therapeutic efficacy, and other factors, the opportunity for cures in the near term remains elusive. Moreover, the incidence of cancer continues to rise with an approximate 4% increase predicted for 2003 in the United States by the American Cancer Society such that over 1.3 million new cancer cases are estimated. In addition, with advances in diagnosis such as mammography for breast cancer and PSA tests for prostate cancer, more patients are being diagnosed at a younger age. For difficult to treat cancers, a patient's treatment options are often exhausted quickly resulting in a desperate need for additional treatment regimens. Even for the most limited of patient populations, any additional treatment opportunities would be of considerable value. This invention focuses on inventive compositions and methods for improving therapeutic benefit of suboptimally administered chemical compounds including substituted hexitols such as dianhydrogalactitol.

Non-small-cell lung carcinoma (NSCLC) includes several types of lung cancer, including squamous cell carcinoma, large cell carcinoma, and adenocarcinoma, as well as other types of lung cancer. Although smoking is apparently the most frequent cause of squamous cell carcinoma, when lung cancer occurs in patients without any history of prior tobacco smoking, it is frequently adenocarcinoma. In many cases, NSCLC is refractory to chemotherapy, so surgical resection of the tumor mass is typically the treatment of choice, particularly if the malignancy is diagnosed early. However, chemotherapy and radiation therapy are frequently attempted, particularly if the diagnosis cannot be made at an early stage of the malignancy. Other treatments include radiofrequency ablation and chemoembolization. A wide variety of chemotherapeutic treatments has been tried for advanced or metastatic NSCLC. Some patients with particular mutations in the EGFR gene respond to EGFR tyrosine kinase inhibitors such as gefitinib (M. G. Kris, “How Today's Developments in the Treatment of Non-Small Cell Lung Cancer Will Change Tomorrow's Standards of Care,” Oncologist 10 (Suppl. 2): 23-29 (2005)). However, acquired resistance to tyrosine kinase inhibitors is frequent and often linked to the occurrence of T790M mutation in the EGFR gene. Cisplatin has frequently been used as ancillary therapy together with surgery, and while often initially effective, resistance often arises and continues to be a challenge. About 7% of NSCLC have EML4-ALK translocations, and such patients may benefit from ALK inhibitors such as crizotinib. Other therapies, including the vaccine TG4010, motesanib diphosphate, tivantinib, belotecan, eribulin mesylate, ramucirumab, necitumumab, the vaccine GSK1572932A, custirsen sodium, the liposome-based vaccine BLP25, nivolumab, EMD531444, dacomitinib, and genetespib, are being evaluated, particularly for advanced or metastatic NSCLC.

However, there is still a need for effective therapies against NSCLC, especially against advanced or metastatic NSCLC. Preferably, such therapies should be well-tolerated and with side effects, if any, that could be easily controlled. Also, preferably, such therapies should be compatible with other chemotherapeutic approaches and with surgery or radiation. Additionally, and preferably, such therapies should be able to exert a synergistic effect on other treatment modalities.

Glioblastoma is the most common and aggressive malignant primary brain tumor occurring in humans. Glioblastoma involves glial cells; it accounts for 52% of all functional tissue brain tumor cases and 20% of all intracranial tumors. Its estimated frequency of occurrence is 2-3 cases per 100,000 people in Europe and North America.

Glioblastoma has an extremely poor prognosis, despite various treatment methods including open craniotomy with surgical resection of as much of the tumor as possible, followed by sequential or concurrent chemoradiotherapy, antiangiogenic therapy with bevacizumab, gamma knife radiosurgery, and symptomatic management with corticosteroids. The median survival time for glioblastoma is only 14 months.

Common symptoms of glioblastoma include seizures, nausea, vomiting, headache, and hem iparesis. However, the most prevalent symptoms of glioblastoma are progressive memory, personality, or neurological deficit due to involvement of the temporal or frontal lobe of the brain. The kind of symptoms produced by glioblastoma depends highly on the location of the tumor and less on its exact pathology. The tumor can start producing symptoms quickly, but occasionally is asymptomatic until it reaches an extremely large size.

The etiology of glioblastoma is largely unknown. For unknown reasons, glioblastoma occurs more frequently in males. Most glioblastoma tumors appear to be sporadic, without any significant genetic predisposition. No links have been found between glioblastoma and several known carcinogenic risk factors, including diet, smoking, and exposure to electromagnetic fields. There have been some suggestions of a viral etiology, possibly SV40 or cytomegalovirus. There may also be some association between exposure to ionizing radiation and glioblastoma. Additionally, it has been proposed that there is a link between polyvinyl chloride exposure and glioblastoma; lead exposure in the workplace has also been suggested as a possible cause. There is an association of brain tumor incidence and malaria, suggesting that the anopheles mosquito, the carrier of malaria, might transmit a virus or other causative agent of glioblastoma.

Glioblastoma is also relatively more common in people over 50 years of age, in Caucasians or Asians, and in patients that have already developed a low-grade astrocytoma which can develop into a higher grade tumor. Additionally, having one of the following genetic disorders is associated with an increased incidence of glioblastoma: neurofibromatosis, tuberous sclerosis, Von Hippel-Lindau disease, Li-Fraumeni syndrome, or Turcot syndrome.

Glioblastoma tumors are typically characterized by the presence of small areas of necrotizing tissue that are surrounded by anaplastic cells. These characteristics, together with the presence of hyperplastic blood vessels, differentiate these malignancies from Grade 3 astrocytomas, which do not have these features.

There are four subtypes of glioblastoma. An extremely large fraction (97%) of tumors in the so-called “classical” subtype carry extra copies of the epidermal growth factor receptor (EGFR) gene and most of these tumors have higher than normal expression of EGFR, whereas the gene TP53, a tumor suppressor gene that has a number of anticancer activities, and which is often mutated in glioblastoma, is rarely mutated in this subtype. In contrast, the proneural subtype often has high rates of alteration in TP53 and in PDGFRA, the gene encoding the α-type platelet-derived growth factor receptor, as well as in IDH1, the gene encoding isocitrate dehydrogenase-1. The mesenchymal subtype is characterized by high rates of mutations or alterations in NF1, the gene encoding Neurofibromin type 1 and fewer alterations in the EGFR gene and less expression of EGFR than the other subtypes. Missing the fourth subtype: Neural subtype

Glioblastoma usually forms in the cerebral white matter, grows quickly, and can become very large before producing symptoms. Less than 10% of glioblastomas form more slowly following degeneration of low-grade astrocytoma or anaplastic astrocytoma; such tumors are called secondary glioblastomas and are relatively more common in younger patients. The tumor may extend into the meninges or the ventricular wall leading to abnormally high protein content in the cerebrospinal fluid (CSF) (>100 mg/dL), as well as an occasional pleocytosis of 10 to 100 cells, mostly lymphocytes. Malignant cells present in the CSF can rarely spread to the spinal cord or cause meningeal gliomatosis; however, metastasis of glioblastoma beyond the central nervous system is extremely unusual. About 50% of glioblastoma tumors occupy more than one lobe of a hemisphere or are bilateral. Tumors of this type usually arise from the cerebrum and may rarely exhibit the classic infiltration across the corpus callosum, producing a bilateral (“butterfly”) glioma. The tumor can take on a variety of appearances, depending on the amount of hemorrhage or necrosis present or the age of the tumor. A CT scan of a glioblastoma tumor will usually show an inhomogeneous mass with a hypodense center and a variable ring of enhancement surrounded by edema. The mass effect from the tumor and the surrounding edema may compress the ventricles and cause hydrocephalus.

Cancer cells with stem-cell-like properties have been found in glioblastomas. This may be one cause of their resistance to conventional treatments and their high recurrence rate.

Glioblastoma often presents typical features on MRI, but these features are not specific for glioblastoma and may be caused by other conditions. Specifically, when viewed with MRI, glioblastomas often appear as ring-enhancing lesions. However, other lesions such as abscesses, metastases of malignancies arising outside the central nervous system, tumefactive multiple sclerosis, or other conditions may have a similar appearance. The definitive diagnosis of a suspected glioblastoma on CT or MRI requires a stereotactic biopsy or a craniotomy with tumor resection and pathologic confirmation. Because the grade of the tumor is based on the most malignant portion of the tumor, biopsy or subtotal tumor resection can result in undergrading of the tumor. Imaging of tumor blood flow using perfusion MRI and measuring tumor metabolite concentration with MR spectroscopy may add value to standard MRI, but pathology remains the gold standard for glioblastoma diagnosis.

The treatment of glioblastoma is extremely difficult due to several factors: (1) the tumor cells are very resistant to conventional therapies; (2) the brain is susceptible to damage using conventional therapy; (3) the brain has a very limited capacity for self-repair; and (4) many therapeutic drugs cannot cross the blood-brain barrier to act on the tumor. Symptomatic therapy, including the use of corticosteroids and anticonvulsant agents, focuses on relieving symptoms and improving the patient's neurologic function. However, such symptomatic therapy does nothing to slow the progression of the tumor, and, in the case of administration of phenytoin concurrently with radiation therapy, can result in substantial side effects including erythema multiforme and Stevens-Johnson syndrome.

Palliative therapy usually is conducted to improve quality of life and to achieve a longer survival time. Palliative therapy can include surgery, radiation therapy, and chemotherapy. A maximally feasible resection with maximally tumor-free margins is generally performed along with external beam radiation and chemotherapy. Gross total resection of tumor is associated with better prognoses.

Surgery is the first stage of treatment of glioblastoma. An average glioblastoma tumor contains 10¹¹ cells, which is on average reduced to 10⁹ cells after surgery (a reduction of 99%). Surgery is used to take a section for a pathological diagnosis, to remove some of the symptoms of a large mass pressing against the brain, to remove disease before secondary resistance to radiotherapy and chemotherapy, and to prolong survival. The greater the extent of tumor removal, the better is the outcome. Removal of 98% or more of the tumor has been associated with a significantly longer and healthier survival time than if less than 98% of the tumor is removed. The chances of near-complete initial removal of the tumor can be greatly increased if the surgery is guided by a fluorescent dye known as 5-aminolevulinic acid. Glioblastoma cells are widely infiltrative through the brain at diagnosis, and so despite a “total resection” of all obvious tumor, most people with glioblastoma later develop recurrent tumors either near the original site or at more distant “satellite lesions” within the brain. Other modalities, including radiation, are used after surgery in an effort to suppress and slow recurrent disease.

After surgery, radiotherapy is the mainstay of treatment for people with glioblastoma. A pivotal clinical trial carried out in the early 1970s showed that among 303 glioblastoma patients randomized to radiation or nonradiation therapy, those who received radiation had a median survival more than double those who did not. Subsequent clinical research has attempted to build on the backbone of surgery followed by radiation. On average, radiotherapy after surgery can reduce the tumor size to 10⁷ cells. Whole brain radiotherapy does not improve the results when compared to the more precise and targeted three-dimensional conformal radiotherapy. A total radiation dose of 60-65 Gy has been found to be optimal for treatment.

The use of chemotherapy in glioblastoma in addition to radiation has thus far only resulted in marginal improvements in survival as compared with radiation alone. In the treatment of other malignancies, the addition of chemotherapy to radiation has resulted in substantial improvements in survival, but this has not yet proven to be the case for glioblastoma. One drug that does show results in connection with radiation is temozolomide (TMZ). TMZ plus radiation is now standard for most cases of glioblastoma. TMZ seems to work by sensitizing the tumor cells to radiation.

However, TMZ is often ineffective due to drug resistance as the result of the catalytic activity of the enzyme O⁶-methylguanine-DNA methyltransferase (MGMT), which results in repair of the lesion at O⁶ of the guanine of DNA molecules. Chemoresistance to TMZ as a result of the activity of MGMT is frequently associated with poor outcomes in TMZ-treated patients, and patients in whom TMZ or bevacizumab is ineffective are left with few if any treatment options.

Additionally, cancer stem cells (CSC) are a subpopulation of the tumor that resist therapy and give rise to relapse.

Another therapeutic approach involves the use of the monoclonal antibody bevacizumab, which is a humanized monoclonal antibody that inhibits vascular endothelial growth factor A (VEGF-A) and thus acts as an angiogenesis inhibitor. Although bevacizumab may retard the progression of the disease, the first-line use of bevacizumab does not improve overall survival in patients with newly diagnosed glioblastoma (M. R. Gilbert et al., “A Randomized Trial of Bevacizumab for Newly Diagnosed Glioblastoma,” New Engl. J. Med. 370: 699-708 (2014)). Additionally, unlike some other malignancies in which the use of bevacizumab results in a potentiation of chemotherapy, in glioblastoma, the addition of chemotherapy to bevacizumab did not improve on results from bevacizumab alone. Bevacizumab reduces brain edema and consequent symptoms, and it may be that the benefit from this drug is due to its action against edema rather than any action against the tumor itself. Some patients with brain edema do not actually have any active tumor remaining, but rather develop the edema as a late effect of prior radiation treatment. This type of edema is difficult to distinguish from that due to tumor, and both may coexist. Both respond to bevacizumab. However, patients in which both temozolomide and bevacizumab have been ineffective have few if any treatment options.

Another approach that has been proposed is gene transfer. Although gene transfer therapy has the potential to kill cancer cells while leaving healthy cells unharmed, this approach has been beset with many difficulties in other diseases, including the possibility for induction of other types of malignancies and interference with the functioning of the immune system.

Still other treatment modalities have been proposed for glioblastoma, including the use of protein therapeutics, including the soluble CD95-Fc fusion protein APG101, immunotherapy with tumor vaccines, alternating electrical fields, and metabolic therapy. The value of these treatment modalities remains to be determined.

In glioblastoma, the median survival time from the time of diagnosis without any treatment is 3 months, but with treatment survival of 1-2 years is common. Increasing age (>60 years of age) carries a worse prognostic risk. Death is usually due to cerebral edema or increased intracranial pressure.

A good initial Karnofsky Performance Status (KPS) and methylation of the promoter of the O⁶-methylguanine-DNA methyltransferase (MGMT) gene are associated with longer survival. A DNA test can be carried out on glioblastomas to determine whether the promoter of the MGMT gene is methylated. Even in patients less than 50 years of age with a KPS (Karnofsky Performance Status) of equal to or greater than 90%, the 5-year survival rate is only 14%.

Therefore, there is a need for improved therapies for glioblastoma that provide improved survival with reduced side effects and impairment of function in surviving patients.

There is a particular need for therapeutic modalities that can cross the blood-brain barrier (BBB), that can suppress the growth and division of cancer stem cells (CSC), and that can avoid inactivation by O⁶-methylguanine-DNA methyltransferase (MGMT). There is also a particular need for therapeutic modalities that yield increased response rates and improved quality of life for patients with these malignancies. There is also a particular need for therapeutic modalities that are effective in patients in which either or both of temozolomide and bevacizumab have proven ineffective.

Ovarian cancer is a relatively common malignancy that has a relatively poor prognosis. One factor that contributes to the poor prognosis of ovarian cancer is the fact that there is no clear early detection or screening test for this form of cancer, which means that many cases are only diagnosed in a relatively advanced stage, by which time most treatment options are ineffective. The early symptoms of ovarian cancer, such as bloating and pelvic pain, are nonspecific and can be associated with many other conditions. Ovarian cancer metastasizes early in its development, often before it has been diagnosed. High-grade tumors metastasize more readily than low-grade tumors. Typically, tumor cells begin to metastasize by growing in the peritoneal cavity. More than 60% of women presenting with ovarian cancer have stage-III or stage-IV cancer, when it has already spread beyond the ovaries. Ovarian cancers shed cells into the naturally occurring fluid within the abdominal cavity. These cells can then implant on other abdominal (peritoneal) structures, included the uterus, urinary bladder, bowel, lining of the bowel wall, and omentum, forming new tumor growths before cancer is even suspected. The five-year survival rate for all stages of ovarian cancer is 46%; the one-year survival rate is 72% and the ten-year survival rate is 35%. For cases where a diagnosis is made early in the disease, when the cancer is still confined to the primary site, the five-year survival rate is 92.7%. About 70% of women with advanced disease respond to initial treatment, most of whom attain complete remission, but half of these women experience a recurrence 1-4 years after treatment.

A number of risk factors are known for ovarian cancer, including the use of postmenopausal hormone replacement therapy, having few or no children, smoking, endometriosis, and genetic factors. The major genetic risk factor for ovarian cancer is a mutation in BRCA1 or BRCA2 DNA mismatch repair genes, which is present in 10% of ovarian cancer cases. Only one allele need be mutated to place a person at high risk, because the risky mutations are autosomal dominant. The gene can be inherited through either the maternal or paternal line, but has variable penetrance. Though mutations in these genes are usually associated with increased risk of breast cancer, they also carry a 30-50% lifetime risk of ovarian cancer, a risk that peaks in a person's 40s and 50s. This risk is also cited as 40-60% and 39-46%. Mutations in BRCA2 are less risky than those with BRCA1, with a lifetime risk of 20-40%. This risk is also cited as 12-20%. On average, BRCA-associated cancers develop 15 years before their sporadic counterparts, because people who inherit the mutations on one copy of their gene only need one mutation to start the process of carcinogenesis, whereas people with two normal genes would need to acquire two mutations. Mutations in BRCA1 or BRCA2 are particularly common in individuals with Ashkenazi Jewish ancestry, but also occur in other ethnic groups. Other genetic markers have been also associated with increased risk of developing ovarian cancer. These genetic markers include, but are not limited to, mutations in AKT1, AKT2, ARIDIA, BRAF, CCDN1, CCND2, CCNE1, CDK12, CDKN2A, CTNNBI, DICER1, DYNLRB1, EGFR, ERBB2, FMS, JAG1, JAG2, KRAS, MAML1, MAML2, MAML3, MLH1, NF1, NOTCH3, NRAS, PIK3C3, PIK3CA, PPP2R1A, PTEN, RBI, TGF-β, TP53, TβR1, TβRII, and USP36.

Treatment for ovarian cancer can involve one or more of surgery, radiation, and chemotherapy. Surgery can include removal of one (unilateral oophorectomy) or both ovaries (bilateral oophorectomy), the Fallopian tubes (salpingectomy), the uterus (hysterectomy), and the omentum (omentectomy). Typically, all of these are removed. Radiotherapy can be used to treat dysgerminomas, but is now less frequently employed; it is generally effective in advanced stages of ovarian cancer. A variety of chemotherapies is employed; chemotherapeutic agents that are used include paclitaxel, cisplatin, topotecan, gemcitabine, docetaxel, carboplatin, bleomycin, etoposide, doxorubicin, cyclophosphamide, trabectedin, and olaparib. Generally, the platinum-containing agents such as cisplatin or carboplatin are the first line of therapy. However, resistance to the platinum-containing agents frequently develops and is difficult to treat. Immunotherapy with the monoclonal antibody bevacizumab is also employed.

Therefore, there is a need for more effective treatment of ovarian cancer, particularly advanced ovarian cancer or ovarian cancer that has developed resistance to the platinum-containing agents.

There is also a need for a more effective treatment of leptomeningeal carcinomatosis (LC). Leptomeningeal carcinomatosis (LC) is a complication of cancer in which the disease spreads to the meninges surrounding the brain and spinal cord. LC occurs in approximately 5% of people with cancer and is usually terminal. If left untreated, median survival is 4-6 weeks; if treated, median survival is 2-3 months. Meningeal symptoms are the first manifestations in some patients (pain and seizures are the most common presenting complaints) and can include the following: headaches, which are usually associated with nausea, vomiting, or light-headedness; gait difficulties from weakness or ataxia; memory problems; incontinence; or sensory abnormalities. LC is generally considered difficult to treat and generally incurable.

Therefore, there is a need for improved methods for treatment of these malignancies. In particular, there is a need for an improved method that can inhibit the replicative cell cycle in tumor cells of these malignancies and can prevent or inhibit DNA repair in these cells, sensitizing the cells to a number of treatment modalities and inducing their apoptosis.

### SUMMARY OF THE INVENTION

The use of a substituted hexitol derivative to treat glioblastoma, non-small-cell lung carcinoma (NSCLC), or ovarian cancer provides an improved therapy for these malignancies that yields increased survival and is substantially free of side effects. The compositions and methods of the present invention can inhibit the replicative cell cycle in tumor cells of these malignancies and can prevent or inhibit DNA repair in these cells, sensitizing the cells to a number of treatment modalities and inducing their apoptosis. In general, the substituted hexitols usable in methods and compositions according to the present invention include galactitols, substituted galacitols, dulcitols, and substituted dulcitols. Typically, the substituted hexitol derivative is selected from the group consisting of dianhydrogalactitol, derivatives of dianhydrogalactitol, diacetyldianhydrogalactitol, derivatives of diacetyldianhydrogalactitol, dibromodulcitol, and derivatives of dibromodulcitol. A particularly preferred substituted hexitol derivative is dianhydrogalactitol (DAG). The substituted hexitol derivative can be employed together with other therapeutic modalities for these malignancies. Dianhydrogalactitol is particularly suited for the treatment of these malignancies because it crosses the blood-brain barrier, because it can suppress the growth of cancer stem cells (CSC), and because it is resistant to drug inactivation by O⁶-methylguanine-DNA methyltransferase (MGMT). The substituted hexitol derivative yields increased response rates and improved quality of life for patients with glioblastoma, NSCLC, and ovarian cancer.

Dianhydrogalactitol is a novel alkylating agent that creates N⁷-methylation in DNA. Specifically, dianhydrogalactitol methylates the N⁷ position of guanine residues in DNA.

Accordingly, one aspect of the present invention is a method to improve the efficacy and/or reduce the side effects of the administration of a substituted hexitol derivative for treatment of glioblastoma, NSCLC, or ovarian cancer comprising the steps of:

(1) identifying at least one factor or parameter associated with the efficacy and/or occurrence of side effects of the administration of the substituted hexitol derivative for treatment of glioblastoma, NSCLC, or ovarian cancer; and

(2) modifying the factor or parameter to improve the efficacy and/or reduce the side effects of the administration of the substituted hexitol derivative for treatment of glioblastoma, NSCLC, or ovarian cancer.

Typically, the factor or parameter is selected from the group consisting of:

(1) dose modification;

(2) route of administration;

(3) schedule of administration;

(4) indications for use;

(5) selection of disease stage;

(6) other indications;

(7) patient selection;

(8) patient/disease phenotype;

(9) patient/disease genotype;

(10) pre/post-treatment preparation

(11) toxicity management;

(12) pharmacokinetic/pharmacodynamic monitoring;

(13) drug combinations;

(14) chemosensitization;

(15) chemopotentiation;

(16) post-treatment patient management;

(17) alternative medicine/therapeutic support;

(18) bulk drug product improvements;

(19) diluent systems;

(20) solvent systems;

(21) excipients;

(22) dosage forms;

(23) dosage kits and packaging;

(24) drug delivery systems;

(25) drug conjugate forms;

(26) compound analogs;

(27) prodrugs;

(28) multiple drug systems;

(29) biotherapeutic enhancement;

(30) biotherapeutic resistance modulation;

(31) radiation therapy enhancement;

(32) novel mechanisms of action;

(33) selective target cell population therapeutics;

(34) use with ionizing radiation;

(35) use with an agent enhancing its activity;

(36) use with an agent that counteracts myelosuppression; and

(37) use with an agent that increases the ability of the substituted hexitol to pass through the blood-brain barrier.

The substituted hexitol derivative can act by promoting DNA damage and also by causing the cell cycle to be arrested in the S phase, enabling additional therapeutic agents to act to cause cell death at that stage.

As detailed above, typically, the substituted hexitol derivative is selected from the group consisting of dianhydrogalactitol, derivatives of dianhydrogalactitol, diacetyldianhydrogalactitol, derivatives of diacetyldianhydrogalactitol, dibromodulcitol, and derivatives of dibromodulcitol. Preferably, the substituted hexitol derivative is dianhydrogalactitol.

Another aspect of the present invention is a composition to improve the efficacy and/or reduce the side effects of suboptimally administered drug therapy employing a substituted hexitol derivative for the treatment of glioblastoma, NSCLC, or ovarian cancer comprising an alternative selected from the group consisting of:

(i) a therapeutically effective quantity of a modified substituted hexitol derivative or a derivative, analog, or prodrug of a substituted hexitol derivative or a modified substituted hexitol derivative, wherein the modified substituted hexitol derivative or the derivative, analog or prodrug of the substituted hexitol derivative or modified substituted hexitol derivative possesses increased therapeutic efficacy or reduced side effects for treatment of glioblastoma, NSCLC, or ovarian cancer as compared with an unmodified substituted hexitol derivative;

(ii) a composition comprising:
(a) a therapeutically effective quantity of a substituted hexitol derivative, a modified substituted hexitol derivative, or a derivative, analog, or prodrug of a substituted hexitol derivative or a modified substituted hexitol derivative; and
(b) at least one additional therapeutic agent, therapeutic agent subject to chemosensitization, therapeutic agent subject to chemopotentiation, diluent, excipient, solvent system, drug delivery system, agent to counteract myelosuppression, or agent that increases the ability of the substituted hexitol to pass through the blood-brain barrier, wherein the composition possesses increased therapeutic efficacy or reduced side effects for treatment of glioblastoma, NSCLC, or ovarian cancer as compared with an unmodified substituted hexitol derivative;

(iii) a therapeutically effective quantity of a substituted hexitol derivative, a modified substituted hexitol derivative or a derivative, analog, or prodrug of a substituted hexitol derivative or a modified substituted hexitol derivative that is incorporated into a dosage form, wherein the substituted hexitol derivative, the modified substituted hexitol derivative or the derivative, analog, or prodrug of a substituted hexitol derivative or a modified substituted hexitol derivative incorporated into the dosage form possesses increased therapeutic efficacy or reduced side effects for treatment of glioblastoma, NSCLC, or ovarian cancer as compared with an unmodified substituted hexitol derivative;

(iv) a therapeutically effective quantity of a substituted hexitol derivative, a modified substituted hexitol derivative or a derivative, analog, or prodrug of a substituted hexitol derivative or a modified substituted hexitol derivative that is incorporated into a dosage kit and packaging, wherein the substituted hexitol derivative, the modified substituted hexitol derivative or the derivative, analog, or prodrug of a substituted hexitol derivative or a modified substituted hexitol derivative incorporated into the dosage kit and packaging possesses increased therapeutic efficacy or reduced side effects for treatment of glioblastoma, NSCLC, or ovarian cancer as compared with an unmodified substituted hexitol derivative; and

(v) a therapeutically effective quantity of a substituted hexitol derivative, a modified substituted hexitol derivative or a derivative, analog, or prodrug of a substituted hexitol derivative or a modified substituted hexitol derivative that is subjected to a bulk drug product improvement, wherein substituted hexitol derivative, a modified substituted hexitol derivative or a derivative, analog, or prodrug of a substituted hexitol derivative or a modified substituted hexitol derivative subjected to the bulk drug product improvement possesses increased therapeutic efficacy or reduced side effects for treatment of glioblastoma, NSCLC, or ovarian cancer as compared with an unmodified substituted hexitol derivative.

As detailed above, typically the unmodified substituted hexitol derivative is selected from the group consisting of dianhydrogalactitol, derivatives of dianhydrogalactitol, diacetyldianhydrogalactitol, derivatives of diacetyldianhydrogalactitol, dibromodulcitol, and derivatives of dibromodulcitol. Preferably, the unmodified substituted hexitol derivative is dianhydrogalactitol.

Another aspect of the present invention is a method of treating glioblastoma, NSCLC, or ovarian cancer comprising the step of administering a therapeutically effective quantity of a substituted hexitol derivative to a patient suffering from the malignancy. As detailed above, the substituted hexitol derivative is selected from the group consisting of dianhydrogalactitol, derivatives of dianhydrogalactitol, diacetyldianhydrogalactitol, derivatives of diacetyldianhydrogalactitol, dibromodulcitol, and derivatives of dibromodulcitol. Preferably, the substituted hexitol derivative is dianhydrogalactitol.

Typically, when the substituted hexitol derivative is dianhydrogalactitol, the therapeutically effective quantity of dianhydrogalactitol is a dosage from about 1 mg/m² to about 40 mg/m². Preferably, the therapeutically effective quantity of dianhydrogalactitol is a dosage from about 5 mg/m² to about 25 mg/m². Other dosages are described below.

Typically, the substituted hexitol derivative, such as dianhydrogalactitol, is administered by a route selected from the group consisting of intravenous and oral. Other potential routes of administration are described below.

The method can further comprise the step of administering a therapeutically effective dose of ionizing radiation. The method can further comprise the step of administering a therapeutically effective quantity of temozolomide, bevacizumab, or a corticosteroid.

The method can further comprise the administration of a therapeutically effective quantity of a tyrosine kinase inhibitor as described below.

The method can further comprise: (i) administration of a therapeutically effective quantity of a topoisomerase inhibitor; and (ii) administration of a therapeutically effective quantity of an inhibitor of CHK1 kinase or CHK2 kinase.

The method can further comprise the administration of a therapeutically effective quantity of an epidermal growth factor receptor (EGFR) inhibitor as described below. The EGFR inhibitor can affect either wild-type binding sites or mutated binding sites, including Variant III, as described below. The method can also further comprise, subsequent to the administration of an initial dose of the substituted hexitol derivative selected from the group consisting of dianhydrogalactitol, a derivative or analog of dianhydrogalactitol, diacetyldianhydrogalactitol, and a derivative or analog of diacetyldianhydrogalactitol: (1) determining the quantity of a protein associated with the activation of the DNA repair pathway to determine the extent of the activation of the DNA repair pathway; and (2) adjusting the dose of the substituted hexitol derivative selected from the group consisting of dianhydrogalactitol, a derivative or analog of dianhydrogalactitol, diacetyldianhydrogalactitol, and a derivative or analog of diacetyldianhydrogalactitol in response to the extent of the DNA repair pathway. Typically, the protein associated with the activation of the DNA repair pathway is selected from the group consisting of phosphorylated ATM, phosphorylated RPA32, and γH2A.X.

Another aspect of the present invention is a method for the treatment of leptomeningeal carcinomatosis (LC) by the induction of double-strand breaks in the DNA of tumor cells by administration of a therapeutically effective quantity of a substituted hexitol derivative selected from the group consisting of dianhydrogalactitol, a derivative or analog of dianhydrogalactitol, diacetyldianhydrogalactitol, and a derivative or analog of diacetyldianhydrogalactitol. The method can comprise the steps of:

(1) identifying at least one factor or parameter associated with the efficacy and/or occurrence of side effects of the administration of the substituted hexitol derivative for treatment of leptomeningeal carcinomatosis (LC); and

(2) modifying the factor or parameter to improve the efficacy and/or reduce the side effects of the administration of the substituted hexitol derivative for treatment of LC.

Typically, the factor or parameter is selected from the group consisting of:

(1) dose modification;

(2) route of administration;

(3) schedule of administration;

(4) indications for use;

(5) selection of disease stage;

(6) other indications;

(7) patient selection;

(8) patient/disease phenotype;

(9) patient/disease genotype;

(10) pre/post-treatment preparation

(11) toxicity management;

(12) pharmacokinetic/pharmacodynamic monitoring;

(13) drug combinations;

(14) chemosensitization;

(15) chemopotentiation;

(16) post-treatment patient management;

(17) alternative medicine/therapeutic support;

(18) bulk drug product improvements;

(19) diluent systems;

(20) solvent systems;

(21) excipients;

(22) dosage forms;

(23) dosage kits and packaging;

(24) drug delivery systems;

(25) drug conjugate forms;

(26) compound analogs;

(27) prodrugs;

(28) multiple drug systems;

(29) biotherapeutic enhancement;

(30) biotherapeutic resistance modulation;

(31) radiation therapy enhancement;

(32) novel mechanisms of action;

(33) selective target cell population therapeutics;

(34) use with ionizing radiation;

(35) use with an agent enhancing its activity;

(36) use with an agent that counteracts myelosuppression; and

(37) use with an agent that increases the ability of the substituted hexitol to pass through the blood-brain barrier.

When LC is being treated, the method can further comprise administration of a therapeutically effective quantity of an additional agent for treatment of LC. Typically, the additional agent for treatment of LC is selected from the group consisting of cytarabine, methotrexate, thiotepa, 4-[(3-chloro-2-fluorophenyl)amino]-7-methoxyquinazolin-6-yl(2R)-2,4-dimethylpiperazine-1-carboxylate, microRNA 199b-5p, interleukin-2, a pyridine STAT3/STAT5 modulator, a substituted quinoxaline inhibitor of inhibiting IKKβ and the NFκB and mTOR pathways, rituximab, irinotecan, taurolidine, taurultam, VEGFR-3 fusion proteins, a reaction product of taurultam with glucose, temozolomide, 4-hydroperoxycyclophosphamide, platinum-transferrin, phenylbenzothiazole, stilbene, biphenylalkyne, pyridine derivatives, 7-benzyl-10-(2-methylbenzyl)-2,6,7,8,9,10-hexahydroimidazo[1,2-a]pyrido[4,3-d]pyrimidin-5(3H)-one, 4-iodo-3-nitrobenzamide, interferon-α, interferon-β, a STAT3 inhibitor, coenzyme Q10, arabino-2′-O-methyl nucleosides and derivatives thereof, ricin mutants, methylol taurinamide, methylol-taurultam, an aminoglycan of taurultam, benzimidazole thiophene compounds, chlorambucil, temozolomide, thalidomide, and lenalidomide.

Yet another aspect of the present invention is a composition to improve the efficacy and/or reduce the side effects of suboptimally administered drug therapy employing a substituted hexitol derivative for the treatment of leptomeningeal carcinomatosis (LC) comprising an alternative selected from the group consisting of:

(1) a therapeutically effective quantity of a modified substituted hexitol derivative or a derivative, analog, or prodrug of a substituted hexitol derivative or a modified substituted hexitol derivative, wherein the modified substituted hexitol derivative or the derivative, analog or prodrug of the substituted hexitol derivative or modified substituted hexitol derivative possesses increased therapeutic efficacy or reduced side effects for treatment of LC as compared with an unmodified substituted hexitol derivative;

(2) a composition comprising:
(i) a therapeutically effective quantity of a substituted hexitol derivative, a modified substituted hexitol derivative, or a derivative, analog, or prodrug of a substituted hexitol derivative or a modified substituted hexitol derivative; and
(ii) at least one additional therapeutic agent, therapeutic agent subject to chemosensitization, therapeutic agent subject to chemopotentiation, diluent, excipient, solvent system, drug delivery system, agent to counteract myelosuppression, or agent that increases the ability of the substituted hexitol to pass through the blood-brain barrier, wherein the composition possesses increased therapeutic efficacy or reduced side effects for treatment of LC as compared with an unmodified substituted hexitol derivative;

(3) a therapeutically effective quantity of a substituted hexitol derivative, a modified substituted hexitol derivative or a derivative, analog, or prodrug of a substituted hexitol derivative or a modified substituted hexitol derivative that is incorporated into a dosage form, wherein the substituted hexitol derivative, the modified substituted hexitol derivative or the derivative, analog, or prodrug of a substituted hexitol derivative or a modified substituted hexitol derivative incorporated into the dosage form possesses increased therapeutic efficacy or reduced side effects for treatment of LC as compared with an unmodified substituted hexitol derivative;

(4) a therapeutically effective quantity of a substituted hexitol derivative, a modified substituted hexitol derivative or a derivative, analog, or prodrug of a substituted hexitol derivative or a modified substituted hexitol derivative that is incorporated into a dosage kit and packaging, wherein the substituted hexitol derivative, the modified substituted hexitol derivative or the derivative, analog, or prodrug of a substituted hexitol derivative or a modified substituted hexitol derivative incorporated into the dosage kit and packaging possesses increased therapeutic efficacy or reduced side effects for treatment of LC as compared with an unmodified substituted hexitol derivative; and

(5) a therapeutically effective quantity of a substituted hexitol derivative, a modified substituted hexitol derivative or a derivative, analog, or prodrug of a substituted hexitol derivative or a modified substituted hexitol derivative that is subjected to a bulk drug product improvement, wherein substituted hexitol derivative, a modified substituted hexitol derivative or a derivative, analog, or prodrug of a substituted hexitol derivative or a modified substituted hexitol derivative subjected to the bulk drug product improvement possesses increased therapeutic efficacy or reduced side effects for treatment of LC as compared with an unmodified substituted hexitol derivative.

The composition can further comprise a therapeutically effective quantity of an additional therapeutic agent for treatment of leptomeningeal carcinomatosis. Typically, the additional therapeutic agent for treatment of leptomeningeal carcinomatosis is selected from the group consisting of cytarabine, methotrexate, thiotepa, 4-[(3-chloro-2-fluorophenyl)amino]-7-methoxyquinazolin-6-yl (2R)-2,4-dimethylpiperazine-1-carboxylate, microRNA 199b-5p, interleukin-2, a pyridine STAT3/STAT5 modulator, a substituted quinoxaline inhibitor of inhibiting IKKβ and the NFκB and mTOR pathways, rituximab, irinotecan, taurolidine, taurultam, VEGFR-3 fusion proteins, a reaction product of taurultam with glucose, temozolomide, 4-hydroperoxycyclophosphamide, platinum-transferrin, phenylbenzothiazole, stilbene, biphenylalkyne, pyridine derivatives, 7-benzyl-10-(2-methylbenzyl)-2,6,7,8,9,10-hexahydroimidazo[1,2-a]pyrido[4,3-d]pyrimidin-5(3H)-one, 4-iodo-3-nitrobenzamide, interferon-α, interferon-β, a STAT3 inhibitor, coenzyme Q10, arabino-2′-O-methyl nucleosides and derivatives thereof, ricin mutants, methylol taurinamide, methylol-taurultam, an aminoglycan of taurultam, benzimidazole thiophene compounds, chlorambucil, temozolomide, thalidomide, and lenalidomide. The method can also further comprise, subsequent to the administration of an initial dose of the substituted hexitol derivative selected from the group consisting of dianhydrogalactitol, a derivative or analog of dianhydrogalactitol, diacetyldianhydrogalactitol, and a derivative or analog of diacetyldianhydrogalactitol: (1) determining the quantity of a protein associated with the activation of the DNA repair pathway to determine the extent of the activation of the DNA repair pathway; and (2) adjusting the dose of the substituted hexitol derivative selected from the group consisting of dianhydrogalactitol, a derivative or analog of dianhydrogalactitol, diacetyldianhydrogalactitol, and a derivative or analog of diacetyldianhydrogalactitol in response to the extent of the DNA repair pathway. Typically, the protein associated with the activation of the DNA repair pathway is selected from the group consisting of phosphorylated ATM, phosphorylated RPA32, and 7H2A.X.

### BRIEF DESCRIPTION OF THE DRAWINGS

The following invention will become better understood with reference to the specification, appended claims, and accompanying drawings, where:

FIG. 1 is a diagram showing the activity of dianhydrogalactitol in inducing N⁷-guanine inter-strand DNA crosslinking.

FIG. 2 is a diagram showing DNA damage repair signaling pathways.

FIG. 3 is a diagram showing the two most common DNA double-strand break repair pathways in mammalian cells; homologous recombination (HR) and non-homologous end joining (NHEJ).

FIG. 4 is a diagram showing a crystal violet assay for viability following administration of VAL-083 for 72 hours for six human cell lines: prostate cancer cell lines PC3 and LNCaP in the top panel; NSCLC cell lines A549, H23, H1792, and H2122 in the bottom panel.

FIG. 5 is a diagram with graphs showing the effect of VAL-083 treatment for 72 hours at various concentrations on growth inhibition for PC3, LNCaP, H1792, and H2122, showing IC₅₀ for VAL-083 for these cell lines.

FIG. 6 shows cell cycle analyses for LNCaP treated with 1 μM, 2.5 μM, 5 μM, and 10 μM of VAL-083 for 24 hours or 48 hours, and a control with no treatment, showing the proportions of the cells in G1, S, and G2/M phase.

FIG. 7 shows cell cycle analysis for LNCaP treated with 1 μM, 2.5 μM, 5 μM, and 10 μM of VAL-083 or cisplatin for 24 hours, 48 hours, or 72 hours, together with controls with no treatment, showing the proportions of the cells in G1, S, and G2/M phase.

FIG. 8 shows cell cycle analysis for PC3 treated with 1 μM, 2.5 μM, 5 μM, and 10 μM of VAL-083 or cisplatin for 24 hours, 48 hours, or 72 hours, together with controls with no treatment, showing the proportions of the cells in G1, S, and G2/M phase.

FIG. 9 shows that VAL-083 treatment induces DNA double strand breaks (DSB) in PC3 cells, H1792 cells, and H2122 cells. DSB triggers the phosphorylation of the histone variant H2AX (γH2AX) which plays critical roles in DNA damage response, and the accumulation of γH2AX in PC3 cells, H1792 cells, and H2122 cells after VAL-083 treatment is shown in Western blots. GAPDH is shown as a control. FIG. 9 shows that γH2AX is detectable at around 24 hours and lasted for 48-72 hours after removal of the cells from the medium.

FIG. 10 shows that VAL-083 treatment activated DNA damage signaling pathways as demonstrated by expression of phospho-ATM (S1981) and phospho-RPA32 (S33), especially in PC3 and H2122 cells. In the left panel, results for PC3 cells (VAL-083 at 51.4 μM) and LNCaP cells (VAL-083 at 9.18 μM) are shown. In the right panel, results for A549 cells (VAL-083 at 6.89 μM) and H2122 cells (VAL-083 at 24.46 μM) are shown. For each cell line, a control is shown, and results are shown (Western blots) for 1 hour of treatment, 1 hour of treatment followed by a 19-hour washout, and 1 hour of treatment followed by a 24-hour washout, respectively. Results are shown for each time point for p-ATM (S1981), total ATM, p-RPA32 (33), total RPA32, γH2A.X, and total H2A.X.

FIG. 11 shows the results of immunofluorescent staining after VAL-083 treatment in PC3 cells (left panel) and A549 cells (right panel). The results show increased γH2A.X and late S/G2 phase cell cycle arrest after VAL-083 treatment in PC3 cells. VAL-083 was administered at 2×ID₅₀ for 1 hour. In each panel, the results, in a clockwise direction, are shown for untreated cells at 1 hour, VAL-083 treatment for 1 hour, VAL-083 treatment for 1 hour followed by a 24-hour washout (WO), and untreated cells for 24 hours. Cyclin A2 is also shown.

FIG. 12 shows VAL-083 induced activation of γH2AX at around 24 hours of treatment.

FIG. 13 shows PI staining and shows that VAL-083 treatment led to cell cycle arrest at S/G2 phase.

FIG. 14 shows PI staining and shows the results of serum starvation for 24 hours before 5 μM VAL-083 treatment.

FIG. 15 shows IC₅₀ analysis by crystal violet assay after VAL-083 treatment for 72 hours.

FIG. 16 shows the persistence of γH2AX activation for 24-72 hours after VAL083 treatment (IC₅₀) for 24 hours.

FIG. 17 shows the ATM-Chk2 and ATR-Chk1 pathways.

FIG. 18 shows that ATM is recruited to the DSB sites and triggers autophosphorylation.

FIG. 19 shows that VAL-083 treatment for 1 hour activated p-ATM (S1981) and p-RPA32 (S33).

FIG. 20 shows the cell cycle and its association with cyclin expression.

FIG. 21 shows that VAL-083 pulse treatment strongly increased γH2AX and cyclin A2 expression with cell cycle arrest at S/G2 phase.

FIG. 22 shows that VAL-083 treatment (51.4 μM) activated p-ATM (S1981).

FIG. 23 shows that VAL-083 treatment induced activation of pChk2 (T68).

FIG. 24 shows that there was no activation of pChk1 (S345) with VAL-083 treatment.

FIG. 25 depicts a genome-scale CRISPR-Cas9 knockout (GeCKO) library.

FIG. 26 depicts the experimental procedures for developing the genome-scale CRISPR-Cas9 knockout (GeCKO) library of FIG. 25.

FIG. 27 depicts cloning and proposed experiments.

FIG. 28 is a graph showing the IC₅₀ of several bladder cancer cell lines with treatment by dianhydrogalactitol (“VAL-083”) for 72 hours, including 253JBV, UC16, UC13, UC3, T24, and UC14.

### DETAILED DESCRIPTION OF THE INVENTION

The compound dianhydrogalactitol (DAG) has been shown to have substantial efficacy in inhibiting the growth of glioblastoma cells. In the case of glioblastoma, DAG has proven to be more effective in suppressing the growth of glioblastoma cells than temozolomide (TMZ), the current chemotherapy of choice for glioblastoma. As detailed below, DAG can effectively cross the blood-brain barrier and can effectively suppress the growth of cancer stem cells (CSCs). DAG acts independently of the MGMT repair mechanism. DAG has also shown efficacy in treating non-small-cell lung carcinoma (NSCLC) and ovarian cancer, as detailed further below.

As detailed further below, nine cancer cell lines were evaluated by cell proliferation assay for DAG sensitivity. Relatively resistant cell lines (PC3 and H2122) and relatively sensitive cells (LNCaP and H1792) were chosen to investigate DNA damage response induced by DAG. DAG treatment led to cell cycle arrest at S and G₂ phase. The data also showed activation of phosphorylation of histone variant H2A.X (γH2A.X) due to DNA damage response to DAG-induced double strand breaks. Alterations in DNA damage repair signaling pathways may be responsible for the sensitivity or resistance to DAG in cancer cells.

The structure of dianhydrogalactitol (DAG) is shown in Formula (I), below.

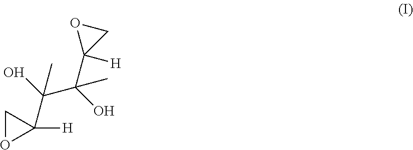

As detailed below, other substituted hexitols can be used in methods and compositions according to the present invention. In general, the substituted hexitols usable in methods and compositions according to the present invention include galactitols, substituted galacitols, dulcitols, and substituted dulcitols, including dianhydrogalactitol, diacetyldianhydrogalactitol, dibromodulcitol, and derivatives and analogs thereof. Typically, the substituted hexitol derivative is selected from the group consisting of dianhydrogalactitol, derivatives of dianhydrogalactitol, diacetyldianhydrogalactitol, derivatives of diacetyldianhydrogalactitol, dibromodulcitol, and derivatives of dibromodulcitol. Preferably, the substituted hexitol derivative is dianhydrogalactitol. These galactitols, substituted galacitols, dulcitols, and substituted dulcitols are either alkylating agents or prodrugs of alkylating agents, as discussed further below.

Also within the scope of the invention are derivatives of dianhydrogalactitol that, for example, have one or both hydrogens of the two hydroxyl groups of dianhydrogalactitol replaced with lower alkyl, have one or more of the hydrogens attached to the two epoxide rings replaced with lower alkyl, or have the methyl groups present in dianhydrogalactitol and that are attached to the same carbons that bear the hydroxyl groups replaced with C₂-C₆ lower alkyl or substituted with, for example, halo groups by replacing a hydrogen of the methyl group with, for example a halo group. As used herein, the term “halo group,” without further limitation, refers to one of fluoro, chloro, bromo, or iodo. As used herein, the term “lower alkyl,” without further limitation, refers to C₁-C₆ groups and includes methyl. The term “lower alkyl” can be further limited, such as “C₂-C₆ lower alkyl,” which excludes methyl. The term “lower alkyl”, unless further limited, refers to both straight-chain and branched alkyl groups. These groups can, optionally, be further substituted, as described below.

The structure of diacetyldianhydrogalactitol is shown in Formula (II), below.

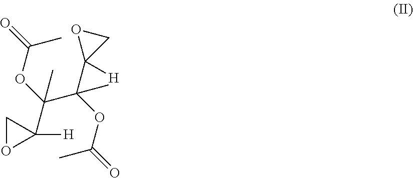 [SMILES: CC(=O)OC(C)(C1CO1)C(C)(OC(C)=O)C1CO1]

Also within the scope of the invention are derivatives of diacetyldianhydrogalactitol that, for example, have one or both of the methyl groups that are part of the acetyl moieties replaced with C₂-C₆ lower alkyl, have one or both of the hydrogens attached to the epoxide ring replaced with lower alkyl, or have the methyl groups attached to the same carbons that bear the acetyl groups replaced with lower alkyl or substituted with, for example, halo groups by replacing a hydrogen with, for example, a halo group.

The structure of dibromodulcitol is shown in Formula (III), below. Dibromodulcitol can be produced by the reaction of dulcitol with hydrobromic acid at elevated temperatures, followed by crystallization of the dibromodulcitol. Some of the properties of dibromodulcitol are described in N. E. Mischler et al., “Dibromoducitol,” Cancer Treat. Rev. 6: 191-204 (1979). In particular, dibromodulcitol, as an α, ω-dibrominated hexitol, dibromodulcitol shares many of the biochemical and biological properties of similar drugs such as dibromomannitol and mannitol myleran. Activation of dibromodulcitol to the diepoxide dianhydrogalactitol occurs in vivo, and dianhydrogalactitol may represent a major active form of the drug; this means that dibromogalactitol has many of the properties of a prodrug. Absorption of dibromodulcitol by the oral route is rapid and fairly complete. Dibromodulcitol has known activity in melanoma, lymphoma (both Hodgkins and non-Hodgkins), colorectal cancer, acute lymphoblastic leukemia and has been shown to lower the incidence of central nervous system leukemia, non-small cell lung cancer, cervical carcinoma, bladder carcinoma, and metastatic hemangiopericytoma.

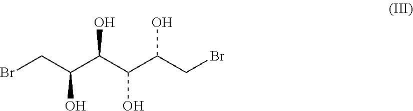 [SMILES: O[C@H]([C@H](O)[C@@H](O)CBr)[C@H](O)CBr]

Also within the scope of the invention are derivatives of dibromodulcitol that, for example, have one or more hydrogens of the hydroxyl groups replaced with lower alkyl, or have one or both of the bromo groups replaced with another halo group such as chloro, fluoro, or iodo.

In general, for optional substituents at saturated carbon atoms such as those that are part of the structures of dianhydrogalactitol, derivatives of dianhydrogalactitol, diacetyldianhydrogalactitol, derivatives of diacetyldianhydrogalactitol, dibromodulcitol, and derivatives of dibromodulcitol, the following substituents can be employed: C₆-C₁₀ aryl, heteroaryl containing 1-4 heteroatoms selected from N, O, and S, C₁-C₁₀ alkyl, C₁-C₁₀ alkoxy, cycloalkyl, F, amino (NR¹R²), nitro, —SR, —S(O)R, —S(O₂)R, —S(O₂)NR¹R², and —CONR¹R², which can in turn be optionally substituted. Further descriptions of potential optional substituents are provided below.

Optional substituents as described above that are within the scope of the present invention do not substantially affect the activity of the derivative or the stability of the derivative, particularly the stability of the derivative in aqueous solution. Definitions for a number of common groups that can be used as optional substituents are provided below; however, the omission of any group from these definitions cannot be taken to mean that such a group cannot be used as an optional substituent as long as the chemical and pharmacological requirements for an optional substituent are satisfied.

As used herein, the term “alkyl” refers to an unbranched, branched, or cyclic saturated hydrocarbyl residue, or a combination thereof, of from 1 to 12 carbon atoms that can be optionally substituted; the alkyl residues contain only C and H when unsubstituted. Typically, the unbranched or branched saturated hydrocarbyl residue is from 1 to 6 carbon atoms, which is referred to herein as “lower alkyl.” When the alkyl residue is cyclic and includes a ring, it is understood that the hydrocarbyl residue includes at least three carbon atoms, which is the minimum number to form a ring. As used herein, the term “alkenyl” refers to an unbranched, branched or cyclic hydrocarbyl residue having one or more carbon-carbon double bonds. As used herein, the term “alkynyl” refers to an unbranched, branched, or cyclic hydrocarbyl residue having one or more carbon-carbon triple bonds; the residue can also include one or more double bonds. With respect to the use of “alkenyl” or “alkynyl,” the presence of multiple double bonds cannot produce an aromatic ring. As used herein, the terms “hydroxyalkyl,” “hydroxyalkenyl,” and “hydroxyalkynyl,” respectively, refer to an alkyl, alkenyl, or alkynyl group including one or more hydroxyl groups as substituents; as detailed below, further substituents can be optionally included. As used herein, the term “aryl” refers to a monocyclic or fused bicyclic moiety having the well-known characteristics of aromaticity; examples include phenyl and naphthyl, which can be optionally substituted. As used herein, the term “hydroxyaryl” refers to an aryl group including one or more hydroxyl groups as substituents; as further detailed below, further substituents can be optionally included. As used herein, the term “heteroaryl” refers to monocyclic or fused bicylic ring systems that have the characteristics of aromaticity and include one or more heteroatoms selected from O, S, and N. The inclusion of a heteroatom permits aromaticity in 5-membered rings as well as in 6-membered rings. Typical heteroaromatic systems include monocyclic C₅-C₆ heteroaromatic groups such as pyridyl, pyrimidyl, pyrazinyl, thienyl, furanyl, pyrrolyl, pyrazolyl, thiazolyl, oxazolyl, triazolyl, triazinyl, tetrazolyl, tetrazinyl, and imidazolyl, as well as the fused bicyclic moieties formed by fusing one of these monocyclic heteroaromatic groups with a phenyl ring or with any of the heteroaromatic monocyclic groups to form a C₈-C₁₀ bicyclic group such as indolyl, benzimidazolyl, indazolyl, benzotriazolyl, isoquinolyl, quinolyl, benzothiazolyl, benzofuranyl, pyrazolylpyridyl, quinazolinyl, quinoxalinyl, cinnolinyl, and other ring systems known in the art. Any monocyclic or fused ring bicyclic system that has the characteristics of aromaticity in terms of delocalized electron distribution throughout the ring system is included in this definition. This definition also includes bicyclic groups where at least the ring that is directly attached to the remainder of the molecule has the characteristics of aromaticity, including the delocalized electron distribution that is characteristic of aromaticity. Typically the ring systems contain 5 to 12 ring member atoms and up to four heteroatoms, wherein the heteroatoms are selected from the group consisting of N, O, and S. Frequently, the monocyclic heteroaryls contain 5 to 6 ring members and up to three heteroatoms selected from the group consisting of N, O, and S; frequently, the bicyclic heteroaryls contain 8 to 10 ring members and up to four heteroatoms selected from the group consisting of N, O, and S. The number and placement of heteroatoms in heteroaryl ring structures is in accordance with the well-known limitations of aromaticity and stability, where stability requires the heteroaromatic group to be stable enough to be exposed to water at physiological temperatures without rapid degradation. As used herein, the term “hydroxheteroaryl” refers to a heteroaryl group including one or more hydroxyl groups as substituents; as further detailed below, further substituents can be optionally included. As used herein, the terms “haloaryl” and “haloheteroaryl” refer to aryl and heteroaryl groups, respedively, substituted with at least one halo group, where “halo” refers to a halogen selected from the group consisting of fluorine, chlorine, bromine, and iodine, typically, the halogen is selected from the group consisting of chlorine, bromine, and iodine; as detailed below, further substituents can be optionally included. As used herein, the terms “haloalkyl,” “haloalkenyl,” and “haloalkynyl” refer to alkyl, alkenyl, and alkynyl groups, respectively, substituted with at least one halo group, where “halo” refers to a halogen selected from the group consisting of fluorine, chlorine, bromine, and iodine, typically, the halogen is selected from the group consisting of chlorine, bromine, and iodine; as detailed below, further substituents can be optionally included.

As used herein, the term “optionally substituted” indicates that the particular group or groups referred to as optionally substituted may have no non-hydrogen substituents, or the group or groups may have one or more non-hydrogen substituents consistent with the chemistry and pharmacological activity of the resulting molecule. If not otherwise specified, the total number of such substituents that may be present is equal to the total number of hydrogen atoms present on the unsubstituted form of the group being described; fewer than the maximum number of such substituents may be present. Where an optional substituent is attached via a double bond, such as a carbonyl oxygen (C═O), the group takes up two available valences on the carbon atom to which the optional substituent is attached, so the total number of substituents that may be included is reduced according to the number of available valiences. As used herein, the term “substituted,” whether used as part of “optionally substituted” or otherwise, when used to modify a specific group, moiety, or radical, means that one or more hydrogen atoms are, each, independently of each other, replaced with the same or different substituent or substituents.

Substituent groups useful for substituting saturated carbon atoms in the specified group, moiety, or radical include, but are not limited to, —Z^{a}, ═O, —OZ^{b}, —SZ^{b}, ═S⁻, —NZ^{c}Z^{c}, ═NZ^{b}, ═N—OZ^{b}, trihalomethyl, —CF₃, —CN, —OCN, —SCN, —NO, —NO₂, ═N₂, —N₃, —S(O)₂Z^{b}, —S(O)₂NZ^{b}, —S(O₂)O⁻, —S(O₂)OZ^{b}, —OS(O₂)OZ^{b}, —OS(O₂)O⁻, —OS(O₂)OZ^{b}, —P(O)(O⁻)₂, —P(O)(OZ^{b})(O⁻), —P(O)(OZ^{b})(OZ^{b}), —C(O)Z^{b}, —C(S)Z^{b}, —C(NZ^{b})Z^{b}, —C(O)O⁻, —C(O)OZ^{b}, —C(S)OZ^{b}, —C(O)NZ^{c}Z^{c}, —C(NZ^{b})NZ^{c}Z^{c}, —OC(O)Z^{b}, —OC(S)Z^{b}, —OC(O)O⁻, —OC(O)OZ^{b}, —OC(S)OZ^{b}, —NZ^{b}C(O)Z^{b}, —NZ^{b}C(S)Z^{b}, —NZ^{b}C(O)O⁻, —NZ^{b}C(O)OZ^{b}, —NZ^{b}C(S)OZ^{b}, —NZ^{b}C(O)NZ^{c}Z^{c}, —NZ^{b}C(NZ^{b})Z^{b}, —NZ^{b}C(NZ^{b})NZ^{c}Z^{c}, wherein Z^{a} is selected from the group consisting of alkyl, cycloalkyl, heteroalkyl, cycloheteroalkyl, aryl, arylalkyl, heteroaryl and heteroarylalkyl; each Z^{b} is independently hydrogen or Z^{a}; and each Z^{c} is independently Z^{b} or, alternatively, the two Z^{c′}s may be taken together with the nitrogen atom to which they are bonded to form a 4-, 5-, 6-, or 7-membered cycloheteroalkyl ring structure which may optionally include from 1 to 4 of the same or different heteroatoms selected from the group consisting of N, O, and S. As specific examples, —NZ^{c}Z^{c} is meant to include —NH₂, —NH-alkyl, —N-pyrrolidinyl, and —N-morpholinyl, but is not limited to those specific alternatives and includes other alternatives known in the art. Similarly, as another specific example, a substituted alkyl is meant to include -alkylene-O-alkyl, -alkylene-heteroaryl, -alkylene-cycloheteroaryl, -alkylene-C(O)OZ^{b}, -alkylene-C(O)NZ^{b}Z^{b}, and —CH₂—CH₂—C(O)—CH₃, but is not limited to those specific alternatives and includes other alternatives known in the art. The one or more substituent groups, together with the atoms to which they are bonded, may form a cyclic ring, including, but not limited to, cycloalkyl and cycloheteroalkyl.

Similarly, substituent groups useful for substituting unsaturated carbon atoms in the specified group, moiety, or radical include, but are not limited to, —Z^{a}, halo, O⁻, —OZ^{b}, —SZ^{b}, —S⁻, —NZ^{c}Z^{c}, trihalomethyl, —CF₃, —CN, —OCN, —SCN, —NO, —NO₂, —N₃, —S(O)₂Z^{b}, —S(O₂)O, —S(O₂)OZ^{b}, —OS(O₂)OZ^{b}, —OS(O₂)O⁻, —P(O)(O⁻)₂, —P(O)(OZ^{b})(O), —P(O)(OZ^{b})(OZ^{b}), —C(O)Z^{b}, —C(S)Z^{b}, —C(NZ^{b})Z^{b}, —C(O)O⁻, —C(O)OZ^{b}, —C(S)OZ^{b}, —C(O)NZ^{c}Z^{c}, —C(NZ^{b})NZ^{c}Z^{c}, —OC(O)Z^{b}, —OC(S)Z^{b}, —OC(O)O⁻, —OC(O)OZ^{b}, —OC(S)OZ^{b}, —NZ^{b}C(O)OZ^{b}, —NZ^{b}C(S)OZ^{b}, —NZ^{b}C(O)NZ^{c}Z^{c}, —NZ^{b}C(NZ^{b})Z^{b}, and —NZ^{b}C(NZ^{b})NZ^{c}Z^{c}, wherein Z^{a}, Z^{b}, and Z^{c} are as defined above.

Similarly, substituent groups useful for substituting nitrogen atoms in heteroalkyl and cycloheteroalkyl groups include, but are not limited to, —Z^{a}, halo, —O⁻, —OZ^{b}, —SZ^{b}, —S⁻, —NZ^{c}Z^{c}, trihalomethyl, —CF₃, —CN, —OCN, —SCN, —NO, —NO₂, —S(O)₂Z^{b}, —S(O₂)O⁻, —S(O₂)OZ^{b}, —OS(O₂)OZ^{b}, —OS(O₂)O⁻, −P(O)(O⁻)₂, —P(O)(OZ^{b})(O), —P(O)(OZ^{b})(OZ^{b}), —C(O)Z^{b}, —C(S)Z^{b}, —C(NZ^{b})Z^{b}, —C(O)OZ^{b}, —C(S)OZ^{b}, —C(O)NZ^{c}Z^{c}, —C(NZ^{b})NZ^{c}Z^{c}, —OC(O)Z^{b}, —OC(S)Z^{b}, —OC(O)OZ^{b}, —OC(S)OZ^{b}, —NZ^{b}C(O)Z^{b}, —NZ^{b}C(S)Z^{b}, —NZ^{b}C(O)OZ^{b}, —NZ^{b}C(S)OZ^{b}, —NZ^{b}C(O)NZ^{c}Z^{c}, —NZ^{b}C(NZ^{b})Z^{b}, and —NZ^{b}C(NZ^{b})NZ^{c}Z^{c}, wherein Z^{a}, Z^{b}, and Z^{c} are as defined above.

The compounds described herein may contain one or more chiral centers and/or double bonds and therefore, may exist as stereoisomers, such as double-bond isomers (i.e., geometric isomers such as E and Z), enantiomers or diastereomers. The invention includes each of the isolated stereoisomeric forms (such as the enantiomerically pure isomers, the E and Z isomers, and other alternatives for stereoisomers) as well as mixtures of stereoisomers in varying degrees of chiral purity or percentage of E and Z, including racemic mixtures, mixtures of diastereomers, and mixtures of E and Z isomers. Accordingly, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The invention includes each of the isolated stereoisomeric forms as well as mixtures of stereoisomers in varying degrees of chiral purity, including racemic mixtures. It also encompasses the various diastereomers. Other structures may appear to depict a specific isomer, but that is merely for convenience, and is not intended to limit the invention to the depicted isomer. When the chemical name does not specify the isomeric form of the compound, it denotes any one of the possible isomeric forms or mixtures of those isomeric forms of the compound. In the case of enantiomers, they may be designated R- or S- according to the conventionally used Cahn-Ingold-Prelog nomenclature; in some cases as is conventional, such as for amino acids and carbohydrates, they may be designated D- and L-.

The compounds may also exist in several tautomeric forms, and the depiction herein of one tautomer is for convenience only, and is also understood to encompass other tautomers of the form shown. Accordingly, the chemical structures depicted herein encompass all possible tautomeric forms of the illustrated compounds. The term “tautomer” as used herein refers to isomers that change into one another with great ease so that they can exist together in equilibrium; the equilibrium may strongly favor one of the tautomers, depending on stability considerations. For example, ketone and enol are two tautomeric forms of one compound.

As used herein, the term “solvate” means a compound formed by solvation (the combination of solvent molecules with molecules or ions of the solute), or an aggregate that consists of a solute ion or molecule, i.e., a compound of the invention, with one or more solvent molecules. When water is the solvent, the corresponding solvate is “hydrate.” Examples of hydrate include, but are not limited to, hem ihydrate, monohydrate, dihydrate, trihydrate, hexahydrate, and other water-containing species. It should be understood by one of ordinary skill in the art that the pharmaceutically acceptable salt, and/or prodrug of the present compound may also exist in a solvate form. The solvate is typically formed via hydration which is either part of the preparation of the present compound or through natural absorption of moisture by the anhydrous compound of the present invention.

As used herein, the term “ester” means any ester of a present compound in which any of the —COOH functions of the molecule is replaced by a —COOR function, in which the R moiety of the ester is any carbon-containing group which forms a stable ester moiety, including but not limited to alkyl, alkenyl, alkynyl, cycloalkyl, cycloalkylalkyl, aryl, arylalkyl, heterocyclyl, heterocyclylalkyl and substituted derivatives thereof. The hydrolysable esters of the present compounds are the compounds whose carboxyls are present in the form of hydrolyzable ester groups. That is, these esters are pharmaceutically acceptable and can be hydrolyzed to the corresponding carboxyl acid in vivo.

In addition to the substituents described above, alkyl, alkenyl and alkynyl groups can alternatively or in addition be substituted by C₁-C₈ acyl, C₂-C₈ heteroacyl, C₆-C₁₀ aryl, C₃-C₈ cycloalkyl, C₃-C₈ heterocyclyl, or C₅-C₁₀ heteroaryl, each of which can be optionally substituted. Also, in addition, when two groups capable of forming a ring having 5 to 8 ring members are present on the same or adjacent atoms, the two groups can optionally be taken together with the atom or atoms in the substituent groups to which they are attached to form such a ring. “Heteroalkyl,” “heteroalkenyl,” and “heteroalkynyl” and the like are defined similarly to the corresponding hydrocarbyl (alkyl, alkenyl and alkynyl) groups, but the ‘hetero’ terms refer to groups that contain 1-3 O, S or N heteroatoms or combinations thereof within the backbone residue; thus at least one carbon atom of a corresponding alkyl, alkenyl, or alkynyl group is replaced by one of the specified heteroatoms to form, respectively, a heteroalkyl, heteroalkenyl, or heteroalkynyl group. For reasons of chemical stability, it is also understood that, unless otherwise specified, such groups do not include more than two contiguous heteroatoms except where an oxo group is present on N or S as in a nitro or sulfonyl group. While “alkyl” as used herein includes cycloalkyl and cycloalkylalkyl groups, the term “cycloalkyl” may be used herein to describe a carbocyclic non-aromatic group that is connected via a ring carbon atom, and “cycloalkylalkyl” may be used to describe a carbocyclic non-aromatic group that is connected to the molecule through an alkyl linker.

“Heteroalkyl,” “heteroalkenyl,” and “heteroalkynyl” and the like are defined similarly to the corresponding hydrocarbyl (alkyl, alkenyl and alkynyl) groups, but the ‘hetero’ terms refer to groups that contain 1-3 O, S or N heteroatoms or combinations thereof within the backbone residue; thus at least one carbon atom of a corresponding alkyl, alkenyl, or alkynyl group is replaced by one of the specified heteroatoms to form, respectively, a heteroalkyl, heteroalkenyl, or heteroalkynyl group. For reasons of chemical stability, it is also understood that, unless otherwise specified, such groups do not include more than two contiguous heteroatoms except where an oxo group is present on N or S as in a nitro or sulfonyl group.

While “alkyl” as used herein includes cycloalkyl and cycloalkylalkyl groups, the term “cycloalkyl” may be used herein to describe a carbocyclic non-aromatic group that is connected via a ring carbon atom, and “cycloalkylalkyl” may be used to describe a carbocyclic non-aromatic group that is connected to the molecule through an alkyl linker.

Similarly, “heterocyclyl” may be used to describe a non-aromatic cyclic group that contains at least one heteroatom (typically selected from N, O and S) as a ring member and that is connected to the molecule via a ring atom, which may be C (carbon-linked) or N (nitrogen-linked); and “heterocyclylalkyl” may be used to describe such a group that is connected to another molecule through a linker. The heterocyclyl can be fully saturated or partially saturated, but non-aromatic. The sizes and substituents that are suitable for the cycloalkyl, cycloalkylalkyl, heterocyclyl, and heterocyclylalkyl groups are the same as those described above for alkyl groups. The heterocyclyl groups typically contain 1, 2 or 3 heteroatoms, selected from N, O and S as ring members; and the N or S can be substituted with the groups commonly found on these atoms in heterocyclic systems. As used herein, these terms also include rings that contain a double bond or two, as long as the ring that is attached is not aromatic. The substituted cycloalkyl and heterocyclyl groups also include cycloalkyl or heterocyclic rings fused to an aromatic ring or heteroaromatic ring, provided the point of attachment of the group is to the cycloalkyl or heterocyclyl ring rather than to the aromatic/heteroaromatic ring.

As used herein, “acyl” encompasses groups comprising an alkyl, alkenyl, alkynyl, aryl or arylalkyl radical attached at one of the two available valence positions of a carbonyl carbon atom, and heteroacyl refers to the corresponding groups wherein at least one carbon other than the carbonyl carbon has been replaced by a heteroatom chosen from N, O and S. Acyl and heteroacyl groups are bonded to any group or molecule to which they are attached through the open valence of the carbonyl carbon atom. Typically, they are C₁-C₈ acyl groups, which include formyl, acetyl, pivaloyl, and benzoyl, and C₂-C₈ heteroacyl groups, which include methoxyacetyl, ethoxycarbonyl, and 4-pyridinoyl.

Acyl and heteroacyl groups are bonded to any group or molecule to which they are attached through the open valence of the carbonyl carbon atom. Typically, they are C₁-C₈ acyl groups, which include formyl, acetyl, pivaloyl, and benzoyl, and C₂-C₈ heteroacyl groups, which include methoxyacetyl, ethoxycarbonyl, and 4-pyridinoyl.

Similarly, “arylalkyl” and “heteroarylalkyl” refer to aromatic and heteroaromatic ring systems which are bonded to their attachment point through a linking group such as an alkylene, including substituted or unsubstituted, saturated or unsaturated, cyclic or acyclic linkers. Typically the linker is C₁-C₈ alkyl. These linkers may also include a carbonyl group, thus making them able to provide substituents as an acyl or heteroacyl moiety. An aryl or heteroaryl ring in an arylalkyl or heteroarylalkyl group may be substituted with the same substituents described above for aryl groups. Preferably, an arylalkyl group includes a phenyl ring optionally substituted with the groups defined above for aryl groups and a C₁-C₄ alkylene that is unsubstituted or is substituted with one or two C₁-C₄ alkyl groups or heteroalkyl groups, where the alkyl or heteroalkyl groups can optionally cyclize to form a ring such as cyclopropane, dioxolane, or oxacyclopentane. Similarly, a heteroarylalkyl group preferably includes a C₅-C₆ monocyclic heteroaryl group that is optionally substituted with the groups described above as substituents typical on aryl groups and a C₁-C₄ alkylene that is unsubstituted or is substituted with one or two C₁-C₄ alkyl groups or heteroalkyl groups, or it includes an optionally substituted phenyl ring or C₅-C₆ monocyclic heteroaryl and a C₁-C₄ heteroalkylene that is unsubstituted or is substituted with one or two C₁-C₄ alkyl or heteroalkyl groups, where the alkyl or heteroalkyl groups can optionally cyclize to form a ring such as cyclopropane, dioxolane, or oxacyclopentane. Where an arylalkyl or heteroarylalkyl group is described as optionally substituted, the substituents may be on either the alkyl or heteroalkyl portion or on the aryl or heteroaryl portion of the group. The substituents optionally present on the alkyl or heteroalkyl portion are the same as those described above for alkyl groups generally; the substituents optionally present on the aryl or heteroaryl portion are the same as those described above for aryl groups generally.

“Arylalkyl” groups as used herein are hydrocarbyl groups if they are unsubstituted, and are described by the total number of carbon atoms in the ring and alkylene or similar linker. Thus a benzyl group is a C7-arylalkyl group, and phenylethyl is a C8-arylalkyl.

“Heteroarylalkyl” as described above refers to a moiety comprising an aryl group that is attached through a linking group, and differs from “arylalkyl” in that at least one ring atom of the aryl moiety or one atom in the linking group is a heteroatom selected from N, O and S. The heteroarylalkyl groups are described herein according to the total number of atoms in the ring and linker combined, and they include aryl groups linked through a heteroalkyl linker; heteroaryl groups linked through a hydrocarbyl linker such as an alkylene; and heteroaryl groups linked through a heteroalkyl linker. Thus, for example, C7-heteroarylalkyl would include pyridylmethyl, phenoxy, and N-pyrrolylmethoxy.

“Alkylene” as used herein refers to a divalent hydrocarbyl group; because it is divalent, it can link two other groups together. Typically it refers to —(CH₂)ₙ— where n is 1-8 and preferably n is 1-4, though where specified, an alkylene can also be substituted by other groups, and can be of other lengths, and the open valences need not be at opposite ends of a chain.

In general, any alkyl, alkenyl, alkynyl, acyl, or aryl or arylalkyl group that is contained in a substituent may itself optionally be substituted by additional substituents. The nature of these substituents is similar to those recited with regard to the primary substituents themselves if the substituents are not otherwise described.

“Amino” as used herein refers to —NH₂, but where an amino is described as “substituted” or “optionally substituted”, the term includes NR′R″ wherein each R′ and R″ is independently H, or is an alkyl, alkenyl, alkynyl, acyl, aryl, or arylalkyl group, and each of the alkyl, alkenyl, alkynyl, acyl, aryl, or arylalkyl groups is optionally substituted with the substituents described herein as suitable for the corresponding group; the R′ and R″ groups and the nitrogen atom to which they are attached can optionally form a 3- to 8-membered ring which may be saturated, unsaturated or aromatic and which contains 1-3 heteroatoms independently selected from N, O and S as ring members, and which is optionally substituted with the substituents described as suitable for alkyl groups or, if NR′R″ is an aromatic group, it is optionally substituted with the substituents described as typical for heteroaryl groups.

As used herein, the term “carbocycle,” “carbocyclyl,” or “carbocyclic” refers to a cyclic ring containing only carbon atoms in the ring, whereas the term “heterocycle” or “heterocyclic” refers to a ring comprising a heteroatom. The carbocyclyl can be fully saturated or partially saturated, but non-aromatic. For example, the carbocyclyl encompasses cycloalkyl. The carbocyclic and heterocyclic structures encompass compounds having monocyclic, bicyclic or multiple ring systems; and such systems may mix aromatic, heterocyclic, and carbocyclic rings. Mixed ring systems are described according to the ring that is attached to the rest of the compound being described.

As used herein, the term “heteroatom” refers to any atom that is not carbon or hydrogen, such as nitrogen, oxygen or sulfur. When it is part of the backbone or skeleton of a chain or ring, a heteroatom must be at least divalent, and will typically be selected from N, O, P, and S.

As used herein, the term “alkanoyl” refers to an alkyl group covalently linked to a carbonyl (C═O) group. The term “lower alkanoyl” refers to an alkanoyl group in which the alkyl portion of the alkanoyl group is C₁-C₆. The alkyl portion of the alkanoyl group can be optionally substituted as described above. The term “alkylcarbonyl” can alternatively be used. Similarly, the terms “alkenylcarbonyl” and “alkynylcarbonyl” refer to an alkenyl or alkynyl group, respectively, linked to a carbonyl group.

As used herein, the term “alkoxy” refers to an alkyl group covalently linked to an oxygen atom; the alkyl group can be considered as replacing the hydrogen atom of a hydroxyl group. The term “lower alkoxy” refers to an alkoxy group in which the alkyl portion of the alkoxy group is C₁-C₆. The alkyl portion of the alkoxy group can be optionally substituted as described above. As used herein, the term “haloalkoxy” refers to an alkoxy group in which the alkyl portion is substituted with one or more halo groups.

As used herein, the term “sulfo” refers to a sulfonic acid (—SO₃H) substituent.

As used herein, the term “sulfamoyl” refers to a substituent with the structure —S(O₂)NH₂, wherein the nitrogen of the NH₂ portion of the group can be optionally substituted as described above.

As used herein, the term “carboxyl” refers to a group of the structure —C(O₂)H.

As used herein, the term “carbamyl” refers to a group of the structure —C(O₂)NH₂, wherein the nitrogen of the NH₂ portion of the group can be optionally substituted as described above.

As used herein, the terms “monoalkylaminoalkyl” and “dialkylaminoalkyl” refer to groups of the structure -Alk₁-NH-Alk₂ and -Alk₁-N(Alk₂)(Alk₃), wherein Alk₁, Alk₂, and Alk₃ refer to alkyl groups as described above.

As used herein, the term “alkylsulfonyl” refers to a group of the structure —S(O)₂-Alk wherein Alk refers to an alkyl group as described above. The terms “alkenylsulfonyl” and “alkynylsulfonyl” refer analogously to sulfonyl groups covalently bound to alkenyl and alkynyl groups, respectively. The term “arylsulfonyl” refers to a group of the structure —S(O)₂—Ar wherein Ar refers to an aryl group as described above. The term “aryloxyalkylsulfonyl” refers to a group of the structure —S(O)₂-Alk-O—Ar, where Alk is an alkyl group as described above and Ar is an aryl group as described above. The term “arylalkylsulfonyl” refers to a group of the structure —S(O)₂-AlkAr, where Alk is an alkyl group as described above and Ar is an aryl group as described above.

As used herein, the term “alkyloxycarbonyl” refers to an ester substituent including an alkyl group wherein the carbonyl carbon is the point of attachment to the molecule. An example is ethoxycarbonyl, which is CH₃CH₂OC(O)—. Similarly, the terms “alkenyloxycarbonyl,” “alkynyloxycarbonyl,” and “cycloalkylcarbonyl” refer to similar ester substituents including an alkenyl group, alkenyl group, or cycloalkyl group respectively. Similarly, the term “aryloxycarbonyl” refers to an ester substituent including an aryl group wherein the carbonyl carbon is the point of attachment to the molecule. Similarly, the term “aryloxyalkylcarbonyl” refers to an ester substituent including an alkyl group wherein the alkyl group is itself substituted by an aryloxy group.

Other combinations of substituents are known in the art and, are described, for example, in U.S. Pat. No. 8,344,162 to Jung et al. For example, the term “thiocarbonyl” and combinations of substituents including “thiocarbonyl” include a carbonyl group in which a double-bonded sulfur replaces the normal double-bonded oxygen in the group. The term “alkylidene” and similar terminology refer to an alkyl group, alkenyl group, alkynyl group, or cycloalkyl group, as specified, that has two hydrogen atoms removed from a single carbon atom so that the group is double-bonded to the remainder of the structure.

For the aspects described below relating to improvement in the therapeutic employment of a substituted hexitol derivative, typically, the substituted hexitol derivative is selected from the group consisting of dianhydrogalactitol, derivatives of dianhydrogalactitol, diacetyldianhydrogalactitol, derivatives of diacetyldianhydrogalactitol, dibromodulcitol, and derivatives of dibromodulcitol, unless otherwise specified. Preferably, the substituted hexitol derivative is dianhydrogalactitol, unless otherwise specified. In some cases, derivatives of dianhydrogalactitol such as compound analogs or prodrugs are preferred, as stated below.

One aspect of the present invention is an improvement in the therapeutic employment of a substituted hexitol derivative such as dianhydrogalactitol for treatment of glioblastoma, NSCLC, or ovarian cancer made by alterations to the time that the compound is administered, the use of dose-modifying agents that control the rate of metabolism of the compound, normal tissue protective agents, and other alterations. General examples include: variations of infusion schedules (e.g., bolus i.v. versus continuous infusion), the use of lymphokines (e.g., G-CSF, GM-CSF, EPO) to increase leukocyte count for improved immune response or for preventing anemia caused by myelosuppressive agents, or the use of rescue agents such as leucovorin for 5-FU or thiosulfate for cisplatin treatment. Specific inventive examples for a substituted hexitol derivative such as dianhydrogalactitol for treatment of glioblastoma, NSCLC, or ovarian cancer include: continuous i.v. infusion for hours to days; biweekly administration; doses greater than 5 mg/m²/day; progressive escalation of dosing from 1 mg/m²/day based on patient tolerance; doses less than 1 mg/m² for greater than 14 days; use of caffeine to modulate metabolism; use of isoniazid to modulate metabolism; single and multiple doses escalating from 5 mg/m²/day via bolus; oral doses below 30 or above 130 mg/m²; oral dosages up to 40 mg/m² for 3 days and then a nadir/recovery period of 18-21 days; dosing at a lower level for an extended period (e.g., 21 days); dosing at a higher level; dosing with a nadir/recovery period longer than 21 days; dosing at a level to achieve a concentration of the substituted hexitol derivative such as dianhydrogalactitol in the cerebrospinal fluid (CSF) of equal to or greater than 5 μM; dosing at a level to achieve a cytotoxic concentration in the CSF for treatment of glioblastoma; the use of a substituted hexitol derivative such as dianhydrogalactitol as a single cytotoxic agent; administration on a 33-day cycle with a cumulative dose of about 9 mg/m²; administration on a 33-day cycle with a cumulative dose of about 10 mg/m²; administration on a 33-day cycle with a cumulative dose of about 20 mg/m²; administration on a 33-day cycle with a cumulative dose of about 40 mg/m²; administration on a 33-day cycle with a cumulative dose of about 80 mg/m²; administration on a 33-day cycle with a cumulative dose of about 160 mg/m²; administration on a 33-day cycle with a cumulative dose of about 240 mg/m²; administration so that the plasma half-life is about 1-2 hours; administration so that the Cₘₐₓ is <200 ng/ml; and administration so that the substituted hexitol derivative has a half life of >20 hours in the cerebrospinal fluid for treatment of glioblastoma.

Another aspect of the invention is an improvement in the therapeutic employment of a substituted hexitol derivative such as dianhydrogalactitol for treatment of glioblastoma, NSCLC, or ovarian cancer made by alterations in the route by which the compound is administered. General examples include: changing route from oral to intravenous administration and vice versa; or the use of specialized routes such as subcutaneous, intramuscular, intraarterial, intraperitoneal, intralesional, intralymphatic, intratumoral, intrathecal, intravesicular, intracranial. Specific inventive examples for a substituted hexitol derivative such as dianhydrogalactitol for treatment of glioblastoma, NSCLC, or ovarian cancer include: topical administration; oral administration; slow-release oral delivery; intrathecal administration; intraarterial administration; continuous infusion; intermittent infusion; intravenous administration; administration through a longer infusion; administration through IV push; and administration to maximize the concentration of the substituted hexitol derivative in the CSF for treatment of glioblastoma.

Yet another aspect of the invention is an improvement in the therapeutic employment of a substituted hexitol derivative such as dianhydrogalactitol made by changes in the schedule of administration. General examples include: daily administration, biweekly administration, or weekly administration. Specific inventive examples for a substituted hexitol derivative such as dianhydrogalactitol for treatment of glioblastoma, NSCLC, or ovarian cancer include: daily administration; weekly administration; weekly administration for three weeks; biweekly administration; biweekly administration for three weeks with a 1-2 week rest period; intermittent boost dose administration; daily administration for one week for multiple weeks; or administration on days 1, 2, and 3 of a 33-day cycle.

Yet another aspect of the invention is an improvement in the therapeutic employment of a substituted hexitol derivative such as dianhydrogalactitol for treatment of glioblastoma, NSCLC, or ovarian cancer made by alterations in the stage of disease at diagnosis/progression that the compound is administered. General examples include: the use of chemotherapy for non-resectable local disease, prophylactic use to prevent metastatic spread or inhibit disease progression or conversion to more malignant stages. Specific inventive examples for a substituted hexitol derivative such as dianhydrogalactitol for treatment of glioblastoma, NSCLC, or ovarian cancer include: use in an appropriate disease stage for glioblastoma, NSCLC, or ovarian cancer; use of the substituted hexitol derivative such as dianhydrogalactitol with angiogenesis inhibitors such as Avastin, a VEGF inhibitor, to prevent or limit metastatic spread, especially in the central nervous system; the use of a substituted hexitol derivative such as dianhydrogalactitol for newly diagnosed disease; the use of a substituted hexitol derivative such as dianhydrogalactitol for recurrent disease; the use of a substituted hexitol derivative such as dianhydrogalactitol for resistant or refractory disease; or the use of a substituted hexitol derivative such as dianhydrogalactitol for childhood glioblastoma.

Yet another aspect of the invention is an improvement in the therapeutic employment of a substituted hexitol derivative such as dianhydrogalactitol for treatment of glioblastoma, NSCLC, or ovarian cancer made by alterations to the type of patient that would best tolerate or benefit from the use of the compound. General examples include: use of pediatric doses for elderly patients, altered doses for obese patients; exploitation of co-morbid disease conditions such as diabetes, cirrhosis, or other conditions that may uniquely exploit a feature of the compound. Specific inventive examples for a substituted hexitol derivative such as dianhydrogalactitol for treatment of glioblastoma, NSCLC, or ovarian cancer include: patients with a disease condition characterized by a high level of a metabolic enzyme selected from the group consisting of histone deacetylase and ornithine decarboxylase; patients with a low or high susceptibility to a condition selected from the group consisting of thrombocytopenia and neutropenia; patients intolerant of GI toxicities; patients characterized by over- or under-expression of a gene selected from the group consisting of c-Jun, a GPCR, a signal transduction protein, VEGF, a prostate-specific gene, and a protein kinase; patients characterized by carrying extra copies of the EGFR gene for glioblastoma, NSCLC, or ovarian cancer; patients characterized by mutations in at least one gene selected from the group consisting of TP53, PDGFRA, IDH1, and NF1 for glioblastoma, NSCLC, or ovarian cancer; patients characterized by methylation or lack of methylation of the promoter of the MGMT gene; patients characterized by the presence of IDH1 wild-type gene; patients characterized by the presence of 1p/19q co-deletion; patients characterized by a high expression of MGMT; patients characterized by a low expression of MGMT; or patients characterized by a mutation in EGFR including, but not limited to, EGFR Variant III.

Yet another aspect of the invention is an improvement in the therapeutic employment of a substituted hexitol derivative such as dianhydrogalactitol for treatment of glioblastoma, NSCLC, or ovarian cancer made by more precise identification of a patient's ability to tolerate, metabolize and exploit the use of the compound as associated with a particular phenotype of the patient. General examples include: use of diagnostic tools and kits to better characterize a patient's ability to process/metabolize a chemotherapeutic agent or the susceptibility of the patient to toxicity caused by potential specialized cellular, metabolic, or organ system phenotypes. Specific inventive examples for a substituted hexitol derivative such as dianhydrogalactitol for treatment of glioblastoma, NSCLC, or ovarian cancer include: use of a diagnostic tool, a diagnostic technique, a diagnostic kit, or a diagnostic assay to confirm a patient's particular phenotype; use of a method for measurement of a marker selected from the group consisting of histone deacetylase, ornithine decarboxylase, VEGF, a protein that is a gene product of jun, and a protein kinase; surrogate compound testing; or low dose pre-testing for enzymatic status.

Yet another aspect of the invention is an improvement in the therapeutic employment of a substituted hexitol derivative such as dianhydrogalactitol for treatment of glioblastoma, NSCLC, or ovarian cancer made by more precise identification of a patient's ability to tolerate, metabolize and exploit the use of the compound as associated with a particular genotype of the patient. General examples include: biopsy samples of tumors or normal tissues (e.g., glial cells or other cells of the central nervous system) that may also be taken and analyzed to specifically tailor or monitor the use of a particular drug against a gene target; studies of unique tumor gene expression patterns; or analysis of SNP's (single nucleotide polymorphisms), to enhance efficacy or to avoid particular drug-sensitive normal tissue toxicities. Specific inventive examples for a substituted hexitol derivative such as dianhydrogalactitol for treatment of glioblastoma, NSCLC, or ovarian cancer include: diagnostic tools, techniques, kits and assays to confirm a patient's particular genotype; gene/protein expression chips and analysis; Single Nucleotide Polymorphisms (SNP's) assessment; SNP's for histone deacetylase, ornithine decarboxylase, GPCRs, protein kinases, telomerase, or jun; identification and measurement of metabolism enzymes and metabolites; determination of mutation of PDGFRA gene; determination of mutation of IDH1 gene; determination of mutation of NF1 gene; determination of copy number of the EGFR gene; determination of status of methylation of promoter of MGMT gene; use for disease characterized by an IDH1 mutation; use for disease characterized by IDH1 wild-type; use for disease characterized by 1p/19q co-deletion; use for disease where the 1p/19q co-deletion is not present; use for disease characterized by an unmethylated promoter region of the MGMT gene; use for disease characterized by a methylated promoter region of the MGMT gene; use for disease characterized by high expression of MGMT; or use for disease characterized by low expression of MGMT.

Yet another aspect of the invention is an improvement in the therapeutic employment of a substituted hexitol derivative such as dianhydrogalactitol for treatment of glioblastoma, NSCLC, or ovarian cancer made by specialized preparation of a patient prior to or after the use of a chemotherapeutic agent. General examples include: induction or inhibition of metabolizing enzymes, specific protection of sensitive normal tissues or organ systems. Specific inventive examples for a substituted hexitol derivative such as dianhydrogalactitol for treatment of glioblastoma, NSCLC, or ovarian cancer include: the use of colchicine or analogs; use of diuretics such as probenecid; use of a uricosuric; use of uricase; non-oral use of nicotinamide; sustained release forms of nicotinamide; use of inhibitors of poly (ADP ribose) polymerase; use of caffeine; leucovorin rescue; infection control; antihypertensives.

Yet another aspect of the invention is an improvement in the therapeutic employment of a substituted hexitol derivative such as dianhydrogalactitol for treatment of glioblastoma, NSCLC, or ovarian cancer made by use of additional drugs or procedures to prevent or reduce potential side-effects or toxicities. General examples include: the use of anti-emetics, anti-nausea, hematological support agents to limit or prevent neutropenia, anemia, thrombocytopenia, vitamins, antidepressants, treatments for sexual dysfunction, and other supportive techniques. Specific inventive examples for a substituted hexitol derivative such as dianhydrogalactitol for treatment of glioblastoma, NSCLC, or ovarian cancer include: the use of colchicine or analogs; use of diuretics such as probenecid; use of a uricosuric; use of uricase; non-oral use of nicotinamide; use of sustained release forms of nicotinamide; use of inhibitors of poly ADP-ribose polymerase; use of caffeine; leucovorin rescue; use of sustained release allopurinol; non-oral use of allopurinol; use of bone marrow transplants; use of a blood cell stimulant; use of blood or platelet infusions; use of filgrastim, G-CSF, or GM-CSF; use of pain management techniques; use of anti-inflammatories; use of fluids; use of corticosteroids; use of insulin control medications; use of antipyretics; use of anti-nausea treatments; use of anti-diarrheal treatment; use of N-acetylcysteine; or use of antihistamines.

Yet another aspect of the invention is an improvement in the therapeutic employment of a substituted hexitol derivative such as dianhydrogalactitol for treatment of glioblastoma, NSCLC, or ovarian cancer made by the use of monitoring drug levels after dosing in an effort to maximize a patient's drug plasma level, to monitor the generation of toxic metabolites, monitoring of ancillary medicines that could be beneficial or harmful in terms of drug-drug interactions. General examples include: the monitoring of drug plasma protein binding, and monitoring of other pharmacokinetic or pharmacodynamic variables. Specific inventive examples for a substituted hexitol derivative such as dianhydrogalactitol for treatment of glioblastoma, NSCLC, or ovarian cancer include: multiple determinations of drug plasma levels; or multiple determinations of metabolites in the blood or urine.

Yet another aspect of the invention is an improvement in the therapeutic employment of a substituted hexitol derivative such as dianhydrogalactitol for treatment of glioblastoma, NSCLC, or ovarian cancer made by exploiting unique drug combinations that may provide a more than additive or synergistic improvement in efficacy or side-effect management. Specific inventive examples for a substituted hexitol derivative such as dianhydrogalactitol for treatment of glioblastoma, NSCLC, or ovarian cancer include: in combination with topoisomerase inhibitors; use with fraudulent nucleosides; use with fraudulent nucleotides; use with thymidylate synthetase inhibitors; use with signal transduction inhibitors; use with cisplatin or platinum analogs; use with alkylating agents such as the nitrosoureas (BCNU, Gliadel wafers, CCNU, nimustine (ACNU), bendamustine (Treanda)); use with alkylating agents that damage DNA at a different place than does dianhydrogalactitol or another alkylating hexitol derivative (TMZ, BCNU, CCNU, and other alkylating agents all damage DNA at O⁶ of guanine, whereas dianhydrogalactitol cross-links at N⁷); use with a monofunctional alkylating agent; use with a bifunctional alkylating agent; use with anti-tubulin agents; use with antimetabolites; use with berberine; use with apigenin; use with amonafide; use with colchicine or an analog thereof; use with genistein; use with etoposide; use with cytarabine; use with cam pothecins; use with vinca alkaloids; use with 5-fluorouracil; use with curcumin; use with NF-κB inhibitors; use with rosmarinic acid; use with mitoguazone; use with tetrandrine; use with temozolomide (TMZ); use in combination with biological therapies such as antibodies such as Avastin (a VEGF inhibitor), Rituxan, Herceptin, Erbitux; use in combination with cancer vaccine therapy; use with epigenetic modulators; use with transcription factor inhibitors; use with taxol; use with homoharringtonine; use with pyridoxal; use with spirogermanium; use with caffeine; use with nicotinamide; use with methylglyoxalbisguanylhydrazone; use with Rho kinase inhibitors; use with 1,2,4-benzotriazine oxides; use with an alkylglycerol; use with an inhibitor of a Mer, Ax1, or Tyro-3 receptor kinase; use with an inhibitor of ATR kinase; use with a modulator of Fms kinase, Kit kinase, MAP4K4 kinase, TrkA kinase, or TrkB kinase; use with endoxifen; use with a mTOR inhibitor; use with an inhibitor of Mnk1a kinase, Mkn1b kinase, Mnk2a kinase, or Mnk2b kinase; use with a modulator of pyruvate kinase M2; use with a modulator of phosphoinositide 3-kinases; use with a cysteine protease inhibitor; use with phenformin; use with Sindbis virus-based vectors; use with peptidomimetics that act as mimetics of Smac and inhibit IAPs to promote apoptosis; use with a Raf kinase inhibitor; use with a nuclear transport modulator; use with an acid ceramidase inhibitor and a choline kinase inhibitor; use with tyrosine kinase inhibitors; use with anti-CS1 antibodies; use with inhibitors of protein kinase CK2; use with anti-guanylyl cyclase C (GCC) antibodies; use with histone deacetylase inhibitors; use with cannabinoids; use with glucagon-like peptide-1 (GLP-1) receptor agonists; use with inhibitors of Bcl-2 or Bcl-xL; use with Stat3 pathway inhibitors; use with inhibitors of polo-like kinase 1 (Plk1); use with GBPAR1 activators; use with modulators of serine-threonine protein kinase and poly(ADP-ribose) polymerase (PARP) activity; use with taxanes; use with inhibitors of dihydrofolate reductase; use with inhibitors of aromatase; use with benzimidazole-based anti-neoplastic agents; use with an 06-methylguanine-DNA-methyltransferase (MGMT) inhibitor; use with CCR9 inhibitors; use with acid sphingomyelinase inhibitors; use with peptidomimetic macrocycles; use with cholanic acid amides; use with substituted oxazaphosphorines; use with anti-TWEAK receptor antibodies; use with an ErbB3 binding protein; use with a glutathione S-transferase-activated anti-neoplastic compound; use with substituted phosphorodiamidates; use with inhibitors of MEKK protein kinase; use with COX-2 inhibitors; use with cimetidine and a cysteine derivative; use with anti-IL-6 receptor antibody; use with an antioxidant; use with an isoxazole inhibitor of tubulin polymerization; use with PARP inhibitors; use with Aurora protein kinase inhibitors; use with peptides binding to prostate-specific membrane antigen; use with CD19 binding agents; use with benzodiazepines; use with Toll-like receptor (TLR) agonists; use with bridged bicyclic sulfamides; use with inhibitors of epidermal growth factor receptor kinase; use with a ribonuclease of the T2 family having actin-binding activity; use with myrsinoic acid A or an analog thereof; use with inhibitors of a cyclin-dependent kinase; use with inhibitors of the interaction between p53 and MDM2; use with inhibitors of the receptor tyrosine kinase MET; use with largazole or largazole analogs; use with inhibitors of AKT protein kinase; use with 2′-fluoro-5-methyl-β-L-arabinofuranosyluridine or L-deoxythymidine; use with HSP90 modulators; use with inhibitors of JAK kinases; use with inhibitors of PDK1 protein kinase; use with PDE4 inhibitors; use with inhibitors of proto-oncogene c-Met tyrosine kinase; use with inhibitors of indoleamine 2,3-dioxygenase; use with agents that inhibit expression of ATDC (TRIM29); use with proteomimetic inhibitors of the interaction of nuclear receptor with coactivator peptides; use with antagonists of XIAP family proteins; use with tumor-targeted superantigens; use with inhibitors of Pim kinases; use with inhibitors of CHK1 or CH2 kinases; use with inhibitors of angiopoietin-like 4 protein; use with Smo antagonists; use with nicotinic acetylcholine receptor antagonists; use with farnesyl protein transferase inhibitors; use with adenosine A3 receptor antagonists; use with a Src inhibitor; or use with an agent that suppresses growth or replication of glioma cancer stem cells.

Yet another aspect of the invention is an improvement in the therapeutic employment of a substituted hexitol derivative such as dianhydrogalactitol for treatment of glioblastoma, NSCLC, or ovarian cancer made by exploiting the substituted hexitol derivative such as dianhydrogalactitol as a chemosensitizer where no measurable activity is observed when used alone but in combination with other therapeutics a more than additive or synergistic improvement in efficacy is observed. Specific inventive examples for a substituted hexitol derivative such as dianhydrogalactitol for treatment of glioblastoma, NSCLC, or ovarian cancer include: as a chemosensitizer in combination with topoisomerase inhibitors; as a chemosensitizer in combination with fraudulent nucleosides; as a chemosensitizer in combination with fraudulent nucleotides; as a chemosensitizer in combination with thymidylate synthetase inhibitors; as a chemosensitizer in combination with signal transduction inhibitors; as a chemosensitizer in combination with cisplatin or platinum analogs; as a chemosensitizer in combination with alkylating agents such as BCNU, BCNU wafers, Gliadel, CCNU, bendamustine (Treanda), or Temozolomide (Temodar); as a chemosensitizer in combination with anti-tubulin agents; as a chemosensitizer in combination with antimetabolites; as a chemosensitizer in combination with berberine; as a chemosensitizer in combination with apigenin; as a chemosensitizer in combination with amonafide; as a chemosensitizer in combination with colchicine or analogs; as a chemosensitizer in combination with genistein; as a chemosensitizer in combination with etoposide; as a chemosensitizer in combination with cytarabine; as a chemosensitizer in combination with camptothecins; as a chemosensitizer in combination with vinca alkaloids; (This has been mentioned on Page 52, line 24) as a chemosensitizer in combination with 5-fluorouracil; as a chemosensitizer in combination with curcumin; as a chemosensitizer in combination with NF-κB inhibitors; as a chemosensitizer in combination with rosmarinic acid; as a chemosensitizer in combination with mitoguazone; as a chemosensitizer in combination with tetrandrine; as a chemosensitizer in combination with a tyrosine kinase inhibitor; as a chemosensitizer in combination with an EGFR inhibitor; or as a chemosensitizer in combination with an inhibitor of poly (ADP-ribose) polymerase (PARP).

Yet another aspect of the invention is an improvement in the therapeutic employment of a substituted hexitol derivative such as dianhydrogalactitol for treatment of glioblastoma, NSCLC, or ovarian cancer made by exploiting the substituted hexitol derivative such as dianhydrogalactitol as a chemopotentiator where minimal therapeutic activity is observed alone but in combination with other therapeutics a more than additive or synergistic improvement in efficacy is observed. Specific inventive examples for a substituted hexitol derivative such as dianhydrogalactitol for treatment of glioblastoma, NSCLC, or ovarian cancer include: as a chemopotentiator in combination with topoisomerase inhibitors; as a chemopotentiator in combination with fraudulent nucleosides; as a chemopotentiator in combination with fraudulent nucleotides; as a chemopotentiator in combination with thymidylate synthetase inhibitors; as a chemopotentiator in combination with signal transduction inhibitors; as a chemopotentiator in combination with cisplatin or platinum analogs; as a chemopotentiator in combination with use with alkylating agents such as BCNU, BCNU wafers, Gliadel, or bendamustine (Treanda); as a chemopotentiator in combination with anti-tubulin agents; as a chemopotentiator in combination with antimetabolites; as a chemopotentiator in combination with berberine; as a chemopotentiator in combination with apigenin; as a chemopotentiator in combination with amonafide; as a chemopotentiator in combination with colchicine or analogs; as a chemopotentiator in combination with genistein; as a chemopotentiator in combination with etoposide; as a chemopotentiator in combination with cytarabine; as a chemopotentiator in combination with camptothecins; as a chemopotentiator in combination with vinca alkaloids; as a chemopotentiator in combination with 5-fluorouracil; as a chemopotentiator in combination with curcumin; as a chemopotentiator in combination with NF-κB inhibitors; as a chemopotentiator in combination with rosmarinic acid; as a chemopotentiator in combination with mitoguazone; as a chemopotentiator in combination with tetrandrine; as a chemopotentiator in combination with a tyrosine kinase inhibitor; as a chemopotentiator in combination with an EGFR inhibitor; or as a chemopotentiator in combination with an inhibitor of poly (ADP-ribose) polymerase (PARP).

Yet another aspect of the invention is an improvement in the therapeutic employment of a substituted hexitol derivative such as dianhydrogalactitol for treatment of glioblastoma, NSCLC, or ovarian cancer made by drugs, treatments and diagnostics to allow for the maximum benefit to patients treated with a compound. General examples include: pain management, nutritional support, anti-emetics, anti-nausea therapies, anti-anemia therapy, anti-inflammatories. Specific inventive examples for a substituted hexitol derivative such as dianhydrogalactitol for treatment of glioblastoma, NSCLC, or ovarian cancer include: use with therapies associated with pain management; nutritional support; anti-emetics; anti-nausea therapies; anti-anemia therapy; anti-inflammatories: antipyretics; immune stimulants.

Yet another aspect of the invention is an improvement in the therapeutic employment of a substituted hexitol derivative such as dianhydrogalactitol for treatment of glioblastoma, NSCLC, or ovarian cancer made by the use of complementary therapeutics or methods to enhance effectiveness or reduce side effects. Specific inventive examples for a substituted hexitol derivative such as dianhydrogalactitol for treatment of glioblastoma, NSCLC, or ovarian cancer include: hypnosis; acupuncture; meditation; herbal medications created either synthetically or through extraction including NF-κB inhibitors (such as parthenolide, curcumin, rosmarinic acid); natural anti-inflammatories (including rhein, parthenolide); immunostimulants (such as those found in Echinacea); antimicrobials (such as berberine); flavonoids, isoflavones, and flavones (such as apigenenin, genistein, genistin, 6″-O-malonylgenistin, 6″-O-acetylgenistin, daidzein, daidzin, 6″-O-malonyldaidzin, 6″-O-acetylgenistin, glycitein, glycitin, 6″-O-malonylglycitin, and 6-O-acetylglycitin); applied kinesiology.

Yet another aspect of the invention is an improvement in the therapeutic employment of a substituted hexitol derivative such as dianhydrogalactitol for treatment of glioblastoma, NSCLC, or ovarian cancer made by alterations in the pharmaceutical bulk substance. General examples include: salt formation, homogeneous crystalline structure, pure isomers. Specific inventive examples for a substituted hexitol derivative such as dianhydrogalactitol for treatment of glioblastoma, NSCLC, or ovarian cancer include: salt formation; homogeneous crystalline structure; pure isomers; increased purity; lower residual solvents; or lower heavy metals.

Yet another aspect of the invention is an improvement in the therapeutic employment of a substituted hexitol derivative such as dianhydrogalactitol for treatment of glioblastoma, NSCLC, or ovarian cancer made by alterations in the diluents used to solubilize and deliver/present the compound for administration. General examples include: Cremophor-EL, cyclodextrins for poorly water soluble compounds. Specific inventive examples for a substituted hexitol derivative such as dianhydrogalactitol for treatment of glioblastoma, NSCLC, or ovarian cancer include: use of emulsions; dimethyl sulfoxide (DMSO); N-methylformamide (NMF); dimethylformamide (DMF); dimethylacetamide (DMA); ethanol; benzyl alcohol; dextrose containing water for injection; Cremophor; cyclodextrins; PEG.

Yet another aspect of the invention is an improvement in the therapeutic employment of a substituted hexitol derivative such as dianhydrogalactitol for treatment of glioblastoma, NSCLC, or ovarian cancer made by alterations in the solvents used or required to solubilize a compound for administration or for further dilution. General examples include: ethanol, dimethylacetamide (DMA). Specific inventive examples for a substituted hexitol derivative such as dianhydrogalactitol for treatment of glioblastoma, NSCLC, or ovarian cancer include: the use of emulsions; DMSO; NMF; DMF; DMA; ethanol; benzyl alcohol; dextrose containing water for injection; Cremophor; cyclodextrin; or PEG.

Yet another aspect of the invention is an improvement in the therapeutic employment of a substituted hexitol derivative such as dianhydrogalactitol for treatment of glioblastoma, NSCLC, or ovarian cancer made by alterations in the materials/excipients, buffering agents, or preservatives required to stabilize and present a chemical compound for proper administration. General examples include: mannitol, albumin, EDTA, sodium bisulfite, benzyl alcohol. Specific inventive examples for a substituted hexitol derivative such as dianhydrogalactitol for treatment of glioblastoma, NSCLC, or ovarian cancer include: the use of mannitol; albumin; EDTA; sodium bisulfite; benzyl alcohol; carbonate buffers; phosphate buffers.

Yet another aspect of the invention is an improvement in the therapeutic employment of a substituted hexitol derivative such as dianhydrogalactitol for treatment of glioblastoma, NSCLC, or ovarian cancer made by alterations in the potential dosage forms of the compound dependent on the route of administration, duration of effect, plasma levels required, exposure to side-effect normal tissues and metabolizing enzymes. General examples include: tablets, capsules, topical gels, creams, patches, suppositories. Specific inventive examples for a substituted hexitol derivative such as dianhydrogalactitol for treatment of glioblastoma, NSCLC, or ovarian cancer include: the use of tablets; capsules; topical gels; topical creams; patches; suppositories; lyophilized dosage fills.

Yet another aspect of the invention is an improvement in the therapeutic employment of a substituted hexitol derivative such as dianhydrogalactitol for treatment of glioblastoma, NSCLC, or ovarian cancer made by alterations in the dosage forms, container/closure systems, accuracy of mixing and dosage preparation and presentation. General examples include: amber vials to protect from light, stoppers with specialized coatings. Specific inventive examples for a substituted hexitol derivative such as dianhydrogalactitol for treatment of glioblastoma, NSCLC, or ovarian cancer include: the use of amber vials to protect from light; stoppers with specialized coatings to improve shelf-life stability.

Yet another aspect of the invention is an improvement in the therapeutic employment of a substituted hexitol derivative such as dianhydrogalactitol for treatment of glioblastoma, NSCLC, or ovarian cancer made by the use of delivery systems to improve the potential attributes of a pharmaceutical product such as convenience, duration of effect, reduction of toxicities. General examples include: nanocrystals, bioerodible polymers, liposomes, slow release injectable gels, microspheres. Specific inventive examples for a substituted hexitol derivative such as dianhydrogalactitol for treatment of glioblastoma, NSCLC, or ovarian cancer include: the use of nanocrystals; bioerodible polymers; liposomes; slow release injectable gels; microspheres.

Yet another aspect of the invention is an improvement in the therapeutic employment of a substituted hexitol derivative such as dianhydrogalactitol for treatment of glioblastoma, NSCLC, or ovarian cancer made by alterations to the parent molecule with covalent, ionic, or hydrogen bonded moieties to alter the efficacy, toxicity, pharmacokinetics, metabolism, or route of administration. General examples include: polymer systems such as polyethylene glycols, polylactides, polyglycolides, amino acids, peptides, or multivalent linkers. Specific inventive examples for a substituted hexitol derivative such as dianhydrogalactitol for treatment of glioblastoma, NSCLC, or ovarian cancer include: the use of polymer systems such as polyethylene glycols; polylactides; polyglycolides; amino acids; peptides; multivalent linkers.

Yet another aspect of the invention is an improvement in the therapeutic employment of a substituted hexitol derivative such as dianhydrogalactitol for treatment of glioblastoma, NSCLC, or ovarian cancer made by alterations to the molecule such that improved pharmaceutical performance is gained with a variant of the active molecule in that after introduction into the body a portion of the molecule is cleaved to reveal the preferred active molecule. General examples include: enzyme sensitive esters, dimers, Schiff bases. Specific inventive examples for a substituted hexitol derivative such as dianhydrogalactitol for treatment of glioblastoma, NSCLC, or ovarian cancer include: the use of enzyme sensitive esters; dimers; Schiff bases; pyridoxal complexes; caffeine complexes.

Yet another aspect of the invention is an improvement in the therapeutic employment of a substituted hexitol derivative such as dianhydrogalactitol for treatment of glioblastoma, NSCLC, or ovarian cancer made by the use of additional compounds, biological agents that, when administered in the proper fashion, a unique and beneficial effect can be realized. General examples include: inhibitors of multi-drug resistance, specific drug resistance inhibitors, specific inhibitors of selective enzymes, signal transduction inhibitors, repair inhibition. Specific inventive examples for a substituted hexitol derivative such as dianhydrogalactitol for treatment of glioblastoma, NSCLC, or ovarian cancer include: the use of inhibitors of multi-drug resistance; specific drug resistance inhibitors; specific inhibitors of selective enzymes; signal transduction inhibitors; repair inhibition; topoisomerase inhibitors with non-overlapping side effects.

Yet another aspect of the invention is an improvement in the therapeutic employment of a substituted hexitol derivative such as dianhydrogalactitol for treatment of glioblastoma, NSCLC, or ovarian cancer made by the use of the substituted hexitol derivative such as dianhydrogalactitol in combination as sensitizers/potentiators with biological response modifiers. General examples include: use in combination as sensitizers/potentiators with biological response modifiers, cytokines, lymphokines, therapeutic antibodies, antisense therapies, gene therapies. Specific inventive examples for a substituted hexitol derivative such as dianhydrogalactitol for treatment of glioblastoma, NSCLC, or ovarian cancer include: use in combination as sensitizers/potentiators with biological response modifiers; cytokines; lymphokines; therapeutic antibodies such as Avastin, Herceptin, Rituxan, and Erbitux; antisense therapies; gene therapies; ribozymes; RNA interference; or vaccines.

Yet another aspect of the invention is an improvement in the therapeutic employment of a substituted hexitol derivative such as dianhydrogalactitol for treatment of glioblastoma, NSCLC, or ovarian cancer made by exploiting the selective use of the substituted hexitol derivative such as dianhydrogalactitol to overcome developing or complete resistance to the efficient use of biotherapeutics. General examples include: tumors resistant to the effects of biological response modifiers, cytokines, lymphokines, therapeutic antibodies, antisense therapies, gene therapies. Specific inventive examples for a substituted hexitol derivative such as dianhydrogalactitol for treatment of glioblastoma, NSCLC, or ovarian cancer include: the use against tumors resistant to the effects of biological response modifiers; cytokines; lymphokines; therapeutic antibodies such as Avastin, Rituxan, Herceptin, Erbitux; antisense therapies; gene therapies; ribozymes; RNA interference; and vaccines.

Yet another aspect of the invention is an improvement in the therapeutic employment of a substituted hexitol derivative such as dianhydrogalactitol for treatment of glioblastoma, NSCLC, or ovarian cancer made by exploiting their use in combination with ionizing radiation, phototherapies, heat therapies, or radio-frequency generated therapies. General examples include: hypoxic cell sensitizers, radiation sensitizers/protectors, photosensitizers, radiation repair inhibitors. Specific inventive examples for a substituted hexitol derivative such as dianhydrogalactitol for treatment of glioblastoma, NSCLC, or ovarian cancer include: use in combination with ionizing radiation; use in combination with hypoxic cell sensitizers; use in combination with radiation sensitizers/protectors; use in combination with photosensitizers; use in combination with radiation repair inhibitors; use in combination with thiol depletion; use in combination with vaso-targeted agents; use in combination with use with radioactive seeds; use in combination with radionuclides; use in combination with radiolabeled antibodies; use in combination with brachytherapy. This is useful because radiation therapy is almost always undertaken early in the treatment of glioblastoma and NSCLC and may also be used in the treatment of ovarian cancer, especially in early stages, and improvements in the efficacy of such radiation therapy or the ability to exert a synergistic effect by combining radiation therapy with the administration of a substituted hexitol derivative such as dianhydrogalactitol is significant.

Yet another aspect of the invention is an improvement in the therapeutic employment of a substituted hexitol derivative such as dianhydrogalactitol for treatment of glioblastoma, NSCLC, or ovarian cancer made by optimizing its utility by determining the various mechanisms of action, biological targets of a compound for greater understanding and precision to better exploit the utility of the molecule. Specific inventive examples for a substituted hexitol derivative such as dianhydrogalactitol for treatment of glioblastoma, NSCLC, or ovarian cancer include: the use with inhibitors of poly-ADP ribose polymerase; agents that effect vasculature or vasodilation; oncogenic targeted agents; signal transduction inhibitors; EGFR inhibition; Protein Kinase C inhibition; Phospholipase C downregulation; Jun downregulation; histone genes; VEGF; ornithine decarboxylase; ubiquitin C; jun D; v-jun; GPCRs; protein kinase A; telomerase, prostate specific genes; protein kinases other than protein kinase A; histone deacetylase; and tyrosine kinase inhibitors.

Yet another aspect of the invention is an improvement in the therapeutic employment of a substituted hexitol derivative such as dianhydrogalactitol for treatment of glioblastoma, NSCLC, or ovarian cancer made by more precise identification and exposure of the compound to those select cell populations where the compound's effect can be maximally exploited, particularly glioblastoma, NSCLC, or ovarian cancer tumor cells. Specific inventive examples for a substituted hexitol derivative such as dianhydrogalactitol for treatment of glioblastoma, NSCLC, or ovarian cancer include: use against radiation sensitive cells; use against radiation resistant cells; or use against energy depleted cells.

Yet another aspect of the invention is an improvement in the therapeutic employment of a substituted hexitol derivative such as dianhydrogalactitol for treatment of glioblastoma, NSCLC, or ovarian cancer made by use of an agent to enhance activity of the substituted hexitol derivative. General examples include: use with nicotinamide, caffeine, tetandrine, or berberine. Specific inventive examples for a substituted hexitol for treatment of a malignancy such as glioblastoma, NSCLC, or ovarian cancer include: use with nicotinamide; use with caffeine; use with tetandrine; or use with berberine.

Yet another aspect of the invention is an improvement in the therapeutic employment of a substituted hexitol derivative such as dianhydrogalactitol for treatment of glioblastoma, NSCLC, or ovarian cancer made by use of an agent to counteract myelosuppression. Specific inventive examples for a substituted hexitol derivative such as dianhydrogalactitol for treatment of a malignancy such as glioblastoma, NSCLC, or ovarian cancer include use of dithiocarbamates to counteract myelosuppression.

Yet another aspect of the invention is an improvement in the therapeutic employment of a substituted hexitol derivative such as dianhydrogalactitol for treatment of glioblastoma made by use of an agent that increases the ability of the substituted hexitol to pass through the blood-brain barrier. Specific examples for a substituted hexitol derivative such as dianhydrogalactitol for treatment of glioblastoma include chimeric peptides; compositions comprising either avidin or an avidin fusion protein bonded to a biotinylated substituted hexitol derivative; neutral liposomes that are pegylated and that incorporate the substituted hexitol derivative and wherein the polyethylene glycol strands are conjugated to at least one transportable peptide or targeting agent; a humanized murine antibody that binds to the human insulin receptor linked to the substituted hexitol derivative through an avidin-biotin linkage; and a fusion protein linked to the hexitol through an avidin-biotin linkage. These methods are also useful in the treatment of brain metastases that occur in NSCLC or ovarian cancer.

The methods of the present invention can also be employed to improve the efficacy and/or reduce the side effects of the administration of a substituted hexitol derivative such as dianhydrogalactitol for treatment of leptomeningeal carcinomatosis (LC) as further described below.

Accordingly, one aspect of the present invention is a method to improve the efficacy and/or reduce the side effects of the administration of a substituted hexitol derivative such as dianhydrogalactitol for treatment of glioblastoma, NSCLC, or ovarian cancer, comprising the steps of:

(1) identifying at least one factor or parameter associated with the efficacy and/or occurrence of side effects of the administration of the substituted hexitol derivative such as dianhydrogalactitol for treatment of glioblastoma, NSCLC, or ovarian cancer; and

(2) modifying the factor or parameter to improve the efficacy and/or reduce the side effects of the administration of the substituted hexitol derivative such as dianhydrogalactitol for treatment of glioblastoma, NSCLC, or ovarian cancer.

Typically, the factor or parameter is selected from the group consisting of:

(1) dose modification;

(2) route of administration;

(3) schedule of administration;

(4) indications for use;

(5) selection of disease stage;

(6) other indications;

(7) patient selection;

(8) patient/disease phenotype;

(9) patient/disease genotype;

(10) pre/post-treatment preparation

(11) toxicity management;

(12) pharmacokinetic/pharmacodynamic monitoring;

(13) drug combinations;

(14) chemosensitization;

(15) chemopotentiation;

(16) post-treatment patient management;

(17) alternative medicine/therapeutic support;

(18) bulk drug product improvements;

(19) diluent systems;

(20) solvent systems;

(21) excipients;

(22) dosage forms;

(23) dosage kits and packaging;

(24) drug delivery systems;

(25) drug conjugate forms;

(26) compound analogs;

(27) prodrugs;

(28) multiple drug systems;

(29) biotherapeutic enhancement;

(30) biotherapeutic resistance modulation;

(31) radiation therapy enhancement;

(32) novel mechanisms of action;

(33) selective target cell population therapeutics;

(34) use with ionizing radiation;

(35) use with an agent enhancing its activity;

(36) use with an agent that counteracts myelosuppression; and

(37) use with an agent that increases the ability of the substituted hexitol to pass through the blood-brain barrier.

Similarly, another aspect of the present invention is a method to improve the efficacy and/or reduce the side effects of the administration of a substituted hexitol derivative such as dianhydrogalactitol for treatment of leptomeningeal carcinomatosis (LC) comprising the steps of:

(1) identifying at least one factor or parameter associated with the efficacy and/or occurrence of side effects of the administration of the substituted hexitol derivative such as dianhydrogalactitol for treatment of LC; and

(2) modifying the factor or parameter to improve the efficacy and/or reduce the side effects of the administration of the substituted hexitol derivative such as dianhydrogalactitol for treatment of LC.

Typically, the factor or parameter is selected from the group consisting of:

(1) dose modification;

(2) route of administration;

(3) schedule of administration;

(4) indications for use;

(5) selection of disease stage;

(6) other indications;

(7) patient selection;

(8) patient/disease phenotype;

(9) patient/disease genotype;

(10) pre/post-treatment preparation

(11) toxicity management;

(12) pharmacokinetic/pharmacodynamic monitoring;

(13) drug combinations;

(14) chemosensitization;

(15) chemopotentiation;

(16) post-treatment patient management;

(17) alternative medicine/therapeutic support;

(18) bulk drug product improvements;

(19) diluent systems;

(20) solvent systems;

(21) excipients;

(22) dosage forms;

(23) dosage kits and packaging;

(24) drug delivery systems;

(25) drug conjugate forms;

(26) compound analogs;

(27) prodrugs;

(28) multiple drug systems;

(29) biotherapeutic enhancement;

(30) biotherapeutic resistance modulation;

(31) radiation therapy enhancement;

(32) novel mechanisms of action;

(33) selective target cell population therapeutics;

(34) use with ionizing radiation;

(35) use with an agent enhancing its activity;

(36) use with an agent that counteracts myelosuppression; and

(37) use with an agent that increases the ability of the substituted hexitol to pass through the blood-brain barrier.

As detailed above, in general, the substituted hexitol derivative usable in methods and compositions according to the present invention include galactitols, substituted galacitols, dulcitols, and substituted dulcitols, including dianhydrogalactitol, diacetyldianhydrogalactitol, dibromodulcitol, and derivatives and analogs thereof. Typically, the substituted hexitol derivative is selected from the group consisting of dianhydrogalactitol, derivatives of dianhydrogalactitol, diacetyldianhydrogalactitol, derivatives of diacetyldianhydrogalactitol, dibromodulcitol, and derivatives of dibromodulcitol. Preferably, the substituted hexitol derivative is dianhydrogalactitol.

When the improvement is made by dose modification, the dose modification can be, but is not limited to, at least one dose modification selected from the group consisting of:

(a) continuous i.v. infusion for hours to days;

(b) biweekly administration;

(c) doses greater than 5 mg/m²/day;

(d) progressive escalation of dosing from 1 mg/m²/day based on patient tolerance;

(e) use of caffeine to modulate metabolism;

(f) use of isoniazid to modulate metabolism;

(g) selected and intermittent boosting of dosage administration;

(h) administration of single and multiple doses escalating from 5 mg/m²/day via bolus;

(i) oral dosages of below 30 mg/m²;

(j) oral dosages of above 130 mg/m²;

(k) oral dosages up to 40 mg/m² for 3 days and then a nadir/recovery period of 18-21 days;

(l) dosing at a lower level for an extended period (e.g., 21 days);

(m) dosing at a higher level;

(n) dosing with a nadir/recovery period longer than 21 days;

(o) dosing at a level to achieve a concentration of the substituted hexitol derivative such as dianhydrogalactitol in the cerebrospinal fluid (CSF) of equal to or greater than 5 μM;

(p) dosing at a level to achieve a cytotoxic concentration in the CSF;

(q) the use of a substituted hexitol derivative such as dianhydrogalactitol as a single cytotoxic agent;

(r) administration on a 33-day cycle with a cumulative dose of about 9 mg/m²;

(s) administration on a 33-day cycle with a cumulative dose of about 10 mg/m²;

(t) administration on a 33-day cycle with a cumulative dose of about 20 mg/m²;

(u) administration on a 33-day cycle with a cumulative dose of about 40 mg/m²;

(v) administration on a 33-day cycle with a cumulative dose of about 80 mg/m²;

(w) administration on a 33-day cycle with a cumulative dose of about 160 mg/m²;

(x) administration on a 33-day cycle with a cumulative dose of about 240 mg/m²;

(y) administration so that the plasma half-life is about 1-2 hours;

(z) administration so that the Cₘₐₓ is <200 ng/ml; and

(aa) administration so that the substituted hexitol derivative has a half-life of >20 hours in the cerebrospinal fluid.

When the improvement is made by route of administration, the route of administration can be, but is not limited to, at least one route of administration selected from the group consisting of:

(a) topical administration;

(b) oral administration;

(c) slow release oral delivery;

(d) intrathecal administration;

(e) intraarterial administration;

(f) continuous infusion;

(g) intermittent infusion;

(h) intravenous administration, such as intravenous administration for 30 minutes;

(i) administration through a longer infusion;

(j) administration through IV push; and

(k) administration to maximize the concentration of the substituted hexitol derivative such as dianhydrogalactitol in the CSF.

When the improvement is made by schedule of administration, the schedule of administration can be, but is not limited to, at least one schedule of administration selected from the group consisting of:

(a) daily administration;

(b) weekly administration;

(c) weekly administration for three weeks;

(d) biweekly administration;

(e) biweekly administration for three weeks with a 1-2 week rest period;

(f) intermittent boost dose administration;

(g) daily administration for one week for multiple weeks; and

(h) administration on days 1, 2, and 3 of a 33-day cycle.

When the improvement is made by selection of disease stage, the selection of disease stage can be, but is not limited to, at least one selection of disease stage selected from the group consisting of:

(a) use in an appropriate disease stage for glioblastoma, NSCLC, or ovarian cancer;

(b) use with an angiogenesis inhibitor to prevent or limit metastatic spread;

(c) use for newly diagnosed disease;

(d) use for recurrent disease;

(e) use for resistant or refractory disease; and

(f) use for childhood glioblastoma.

When the improvement is made by patient selection, the patient selection can be, but is not limited to, a patient selection carried out by a criterion selected from the group consisting of:

(a) selecting patients with a disease condition characterized by a high level of a metabolic enzyme selected from the group consisting of histone deacetylase and ornithine decarboxylase;

(b) selecting patients with a low or high susceptibility to a condition selected from the group consisting of thrombocytopenia and neutropenia;

(c) selecting patients intolerant of GI toxicities;

(d) selecting patients characterized by over- or under-expression of a gene selected from the group consisting of c-Jun, a GPCR, a signal transduction protein, VEGF, a prostate-specific gene, and a protein kinase.

(e) selecting patients characterized by carrying extra copies of the EGFR gene for glioblastoma, NSCLC, or ovarian cancer;

(f) selecting patients characterized by mutations in at least one gene selected from the group consisting of TP53, PDGFRA, IDH1, and NF1 for glioblastoma, NSCLC, or ovarian cancer;

(g) selecting patients characterized by methylation or lack of methylation of the promoter of the MGMT gene;

(h) selecting patients characterized by the existence of an IDH1 mutation;

(i) selecting patients characterized by the presence of IDH1 wild-type gene;

(j) selecting patients characterized by the presence of 1p/19q co-deletion;

(k) selecting patients characterized by the absence of an 1p/19q co-deletion;

(l) selecting patients characterized by an unmethylated promoter region of MGMT (O⁶-methylguanine methyltransferase);

(m) selecting patients characterized by a methylated promoter region of MGMT;

(n) selecting patients characterized by a high expression of MGMT;

(o) selecting patients characterized by a low expression of MGMT; and

(p) selecting patients characterized by a mutation in EGFR, including, but not limited to EGFR Variant III.

The cellular proto-oncogene c-Jun encodes a protein that, in combination with c-Fos, forms the AP-1 early response transcription factor. This proto-oncogene plays a key role in transcription and interacts with a large number of proteins affecting transcription and gene expression. It is also involved in proliferation and apoptosis of cells that form part of a number of tissues, including cells of the endometrium and glandular epithelial cells.

G-protein coupled receptors (GPCRs) are important signal transducing receptors. The superfamily of G protein coupled receptors includes a large number of receptors. These receptors are integral membrane proteins characterized by amino acid sequences that contain seven hydrophobic domains, predicted to represent the transmembrane spanning regions of the proteins. They are found in a wide range of organisms and are involved in the transmission of signals to the interior of cells as a result of their interaction with heterotrimeric G proteins. They respond to a diverse range of agents including lipid analogues, amino acid derivatives, small molecules such as epinephrine and dopamine, and various sensory stimuli. The properties of many known GPCR are summarized in S. Watson & S. Arkinstall, “The G-Protein Linked Receptor Facts Book” (Academic Press, London, 1994). GPCR receptors include, but are not limited to, acetylcholine receptors, β-adrenergic receptors, β₃-adrenergic receptors, serotonin (5-hydroxytryptamine) receptors, dopamine receptors, adenosine receptors, angiotensin Type II receptors, bradykinin receptors, calcitonin receptors, calcitonin gene-related receptors, cannabinoid receptors, cholecystokinin receptors, chemokine receptors, cytokine receptors, gastrin receptors, endothelin receptors, γ-aminobutyric acid (GABA) receptors, galanin receptors, glucagon receptors, glutamate receptors, luteinizing hormone receptors, choriogonadotrophin receptors, follicle-stimulating hormone receptors, thyroid-stimulating hormone receptors, gonadotrophin-releasing hormone receptors, leukotriene receptors, Neuropeptide Y receptors, opioid receptors, parathyroid hormone receptors, platelet activating factor receptors, prostanoid (prostaglandin) receptors, somatostatin receptors, thyrotropin-releasing hormone receptors, vasopressin and oxytocin receptors.

EGFR mutations can be associated with sensitivity to therapeutic agents such as gefitinib, as described in J. G. Paez et al., “EGFR Mutations in Lung Cancer: Correlation with Clinical Response to Gefitinib,” Science 304: 1497-1500 (2004). One specific mutation in EGFR that is associated with resistance to tyrosine kinase inhibitors is known as EGFR Variant III, which is described in C. A. Learn et al., “Resistance to Tyrosine Kinase Inhibition by Mutant Epidermal Growth Factor Variant III Contributes to the Neoplastic Phenotype of Glioblastoma Multiforme,” Clin. Cancer Res. 10: 3216-3224 (2004). EGFR Variant III is characterized by a consistent and tumor-specific in-frame deletion of 801 bp from the extracellular domain that splits a codon and produces a novel glycine at the fusion junction. This mutation encodes a protein with a constituently active thymidine kinase that enhances the tumorigenicity of the cells carrying this mutation. This mutated protein sequence is clonally expressed on a significant proportion of glioblastomas but is absent from normal tissues.

When the improvement is made by analysis of patient or disease phenotype, the analysis of patient or disease phenotype can be, but is not limited to, a method of analysis of patient or disease phenotype carried out by a method selected from the group consisting of:

(a) use of a diagnostic tool, a diagnostic technique, a diagnostic kit, or a diagnostic assay to confirm a patient's particular phenotype;

(b) use of a method for measurement of a marker selected from the group consisting of histone deacetylase, ornithine decarboxylase, VEGF, a protein that is a gene product of jun, and a protein kinase;

(c) surrogate compound dosing; and

(d) low dose pre-testing for enzymatic status.

When the improvement is made by analysis of patient or disease genotype, the analysis of patient or disease genotype can be, but is not limited to, a method of analysis of patient or disease genotype carried out by a method selected from the group consisting of:

(a) use of a diagnostic tool, a diagnostic technique, a diagnostic kit, or a diagnostic assay to confirm a patient's particular genotype;

(b) use of a gene chip;

(c) use of gene expression analysis;

(d) use of single nucleotide polymorphism (SNP) analysis;

(e) measurement of the level of a metabolite or a metabolic enzyme;

(f) determination of mutation of PDGFRA gene;

(g) determination of mutation of NF1 gene;

(h) determination of copy number of the EGFR gene;

(i) determination of status of methylation of promoter of MGMT gene;

(j) determination of the existence of an IDH1 mutation;

(k) determination of the existence of IDH1 wild-type;

(l) determination of the existence of a 1p/19q co-deletion;

(m) determination of the absence of a 1p/19q co-deletion;

(n) determination of the existence of an unmethylated promoter region of the MGMT gene;

(o) determination of the existence of a methylated promoter region of the MGMT gene;

(p) determination of the existence of high expression of MGMT;

and

(q) determination of the existence of low expression of MGMT.

The use of gene chips is described in A. J. Lee & S. Ramaswamy, “DNA Microarrays in Biological Discovery and Patient Care” in Essentials of Genomic and Personalized Medicine (G. S. Ginsburg & H. F. Willard, eds., Academic Press, Amsterdam, 2010), ch. 7, pp. 73-88.

When the method is the use of single nucleotide polymorphism (SNP) analysis, the SNP analysis can be carried out on a gene selected from the group consisting of histone deacetylase, ornithine decarboxylase, VEGF, a prostate specific gene, c-Jun, and a protein kinase. The use of SNP analysis is described in S. Levy and Y.-H. Rogers, “DNA Sequencing for the Detection of Human Genome Variation” in Essentials of Genomic and Personalized Medicine (G. S. Ginsburg & H. F. Willard, eds., Academic Press, Amsterdam, 2010), ch. 3, pp. 27-37.

Still other genomic techniques such as copy number variation analysis and analysis of DNA methylation can be employed. Copy number variation analysis is described in C. Lee et al., “Copy Number Variation and Human Health” in Essentials of Genomic and Personalized Medicine (G. S. Ginsburg & H. F. Willard, eds., Academic Press, Amsterdam, 2010), ch. 5, pp. 46-59. This is particularly significant for glioblastoma as an increase in copy number of EGFR is associated with particular subtypes of glioblastoma, and may be useful in other malignancies treatable by compositions and methods according to the present invention. DNA methylation analysis is described in S. Cottrell et al., “DNA Methylation Analysis: Providing New Insight into Human Disease” in Essentials of Genomic and Personalized Medicine (G. S. Ginsburg & H. F. Willard, eds., Academic Press, Amsterdam, 2010), ch. 6, pp. 60-72. This is particularly significant for glioblastoma in that the prognosis for glioblastoma varies with the degree of methylation of the promoter of the MGMT gene, and may be useful in other malignancies treatable by compositions and methods according to the present invention.

When the improvement is made by pre/post-treatment preparation, the pre/post-treatment preparation can be, but is not limited to, a method of pre/post treatment preparation selected from the group consisting of:

(a) the use of colchicine or an analog thereof;

(b) the use of a diuretic;

(c) the use of a uricosuric;

(d) the use of uricase;

(e) the non-oral use of nicotinamide;

(f) the use of a sustained-release form of nicotinamide;

(g) the use of an inhibitor of poly-ADP ribose polymerase;

(h) the use of caffeine;

(i) the use of leucovorin rescue;

(j) infection control; and

(k) the use of an anti-hypertensive agent.

Uricosurics include, but are not limited to, probenecid, benzbromarone, and sulfinpyrazone. A particularly preferred uricosuric is probenecid. Uricosurics, including probenecid, may also have diuretic activity. Other diuretics are well known in the art, and include, but are not limited to, hydrochlorothiazide, carbonic anhydrase inhibitors, furosemide, ethacrynic acid, amiloride, and spironolactone.

Poly-ADP ribose polymerase inhibitors are described in G. J. Southan & C. Szabo, “Poly(ADP-Ribose) Inhibitors,” Curr. Med. Chem. 10: 321-240 (2003), and include nicotinamide, 3-aminobenzamide, substituted 3,4-dihydroisoquinolin-1(2H)-ones and isoquinolin-1(2H)-ones, benzimidazoles, indoles, phthalazin-1(2H)-ones, quinazolinones, isoindolinones, phenanthridinones, and other compounds.

Leucovorin rescue comprises administration of folinic acid (leucovorin) to patients in which methotrexate has been administered. Leucovorin is a reduced form of folic acid that bypasses dihydrofolate reductase and restores hematopoietic function. Leucovorin can be administered either intravenously or orally.

In one alternative, wherein the pre/post treatment is the use of a uricosuric, the uricosuric is probenecid or an analog thereof.

When the improvement is made by toxicity management, the toxicity management can be, but is not limited to, a method of toxicity management selected from the group consisting of:

(a) the use of colchicine or an analog thereof;

(b) the use of a diuretic;

(c) the use of a uricosuric;

(d) the use of uricase;

(e) the non-oral use of nicotinamide;

(f) the use of a sustained-release form of nicotinamide;

(g) the use of an inhibitor of poly-ADP ribose polymerase;

(h) the use of caffeine;

the use of leucovorin rescue;

(j) the use of sustained-release allopurinol;

(k) the non-oral use of allopurinol;

(l) the use of bone marrow transplants;

(m) the use of a blood cell stimulant;

(n) the use of blood or platelet infusions;

(o) the administration of an agent selected from the group consisting of filgrastim, G-CSF, and GM-CSF;

(p) the application of a pain management technique;

(q) the administration of an anti-inflammatory agent;

(r) the administration of fluids;

(s) the administration of a corticosteroid;

(t) the administration of an insulin control medication;

(u) the administration of an antipyretic;

(v) the administration of an anti-nausea treatment;

(w) the administration of an anti-diarrheal treatment;

(x) the administration of N-acetylcysteine; and

(y) the administration of an antihistamine.

Filgrastim is a granulocytic colony-stimulating factor (G-CSF) analog produced by recombinant DNA technology that is used to stimulate the proliferation and differentiation of granulocytes and is used to treat neutropenia; G-CSF can be used in a similar manner. GM-CSF is granulocyte macrophage colony-stimulating factor and stimulates stem cells to produce granulocytes (eosinophils, neutrophils, and basophils) and monocytes; its administration is useful to prevent or treat infection.

Anti-inflammatory agents are well known in the art and include corticosteroids and non-steroidal anti-inflammatory agents (NSAIDs). Corticosteroids with anti-inflammatory activity include, but are not limited to, hydrocortisone, cortisone, beclomethasone dipropionate, betamethasone, dexamethasone, prednisone, methylprednisolone, triamcinolone, fluocinolone acetonide, and fludrocortisone. Non-steroidal anti-inflammatory agents include, but are not limited to, acetylsalicylic acid (aspirin), sodium salicylate, choline magnesium trisalicylate, salsalate, diflunisal, sulfasalazine, olsalazine, acetaminophen, indomethacin, sulindac, tolmetin, diclofenac, ketorolac, ibuprofen, naproxen, flurbiprofen, ketoprofen, fenoprofin, oxaprozin, mefenamic acid, meclofenamic acid, piroxicam, meloxicam, nabumetone, rofecoxib, celecoxib, etodolac, nimesulide, aceclofenac, alclofenac, alminoprofen, amfenac, ampiroxicam, apazone, araprofen, azapropazone, bendazac, benoxaprofen, benzydamine, bermoprofen, benzpiperylon, bromfenac, bucloxic acid, bumadizone, butibufen, carprofen, cimicoxib, cinmetacin, cinnoxicam, clidanac, clofezone, clonixin, clopirac, darbufelone, deracoxib, droxicam, eltenac, enfenamic acid, epirizole, esflurbiprofen, ethenzamide, etofenamate, etoricoxib, felbinac, fenbufen, fenclofenac, fenclozic acid, fenclozine, fendosal, fentiazac, feprazone, filenadol, flobufen, florifenine, flosulide, flubichin methanesulfonate, flufenamic acid, flufenisal, flunixin, flunoxaprofen, fluprofen, fluproquazone, furofenac, ibufenac, imrecoxib, indoprofen, isofezolac, isoxepac, isoxicam, licofelone, lobuprofen, lomoxicam, lonazolac, loxaprofen, lumaricoxib, mabuprofen, miroprofen, mofebutazone, mofezolac, morazone, nepafanac, niflumic acid, nitrofenac, nitroflurbiprofen, nitronaproxen, orpanoxin, oxaceprol, oxindanac, oxpinac, oxyphenbutazone, pamicogrel, parcetasal, parecoxib, parsalmide, pelubiprofen, pemedolac, phenylbutazone, pirazolac, pirprofen, pranoprofen, salicin, salicylamide, salicylsalicylic acid, satigrel, sudoxicam, suprofen, talmetacin, talniflumate, tazofelone, tebufelone, tenidap, tenoxicam, tepoxalin, tiaprofenic acid, tiaramide, tilmacoxib, tinoridine, tiopinac, tioxaprofen, tolfenamic acid, triflusal, tropesin, ursolic acid, valdecoxib, ximoprofen, zaltoprofen, zidometacin, and zomepirac, and the salts, solvates, analogues, congeners, bioisosteres, hydrolysis products, metabolites, precursors, and prodrugs thereof.

The clinical use of corticosteroids is described in B. P. Schimmer & K. L. Parker, “Adrenocorticotropic Hormone; Adrenocortical Steroids and Their Synthetic Analogs; Inhibitors of the Synthesis and Actions of Adrenocortical Hormones” in Goodman & Gilman's The Pharmacological Basis of Therapeutics (L. L. Brunton, ed., 11^{th} ed., McGraw-Hill, New York, 2006), ch. 59, pp. 1587-1612.

Anti-nausea treatments include, but are not limited to, ondansetron, metoclopramide, promethazine, cyclizine, hyoscine, dronabinol, dimenhydrinate, diphenhydramine, hydroxyzine, medizine, dolasetron, granisetron, palonosetron, ramosetron, domperidone, haloperidol, chlorpromazine, fluphenazine, perphenazine, prochlorperazine, betamethasone, dexamethasone, lorazepam, and thiethylperazine.

Anti-diarrheal treatments include, but are not limited to, diphenoxylate, difenoxin, loperamide, codeine, racecadotril, octreoside, and berberine.

N-acetylcysteine is an antioxidant and mucolytic that also provides biologically accessible sulfur.

Poly-ADP ribose polymerase (PARP) inhibitors include, but are not limited to: (1) derivatives of tetracycline as described in U.S. Pat. No. 8,338,477 to Duncan et al.; (2) 3,4-dihydro-5-methyl-1(2H)-isoquinoline, 3-aminobenzamide, 6-aminonicotinamide, and 8-hydroxy-2-methyl-4(3H)-quinazolinone, as described in U.S. Pat. No. 8,324,282 by Gerson et al.; (3) 6-(5H)-phenanthridinone and 1,5-isoquinolinediol, as described in U.S. Pat. No. 8,324,262 by Yuan et al.; (4) (R)-3-[2-(2-hydroxymethylpyrrolidin-1-yl)ethyl]-5-methyl-2H-isoquinolin-1-one, as described in U.S. Pat. No. 8,309,573 to Fujio et al.; (5) 6-alkenyl-substituted 2-quinolinones, 6-phenylalkyl-substituted quinolinones, 6-alkenyl-substituted 2-quinoxalinones, 6-phenylalkyl-substituted 2-quinoxalinones, substituted 6-cyclohexylalkyl substituted 2-quinolinones, 6-cyclohexylalkyl substituted 2-quinoxalinones, substituted pyridones, quinazolinone derivatives, phthalazine derivatives, quinazolinedione derivatives, and substituted 2-alkyl quinazolinone derivatives, as described in U.S. Pat. No. 8,299,256 to Vialard et al.; (6) 5-bromoisoquinoline, as described in U.S. Pat. No. 8,299,088 to Mateucci et al.; (7) 5-bis-(2-chloroethyl)amino]-1-methyl-2-benzimidazolebutyric acid, 4-iodo-3-nitrobenzamide, 8-fluoro-5-(4-((methylamino)methyl)phenyl)-3,4-dihydro-2H-azepino[5,4,3-cd]indol-1(6H)-one phosphoric acid, and N-[3-(3,4-dihydro-4-oxo-1-phthalazinyl)phenyl]-4-morpholinebutanamide methanesulfonate, as described in U.S. Pat. No. 8,227,807 to Gallagher et al.; (8) pyridazinone derivatives, as described in U.S. Pat. No. 8,268,827 to Branca et al.; (9) 4-[3-(4-cyclopropanecarbonyl-piperazine-1-carbonyl)-4-fluorobenzyl]-2H-phthalazin-1-one, as described in U.S. Pat. No. 8,247,416 to Menear et al.; (10) tetraaza phenalen-3-one compounds, as described in U.S. Pat. No. 8,236,802 to Xu et al.; (11) 2-substituted-1H-benzimidazole-4-carboxamides, as described in U.S. Pat. No. 8,217,070 to Zhu et al.; (12) substituted 2-alkyl quinazolinones, as described in U.S. Pat. No. 8,188,103 to Van der Aa et al.; (13) 1H-benzimidazole-4-carboxamides, as described in U.S. Pat. No. 8,183,250 to Penning et al.; (14) indenoisoquinolinone analogs, as described in U.S. Pat. No. 8,119,654 to Jagtap et al.; (15) benzoxazole carboxamides, described in U.S. Pat. No. 8,088,760 to Chu et al; (16) diazabenzo[de] anthracen-3-one compounds, described in U.S. Pat. No. 8,058,075 to Xu et al.; (17) dihydropyridophthalazinones, described in U.S. Pat. No. 8,012,976 to Wang et al., (18) substituted azaindoles, described in U.S. Pat. No. 8,008,491 to Jiang et al.; (19) fused tricyclic compounds, described in U.S. Pat. No. 7,956,064 to Chua et al.; (20) substituted 6a,7,8,9-tetrahydropyrido[3,2-e]pyrrolo[1,2-a]pyrazin-6(5H)-ones, described in U.S. Pat. No. 7,928,105 to Gangloff et al.; and (21) thieno[2,3-c] isoquinolines, described in U.S. Pat. No. 7,825,129. Other PARP inhibitors are known in the art.

When the improvement is made by pharmacokinetic/pharmacodynamic monitoring, the pharmacokinetic/pharmacodynamic monitoring can be, but is not limited to a method selected from the group consisting of:

(a) multiple determinations of blood plasma levels; and

(b) multiple determinations of at least one metabolite in blood or urine.

Typically, determination of blood plasma levels or determination of at least one metabolite in blood or urine is carried out by immunoassays. Methods for performing immunoassays are well known in the art, and include radioimmunoassay, ELISA (enzyme-linked immunosorbent assay), competitive immunoassay, immunoassay employing lateral flow test strips, and other assay methods.

When the improvement is made by drug combination, the drug combination can be, but is not limited to, a drug combination selected from the group consisting of:

(a) use with fraudulent nucleosides;

(b) use with fraudulent nucleotides;

(c) use with thymidylate synthetase inhibitors;

(d) use with signal transduction inhibitors;

(e) use with cisplatin or platinum analogs;

(f) use with alkylating agents;

(g) use with anti-tubulin agents;

(h) use with antimetabolites;

(i) use with berberine;

(j) use with apigenin;

(k) use with colchicine or an analog thereof;

(l) use with genistein;

(m) use with etoposide;

(n) use with cytarabine;

(o) use with camptothecins;

(p) use with vinca alkaloids;

(q) use with topoisomerase inhibitors;

(r) use with 5-fluorouracil;

(s) use with curcumin;

(t) use with NF-κB inhibitors;

(u) use with rosmarinic acid;

(v) use with mitoguazone;

(w) use with meisoindigo;

(x) use with imatinib;

(y) use with dasatinib;

(z) use with nilotinib;

(aa) use with epigenetic modulators;

(ab) use with transcription factor inhibitors;

(ac) use with taxol;

(ad) use with homoharringtonine;

(ae) use with pyridoxal;

(af) use with spirogermanium;

(ag) use with caffeine;

(ah) use with nicotinamide;

(ai) use with methylglyoxalbisguanylhydrazone;

(aj) use with Rho kinase inhibitors;

(ak) use with 1,2,4-benzotriazine oxides;

(al) use with an alkylglycerol;

(am) use with an inhibitor of a Mer, Ax1, or Tyro-3 receptor kinase;

(an) use with an inhibitor of ATR kinase;

(ao) use with a modulator of Fms kinase, Kit kinase, MAP4K4 kinase, TrkA kinase, or TrkB kinase;

(ap) use with endoxifen;

(aq) use with a mTOR inhibitor;

(ar) use with an inhibitor of Mnk1a kinase, Mkn1b kinase, Mnk2a kinase, or Mnk2b kinase;

(as) use with a modulator of pyruvate kinase M2;

(at) use with a modulator of phosphoinositide 3-kinases;

(au) use with a cysteine protease inhibitor;

(av) use with phenformin;

(aw) use with Sindbis virus-based vectors;

(ax) use with peptidomimetics that act as mimetics of Smac and inhibit IAPs to promote apoptosis;

(ay) use with a Raf kinase inhibitor;

(az) use with a nuclear transport modulator;

(ba) use with an acid ceramidase inhibitor and a choline kinase inhibitor;

(bb) use with tyrosine kinase inhibitors;

(bc) use with anti-CS1 antibodies;

(bd) use with inhibitors of protein kinase CK2;

(be) use with anti-guanylyl cyclase C (GCC) antibodies;

(bf) use with histone deacetylase inhibitors;

(bg) use with cannabinoids;

(bh) use with glucagon-like peptide-1 (GLP-1) receptor agonists;

(bi) use with inhibitors of Bcl-2 or Bcl-xL;

(bj) use with Stat3 pathway inhibitors;

(bk) use with inhibitors of polo-like kinase 1 (Plk1);

(bl) use with GBPAR1 activators;

(bm) use with modulators of serine-threonine protein kinase and poly(ADP-ribose) polymerase (PARP) activity;

(bn) use with taxanes;

(bo) use with inhibitors of dihydrofolate reductase;

(bp) use with inhibitors of aromatase;

(bq) use with benzimidazole-based anti-neoplastic agents;

(br) use with an 06-methylguanine-DNA-methyltransferase (MGMT) inhibitor;

(bs) use with CCR9 inhibitors;

(bt) use with acid sphingomyelinase inhibitors;

(bu) use with peptidomimetic macrocycles;

(bv) use with cholanic acid amides;

(bw) use with substituted oxazaphosphorines;

(bx) use with anti-TWEAK receptor antibodies;

(by) use with an ErbB3 binding protein;

(bz) use with a glutathione S-transferase-activated anti-neoplastic compound;

(ca) use with substituted phosphorodiamidates;

(cb) use with inhibitors of MEKK protein kinase;

(cd) use with COX-2 inhibitors;

(ce) use with cimetidine and a cysteine derivative;

(cf) use with anti-IL-6 receptor antibody;

(cg) use with an antioxidant;

(ch) use with an isoxazole inhibitor of tubulin polymerization;

(ci) use with PARP inhibitors;

(cj) use with Aurora protein kinase inhibitors;

(ck) use with peptides binding to prostate-specific membrane antigen;

(cl) use with CD19 binding agents;

(cm) use with benzodiazepines;

(cn) use with Toll-like receptor (TLR) agonists;

(co) use with bridged bicyclic sulfamides;

(cp) use with inhibitors of epidermal growth factor receptor kinase;

(cq) use with a ribonuclease of the T2 family having actin-binding activity;

(cr) use with myrsinoic acid A or an analog thereof;

(cs) use with inhibitors of a cyclin-dependent kinase;

(ct) use with inhibitors of the interaction between p53 and MDM2;

(cu) use with inhibitors of the receptor tyrosine kinase MET;

(cv) use with largazole or largazole analogs;

(cw) use with inhibitors of AKT protein kinase;

(cx) use with 2′-fluoro-5-methyl-β-L-arabinofuranosyluridine or L-deoxythymidine;

(cy) use with HSP90 modulators;

(cz) use with inhibitors of JAK kinases;

(da) use with inhibitors of PDK1 protein kinase;

(db) use with PDE4 inhibitors;

(de) use with inhibitors of proto-oncogene c-Met tyrosine kinase;

(df) use with inhibitors of indoleamine 2,3-dioxygenase;

(dg) use with agents that inhibit expression of ATDC (TRIM29);

(dh) use with proteomimetic inhibitors of the interaction of nuclear receptor with coactivator peptides;

(di) use with antagonists of XIAP family proteins;

(dj) use with tumor-targeted superantigens;

(dk) use with inhibitors of Pim kinases;

(dl) use with inhibitors of CHK1 or CHK2 kinases;

(dm) use with inhibitors of angiopoietin-like 4 protein;

(dn) use with Smo antagonists;

(do) use with nicotinic acetylcholine receptor antagonists;

(dp) use with farnesyl protein transferase inhibitors;

(dq) use with adenosine A3 receptor antagonists;

(dr) use with a cancer vaccine;

(ds) use with a JAK2 inhibitor; and

(dt) use with a Src inhibitor.

Topoisomerase inhibitors include topoisomerase I inhibitors and topoisomerase II inhibitors. Topoisomerase I inhibitors include the camptothecins and lamellarin D. Topoisomerase II inhibitors include, in addition to amonafide and derivatives and analogs thereof, etoposide, teniposide, doxorubicin, daunorubicin, mitoxantrone, amsacrine, ellipticines, aurintricarboxylic acid, and ICRF-193 (4-[2-(3,5-dioxo-1-piperazinyl)-1-methylpropyl]piperazine-2,6-dione). A number of plant-derived naturally-occurring phenolic compounds, such as genistein, quercetin, and resveratrol, exhibit inhibitory activity toward both topoisomerase I and topoisomerase II.

Fraudulent nucleosides include, but are not limited to, cytosine arabinoside, gemcitabine, and fludarabine; other fraudulent nucleosides are known in the art.

Fraudulent nucleotides include, but are not limited to, tenofovir disoproxil fumarate and adefovir dipivoxil; other fraudulent nucleotides are known in the art.

Thymidylate synthetase inhibitors include, but are not limited to, raltitrexed, pemetrexed, nolatrexed, plevitrexed, GS7094L, fluorouracil, and N-[4-[2-propyn-1-yl[6S)-4,6,7,8-tetrahydro-2-(hydroxymethyl)-4-oxo-3H-cyclopenta[g]quinazolin-6-yl]amino]benzoyl]-1-γ-glutamyl-D-glutamic acid (BGC 945).

Signal transduction inhibitors are described in A. V. Lee et al., “New Mechanisms of Signal Transduction Inhibitor Action: Receptor Tyrosine Kinase Down-Regulation and Blockade of Signal Transactivation,” Clin. Cancer Res. 9: 516s (2003).

Alkylating agents include, but are not limited to, Shionogi 254-S(cis-diammine(glycolato)platinum), aldo-phosphamide analogues, altretamine, anaxirone, Boehringer Mannheim BBR-2207, bendamustine, bestrabucil, budotitane, Wakunaga CA-102, carboplatin, carmustine, Chinoin-139, Chinoin-153, chlorambucil, cisplatin, cyclophosphamide, zeniplatin, ecomustine, cyplatate, Degussa D-19-384, Sum imoto DACHP(Myr)₂, diphenylspiromustine, diplatinum cytostatic, Erba distamycin derivatives, Chugai DWA-2114R ((R)-(+1,1-(2-amino-methylpyrrorodine)-platinum(II)), ITI E09, elmustine, Erbamont FCE-24517 (β-[1-methyl-4-(1-methyl-4-[1-methyl-4-(4-N,N-bis(2-chloroethyl) amino-benzene-1-carboxy-amido) pyrrole-2-carboxyamido]pyrrole-2-carboxyamido)pyrrole-2-carboxyamido]) propionamidine hydrochloride), estramustine phosphate sodium, fotemustine, Unimed G-6-M, Chinoin GYKI-17230 ([(2R,3R,4R,5R)-2,5-dihydroxy-3,4-dimethoxy-6-methylsulfonyloxy-hexyl] methanesulfonate), hepsulfam, ifosfamide, iproplatin, lomustine, mafosfamide, melphalan, mitolactol, Nippon Kayaku NK-121, NCI NSC-264395 (N-(2-chloroethyl N′-(2,6-dihydroxycyclohexyl)-N-nitrosourea), NCI NSC-342215 (d-1-deoxy-1-(bis(2-chloroethyl)amino)-2,3,4,6-O-tetraacetylglucopyranose), oxaliplatin, Upjohn PCNU, prednimustine, Proter PTT-119 (3-p-fluorophenyl-L-alanyl-3-m-bis-(2-chloroethyl)-aminophenyl-L-alanyl-L methionine ethyl ester hydrochloride), ranimustine, semustine, SmithKline SK&F-101772, Yakult Honsha SN-22, spiromustine, Tanabe Seiyaku TA-077 (1-(2-chloroethyl)-3-isobutyl-3-(beta-maltosyl)-1-nitrosourea), tauromustine, temozolomide, teroxirone, tetraplatin and trimelamol, as described in U.S. Pat. No. 7,446,122 by Chao et al.

Anti-tubulin agents include, but are not limited to, vinca alkaloids, taxanes, podophyllotoxin, halichondrin B, and homohalichondrin B.

Antimetabolites include, but are not limited to: methotrexate, pemetrexed, 5-fluorouracil, capecitabine, cytarabine, gemcitabine, 6-mercaptopurine, and pentostatin, alanosine, AG2037 (Pfizer), 5-FU-fibrinogen, acanthifolic acid, aminothiadiazole, brequinar sodium, carmofur, Ciba-Geigy CGP-30694, cyclopentyl cytosine, cytarabine phosphate stearate, cytarabine conjugates, Lilly DATHF, Merrill-Dow DDFC, deazaguanine, dideoxycytidine, dideoxyguanosine, didox, Yoshitomi DMDC, doxifluridine, Wellcome EHNA, Merck & Co. EX-015, fazarabine, floxuridine, fludarabine phosphate, N-(2′-furanidyl)-5-fluorouracil, Daiichi Seiyaku FO-152, isopropyl pyrrolizine, gemcitabine, lometrexol, methobenzaprim, methotrexate, Wellcome MZPES, norspermidine, 5-aza-2′deoxycytidine, 5,6-dihydro-5-azacytidine, 5-iodo-2′-deoxyuridine, aldophosphamide perhydrothiazine, Warner-Lambert PALA, piritrexim, plicamycin, Asahi Chemical PL-AC, Takeda TAC-788, thioguanine, tiazofurin, Erbamont TIF, trimetrexate, tyrosine kinase inhibitors, tyrosine protein kinase inhibitors, Taiho UFT and uricytin.

Berberine has antibiotic activity and prevents and suppresses the expression of pro-inflammatory cytokines and E-selectin, as well as increasing adiponectin expression.

Apigenin is a flavone that can reverse the adverse effects of cyclosporine and has chemoprotective activity, either alone or derivatized with a sugar.

Colchicine is a tricyclic alkaloid that exerts its activity by binding to the protein tubulin. Analogs of colchicine include, but are not limited to, colchiceinamide, N-desacetylthiocolchicine, demecolcine, N-acetyliodocolchinol, trimethylcolchicinic acid (TMCA) methyl ether, N-acetylcolchinol, TMCA ethyl ether, isocolchicine, isocolchiceinamide, iso-TMCA methyl ether, colchiceine, TMCA, N-benzoyl TMCA, colchicosamide, colchicoside, colchinol and colchinoic acid (M. H. Zweig & C. F. Chignell, “Interaction of Some Colchicine Analogs, Vinblastine and Podophyllotoxin with Rat Brain Microtubule Protein,” Biochem. Pharmacol. 22: 2141-2150 (1973) and B. Yang et al., “Syntheses and Biological Evaluation of Ring C-Modified Colchicine Analogs,” Bioorg. Med. Chem. Lett. 20: 3831-3833 (2010)).

Genistein is an isoflavone with the systemic name 5,7-dihydroxy-3-(4-hydroxyphenyl)chromen-4-one. Genistein has a number of biological activities, including activation of PPARs, inhibition of several tyrosine kinases, inhibition of topoisomerase, antioxidative activity, activation of Nrf2 antioxidative response, activation of estrogen receptor beta, and inhibition of the mammalian hexose transporter GLUT2.

Etoposide is an anticancer agent that acts primarily as a topoisomerase II inhibitor. Etoposide forms a ternary complex with DNA and the topoisomerase II enzyme, prevents re-ligation of the DNA strands and thus induces DNA strand breakage and promotes apoptosis of the cancer cells.

Cytarabine is a nucleoside analog replacing the ribose with arabinose. It can be incorporated into DNA and also inhibits both DNA and RNA polymerases and nucleotide reductase. It is particularly useful in the treatment of acute myeloid leukemia and acute lymphocytic leukemia, but can be used for other malignancies and in various drug combinations.

Camptothecins include camptothecin, homocamptothecin, topotecan, irinotecan, silatecan, karenitecin, exatecan, lurtotecan, gimatecan, and belotecan. These compounds act as topoisomerase I inhibitors and block DNA synthesis in cancer cells.

Vinca alkaloids include vinblastine, vincristine, vindesine, and vinorelbine.

Topoisomerase inhibitors include topoisomerase I inhibitors and topoisomerase II inhibitors. Topoisomerase I inhibitors include the camptothecins and lamellarin D. Topoisomerase II inhibitors include, in addition to amonafide and derivatives and analogs thereof, etoposide, teniposide, doxorubicin, daunorubicin, mitoxantrone, amsacrine, ellipticines, and aurintricarboxylic acid. A number of plant-derived naturally-occurring phenolic compounds, such as genistein, quercetin, and resveratrol, exhibit inhibitory activity toward both topoisomerase I and topoisomerase II. As detailed below, camptothecins and other topoisomerase inhibitors can be used with dianhydrogalactitol or a derivative or analog thereof so that the cell-cycle-dependent activity of the topoisomerase inhibitors is optimized and so that the quantity of the topoisomerase inhibitors required is reduced.

The compound 5-fluorouracil is a base analog that acts as a thymidylate synthase inhibitor and thereby inhibits DNA synthesis. When deprived of a sufficient supply of thymidine, rapidly dividing cancer cells die by a process known as thymineless death.

Curcumin is believed to have anti-neoplastic, anti-inflammatory, antioxidant, anti-ischemic, anti-arthritic, and anti-amyloid properties and also has hepatoprotective activity.

NF-κB inhibitors include, but are not limited to bortezomib.

Rosmarinic acid is a naturally-occurring phenolic antioxidant that also has anti-inflammatory activity.

Mitoguazone is an inhibitor of polyamine biosynthesis through competitive inhibition of S-adenosylmethionine decarboxylase.

Meisoindigo is active via several, possibly novel mechanisms of action. It has cell cycle specific effects, including arrest in G(O)/G1 for AML cell lines and G2/M arrest for HT-29 colorectal cell lines. It also stimulates apoptosis through a number of mechanisms, including the upregulation of p21 and p27 and the downregulation of Bcl-2 in primary AML cells, as well as upregulation of Bak and Bax in AML cells (DKO insensitive to chemotherapy), and a novel caspase-dependent pathway in K562 cells. Meisoindigo also has effects on mitochondria, but with no change in Bcl-2, Bax, and Bid protein expression. Meisoindigo also stimulates the cleavage of pro-caspase 3, 8, 9 and PARP in HL-60 myeloid cells. Meisoindigo also is directed to multiple cellular targets, which are possibly synergistic and complementary. For example, it promotes differentiation of human myeloblastic leukemic cells, accompanied by downregulation of c-myb gene expression. It also promotes inhibition of DNA and RNA synthesis in W256 cells, microtubule assembly, glycogen synthase kinase-3p (GSK-3β) (at 5-50 nM), CDK1/cyclin B, and CDK5/p25 (tau microtubule protein phosphorylation). Additionally, meisoindigo decreases β-catenin and c-myc (HL-60 cells, but not in K562), affects the Wnt pathway through inhibiting GSK-3β and downregulating β-catenin and c-myc protein expression. Meisoindigo also promotes upregulation of CD11b, promoting myeloid differentiation, and upregulation of Ahi-1 in Jurkat cells (inducing phosphorylation of c-Myb). Furthermore, meisoindigo exhibits antiangiogenic effects, including decreased VEGF protection, VCAM-1, tubule formulation in HUVEC, and ECV304 apoptosis.

Imatinib is an inhibitor of the receptor tyrosine kinase enzyme ABL and is used to treat chronic myelogenous leukemia, gastrointestinal stromal tumors, and other hyperproliferative disorders.

Dasatinib is an inhibitor of BCR/ABL and Src family tyrosine kinases and is used to treat chronic myelogenous leukemia and acute lymphoblastic leukemia.

Nilotinib is another tyrosine kinase inhibitor approved for the treatment of chronic myelogenous leukemia; it inhibits the kinases BCR/ABL, KIT, LCK, EPHA3, and a number of other kinases. The use of nilotinib is described in United States Patent Application Publication No. 2011/0028422 by Aloyz et al.

Epigenetic modulators include polyamine-based epigenetic modulators, such as the polyamine-based epigenetic modulators described in S. K. Sharma et al., “Polyamine-Based Small Molecule Epigenetic Modulators,” Med. Chem. Commun. 3: 14-21 (2012), and L. G. Wang & J. W. Chiao, “Prostate Cancer Chemopreventive Activity of Phenethyl Isothiocyanate Through Epigenetic Regulation (Review), Int. J. Oncol. 37: 533-539 (2010).

Transcription factor inhibitors include 1-(4-hexaphenyl)-2-propane-1-one, 3-fluoro-4-[[2-hydroxy-2-(5,5,8,8-tetramethyl-5,6,7,8,-tetrahydro-2-naphthalenyl)acetyl]amino]-benzoic acid (BMS 961), 4-[5-[8-(1-methylethyl)-4-phenyl-2-quinolinyl]-1H-pyrrolo-2-benzoic acid (ER-50891), 7-ethenyl-2-(3-fluoro-4-hydroxyphenyl)-5-benzoxazolol (ERB 041), and other compounds. Trascription factor inhibitors are described in T. Berg, “Inhibition of Transcription Factors with Small Organic Molecules,” Curr. Opin. Chem. Biol. 12: 464-471 (2008).

Tetrandrine has the chemical structure 6,6′,7,12-tetramethoxy-2,2′-dimethyl-1 β-berbaman and is a calcium channel blocker that has anti-inflammatory, immunologic, and antiallergenic effects, as well as an anti-arrhythmic effect similar to that of quinidine. It has been isolated from Stephania tetranda and other Asian herbs.

VEGF inhibitors include bevacizumab (Avastin), which is a monoclonal antibody against VEGF, itraconazole, and suramin, as well as batimastat and marimastat, which are matrix metalloproteinase inhibitors, and cannabinoids and derivatives thereof.

Cancer vaccines are being developed. Typically, cancer vaccines are based on an immune response to a protein or proteins occurring in cancer cells that does not occur in normal cells. Cancer vaccines include Provenge for metastatic hormone-refractory prostate cancer, Oncophage for kidney cancer, CimaVax-EGF for lung cancer, MOBILAN, Neuvenge for Her2/neu expressing cancers such as breast cancer, colon cancer, bladder cancer, and ovarian cancer, Stimuvax for breast cancer, and others. Cancer vaccines are described in S. Pejawar-Gaddy & O. Finn, “Cancer Vaccines: Accomplishments and Challenges,” Crit. Rev. Oncol. Hematol. 67: 93-102 (2008).

The use of methylglyoxalbisguanylhydrazone in cancer therapy has been described in D. D. Von Hoff, “MGBG: Teaching an Old Drug New Tricks,” Ann. Oncol. 5: 487-493 (1994).

The use of Rho kinase inhibitors, such as (R)-(+)-N-(4-pyridyl)-4-(1-aminoethyl)benzamide, ethacrynic acid, 4-[2(2,3,4,5,6-pentafluorophenyl)acryloyl]cinnamic acid, (+)-trans-4-(1-aminoethyl)-1-(4-pyridylcarbamoyl)cyclohexane, (+)-10 trans-N-(1H-pyrrolo[2,3-b]pyridin-4-yl)-4-(1-aminoethyl)cyclohexanecarboxamide, and (R)-(+)-N-(1H-pyrrolo[2,3-b]pyridin-4-yl)-4-(1-aminoethyl)benzamide, as described in U.S. Pat. No. 6,930,115 to Fujii et al.

The use of 1,2,4-benzotriazine oxides, such as 3-hydroxy-1,2,4-benzotriazine 1,4-dioxide, 3-amino-7-trifluoromethyl-1,2,4-benzotriazine 1-oxide, 3-amino-7-carbamyl-1,2,4-benzotriazine 1-oxide, 7-acetyl-3-amino-1,2,4-benzotriazine 1-oxide oxime, 3-amino-6(7)decyl-1,2,4-benzotriazine 1,4-dioxide, 1,2,4-benzotriazine dioxide, 7-chloro-3-hydroxy-1,2,4-benzotriazine 1,4-dioxide, 7-nitro-3-amino-1,2,4-benzotriazine 1,4-dioxide, 3-(3-N,N-diethylaminopropylamino)-1,2,4-benzotriazine 1,4-dioxide, 7-nitro-3-(2-N,N-diethylaminoethylamino)-1,2,4-benzotriazine 1,4-dioxide, 7-allyloxy-1,2,4-benzotriazine 1,4-dioxide, 7-(3-N-ethylacetamido-2-acetoxypropoxy) 1,2,4-benzotriazine 1,4-dioxide, 7-nitro-1,2,4-benzotriazine 1,4-dioxide. 3-propyl-1,2,4-benzotriazine 1,4-dioxide, and 3-(1-hydroxyethyl)-1,2,4-benzotriazine 1,4-dioxide, as described in U.S. Pat. No. 6,277,835 by Brown.

The use of alkylglycerols is described in U.S. Pat. No. 6,121,245 to Firshein.

The use of inhibitors of Mer, Ax1, or Tyro-3 receptor tyrosine kinase is described in United States Patent Application Publication No. 2012/0230991 by Graham et al. These inhibitors can be antibodies, including monoclonal antibodies, or fusion proteins.

The use of inhibitors of ATR kinase is described in United States Patent Application Publication No. 2012/0177748 by Charrier et al., incorporated by these reference. These inhibitors of ATR kinase are substituted pyridine compounds such as 2-amino-N-phenyl-5-(3-pyridyl)pyridine-3-carboxamide, 5-(4-(methylsulfonyl)phenyl-3-(5-phenyl-1,3,4-oxadiazol-2-yl)pyridine-2-amine, and 5-(1-ethylsulfonyl-3,6-dihydro-2H-pyridin-4-yl)-3-(5-phenyl-1,3,4-oxadiazol-2-yl)pyridine-2-amine.

The use of compounds that modulate the activity of one or more of Fms kinase, Kit kinase, MAP4K4 kinase, TrkA kinase, or TrkB kinase is described in United States Patent Application Publication No. 2012/0165329 by Ibrahim et al. These compounds include (6-methoxy-pyridin-3-ylmethyl)[5-(7H-pyrrolo [2,3-d] pyrimidin-5-ylmethyl)-pyrimidin-2-yl]-amine, (5-fluoro-2-methoxy-pyridin-3-ylmethyl)-[5-(7H-pyrrolo[2,3-d]pyrimidin-5-ylmethyl)-pyrimidin-2-y]-amine, and (5-fluoro-6-methoxy-pyridin-3-ylmethyl)-[5-(7H-pyrrolo[2,3-d]pyrimidin-5-ylmethyl)-pyrimidin-2-yl]-amine. Compounds that inhibit Trk kinases, particularly TrkA, are described in United States Patent Application Publication No. 2011/0301133 by Wu et al.

The use of endoxifen is described in United States Patent Application Publication No. 2012/0164075 by Ahmad et al.

The use of a mTOR inhibitor is described in United States Patent Application Publication No. 2012/0129881 by Burke et al. Suitable mTOR inhibitors include, but are not limited to, 40-O-(2-hydroxyethyl)rapamycin. These mTOR inhibitors can be used together with Raf kinase inhibitors, as described in United States Patent Application Publication No. 2011/0301184 by Lane. Raf kinase inhibitors are also described in United States Patent Application Publication No. 2010/0286178 by Ibrahim et al.; these compounds include, but are not limited to, propane-1-sulfonic acid {2,4-difluoro-3-[5-(2-methoxy-pyrimidin-5-yl)-1H-pyrrolo[2,3-b]pyridine-3-carbonyl]-phenyl}-amide, propane-1-sulfonic acid [3-(5-cyano-1H-pyrrolo[2,3-b]pyridine-3-carbonyl)-2,4-difluoro-phenyl]-amide, propane-1-sulfonic acid [3-(5-cyano-1H-pyrrolo[2,3-b]pyridine-3-carbonyl)-2-fluoro-phenyl]-amide, N-[3-(5-cyano-1H-pyrrolo[2,3-b]pyridine-3-carbonyl)-2,4-difluoro-phenyl]-2,5-difluoro-benzenesulfonamide, N-[3-(5-cyano-1H-pyrrolo[2,3-b]pyridine-3-carbonyl)-2,4-difluoro-phenyl]-3-fluoro-benzenesulfonamide, pyrrolidine-1-sulfonic acid [3-(5-cyano-1H-pyrrolo[2,3-b]pyridine-3-carbonyl)-2,4-difluoro-phenyl]-amide, and N,N-dimethylamino-sulfonic acid [3-(5-cyano-1H-pyrrolo[2,3-b]pyridine-3-carbonyl)-2,4-difluoro-phenyl]-amide. These mTOR inhibitors can also be used together with compounds that elevate pAkt levels in malignant cells, as described in United States Patent Application Publication No. 2009/0274698 by Bhagwat et al. A number of compounds that elevate pAkt levels are described, including chemotherapeutic agents, analogs of rapamycin, and other agents. The use of mTOR inhibitors is also described in U.S. Pat. No. 8,268,819 to Jin et al.; these mTOR inhibitors are hexahydrooxazinopterine compounds.

The use of an inhibitor of Mnk1a kinase, Mnk1b kinase, Mnk2a kinase, or Mnk2b kinase is described in United States Patent Application Publication No. 2012/0128686 by Austen et al. These compounds include thienopyrimidines. Additional thienopyrimidine inhibitors of one or more of these kinases are described in United States Patent Application Publication No. 2011/0212103 by Heckel et al. and in United States Patent Application Publication No. 2011/0212102 by Lehmann-Lintz et al.

The use of a modulator of pyruvate kinase M2 is described in United States Patent Application Publication 2012/0122885 by Salituro et al. Suitable modulators of pyruvate kinase M2 include, but are not limited to, 1-(3-chloro-5-(trifluoromethyl)pyridin-2-yl)-N-(3,5-dimethylphenyl)-1H-imidazole-5-sulfonamide; 1-(3-chloro-5-(trifluoromethyl)pyridin-2-yl)-N-(5-methoxyphenyl)-1H-imidazole-5-sulfonamide; and N-(4-methoxyphenyl)-1-(5-(trifluoromethyl)pyridine-2-yl)-H-imidazole-5-sulfonamide.

The use of a modulator of a phosphoinositide 3-kinase is described in United States Patent Application Publication No. 2012/0122838 by Ren et al. Inhibitors of phosphoinositide 3-kinase are also described in United States Patent Application Publication No. 2010/0209420 by Lamb et al., and in United States Patent Application Publication No. 2009/0209340 by Buhr et al.; these inhibitors include pyridopyrimidones. Inhibitors of phosphoinositide 3-kinase are also described in U.S. Pat. No. 8,242,104 to Blaquiere et al.; these inhibitors include benzoxazepines. Inhibitors of phosphoinositide 3-kinase are also described in U.S. Pat. No. 8,193,182 to Ren et al.; these inhibitors include isoquinolin-1(2H)-ones. Inhibitors of phosphoinositide 3-kinase are also described in U.S. Pat. No. 7,928,428 to Do et al.; these inhibitors include benzopyrans and benzoxepines.

The use of a cysteine protease inhibitor is described in United States Patent Application Publication No. 2012/0114765 by Cao et al. Suitable cysteine protease inhibitors include, but are not limited to, 1-[5-(2,4-dichlorophenylsulfanyl)-4-nitro-2-thienyl]ethanone, 1-[5-(2,4-difluorophenylsulfanyl)-4-nitro-2-thienyl]ethanone, and 1-{4-nitro-5-[2-(trifluoromethyl)phenylsulfanyl]-2-thienyl}ethanone.

The use of phenformin is described in United States Patent Application Publication No. 2012/0114676 by Thompson et al.

The use of Sindbis-based virus vectors is described in United States Patent Application Publication No. 2011/0318430 by Meruelo et al. These vectors are capable of binding to solid tumors that express higher levels of high affinity laminin receptors.

The use of peptidomimetics that act as mimetics of Smac and inhibit IAPs to promote apoptosis is described in United States Patent Application Publication No. 2011/0305777 by Condon et al.

The use of nuclear transport modulators, especially inhibitors of Crm1, is described in United States Patent Application Publication No. 2011/0275607 by Shacham et al. These inhibitors of Crm1 include, but are not limited to, (Z)-3-[3-(3-chlorophenyl)[1,2,4]-triazol-1-yl]-acrylic acid ethyl ester, (E)-3-[3-(3-chlorophenyl)[1,2,4]-triazol-1-yl]-acrylic acid ethyl ester, (Z)-3-[3-(3-chlorophenyl)-[1,2,4]-triazol-1-yl]-acrylic acid isopropyl ester, (E)-3-[3-(3-chlorophenyl)-[1,2,4]-triazol-1-yl]-acrylic acid isopropyl ester, (Z)-3-[3-(3-chlorophenyl)-[1,2,4]-triazol-1-yl]-acrylic acid t-butyl ester, (Z)-3-[3-(3-chlorophenyl)-[1,2,4]-triazol-1-yl]-acrylic acid t-butyl ester, (E)-3-[3-(3-chlorophenyl)-[1,2,4]-triazol-1-yl]-N-phenyl-acrylamide, (E)-N-(2-chlorophenyl)-3-[3-(3-chlorophenyl)-[1,2,4]-triazol-1-yl]-acrylamide, (4-{(E)-3-[3-(3-chlorophenyl)[1,2,4]-triazol-1-yl]-acryloylamino}-phenyl+carbamic acid t-butyl ester, (E)-3-[3-(3-chlorophenyl)-[1,2,4]-triazol-1-yl]-N-(4-methoxyphenyl)-acrylamide, (E)-3-[3-(3-chlorophenyl)-[1,2,4]-triazol-1-yl]-N-methyl-N-phenyl-acrylamide, and (E)-N-(4-aminophenyl)-3-[3-(3-chlorophenyl)-[1,2,4]-triazol-1-yl]-acrylamide.

The epidermal growth factor receptor (EGFR) exists on the cell surface of mammalian cells and is activated by binding of the receptor to its specific ligands, including, but not limited to epidermal growth factor and transforming growth factor α. Upon activation by binding to its growth factor ligands, EGFR undergoes a transition from an inactive monomeric form to an active homodimer, although preformed active dimers may exist before ligand binding. In addition to forming active homodimers after ligand binding, EGFR may pair with another member of the ErbB receptor family, such as ErbB2/Her2/neu, to create an activated heterodimer. There is also evidence that clusters of activated EGFRs form, although it is uncertain whether such clustering is important for activation itself or occurs subsequent to activation of individual dimers. EGFR dimerization stimulates its intracellular intrinsic protein-tyrosine kinase activity. As a result, autophosphorylation of several tyrosine residues in the carboxyl-terminal domain of EGFR occurs. These residues include Y992, Y1045, Y1068, Y1148, and Y1171. Such autophosphorylation elicits downstream activation and signaling by several other proteins that associate with the phosphorylated tyrosine residues through their own phosphotyrosine-binding SH2 domains. The signaling of these proteins that associate with the phosphorylated tyrosine residues through their own phosphotyrosine-binding SH2 domains can then initiate several signal transduction cascades and lead to DNA synthesis and cell proliferation. The kinase domain of EGFR can also cross-phosphorylate tyrosine residues of other receptors that it is aggregated with, and can itself be activated in that manner. EGFR is encoded by the c-erbB1 proto-oncogene and has a molecular mass of 170 kDa. It is a transmembrane glycoprotein with a cysteine-rich extracellular region, an intracellular domain containing an uninterrupted tyrosine kinase site, and multiple autophosphorylation sites clustered at the carboxyl-terminal tail as described above. The extracellular portion has been subdivided into four domains: domains I and III, which have 37% sequence identity, are cysteine-poor and conformationally contain the site for ligand (EGF and transforming growing factor α (TGFα) binding. Cysteine-rich domains II and IV contain N-linked glycosylation sites and disulfide bonds, which determine the tertiary conformation of the external domain of the protein molecule. In many human cell lines, TGFα expression has a strong correlation with EGFR overexpression, and therefore TGFα was considered to act in an autocrine manner, stimulating proliferation of the cells in which it is produced via activation of EGFR. Binding of a stimulatory ligand to the EGFR extracellular domain results in receptor dimerization and initiation of intracellular signal transduction, the first step of which is activation of the tyrosine kinase. The earliest consequence of kinase activation is the phosphorylation of its own tyrosine residues (autophosphorylation) as described above. This is followed by association with activation of signal transducers leading to mitogenesis. Mutations that lead to EGFR expression or overactivity have been associated with a number of malignancies, including glioblastoma. A specific mutation of EGFR known as EGFR Variant III has frequently been observed in glioblastoma (C. T. Kuan et al., “EGF Mutant Receptor VIII as a Molecular Target in Cancer Therapy,” Endocr. Relat. Cancer 8: 83-96 (2001)). EGFR is considered an oncogene. Inhibitors of EGFR include, but are not limited to, erlotinib, gefitinib, lapatinib, lapatinib ditosylate, afatinib, canertinib, neratinib, CP-724714, WHI-P154, TAK-285, AST-1306, ARRY-334543, ARRY-380, AG-1478, tyrphostin 9, dacomitinib, desmethylerlotinib, OSI-420, AZD8931, AEE788, pelitinib, CUDC-101, WZ8040, WZ4002, WZ3146, AG-490, XL647, PD153035 HCl, BMS-599626, BIBW 2992, CI 1033, CP 724714, OSI 420, and vandetinib. Particularly preferred EGFR inhibitors include erlotinib, afatinib, and lapatinib.

The use of tyrosine kinase inhibitors is described in United States Patent Application Publication No. 2011/0206661 by Zhang et al., which is directed to trimethoxyphenyl inhibitors of tyrosine kinase, and in United States Patent Application Publication No. 2011/0195066, which is directed to quinoline inhibitors of tyrosine kinase, both of which are incorporated herein by this reference. The use of tyrosine kinase inhibitors is also described in United States Patent Application Publication No. 2011/053968 by Zhang et al., which is directed to aminopyridine inhibitors of tyrosine kinase. The use of tyrosine kinase inhibitors is also described in United States Patent Application Publication No. 2010/0291025, which is directed to indazole inhibitors of tyrosine kinase. The use of tyrosine kinase inhibitors is also described in United States Patent Application Publication No. 2010/0190749 by Ren et al.; these tyrosine kinase inhibitors are benzoxazole compounds; compounds of this class can also inhibit mTOR and lipid kinases such as phosphoinositide 3-kinases. The use of tyrosine kinase inhibitors is also described in U.S. Pat. No. 8,242,270 by Lajeunesse et al.; these tyrosine kinase inhibitors are 2-aminothiazole-5-aromatic carboxamides.

The use of an acid ceramidase inhibitor and a choline kinase inhibitor is described in United States Patent Application Publication No. 2011/0256241 by Ramirez de Molina et al.

The use of anti-CS1 antibodies is described in United States Patent Application Publication No. 2011/0165154 by Afar.

The use of protein kinase CK2 inhibitors is described in United States Patent Application Publication No. 2011/0152240 by Haddach et al. These protein kinase CK2 inhibitors include pyrazolopyrimidines. Additional protein kinase CK2 inhibitors, including tricyclic compounds, are described in United States Patent Application Publication No. 2011/0071136 by Haddach et al.; these protein kinase CK2 inhibitors may also inhibit Pim kinases or other kinases. Additional protein kinase CK2 inhibitors, including heterocycle-substituted lactams, are also described in United States Patent Application Publication No. 2011/0071115 by Haddach et al.; these protein kinase CK2 inhibitors may also inhibit Pim kinases or other kinases.

The use of anti-guanylyl cyclase C (GCC) antibodies is described in United States Patent Application Publication No. 2011/0110936 by Nam et al.

The use of histone deacetylase inhibitors is described in United States Patent Application Publication No. 2011/0105474 by Thaler et al. These histone deacetylase inhibitors include, but are not limited to, (E)-N-hydroxy-3-{4-[(E)-3-(4-methyl-piperazin-1-yl)-3-oxo-propenyl]-phenyl}-acrylamide; (E)-N-hydroxy-3-{3-[(E)-3-(4-methyl-piperazin-1-yl)-3-oxo-propenyl]-phenyl}-acrylamide; (E)-N-hydroxy-3-{3-[(E)-3-oxo-3-(4-phenyl-piperazin-1-yl)-propenyl]-phenyl}-acrylamide; (E)-3-[3-((E)-3-[1,4′]bipiperidinyl-1′-yl-3-oxo-propenyl)-phenyl]-N-hydroxy-acrylamide; (E)-N-hydroxy-3-{3-[(E)-3-oxo-3-(cis-3,4,5-trimethyl-piperazin-1-yl)-propenyl]-phenyl}-acrylamide; (E)-3-{3-[(E)-3-((1S,4S)-5-methyl-2,5-diaza-bicyclo[2.2.1]hept-2-yl)-3-oxo-propenyl]-phenyl}-N-hydroxy-acrylamide; (E)-N-hydroxy-3-{4-[(E)-3-oxo-3-(4-phenyl-piperazin-1-yl)-propenyl]-phenyl}-acrylamide; (E)-3-[4-((E)-3-[1,4′]bipiperidinyl-1′-yl-3-oxo-propenyl)-phenyl]-N-hydroxy-acrylamide; (E)-N-hydroxy-3-{4-[(E)-3-oxo-3-(cis-3,4,5-trimethyl-piperazin-1-yl)-propenyl]-phenyl}-acrylamide; (E)-N-hydroxy-3-{4-[(E)-3-oxo-3-((1S,4S)-5-methyl-2,5-diaza-bicyclo[2.2.1]hept-2-yl)-propenyl]-phenyl}-acrylamide; (E)-N-hydroxy-3-{5-[(E)-3-oxo-3-(4-phenyl-piperazin-1-yl)-propenyl]-pyridin-2-yl}-acrylamide; (E)-N-hydroxy-3-{5-[(E)-3-(4-methyl-piperazin-1-yl)-3-oxo-propenyl]-pyridin-2-yl}-acrylamide; (E)-N-hydroxy-3-{6-[(E)-3-oxo-3-(4-phenyl-piperazin-1-yl)-propenyl]-pyridin-2-yl}-acrylamide; (E)-N-hydroxy-3-{6-[(E)-3-(4-methyl-piperazin-1-yl)-3-oxo-propenyl]-pyridin-2-yl}-acrylamide; (E)-3-(6-{(E)-3-[4-(3-chloro-phenyl)-piperazin-1-yl]-3-oxo-propenyl}-pyridin-2-yl)-N-hydroxy-acrylamide; (E)-3-{6-[(E)-3-(4-benzoyl-piperazin-1-yl)-3-oxo-propenyl]-pyridin-2-yl}-N-hydroxy-acrylamide hydrochloride; (E)-3-(6-{(E)-3-[4-(2-chloro-phenyl)-piperazin-1-yl]-3-oxo-propenyl}-pyridin-2-yl)-N-hydroxy-acrylamide hydrochloride; (E)-N-hydroxy-3-{6-[(E)-3-oxo-3-(4-phenyl-piperidin-1-yl)-propenyl]-pyridin-2-yl}-acrylamide hydrochloride; (E)-N-hydroxy-3-{6-[(E)-3-oxo-3-(4-pyrimidin-2-yl-piperazin-1-yl)-propenyl]-pyridin-2-yl}-acrylamide hydrochloride; (E)-3-(6-{(E)-3-[4-(4-chloro-phenyl)-piperazin-1-yl]-3-oxo-propenyl}-pyridin-2-yl)-N-hydroxy-acrylamide hydrochloride; and (E)-3-{6-[(E)-3-(4-benzyl-piperazin-1-yl)-3-oxo-propenyl]-pyridin-2-yl}-N-hydroxy-acrylamide hydrochloride. Additional histone deacetylase inhibitors, including spirocyclic derivatives, are described in United States Patent Application Publication No. 2011/039840 by Varasi et al. Prodrugs of histone deacetylase inhibitors are described in U.S. Pat. No. 8,227,636 to Miller et al. Histone deacetylase inhibitors are described in U.S. Pat. No. 8,222,451 to Kozikowski et al. Histone deacetylase inhibitors, including disubstituted aniline compounds, are also described in U.S. Pat. No. 8,119,685 to Heidebrecht et al. Histone deacetylase inhibitors, including aryl-fused spirocyclic compounds, are also described in U.S. Pat. No. 8,119,852 to Hamblett et al.

The use of cannabinoids is disclosed in United States Patent Application Publication No. 2011/0086113 by Velasco Diez et al. Suitable cannabinoids include, but are not limited to, tetrahydrocannabinol and cannabidiol.

The use of glucagon-like peptide-1 (GLP-1) receptor agonists is described in United States Patent Application Publication No. 2011/0046071 by Karasik et al. A suitable GLP-1 receptor agonist is exendin-4.

The use of inhibitors of anti-apoptotic proteins Bcl-2 or Bcl-xL is described in United States Patent Application Publication No. 2011/0021440 by Martin et al.

The use of Stat3 pathway inhibitors is described in United States Patent Application Publication No. 2010/0310503 by Li et al. These Stat3 pathway inhibitors include, but are not limited to, 2-(1-hydroxyethyl)-naphtho[2,3-b]furan-4,9-dione, 2-acetyl-7-chloro-naphtho[2,3-b]furan-4,9-dione, 2-acetyl-7-fluoro-naphtho[2,3-b]furan-4,9-dione, 2-acetylnaphtho[2,3-b]furan-4,9-dione, and 2-ethyl-naphtho[2,3-b]furan-4,9-dione.

The use of inhibitors of polo-like kinase 1 (Plk1) is described in United States Patent Application Publication No. 2010/0278833 by Stengel et al. These inhibitors include, but are not limited to, thiophene-imidazopyridines, including, but not limited to, 5-(6-chloro-1H-imidazo[4,5-c]pyridin-1-yl)-3-{[2-(trifluoromethyl)benzyl]oxy}thiophene-2-carboxamide, 5-(1H-imidazo[4,5-c]pyridin-1-yl)-3-{[2-(trifluoromethyl)benzyl]oxy}thiophene-2-carboxamide, 5-(3H-imidazo[4,5-c]pyridin-3-yl)-3-{[2-(trifluoromethyl)benzyl]oxy}thiophene-2-carboxamide, 1-(5-carbamoyl-4-{[2-(trifluoromethyl)benzyl]oxy}-2-thienyl)-N-(2-methoxyethyl)-1H-imidazo[4,5-c]pyridine-6-carboxamide, 1-(5-carbamoyl-4-{[2-(trifluoromethyl)benzyl]oxy}-2-thienyl)-N-(2-morpholin-4-ylethyl)-1H-imidazo[4,5-c]pyridine-6-carboxamide, 5-{6-[diethylamino)methyl]-1H-imidazo[4,5-c]pyridin-1-yl}-3-{[2-(trifluoromethyl)benzyl]oxy}thiophene-2-carboxamide, 5-{6-[(cyclopropylamino)methyl]-1H-imidazo[4,5-c]pyridin-1-yl}-3-{[2-(trifluoromethyl)benzyl]oxy}thiophene-2-carboxamide, 5-{6-[(4-methylpiperazin-1-yl)methyl]-1H-imidazo[4,5-c]pyridin-1-yl}-3-{[2-(trifluoromethyl)benzyl]oxy}thiophene-2-carboxamide, and 5-[6-(hydroxymethyl)-1H-imidazo[4,5-c]pyridin-1-yl]-3-{[2-(trifluoromethyl)benzyl]oxy}thiophene-2-carboxamide.

The use of GBPAR1 activators is described in United States Patent Application Publication No. 2010/0261758 by Arista et al., incorporated by this reference. These GBPAR1 activators include, but are not limited to, heterocyclic amides. These compounds include, but are not limited to, N-(3,5-dichlorophenyl)-3-methyl-N-naphthalen-2-ylmethyl-isonicotinamide, (3,5-dichlorophenyl)-N-(2-methoxybenzyl)-3-methyl-isonicotinamide, 3-methyl-N-phenyl-N-pyridin-3-ylmethyl-isonicotinamide, N-naphthalen-2-ylmethyl-1-oxy-N-phenyl-isonicotinamide, N-(3,5-dichlorophenyl)-3-methyl-N-(2-trifluoromethoxybenzyl)-isonicotinamide, 4-methyl-oxazole-5-carboxylic acid benzyl-phenylamide, N-benzyl-N-phenylisonicotinamide, N-benzyl-N-p-tolylisonicotinamide, N-benzyl-2-fluoro-N-phenylisonicotinamide, N-benzyl-3,5-dichloro-N-phenyl-isonicotinamide, N-benzyl-2-chloro-N-phenyl-isonicotinamide, N-benzyl-2-chloro-6-methyl-N-phenyl-isonicotinamide, N-benzyl-3-methyl-N-phenyl-isonicotinamide, N-benzyl-3-chloro-N-phenyl-isonicotinamide, N-benzyl-2,5-dichloro-N-phenyl-isonicotinamide, N-benzyl-2-methyl-N-phenyl-isonicotinamide, N-benzyl-2-cyano-N-phenyl-isonicotinamide, N-benzyl-N-phenethyl-isonicotinamide, N-benzyl-N-(2-fluoromethoxy-phenyl)-isonicotinamide, and N-benzyl-N-(4-chlorophenyl)-isonicotinamide. Additional GBPAR1 activators are described in United States Patent Application Publication No. 2010/0048579 by Arista, including pyridazine, pyridine, and pyrane derivatives.

The use of modulators of serine-threonine protein kinase and poly(ADP-ribose) polymerase (PARP) activity is described in United States Patent Application Publication No. 2009/0105233 by Chua et al. and in United States Patent Application Publication No. 2010/0173013 by Drygin et al., both incorporated herein by this reference. The serine-threonine protein kinase can be, but is not limited to, CK2, CK2a2, Pim-1, CDK1/cyclinB, c-RAF, Mer, MELK, DYRK2, Flt3, Flt3 (D835Y), Flt4, HIPK3, HIPK2, and ZIPK.

The use of taxanes is described in United States Patent Application Publication No. 2010/0166872 by Singh et al. The taxane can be, but is not limited to, paclitaxel or docitaxel.

The use of inhibitors of dihydrofolate reductase is described in United States Patent Application Publication No. 2010/0150896 by Gant et al. These inhibitors of dihydrofolate reductase include, but are not limited to, diaminoquinazolines.

The use of inhibitors of aromatase is described in United States Patent Application Publication No. 2010/0111901 by Gant et al. These inhibitors of aromatase include, but are not limited to, triazoles.

The use of benzimidazole-based anti-neoplastic agents is described in United States Patent Application Publication No. 2010/0098691 by Goh et al. The benzimidazole-based anti-neoplastic agent can be, but is not limited to, (E)-3-[1-(3-dimethylamino-2,2-dimethyl-propyl)-2-isopropyl-1H-benzimidazol-5-yl]-N-hydroxy-acrylamide, (E)-3-[2-butyl-1-(3-dimethylamino-2,2-dimethyl-propyl)-1H-benzimidazol-5-yl]-N-hydroxy-acrylamide, (E)-3-[1-(3-dimethylamino-2,2-dimethyl-propyl)-2-(2-methylsulfanyl-ethyl)-1H-benzimidazol-5-yl]-N-hydroxy-acrylamide, (E)-3-[1-(3-dimethylamino-2,2-dimethyl-propyl)-2-ethoxymethyl-1H-benzimidazol-5-yl]-N-hydroxy-acrylamide, (E)-3-[1-(3-dimethylamino-2,2-dimethyl-propyl)-2-isobutyl-1H-benzimidazol-5-yl]-N-hydroxy-acrylamide, (E)-3-[1-(2-diethylamino-ethyl)-2-isobutyl-1H-benzimidazol-5-yl]-N-hydroxy-acrylamide, (E)-3-[2-butyl-1-(2-diethylamino-ethyl)-1H-benzimidazol-5-yl]-N-hydroxy-acrylamide, (E)-3-[2-but-3-ynyl-1-(3-dimethylamino-2,2-dimethyl-propyl)-1H-benzimidazol-5-yl]-N-hydroxy-acrylamide, (E)-3-[2-but-3-enyl-1-(3-dimethylamino-2,2-dimethyl-propyl)-1H-benzimidazol-5-yl]-N-hydroxy-acrylamide, (E)-3-[2-but-3-enyl-1-(2-diethylamino-ethyl)-1H-benzimidazol-5-yl]-N-hydroxy-acrylamide, (E)-3-[2-but-3-ynyl-1-(2-diethylamino-ethyl)-1H-benzimidazol-5-yl]-N-hydroxy-acrylamide, (E)-3-[1-(3-dimethylamino-2,2-dimethyl-propyl)-2-(3,3,3-trifluoro-propyl)-1H-benzimidazol-5-yl]-N-hydroxy-acrylamide (E)-3-[1-(2-diethylamino-ethyl)-2-(3,3,3-trifluoro-propyl)-1H-benzimidazol-5-yl]-N-hydroxy-acrylamide, (E)-3-[1-(2-diethylamino-ethyl)-2-ethoxymethyl-1H-benzimidazol-5-yl]-N-hydroxy-acrylamide, (E)-3-[1-(3-dimethylamino-2,2-dimethyl-propyl)-2-methyl-1H-benzimidazol-5-yl]-N-hydroxy-acrylamide, (E)-3-[1-(2-diethylamino-ethyl)-2-(2,2-dimethyl-propyl)-1H-benzimidazol-5-yl]-N-hydroxy-acrylamide, (E)-N-hydroxy-3-[1-(3-isopropylamino-propyl)-2-(3,3,3-trifluoro-propyl)-1-H-benzimidazol-5-yl]-acrylamide, (E)-3-[2-(2,2-dimethyl-propyl)-1-(2-isopropylamino-ethyl)-1H-benzimidazol-5-yl]-N-hydroxy-acrylamide, (E)-3-[1-(2-diisopropylamino-ethyl)-2-(2,2-dimethyl-propyl)-1H-benzimidazol-5-yl]-N-hydroxy-acrylamide, (E)-3-[1-(2-diisopropylamino-ethyl)-2-isobutyl-1H-benzimidazol-5-yl]-N-hydroxy-acrylamide, (E)-3-[1-(3-dimethylamino-2,2-dimethyl-propyl)-2-hex-3-enyl-1H-benzimidazol-5-yl]-N-hydroxy-acrylamide, (E)-3-[1-(3-dimethylamino-2,2-dimethyl-propyl)-2-(2,4,4-trimethyl-pentyl)-1H-benzimidazol-5-yl]-N-hydroxy-acrylamide, (E)-3-[2-cyclohexyl-1-(3-dimethylamino-2,2-dimethyl-propyl)-1H-benzimidazol-5-yl]-N-hydroxy-acrylamide, (E)-3-[2-bicyclo[2.2.1]hept-5-en-2-yl-1-(3-dimethylamino-2,2-dimethyl-propyl)-1H-benzimidazol-5-yl]-N-hydroxy-acrylamide, (E)-3-[1-(2-diethylamino-ethyl)-2-hex-3-enyl-1H-benzimidazol-5-yl]-N-hydroxy-acrylamide, (E)-3-[1-(2-diisopropylamino-ethyl)-2-hex-3-enyl-1H-benzimidazol-5-yl]-N-hydroxy-acrylamide, (E)-3-[2-hex-3-enyl-1-(2-isopropylamino-ethyl)-1H-benzimidazol-5-yl]-N-hydroxy-acrylamide, (E)-3-[2-hex-3-enyl-1-(3-isopropylamino-propyl)-1H-benzimidazol-5-yl]-N-hydroxy-acrylamide, (E)-3-[1-(2-ethylamino-ethyl)-2-hex-3-enyl-1H-benzimidazol-5-yl]-N-hydroxy-acrylamide, (E)-3-[1-(2-diethylamino-ethyl)-2-hexyl-1H-benzimidazol-5-yl]-N-hydroxy-acrylamide, (E)-N-hydroxy-3-[1-(3-isopropylamino-propyl)-2-(2,4,4-trimethyl-pentyl)-1H-benzimidazol-5-yl]-acrylamide, (E)-3-[2-(2,2-dimethyl-propyl)-1-(3-isopropylamino-propyl)-1H-benzimidazol-5-yl]-N-hydroxy-acrylamide, (E)-3-[1-(2-diisopropylamino-ethyl)-2-(3,3,3-trifluoro-propyl)-1H-benzimidazol-5-yl]-N-hydroxy-acrylamide, and (E)-N-hydroxy-3-[2-isobutyl-1-(2-isopropylamino-ethyl)-1H-benzimidazol-5-yl]-acrylamide.

The use of O⁶-methylguanine-DNA-methyltransferase (MGMT) inhibitors is described in United States Patent Application 2010/0093647 by Liu et al. Suitable MGMT inhibitors include, but are not limited to, O⁶-benzylguanine, O⁶-2-fluoropyridinylmethylguanine, O⁶-3-iodobenzyl guanine, O⁶-4-bromophenylguanine, O⁶-5-iodophenylguanine O⁶-benzyl-8-oxoguanine, O⁶-(p-chlorobenzyl)guanine, O⁶-(p-methylbenzyl)guanine, O⁶-(p-bromobenzyl)guanine, O⁶-(p-isopropylbenzyl)guanine, O⁶-(3,5-dimethylbenzyl)guanine, O⁶-(p-n-butylbenzyl)guanine, O⁶-(p-hydroxymethybenzyl)guanine, O⁶-benzylhypoxanthine, N²-acetyl-O⁶-benzylguanine, N²-acetyl-O⁶-benzyl-8-oxo-guanine, 2-amino-6-(p-methyl-benzyl-thio)purine, 2-amino-6-(benzyloxy)-9-[(ethoxycarbonyl)methyl]purine, 2-amino-6-(benzyloxy)-9-(pivaloyloxymethyl)purine, 2-amino-6-(benzyl-thio)purine, O⁶-benzyl-7,8-dihydro-8-oxoguanine, 2,4,5-triamino-6-benzyloxyprimidine, O⁶-benzyl-9-[(3-oxo-5a-androstan-17β-yloxycarbonylmethyl]guanine, O⁶-benzyl-9-[(3-oxo-4-androsten-17β-yloxycarbonyl)methyl(guanine, 8-amino-O⁶-benzylguanine (8-amino-BG), 2,4-diamino-6-benzyloxy-5-nitrosopyrimidine, 2,4-diamino-6-benzyloxy-5-nitropyrimidine, and 2-amino-4-benzyloxy-5-nitropyrimidine.

The use of CCR9 inhibitors is described in United States Patent Application Publication No. 2010/0075963 by Lehr et al. These CCR9 inhibitors include, but are not limited to, benzylsulfonylindoles.

The use of acid sphingomyelinase inhibitors is described in United States Patent Application Publication No. 2010/0022482 by Baumann et al. Typically, these compounds are biphenyl derivatives.

The use of peptidomimetic macrocycles is described in United States Patent Application Publication No. 2009/0275519 by Nash et al.

The use of cholanic acid amides is described in United States Patent Application Publication No. 2009/0258847 by Schreiner et al. These cholanic acid amides include, but are not limited to, substituted 4-(3-hydroxy-10,13-hydroxymethyl-hexadecahydro-cyclopenta(a)-phenanthren-17-yl)pentanoic acid amides.

The use of substituted oxazaphosphorines is described in United States Patent Application Publication No. 2009/0202540. The oxazaphosphorine can be, but is not limited to, ifosphamide and cyclophosphamide.

The use of anti-TWEAK receptor antibodies is described in United States Patent Application Publication No. 2009/0074762 by Culp. The TWEAK receptor is a member of the tumor necrosis receptor superfamily and is expressed on the surface of cancer cells in a number of solid tumors.

The use of ErbB3 binding protein is described in United States Patent Application Publication No. 2008/0269133 by Zhang et al.

The use of a glutathione S-transferase-activated (GST-activated) anti-neoplastic compound is described in United States Patent Application Publication No. 2008/0166428 by Brown et al. A preferred GST-activated anti-neoplastic compound is canfosfamide.

The use of substituted phosphorodiamidates is described in United States Patent Application Publication No. 2008/0125398 by Ma et al., which describes 2-{[2-(substituted amino)ethyl]sulfonyl}ethyl N, N, N′,N′-tetrakis(2-chloroethyl)-phosphorodiamidates, and in United States Patent Application Publication No. 2008/0125397 by Lui et al., which describes 2-({2-oxo-2-[(pyridin-3-ylmethyl)amino]ethyl}sulfonyl)ethyl N,N,N′,N′-tetrakis(2-chloroethyl)phosphorodiamidate. The use of substituted phosphorodiamidates is also described in United States Patent Application Publication No. 2008/0039429 by Allen et al., which describes sulfonylethyl and thioethyl phosphorodiamidates.

The use of inhibitors of MEKK protein kinase is described in United States Patent Application Publication No. 2006/0100226 by Sikorski et al. These inhibitors include, but are not limited to, 2-thiopyrimidinones, such as 2-[3-(3,4-dichloro-benzylamino)-benzylsulfanyl]-4-(3-methoxy-phenyl)-6-oxo-1,6-dihydro-pyrimidine-5-carbonitrile, 2-[3-(3,4-dichloro-benzylamino)-benzylsulfanyl]-4-(3,4-dimethoxy-phenyl)-6-oxo-1,6-dihydro-pyrimidine-5-carbonitrile, and 2-[3-(3,4-dichloro-benzylamino)-benzylsulfanyl-4-(4-methoxy-3-thiophen-2-yl-phenyl)-6-oxo-1,6-dihydro-pyrimidine-5-carbonitrile.

The use of COX-2 inhibitors is described in United States Patent Application Publication No. 2004/0072889 by Masferrer et al. Suitable COX-2 inhibitors include, but are not limited to, celecoxib, parecoxib, deracoxib, rofecoxib, etoricoxib, valdecoxib, and meloxicam.

The use of cimetidine and N-acetylcysteine is described in United States Patent Application Publication No. 2003/0158118 by Weidner. Derivatives of cimetidine or N-acetylcysteine can also be used.

The use of an anti-IL-6 receptor antibody is described in United States Patent Application Publication No. 2002/0131967 by Nakamura et al. The antibody can be a humanized antibody.

The use of an antioxidant is described in United States Patent Application Publication No. 2001/0049349 by Chinery et al. Suitable antioxidants include, but are not limited to, pyrrolidinedithiocarbamate, probucol (4,4′-(isopropylidenedithio)bis(2,6-di-t-butylphenol), vitamin C, vitamin E, and 6-hydroxy-2,5,7,8-tetramethylchroman-2-carboxylic acid.

The use of an isoxazole inhibitor of tubulin polymerization is described in U.S. Pat. No. 8,269,017 by Sun et al. Suitable isoxazole inhibitors of tubulin polymerization include, but are not limited to, 2-amino-N-(2-methoxy-5-[5-(3,4,5-trimethoxyphenyl)-isoxazol-4-yl)-phenyl)acetamide hydrochloride; 2-amino-3-hydroxy-N-(2-methoxy-5-[5-(3,4,5-trimethoxyphenyl)isoxazol-4-yl)-phenyl)propanamide hydrochloride; 2-amino-N-(2-methoxy-5-[5-(3,4,5-trimethoxyphenyl)isoxazol-4-yl)-phenyl)propanamide; 2-amino-N-(2-methoxy-5-[5-(3,4,5-trimethoxyphenyl)-isoxazol-4-yl)-phenyl)-4-(methylthio)butanamide hydrochloride; 2-amino-N-(2-methoxy-5-[5-(3,4,5-trimethoxyphenyl)-isoxazol-4-yl)-phenyl)butanamide; 2-amino-N-(2-methoxy-5-[5-(3,4,5-trimethoxyphenyl)-isoxazol-4-yl)-phenyl)-3-phenylpropanamide hydrochloride; 2-amino-N-(2-methoxy-5-[5-(3,4,5-trimethoxyphenyl)-isoxazol-4-yl)-phenyl)-4-methylpentanamide hydrochloride; 2-amino-N-(2-methoxy-5-[5-(3,4,5-trimethoxy-phenyl)-isoxazol-4-yl)-phenyl)-3-(4-methoxyphenyl)propanamide hydrochloride; 1-{2-methoxy-5-[5-(3,4,5-trimethoxy-phenyl)-isoxazol-4-yl]-phenylcarbamoyl}-2-methyl-propyl-ammonium chloride; 1-{2-methoxy-5-[5-(3,4,5-trimethoxyphenyl)-isoxazol-4-yl]-phenylcarbamoyl}-2-methyl-butyl-ammonium chloride; 2-hydroxy-1-{2-methoxy-5-[5-(3,4,5-trimethoxyphenyl)-isoxazol-4-yl]-phenylcarbamoyl}-propyl-ammonium chloride; 2-(4-hydroxy-phenyl)-1-{2-methoxy-5-[5-(3,4,5-trimethoxyphenyl)-isoxazol-4-yl]-phenylcarbamoyl}-ethyl-ammonium chloride; C-{2-methoxy-5-[5-(3,4,5-trimethoxyphenyl)-isoxazol-4-yl]-phenylcarbamoyl}-C-phenyl-methyl-ammonium chloride; 2-(1H-indol-2-yl)-1-{2-methoxy-5-[5-(3,4,5-trimethoxyphenyl)-isoxazol-4-yl]-phenylcarbamoyl}-ethyl-ammonium chloride; 2-benzofuran-2-yl-1-{2-methoxy-5-[5-(3,4,5-trimethoxyphenyl)-isoxazol-4-yl]-phenylcarbamoyl}-ethyl-ammonium chloride; 2-carboxyl-1-{2-methoxy-5-[5-(3,4,5-trimethoxyphenyl)-isoxazol-4-yl]-phenylcarbamoyl}-ethyl-ammonium chloride; 3-carboxyl-1-{2-methoxy-5-[5-(3,4,5-trimethoxyphenyl)-isoxazol-4-yl]-phenylcarbamoyl}-propyl-ammonium chloride; 3-carbamoyl-1-{2-methoxy-5-[5-(3,4,5-trimethoxyphenyl)-isoxazol-4-yl]-phenylcarbamoyl}-propyl-ammonium chloride; 2-carbamoyl-1-{2-methoxy-5-[5-(3,4,5-trimethoxyphenyl)-isoxazol-4-yl]-phenylcarbamoyl}-ethyl-ammonium chloride; and 2-(3H-imidazol-4-O-1-{2-methoxy-5-[5-(3,4,5-trimethoxyphenyl)-isoxazol-4-yl]-phenylcarbamoyl}-ethyl-ammonium chloride.

The use of pyridazinone PARP inhibitors is described in U.S. Pat. No. 8,268,827 by Branca et al. Pyridazinone PARP inhibitors include, but are not limited to, 6-{4-fluoro-3-[(3-oxo-4-phenylpiperazin-1-yl)carbonyl]benzyl}-4,5-dimethyl-3-oxo-2,3-dihydropyridazin-1-ium trifluoroacetate; 6-{3-[(4-cyclohexyl-3-oxopiperazin-1-yl)carbonyl]-4-fluorobenzyl}-4,5-dimethyl-3-oxo-2,3-dihydropyridazin-1-ium trifluoroacetate; 6-{3-[(4-cyclopentyl-3-oxopiperazin-1-yl)carbonyl]-4-fluorobenzyl}-4,5-dimethylpyridazin-3(2H)-one; 6-{4-fluoro-3-[(3-oxo-4-phenylpiperazin-1-yl)carbonyl]benzyl}-4,5-dimethylpyridazin-3(2H)-one hydrochloride; 4-ethyl-6-{4-fluoro-3-[(3-oxo-4-phenylpiperazin-1-yl)carbonyl]benzyl}pyridazin-3(2H)-one trifluoroacetate; 6-{3-[(4-cyclohexyl-3-oxopiperazin-1-yl)carbonyl]-4-fluorobenzyl}-4-ethylpyridazin-3(2H)-one trifluoroacetate; 3-{4-fluoro-3-[(4-methyl-3-oxopiperazin-1-yl)carbonyl]benzyl}-4,5-dimethyl-6-oxo-1,6-dihydropyridazin-1-ium trifluoroacetate; 3-(4-fluoro-3-{[4-(4-fluorobenzyl)-3-oxopiperazin-1-yl]carbonyl}benzyl)-4,5-dimethyl-6-oxo-1,6-dihydropyridazin-1-ium trifluoroacetate; 6-(3-{[4-(2-chlorophenyl)-3-oxopiperazin-1-yl]carbonyl}-4-fluorobenzyl)-4,5-dimethyl-3-oxo-2,3-dihydropyridazin-1-ium trifluoroacetate; 6-(3-{[4-(3-chloro-4-fluorophenyl)-3-oxopiperazin-1-yl]carbonyl}-4-fluorobenzyl)-4,5-dimethyl-3-oxo-2,3-dihydropyridazin-1-ium trifluoroacetate; and 6-(3-{[4-(3,4-difluorophenyl)-3-oxopiperazin-1-yl]carbonyl}-4-fluorobenzyl)-4,5-dimethyl-3-oxo-2,3-dihydropyridazin-1-ium trifluoroacetate. Other PARP inhibitors are described in U.S. Pat. No. 8,143,447 by Moore et al.; these compounds include nitrobenzamide derivatives.

The use of Aurora protein kinase inhibitors is described in U.S. Pat. No. 8,268,811 to Mortimore et al. The Aurora protein kinase inhibitors include, but are not limited to, thiazoles and pyrazoles. The use of Aurora protein kinase inhibitors is also described in U.S. Pat. No. 8,129,399 to Binch et al.; these Aurora protein kinase inhibitors include, but are not limited to, aminopyridines.

The use of peptides binding to prostate-specific membrane antigen (PSMA) is described in U.S. Pat. No. 8,258,256 to Denmeade et al.

The use of CD19 binding agents is described in U.S. Pat. No. 8,242,252 to McDonagh et al. These CD19 binding agents include, but are not limited to, anti-CD19 antibodies.

The use of benzodiazepines is described in U.S. Pat. No. 8,242,109 to Glick.

The use of Toll-like receptor (TLR) agonists is described in U.S. Pat. No. 8,242,106 to Howbert et al. Suitable TLR agonists include, but are not limited to, (1E, 4E)-2-amino-N,N-dipropyl-8-(4-(pyrrolidine-1-carbonyl)phenyl)-3H-benzo[b]azepine-4-carboxamide.

The use of bridged bicyclic sulfamides is described in U.S. Pat. No. 8,242,103 to Lewis et al.

The use of inhibitors of epidermal growth factor receptor (EGFR) kinase is described in U.S. Pat. No. 8,242,080 to Kuriyan et al. Typically, these inhibitors of EGFR kinase target the asymmetric activating dimer interface.

The use of ribonucleases of the T2 family having actin-binding activity is described in U.S. Pat. No. 8,236,543 to Roiz et al. Typically, the ribonuclease binds actin in either its active or inactive ribonucleolytic form.

The use of myrsinoic acid A or an analog thereof is described in U.S. Pat. No. 8,232,318 to Lee et al.

The use of an inhibitor of a cyclin-dependent kinase is described in U.S. Pat. No. 8,227,605 to Shipps et al.; these inhibitors include, but are not limited to, 2-aminothiazole-4-carboxylic amides. Use of an inhibitor of a cyclin-dependent kinase is also described in U.S. Pat. No. 7,700,773 to Mallams et al.; these inhibitors include, but are not limited to, 4-cyano, 4-amino, and 4-aminomethyl derivatives of pyrazolo[1,5-a]pyridine, pyrazolo[1,5-c]pyrimidine, and 2H-indazole compounds and 5-cyano, 5-amino, and 5-aminomethyl derivatives of imidazo[1,2-a]pyridine and imidazo[1,5-a]pyrazine compounds. Other inhibitors of a cyclin-dependent kinase are known in the art, and include alvocidib, olomoucine, roscovitine, purvalanol, paullone cyclin-dependent kinase inhibitors, butyrolactone, palbociclib, thioflavopiridol cyclin-dependent kinase inhibitors, oxoflavopiridol cyclin-dependent kinase inhibitors, oxindole cyclin-dependent kinase inhibitors, aminothiazole cyclin-dependent kinase inhibitors, benzocarbazole cyclin-dependent kinase inhibitors, pyrimidine cyclin-dependent kinase inhibitors, and seliciclib.

The use of an inhibitor of the interaction between p53 and MDM2 is described in U.S. Pat. No. 8,222,288 to Wang et al.

The use of inhibitors of the receptor tyrosine kinase MET is described in U.S. Pat. No. 8,222,269 to Dinsmore et al. These inhibitors of the receptor tyrosine kinase MET include, but are not limited to, 5H-benzo[4,5]cyclohepta[1,2-b]pyridine derivatives. Inhibitors of the receptor tyrosine kinase MET are also described in U.S. Pat. No. 8,207,186 to Jewell et al. These compounds include, but are not limited to, benzocycloheptapyridines, including 5H-benzo[4,5]cyclohepta[1,2-b]pyridine derivatives.

The use of largazole or largazole analogs is described in U.S. Pat. No. 8,217,076 to Williams et al.

The use of inhibitors of the protein kinase AKT is described in U.S. Pat. No. 8,207,169 to Furuyama et al.; these inhibitors include, but are not limited to, triazolopyridopyridines, including substituted [1,2,4]triazolo[4′,3′:1,6]pyrido[2,3-b]pyrazines.

The use of 2′-fluoro-5-methyl-β-L-arabinofuranosyluridine or L-deoxythymidine is described in U.S. Pat. No. 8,207,143 to Cheng.

The use of compounds that modulate HSP90 activity is described in U.S. Pat. No. 8,188,075 to Ying et al. These compounds include, but are not limited to, substituted triazoles, including 3-(2-hydroxyphenyl)-4-(naphthalen-1-yl)-5-mercaptotriazole; 3-(2,4-dihydroxyphenyl)-4-[4-(2-methoxyethoxy)-naphthalen-1-yl]-5-mercaptotriazole; 3-(2,4-dihydroxyphenyl)-4-(2-methyl-4-bromophenyl)-5-mercaptotriazole; 3-(3,4-dihydroxyphenyl)-4-(6-methoxy-naphthalen-1-yl)-5-mercaptotriazole; 3-(3,4-dihydroxyphenyl)-4-(6-ethoxy-naphthalen-1-yl)-5-mercaptotriazole; 3-(3,4-dihydroxyphenyl)-4-(6-propoxy-naphthalen-1-yl)-5-mercaptotriazole; 3-(2,4-dihydroxy-5-ethyl-phenyl)-4-(5-methoxy-naphthalen-1-yl)-5-mercaptotriazole; 3-(3,4-dihydroxyphenyl)-4-(6-isopropoxy-naphthalen-1-yl)-5-mercaptotriazole; 3-(2,4-dihydroxyphenyl)-4-(2,6-diethylphenyl)-5-mercaptotriazole; 3-(2,4-dihydroxyphenyl)-4-(2-methyl-6-ethylphenyl)-5-mercaptotriazole; 3-(2,4-dihydroxyphenyl)-4-(2,6-diisopropylphenyl)-5-mercaptotriazole; 3-(2,4-dihydroxyphenyl)-4-(1-ethyl-indol-4-yl)-5-mercaptotriazole; and 3-(2,4-dihydroxyphenyl)-4-(2,3-dihydro-benzo[1,4]dioxin-5-yl)-5-mercaptotriazole.

The use of inhibitors of a JAK kinase or PDK kinase is described in U.S. Pat. No. 8,183,245 to Guerin et al. JAK kinases include JAK1, JAK2, JAK3, and TYK2. Suitable inhibitors of these classes of kinases include, but are not limited to, 5-(1-methyl-1H-pyrazol-4-yl)-3-(6-piperazin-1-ylpyrazin-2-yl)-1H-pyrrolo[2,3-b]pyridine; 5-(1-methyl-1H-pyrazol-4-yl)-3-[6-(piperidin-4-yloxy)pyrazin-2-yl]-1H-pyrrolo[2,3-b]pyridine; 3-[6-(cyclohexyloxy)pyrazin-2-yl]-5-(1-methyl-1H-pyrazol-4-yl)-1H-pyrrolo[2,3-b]pyridine; N-methyl-6-[5-(1-methyl-1H-pyrazol-4-yl)-1H-pyrrolo[2,3-b]pyridin-3-yl]-N-piperidin-4-ylpyrazin-2-amine; 3-[6-(piperidin-4-yloxy)pyrazin-2-yl]-5-(1H-pyrazol-4-yl)-1H-pyrrolo[2,3-b]pyridine; 3-{6-[(3R)-piperidin-3-yloxy]pyrazin-2-yl}-5-(1H-pyrazol-4-yl)-1H-pyrrolo[2,3-b]pyridine; and 3-{6-[(3S)-piperidin-3-yloxy]pyrazin-2-yl}-5-(1H-pyrazol-4-yl)-1H-pyrrolo[2,3-b]pyridine.

The use of inhibitors of phosphodiesterase type IV (PDE4) is described in U.S. Pat. No. 8,158,672 to Muller et al. The inhibitors of PDE4 include fluoroalkoxy-substituted 1,3-dihydroisoindolyl compounds.

The use of inhibitors of c-Met proto-oncogene receptor tyrosine kinase is described in U.S. Pat. No. 8,143,251 to Zhuo et al., incorporated by this reference. These inhibitors include, but are not limited to, triazolotriazines, including [1,2,4]triazolo[4,3-b][1,2,4]triazines. Inhibitors of c-Met proto-oncogene receptor tyrosine kinase are also described in U.S. Pat. No. 8,106,197 to Cui et al.; these inhibitors include aminoheteroaryl compounds.

The use of inhibitors of indoleamine 2,3-dioxygenase is described in U.S. Pat. No. 8,088,803 to Combs et al.; these inhibitors include, but are not limited to, 1,2,5-oxadiazole derivatives.

The use of agents that inhibit ATDC (TRIM29) expression is described in U.S. Pat. No. 8,088,749 to Simeone et al. These agents include oligonucleotides that function via RNA interference.

The use of proteomimetic inhibitors of the interaction of nuclear receptor with coactivator peptides is described in U.S. Pat. No. 8,084,471 to Hamilton et al. These inhibitors include, but are not limited to, 2,3′,3″-trisubstituted terphenyls.

The use of antagonists of XIAP family proteins is described in U.S. Pat. No. 7,910,621 to Chen et al. These antagonists include, but are not limited to, embelin.

The use of tumor-targeted superantigens is described in U.S. Pat. No. 7,763,253 to Hedlund et al.

The use of inhibitors of Pim kinases is described in U.S. Pat. No. 7,750,007 to Bearss et al. These inhibitors include, but are not limited to, imidazo[1,2-b]pyridazine and pyrazolo[1,5-a]pyrimidine compounds.

The use of inhibitors of CHK1 or CHK2 kinases is described in U.S. Pat. No. 7,732,436 to Tepe. These inhibitors include, but are not limited to, indoloazepines and acid amine salts thereof. Additional inhibitors of CHK1 kinases are described in: U.S. Pat. No. 9,067,920 to Joseph et al., including aminopyrazoles; U.S. Pat. No. 8,618,121 to Collins et al., including 9H-pyrimido[4,5-b]indoles, 9H-pyrido[4′,3′:4,5]pyrrolo[2,3-d]pyridines, and 9H-1,3,6,9-tetraaza-fluorenes; U.S. Pat. No. 8,455,471 to Wisdom et al., including disubstituted urea compounds; U.S. Pat. No. 8,093,244 to Diaz et al., including 1-[5-bromo-4-methyl-2-S-(morpholin-2-ylmethoxy)-phenyl]-3-(5-methyl-pyrazin-2-yl)-urea; U.S. Pat. No. 7,608,618 to Kesicki et al., including urea- or thiourea-substituted 1,4-pyrazine compounds; U.S. Pat. No. 7,560,462 to Gaudino et al., including 1-(5-methyl-pyrazin-2-yl)-3-(5-methyl-2-pyridin-3-ylethynyl-phenyl)-urea; 1-(5-methyl-pyrazin-2-yl)-3-(5-methyl-2-pyridin-3-yl-phenyl)-urea; 1-(5-methylpyrazin-2-yl)-3-(5-methyl-2-pyridin-4-yl-phenyl)-urea; 1-(5-methyl-pyrazine-2-yl)-3-(2-oxazol-5-yl-phenyl)-urea; 1-(5-methyl-pyrazin-2-yl)-3-(5-methyl-2-thiazol-2-ylphenyl)-urea; and 1-[2-(4-dimethylaminomethyl-thiazol-2-yl)-5-methyl-phenyl]-3-(5-methyl-pyrazin-2-yl)-urea; U.S. Pat. No. 7,094,798 to Booth et al., including pyrrolocarbazoles; and U.S. Pat. No. 7,067,506 to Keegan et al., including aryl- and heteroaryl-substituted urea derivatives. Additional inhibitors of CHK2 kinases are described in: U.S. Pat. No. 8,912,214 to Pommier et al., including diaryl-substituted urea derivatives; and U.S. Pat. No. 8,067,452 to Wu et al., including 3-hydroxyisothiazole-4-carboxamidine derivatives.

The use of inhibitors of angiopoietin-like 4 protein is described in U.S. Pat. No. 7,740,846 to Gerber et al. These inhibitors include, but are not limited to, antibodies, including monoclonal antibodies.

The use of inhibitors of Smo is described in U.S. Pat. No. 7,691,997 to Balkovec et al., incorporated by this reference. Smo, or Smoothened, is a mediator of signaling by hedgehog proteins. Suitable inhibitors include, but are not limited to, 5-(1,1-difluoroethyl)-3-(4-{4-methyl-5-[2-(trifluoromethyl)phenyl]-4H-1,2,4-triazol-3-yl}bicyclo[2.2.2]oct-1-yl)-1,2,4-oxadiazole; 5-(3,3-difluorocyclobutyl)-3-(4-{4-methyl-5-[2-(trifluoromethyl)phenyl]-4H-1,2,4-triazol-3-yl}bicyclo[2.2.2]oct-1-yl)-1,2,4-oxadiazole; 5-(1-fluoro-1-methylethyl)-3-(4-{4-methyl-5-[2-(trifluoromethyl)phenyl]-4H-1,2,4-triazol-3-yl}bicyclo[2.2.2]oct-1-yl)-1,2,4-oxadiazole; 2-(1,1-difluoroethyl)-5-(4-{4-methyl-5-[2-(trifluoromethyl)phenyl]-4H-1,2,4-triazol-3-yl}bicyclo[2.2.2]oct-1-yl)-1,3,4-oxadiazole; 2-(3,3-difluorocyclobutyl)-5-(4-{4-methyl-5-[2-(trifluoromethyl)phenyl]-4H-1,2,4-triazol-3-yl}bicyclo[2.2.2]oct-1-yl)-1,3,4-oxadiazole; and 2-(1-fluoro-1-methylethyl)-5-(4-{4-methyl-5-[2-(trifluoromethyl)phenyl]-4H-1,2,4-triazol-3-yl}bicyclo[2.2.2]oct-1-yl)-1,3,4-oxadiazole.

The use of nicotinic acetylcholine receptor antagonists is disclosed in U.S. Pat. No. 7,652,038 to Cooke et al. Nicotinic acetylcholine receptor antagonists include, but are not limited to, mecamylamine, hexamethonium, dihydro-β-erythroidine, d-tubocurarine, pempidine, chlorisondamine, erysodine, trimethaphan camsylate, pentolinium, bungarotoxin, succinylcholine, tetraethylammonium, trimethaphan, chlorisondamine, and trimethidinium.

The use of farnesyl protein transferase inhibitors is described in U.S. Pat. No. 7,557,107 to Zhu et al. These farnesyl protein transferase inhibitors include tricyclic compounds.

The use of adenosine A3 receptor antagonists is described in U.S. Pat. No. 6,326,390 to Leung et al. These adenosine A3 receptor antagonists include tricyclic non-xanthine antagonists and triazoloquinazolines.

Additional drug combinations can include an alkylating hexitol derivative as described above with at least one agent that suppresses growth or replication of glioma cancer stem cells. Such agents include, but are not limited to: an inhibitor of tailless gene expression or tailless gene activity, as described in U.S. Pat. No. 8,992,923 to Liu et al.; an inhibitor of HDAC1, HDAC7, or phosphorylated HDAC7, as described in U.S. Pat. No. 8,912,156 to Ince et al.; Stat3 inhibitors such as naphtho derivatives, as described in U.S. Pat. No. 8,877,803 to Jiang et al.; a combination of a farnesyl transferase inhibitor and a gamma secretase inhibitor, as described in U.S. Pat. No. 8,853,274 to Wang; inhibitors of electron transport chains or the mitochondrial Krebs cycle as described in U.S. Pat. No. 8,815,844 to Clement et al.; Jak2/STAT3 pathway inhibitors such as caffeic acid derivatives as described in United States Patent Application Publication No. 2015/0094343 by Priebe et al.; inhibitors of the glycine cleavage pathway as described in United States Patent Application Publication No. 2015/0011611 by Kim et al.; and glycosylated ether lipids as described in United States Patent Application Publication No. 2015/0011486 by Arthur et al.

Other additional drug combinations can include an alkylating hexitol derivative as described above with: (1) a topoisomerase inhibitor; and (2) an inhibitor of CHK1 or CHK2 kinases.

In one alternative, when the drug combination is use with an alkylating agent, the alkylating agent can be selected from the group consisting of BCNU, BCNU wafers (Gliadel), ACNU, CCNU, bendamustine (Treanda), lomustine, and temozolimide (Temodar).

When the improvement is made by chemosensitization, the chemosensitization can comprise, but is not limited to, the use of a substituted hexitol derivative as a chemosensitizer in combination with an agent selected from the group consisting of:

(a) topoisomerase inhibitors;

(b) fraudulent nucleosides;

(c) fraudulent nucleotides;

(d) thymidylate synthetase inhibitors;

(e) signal transduction inhibitors;

(f) cisplatin or platinum analogs;

(g) alkylating agents;

(h) anti-tubulin agents;

(i) antimetabolites;

(j) berberine;

(k) apigenin;

(l) amonafide;

(m) colchicine or analogs;

(n) genistein;

(o) etoposide;

(p) cytarabine;

(q) camptothecins;

(r) vinca alkaloids;

(s) 5-fluorouracil;

(t) curcumin;

(u) NF-κB inhibitors;

(v) rosmarinic acid;

(w) mitoguazone;

(x) tetrandrine;

(y) a tyrosine kinase inhibitor;

(z) an inhibitor of EGFR; and

(aa) an inhibitor of PARP.

When the improvement is made by chemopotentiation, the chemopotentiation can comprise, but is not limited to, the use of a substituted hexitol derivative as a chemopotentiator in combination with an agent selected from the group consisting of:

(a) topoisomerase inhibitors;

(b) fraudulent nucleosides;

(c) fraudulent nucleotides;

(d) thymidylate synthetase inhibitors;

(e) signal transduction inhibitors;

(f) cisplatin or platinum analogs;

(g) alkylating agents;

(h) anti-tubulin agents;

(i) antimetabolites;

(j) berberine;

(k) apigenin;

(l) amonafide;

(m) colchicine or analogs;

(n) genistein;

(o) etoposide;

(p) cytarabine;

(q) camptothecins;

(r) vinca alkaloids;

(s) 5-fluorouracil;

(t) curcumin;

(u) NF-κB inhibitors;

(v) rosmarinic acid;

(w) mitoguazone;

(x) tetrandrine;

(y) a tyrosine kinase inhibitor;

(z) an inhibitor of EGFR; and

(aa) an inhibitor of PARP.

In one alternative, when the chemopotentiation involves chemopotentiation of an alkylating agent by the activity of dianhydrogalactitol, the alkylating agent can be selected from the group consisting of BCNU, BCNU wafers (Gliadel), CCNU, bendamustine (Treanda), lomustine, ACNU, and temozolimide (Temodar).

When the improvement is made by post-treatment management, the post-treatment management can be, but is not limited to, a method selected from the group consisting of:

(a) a therapy associated with pain management;

(b) administration of an anti-emetic;

(c) an anti-nausea therapy;

(d) administration of an anti-inflammatory agent;

(e) administration of an anti-pyretic agent; and

(f) administration of an immune stimulant.

When the improvement is made by alternative medicine/post-treatment support, the alternative medicine/post-treatment support can be, but is not limited to, a method selected from the group consisting of:

(a) hypnosis;

(b) acupuncture;

(c) meditation;

(d) a herbal medication created either synthetically or through extraction; and

(e) applied kinesiology.

In one alternative, when the method is a herbal medication created either synthetically or through extraction, the herbal medication created either synthetically or through extraction can be selected from the group consisting of:

(a) a NF-κB inhibitor;

(b) a natural anti-inflammatory;

(c) an immunostimulant;

(d) an antimicrobial; and

(e) a flavonoid, isoflavone, or flavone.

When the herbal medication created either synthetically or through extraction is a NF-κB inhibitor, the NF-κB inhibitor can be selected from the group consisting of parthenolide, curcumin, and rosmarinic acid. When the herbal medication created either synthetically or through extraction is a natural anti-inflammatory, the natural anti-inflammatory can be selected from the group consisting of rhein and parthenolide. When the herbal medication created either synthetically or through extraction is an immunostimulant, the immunostimulant can be a product found in or isolated from Echinacea. When the herbal medication created either synthetically or through extraction is an anti-microbial, the anti-microbial can be berberine. When the herbal medication created either synthetically or through extraction is a flavonoid or flavone, the flavonoid, isoflavone, or flavone can be selected from the group consisting of apigenin, genistein, apigenenin, genistein, genistin, 6″-O-malonylgenistin, 6″-O-acetylgenistin, daidzein, daidzin, 6″-O-malonyldaidzin, 6″-O-acetylgenistin, glycitein, glycitin, 6″-O-malonylglycitin, and 6-O-acetylglycitin.

When the improvement is made by a bulk drug product improvement, the bulk drug product improvement can be, but is not limited to, a bulk drug product improvement selected from the group consisting of:

(a) salt formation;

(b) preparation as a homogeneous crystal structure;

(c) preparation as a pure isomer;

(d) increased purity;

(e) preparation with lower residual solvent content; and

(f) preparation with lower residual heavy metal content.

When the improvement is made by use of a diluent, the diluent can be, but is not limited to, a diluent selected from the group consisting of:

(a) an emulsion;

(b) dimethylsulfoxide (DMSO);

(c) N-methylformamide (NMF)

(d) DMF;

(e) ethanol;

(f) benzyl alcohol;

(g) dextrose-containing water for injection;

(h) Cremophor;

(i) cyclodextrin; and

(j) PEG.

When the improvement is made by use of a solvent system, the solvent system can be, but is not limited to, a solvent system selected from the group consisting of:

(a) an emulsion;

(b) dimethylsulfoxide (DMSO);

(c) N-methylformamide (NMF)

(d) DMF;

(e) ethanol;

(f) benzyl alcohol;

(g) dextrose-containing water for injection;

(h) Cremophor;

(i) cyclodextrin; and

(j) PEG.

When the improvement is made by use of an excipient, the excipient can be, but is not limited to, an excipient selected from the group consisting of:

(a) mannitol;

(b) albumin;

(c) EDTA;

(d) sodium bisulfite;

(e) benzyl alcohol;

(f) carbonate buffers;

(g) phosphate buffers;

(h) PEG;

(i) vitamin A;

(j) vitamin D;

(k) vitamin E;

(l) esterase inhibitors;

(m) cytochrome P450 inhibitors;

(n) multi-drug resistance (MDR) inhibitors;

(o) organic resins;

(p) detergents;

(q) perillyl alcohol or an analog thereof; and

(r) activators of channel-forming receptors.

Suitable esterase inhibitors include, but are not limited to, ebelactone A and ebelactone B.

Suitable cytochrome P450 inhibitors include, but are not limited to, 1-aminobenzotriazole, N-hydroxy-N′-(4-butyl-2-methylphenyl)formamidine, ketoconazole, methoxsalen, metyrapone, roquefortine C, proadifen, 2,3′,4,5′-tetramethylstilbene, and troleandomycin.

Suitable MDR inhibitors include, but are not limited to, 5′-methoxyhydnocarpin, INF 240, INF 271, INF 277, INF 392, INF 55, reserpine, and GG918. MDR inhibitors are described in M. Zloh & S. Gibbons, “Molecular Similarity of MDR9 Inhibitors,” Int. J. Mol. Sci. 5: 37-47 (2004).

Suitable organic resins include, but are not limited to, a partially neutralized polyacrylic acid, as described in U.S. Pat. No. 8,158,616 to Rodgers et al.

Suitable detergents include, but are not limited to, nonionic detergents such as a polysorbate or a poloxamer, and are described in PCT Patent Application Publication No. WO/1997/039768 by Bjorn et al.

The use of perillyl alcohol or an analog thereof to improve transport of anti-neoplastic agents is described in United States Patent Application 2012/0219541 by Chen et al.

The use of activators of channel-forming receptors is described in United States Patent Application Publication No. 2010/0311678 by Bean et al. Such activators of channel-forming receptors include, but are not limited to, capsaicin, lidocaine, eugenol, arvanil (N-arachidonoylvanillamine), anandamide, 2-aminoethoxydiphenyl borate, resiniferatoxin, phorbol 12-phenylacetate 13-acetate 20-homovanillate (PPAHV), olvanil, N-oleoyldopamine, N-arachidonyldopamine, 6′-iodoresiniferatoxin (6′-IRTX), C₁₈ N-acylethanolamines, lipoxygenase derivatives such as 12-hydroperoxyeicosatetraenoic acid, inhibitor cysteine knot (ICK) peptides (vanillotoxins), piperine, N-[2-(3,4-dimethylbenzyl)-3-(pivaloyloxy)propyl]-2-[4-(2-aminoethoxy)-3-methoxyphenyl]acetamide, N-[2-(3,4-dimethylbenzyl)-3-(pivaloyloxy)propyl]-N′-(4-hydroxy-3-methoxybenzyl)thiourea, SU200 N-(4-t-butylbenzyl)-N′-(4-hydroxy-3-methoxybenzyl)thiourea), transacin, cinnamaldehyde, allyl-isothiocyanate, diallyl disulfide, icilin, cinnamon oil, wintergreen oil, clove oil, acrolein, mustard oil, ATP, 2-methylthio-ATP, 2′ and 3′-O-(4-benzoylbenzoyl)-ATP, ATP-5′-O-(3-thiotriphosphate), menthol, eucalyptol, linalool, geraniol, and hydroxycitronellal.

When the improvement is made by use of a dosage form, the dosage form can be, but is not limited to, a dosage form selected from the group consisting of:

(a) tablets;

(b) capsules;

(c) topical gels;

(d) topical creams;

(e) patches;

(f) suppositories;

(g) lyophilized dosage fills;

(h) immediate-release formulations;

(i) slow-release formulations;

(j) controlled-release formulations; and

(k) liquid in capsules.

Formulation of pharmaceutical compositions in tablets, capsules, and topical gels, topical creams or suppositories is well known in the art and is described, for example, in United States Patent Application Publication No. 2004/0023290 by Griffin et al.

Formulation of pharmaceutical compositions as patches such as transdermal patches is well known in the art and is described, for example, in U.S. Pat. No. 7,728,042 to Eros et al.

Lyophilized dosage fills are also well known in the art. One general method for the preparation of such lyophilized dosage fills, applicable to dianhydrogalactitol and derivatives thereof and to diacetyldianhydrogalactitol and derivatives thereof, comprises the following steps:

(1) Dissolve the drug in water for injection precooled to below 10° C. Dilute to final volume with cold water for injection to yield a 40 mg/mL solution.

(2) Filter the bulk solution through an 0.2-μm filter into a receiving container under aseptic conditions. The formulation and filtration should be completed in 1 hour.

(3) Fill nominal 1.0 mL filtered solution into sterilized glass vials in a controlled target range under aseptic conditions.

(4) After the filling, all vials are placed with rubber stoppers inserted in the “lyophilization position” and loaded in the prechilled lyophilizer. For the lyophilizer, shelf temperature is set at +5° C. and held for 1 hour; shelf temperature is then adjusted to −5° C. and held for one hour, and the condenser, set to −60° C., turned on.

(5) The vials are then frozen to 30° C. or below and held for no less than 3 hours, typically 4 hours.

(6) Vacuum is then turned on, the shelf temperature is adjusted to −5° C., and primary drying is performed for 8 hours; the shelf temperature is again adjusted to −5° C. and drying is carried out for at least 5 hours.

(7) Secondary drying is started after the condenser (set at −60° C.) and vacuum are turned on. In secondary drying, the shelf temperature is controlled at +5° C. for 1 to 3 hours, typically 1.5 hours, then at 25° C. for 1 to 3 hours, typically 1.5 hours, and finally at 35-40° C. for at least 5 hours, typically for 9 hours, or until the product is completely dried.

(8) Break the vacuum with filtered inert gas (e.g., nitrogen). Stopper the vials in the lyophilizer.

(9) Vials are removed from the lyophilizer chamber and sealed with aluminum flip-off seals. All vials are visually inspected and labeled with approved labels.

Immediate-release formulations are described in U.S. Pat. No. 8,148,393 to van Dalen et al. Immediate-release formulations can include, for example, conventional film-coated tablets.

Slow-release formulations are described in U.S. Pat. No. 8,178,125 to Wen et al. Slow-release formulations can include, for example, microemulsions or liquid crystals.

Controlled-release formulations are described in U.S. Pat. No. 8,231,898 to Oshlack et al. Controlled-release formulations can include, for example, a matrix that includes a controlled-release material. Such a controlled-release material can include hydrophilic and/or hydrophobic materials, such as gums, cellulose ethers, acrylic resins, protein derived materials, waxes, shellac, and oils such as hydrogenated castor oil or hydrogenated vegetable oil.

However, any pharmaceutically acceptable hydrophobic or hydrophilic controlled-release material which is capable of imparting controlled release of the dianhydrogalactitol or the derivative, analogue, or prodrug of the dianhydrogalactitol or other substituted hexitol derivative as described above may be used in accordance with the present invention. Preferred controlled-release polymers include alkylcelluloses such as ethylcellulose, acrylic and methacrylic acid polymers and copolymers, and cellulose ethers, especially hydroxyalkylcelluloses (e.g., hydroxypropylmethylcellulose) and carboxyalkylcelluloses. Preferred acrylic and methacrylic acid polymers and copolymers include methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer, poly(methyl methacrylate), poly(methacrylic acid) (anhydride), polymethacrylate, polyacrylamide, poly(methacrylic acid anhydride), and glycidyl methacrylate copolymers.

When the improvement is made by use of dosage kits and packaging, the dosage kits and packaging can be, but are not limited to, dosage kits and packaging selected from the group consisting of the use of amber vials to protect from light and the use of stoppers with specialized coatings to improve shelf-life stability.

When the improvement is made by use of a drug delivery system, the drug delivery system can be, but is not limited to, a drug delivery system selected from the group consisting of:

(a) oral dosage forms;

(b) nanocrystals;

(c) nanoparticles;

(d) cosolvents;

(e) slurries;

(f) syrups;

(g) bioerodible polymers;

(h) liposomes;

(i) slow-release injectable gels;

(j) microspheres; and

(k) targeting compositions with epidermal growth factor receptor-binding peptides.

Nanocrystals are described in U.S. Pat. No. 7,101,576 to Hovey et al.

Nanoparticles for drug delivery are described in U.S. Pat. No. 8,258,132 to Bosch et al. Typically, such nanoparticles have an average particle size of the active ingredient of less than about 1000 nm, more preferably, less than about 400 nm, and most preferably, less than about 250 nm. The nanoparticles can be coated with a surface stabilizer, such as, but not limited to, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween 20® and Tween 80® (ICI Speciality Chemicals)); polyethylene glycols (e.g., Carbowaxes 3550® and 934® (Union Carbide)), polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethyl-cellulose, hydroxypropylmethyl-cellulose phthalate, noncrystalline cellulose, magnesium aluminium silicate, triethanolamine, polyvinyl alcohol (PVA), polyvinylpyrrolidone (PVP), 4-(1,1,3,3-tetramethylbutyl)-phenol polymer with ethylene oxide and formaldehyde (also known as tyloxapol, superione, and triton), poloxamers (e.g., Pluronics F68® and F108®, which are block copolymers of ethylene oxide and propylene oxide); poloxamines (e.g., Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.)); Tetronic 1508® (T-1508) (BASF Wyandotte Corporation), dialkylesters of sodium sulfosuccinic acid (e.g., Aerosol OT®, which is a dioctyl ester of sodium sulfosuccinic acid (American Cyanamid)), dioctyl sodium sulfosuccinate (DOSS), docusate sodium (Ashland Chem. Co., Columbus, Ohio); Duponol P®, which is a sodium lauryl sulfate (DuPont); Triton X-200®, which is an alkyl aryl polyether sulfonate (Rohm and Haas); Crodestas F-110®, which is a mixture of sucrose stearate and sucrose distearate (Croda Inc.); p-isononylphenoxy-poly-(glycidol), also known as Olin-IOG® or Surfactant 10-G® (Olin Chemicals, Stamford, Conn.); Crodestas SL-40® (Croda, Inc.); and SA9OHCO, which is C₁₈H₃₇CH₂(CON(CH₃)—OCH₂(CHOH)₄(CH₂OH)₂ (Eastman Kodak Co.); decanoyl-N-methylglucamide; n-decyl β-D-glucopyranoside; n-decyl β-D-maltopyranoside; n-dodecyl 3-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-nonanoyl β-D-glucopyranoside; octanoyl-N-methylglucamide; n-octyl β-D-glucopyranoside; and octyl β-D-thioglucopyranoside.

Nanoparticles for drug delivery are also described in United States Patent Application Publication No. 2010/209479 by Carroll et al. These nanoparticles include carbon nanoparticles such as carbon nanotubes.

Pharmaceutically acceptable cosolvents are described in U.S. Pat. No. 8,207,195 to Navratil et al., and include, but are not limited to, water, methanol, ethanol, 1-propanol, isopropanol, 1-butanol, isobutanol, t-butanol, acetone, methyl ethyl ketone, acetonitrile, ethyl acetate, benzene, toluene, xylene(s), ethylene glycol, dichloromethane, 1,2-dichloroethane, N-methylformamide, N,N-dimethylformamide, N-methylacetamide, pyridine, dioxane, and diethyl ether.

Slurries for use in pharmaceutical formulations are described in United States Patent Application Publication No. 2006/0229277 by Laxminarayan.

Syrups for use in pharmaceutical formulations are described in U.S. Pat. No. 8,252,930 to Stoit et al. Such syrups can include the active ingredient and a syrup-forming component such as sugar or sugar alcohols and a mixture of ethanol, water, glycerol, propylene glycol and polyethylene glycol. If desired, such liquid preparations may contain coloring agents, flavoring agents, preservatives, saccharine and carboxymethyl cellulose or other thickening agents.

Bioerodible polymers are described in U.S. Pat. No. 7,318,931 to Okumu et al. A bioerodible polymer decomposes when placed inside an organism, as measured by a decline in the molecular weight of the polymer over time. Polymer molecular weights can be determined by a variety of methods including size exclusion chromatography (SEC), and are generally expressed as weight averages or number averages. A polymer is bioerodible if, when in phosphate buffered saline (PBS) of pH 7.4 and a temperature of 37° C., its weight-average molecular weight is reduced by at least 25% over a period of 6 months as measured by SEC. Useful bioerodible polymers include polyesters, such as poly(caprolactone), poly(glycolic acid), poly(lactic acid), and poly(hydroxybutryate); polyanhydrides, such as poly(adipic anhydride) and poly(maleic anhydride); polydioxanone; polyamines; polyamides; polyurethanes; polyesteramides; polyorthoesters; polyacetals; polyketals; polycarbonates; polyorthocarbonates; polyphosphazenes; poly(malic acid); poly(amino acids); polyvinylpyrrolidone; poly(methyl vinyl ether); poly(alkylene oxalate); poly(alkylene succinate); polyhydroxycellulose; chitin; chitosan; and copolymers and mixtures thereof.

Liposomes are well known as drug delivery vehicles. Liposome preparation is described in European Patent Application Publication No. EP 1332755 by Weng et al. Liposomes can incorporate short oligopeptide sequences capable of targeting the EGFR receptor, as described in United States Patent Application Publication 2012/0213844 by Huang et al. Alternatively, liposomes can include nuclear localization signal/fusogenic peptide conjugates and form targeted liposome complexes, as described in United States Patent Application Publication 2012/0183596 to Boulikas.

Slow release injectable gels are known in the art and are described, for example, in B. Jeong et al., “Drug Release from Biodegradable Injectable Thermosensitive Hydrogel of PEG-PLGA-PEG Triblock Copolymers,” J. Controlled Release 63: 155-163 (2000).

The use of microspheres for drug delivery is known in the art and is described, for example, in H. Okada & H. Taguchi, “Biodegradable Microspheres in Drug Delivery,” Crit. Rev. Ther. Drug Carrier Sys. 12: 1-99 (1995).

The use of targeting compositions with epidermal growth factor receptor-binding peptides is described in United States Patent Application Publication No. 2010/0151003 by Trikha et al.

When the improvement is made by use of a drug conjugate form, the drug conjugate form can be, but is not limited to, a drug conjugate form selected from the group consisting of:

(a) a polymer system;

(b) polylactides;

(c) polyglycolides;

(d) amino acids;

(e) peptides;

(f) multivalent linkers;

(g) immunoglobulins;

(h) cyclodextrin polymers;

(i) modified transferrin;

(j) hydrophobic or hydrophobic-hydrophilic polymers;

(k) conjugates with a phosphonoformic acid partial ester;

(l) conjugates with a cell-binding agent incorporating a charged cross-linker; and

(m) conjugates with β-glucuronides through a linker.

Polylactide conjugates are well known in the art and are described, for example, in R. Tong & C. Cheng, “Controlled Synthesis of Camptothecin-Polylactide Conjugates and Nanoconjugates,” Bioconjugate Chem. 21: 111-121 (2010).

Polyglycolide conjugates are also well known in the art and are described, for example, in PCT Patent Application Publication No. WO 2003/070823 by Elmaleh et al.

Multivalent linkers are known in the art and are described, for example, in United States Patent Application Publication No. 2007/0207952 by Silva et al. For example, multivalent linkers can contain a thiophilic group for reaction with a reactive cysteine, and multiple nucleophilic groups (such as NH or OH) or electrophilic groups (such as activated esters) that permit attachment of a plurality of biologically active moieties to the linker.

Conjugates with immunoglobulins are disclosed in U.S. Pat. No. 4,925,662 to Oguchi et al. The conjugates are prepared by use of a cross-linking agent such as carbodiimide, glutaraldehyde, or diethyl malonimidate.

Cyclodextrin polymers, their conjugates with therapeutically active agents, and their administration together with particles are described in United States Patent Application Publication Serial No. 2012/0213854 by Fetzer.

Conjugates with modified transferrin are described in United States Patent Application Publication Serial No. 2011/0288023 by Kamei et al.

Conjugates with hydrophobic or hydrophobic-hydrophilic polymers are described in United States Patent Application Publication No. 2011/0268658 by Crawford et al. These polymers can include mono-, di-, or tripeptides. These polymers can also include polylactic acid (PLA), polyglycolic acid (PGA), poly (lactic-co-glycolic) acid (PLGA), polycaprolactone (PCL), polydioxanone (PDO), polyanhydrides, polyorthoesters, or chitosan.

Conjugates with a phosphonoformic acid partial ester are described in United States Patent Application Publication No. 2010/227831 by Saha et al.

Conjugates with a cell-binding agent incorporating a charged cross-linker are described in U.S. Pat. No. 8,236,319 to Chari et al.

Conjugates with β-glucuronides through a linker are described in U.S. Pat. No. 8,039,273 to Jeffrey.

Suitable reagents for cross-linking many combinations of functional groups are known in the art. For example, electrophilic groups can react with many functional groups, including those present in proteins or polypeptides. Various combinations of reactive amino acids and electrophiles are known in the art and can be used. For example, N-terminal cysteines, containing thiol groups, can be reacted with halogens or maleim ides. Thiol groups are known to have reactivity with a large number of coupling agents, such as alkyl halides, haloacetyl derivatives, maleimides, aziridines, acryloyl derivatives, arylating agents such as aryl halides, and others. These are described in G. T. Hermanson, “Bioconjugate Techniques” (Academic Press, San Diego, 1996), pp. 146-150. The reactivity of the cysteine residues can be optimized by appropriate selection of the neighboring amino acid residues. For example, a histidine residue adjacent to the cysteine residue will increase the reactivity of the cysteine residue. Other combinations of reactive amino acids and electrophilic reagents are known in the art. For example, maleim ides can react with amino groups, such as the ε-amino group of the side chain of lysine, particularly at higher pH ranges. Aryl halides can also react with such amino groups. Haloacetyl derivatives can react with the imidazolyl side chain nitrogens of histidine, the thioether group of the side chain of methionine, and the ε-amino group of the side chain of lysine. Many other electrophilic reagents are known that will react with the ε-amino group of the side chain of lysine, including, but not limited to, isothiocyanates, isocyanates, acyl azides, N-hydroxysuccinimide esters, sulfonyl chlorides, epoxides, oxiranes, carbonates, imidoesters, carbodiimides, and anhydrides. These are described in G. T. Hermanson, “Bioconjugate Techniques” (Academic Press, San Diego, 1996), pp. 137-146. Additionally, electrophilic reagents are known that will react with carboxylate side chains such as those of aspartate and glutamate, such as diazoalkanes and diazoacetyl compounds, carbonydilmidazole, and carbodiim ides. These are described in G. T. Hermanson, “Bioconjugate Techniques” (Academic Press, San Diego, 1996), pp. 152-154. Furthermore, electrophilic reagents are known that will react with hydroxyl groups such as those in the side chains of serine and threonine, including reactive haloalkane derivatives. These are described in G. T. Hermanson, “Bioconjugate Techniques” (Academic Press, San Diego, 1996), pp. 154-158. In another alternative embodiment, the relative positions of electrophile and nucleophile (i.e., a molecule reactive with an electrophile) are reversed so that the protein has an amino acid residue with an electrophilic group that is reactive with a nucleophile and the targeting molecule includes therein a nucleophilic group. This includes the reaction of aldehydes (the electrophile) with hydroxylamine (the nucleophile), described above, but is more general than that reaction; other groups can be used as electrophile and nucleophile. Suitable groups are well known in organic chemistry and need not be described further in detail.

Additional combinations of reactive groups for cross-linking are known in the art. For example, amino groups can be reacted with isothiocyanates, isocyanates, acyl azides, N-hydroxysuccinimide (NHS) esters, sulfonyl chlorides, aldehydes, glyoxals, epoxides, oxiranes, carbonates, alkylating agents, imidoesters, carbodiimides, and anhydrides. Thiol groups can be reacted with haloacetyl or alkyl halide derivatives, maleimides, aziridines, acryloyl derivatives, acylating agents, or other thiol groups by way of oxidation and the formation of mixed disulfides. Carboxy groups can be reacted with diazoalkanes, diazoacetyl compounds, carbonyldiimidazole, carbodiim ides. Hydroxyl groups can be reacted with epoxides, oxiranes, carbonyldiimidazole, N,N′-disuccinim idyl carbonate, N-hydroxysuccinim idyl chloroformate, periodate (for oxidation), alkyl halogens, or isocyanates. Aldehyde and ketone groups can react with hydrazines, reagents forming Schiff bases, and other groups in reductive amination reactions or Mannich condensation reactions. Still other reactions suitable for cross-linking reactions are known in the art. Such cross-linking reagents and reactions are described in G. T. Hermanson, “Bioconjugate Techniques” (Academic Press, San Diego, 1996).

When the improvement is made by use of a compound analog, the compound analog can be, but is not limited to, a compound analog selected from the group consisting of:

(a) alteration of side chains to increase or decrease lipophilicity;

(b) addition of an additional chemical functionality to alter a property selected from the group consisting of reactivity, electron affinity, and binding capacity; and

(c) alteration of salt form.

When the improvement is made by use of a prodrug system, the prodrug system can be, but is not limited to, a prodrug system selected from the group consisting of:

(a) the use of enzyme sensitive esters;

(b) the use of dimers;

(c) the use of Schiff bases;

(d) the use of pyridoxal complexes;

(e) the use of caffeine complexes; and

(f) the use of nitric oxide-releasing prodrugs;

(g) the use of prodrugs with fibroblast activation protein α-cleavable oligopeptides;

(h) the use of prodrugs that are products of reaction with an acetylating or carbamylating agent;

(i) the use of prodrugs that are hexanoate conjugates;

(j) the use of prodrugs that are polymer-agent conjugates; and

(k) the use of prodrugs that are subject to redox activation.

As used herein, the term “prodrug” refers to compounds that are transformed in vivo to yield a disclosed compound or a pharmaceutically acceptable form of the compound. In some embodiments, a prodrug is a compound that may be converted under physiological conditions or by solvolysis to a biologically active compound as described herein. Thus, the term “prodrug” refers to a precursor of a biologically active compound that is pharmaceutically acceptable. A prodrug can be inactive when administered to a subject, but is then converted in vivo to an active compound, for example, by hydrolysis (e.g., hydrolysis in blood or a tissue). In certain cases, a prodrug has improved physical and/or delivery properties over a parent compound from which the prodrug has been derived. The prodrug often offers advantages of solubility, tissue compatibility, or delayed release in a mammalian organism (H. Bundgard, Design of Prodrugs (Elsevier, Amsterdam, 1988), pp. 7-9, 21-24). A discussion of prodrugs is provided in T. Higuchi et al., “Pro-Drugs as Novel Delivery Systems,” ACS Symposium Series, Vol. 14 and in E. B. Roche, ed., Bioreversible Carriers in Drug Design (American Pharmaceutical Association & Pergamon Press, 1987). Exemplary advantages of a prodrug can include, but are not limited to its physical properties, such as enhanced water solubility for parenteral administration at physiological pH compared to the parent compound, enhanced absorption from the digestive tract, or enhanced drug stability for long-term storage.

The term “prodrug” is also meant to include any covalently bonded carriers which release the active compound in vivo when the prodrug is administered to a subject. Prodrugs of a therapeutically active compound, as described herein, can be prepared by modifying one or more functional groups present in the therapeutically active compound in such a way that the modifications are cleaved, either in routine manipulation or in vivo, to yield the parent therapeutically active compound. Prodrugs include compounds wherein a hydroxy, amino, or mercapto group is covalently bonded to any group that, when the prodrug of the active compound is administered to a subject, cleaves to form a free hydroxy, free amino, or free mercapto group, respectively. Examples of prodrugs include, but are not limited to, formate or benzoate derivatives of an alcohol or acetamide, formamide or benzamide derivatives of a therapeutically active agent possessing an amine functional group available for reaction, and the like.

For example, if a therapeutically active agent or a pharmaceutically acceptable form of a therapeutically active agent contains a carboxylic acid functional group, a prodrug can comprise an ester formed by the replacement of the hydrogen atom of the carboxylic acid group with a group such as C₁₋₈ alkyl, C₂₋₁₂ alkanoyloxymethyl, 1-(alkanoyloxy)ethyl having from 4 to 9 carbon atoms, 1-methyl-1-(alkanoyloxy)ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, γ-butyrolacton-4-yl, di-N,N(C₁-C₂)alkylamino(C₂-C₃)alkyl (such as (3-dimethylaminoethyl), carbamoyl-(C₁-C₂)alkyl, N,N-di (C₁-C₂)alkylcarbamoyl-(C₁-C₂)alkyl and piperidino-, pyrrolidino-, or morpholino(C₂-C₃)alkyl.

Similarly, if a disclosed compound or a pharmaceutically acceptable form of the compound contains an alcohol functional group, a prodrug can be formed by the replacement of the hydrogen atom of the alcohol group with a group such as (C₁-C₆)alkanoyloxymethyl, 1-((C₁-C₆))alkanoyloxy)ethyl, 1-methyl-1-((C₁-C₆)alkanoyloxy)ethyl (C₁-C₆)alkoxycarbonyloxymethyl, N(C₁-C₆)alkoxycarbonylaminomethyl, succinoyl, (C₁-C₆)alkanoyl, α-amino(C₁-C₄)alkanoyl, arylacyl and α-aminoacyl, or α-aminoacyl-α-aminoacyl, where each α-aminoacyl group is independently selected from the naturally occurring L-amino acids, P(O)(OH)₂, P(O)(O(C₁-C₆)alkyl)₂ or glycosyl (the radical resulting from the removal of a hydroxyl group of the hemiacetal form of a carbohydrate).

If a disclosed compound or a pharmaceutically acceptable form of the compound incorporates an amine functional group, a prodrug can be formed by the replacement of a hydrogen atom in the amine group with a group such as R-carbonyl, RO-carbonyl, NRR′-carbonyl where R and R′ are each independently (C₁-C₁₀)alkyl, (C₃-C₇)cycloalkyl, benzyl, or R-carbonyl is a natural α-aminoacyl or natural α-aminoacyl-natural α-aminoacyl, C(OH)C(O)OY¹ wherein Y¹ is H, (C₁-C₆)alkyl or benzyl, C(OY²)Y³ wherein Y² is (C₁-C₄) alkyl and Y³ is (C₁-C₆)alkyl, carboxy(C₁-C₆)alkyl, amino(C₁-C₄)alkyl or mono-N or di-N,N(C₁-C₆)alkylaminoalkyl,C(Y⁴)Y⁵ wherein Y⁴ is H or methyl and Y⁵ is mono-N or di-N,N(C₁-C₆)alkylamino, morpholino, piperidin-1-yl or pyrrolidin-1-yl.

The use of prodrug systems is described in T. Järvinen et al., “Design and Pharmaceutical Applications of Prodrugs” in Drug Discovery Handbook (S. C. Gad, ed., Wiley-Interscience, Hoboken, N.J., 2005), ch. 17, pp. 733-796. This publication describes the use of enzyme sensitive esters as prodrugs. The use of dimers as prodrugs is described in U.S. Pat. No. 7,879,896 to Allegretti et al. The use of peptides in prodrugs is described in S. Prasad et al., “Delivering Multiple Anticancer Peptides as a Single Prodrug Using Lysyl-Lysine as a Facile Linker,” J. Peptide Sci. 13: 458-467 (2007). The use of Schiff bases as prodrugs is described in U.S. Pat. No. 7,619,005 to Epstein et al. The use of caffeine complexes as prodrugs is described in U.S. Pat. No. 6,443,898 to Unger et al. The use of nitric oxide-releasing prodrugs is described in N. Nath et al., “JS-K, a Nitric Oxide-Releasing Prodrug, Modulates β-Catenin/TCF Signaling in Leukemic Jurkat Cells: Evidence of an S-Nitrosylated Mechanism,” Biochem. Pharmacol. 80: 1641-1649 (2010). The use of prodrugs with fibroblast activation protein α-cleavable oligopeptides is described in United States Patent Application Publication No. 2002/0155565 by Garin-Chesa et al. The use of prodrugs that are products of reaction with an acetylating or carbamylating agent is described in J. H. Lin & J. Y. H. Lu, “Role of Pharmacokinetics and Metabolism in Drug Discovery and Development,” Pharmacol. Rev. 4: 403-449 (1997). The use of hexanoate conjugates is described in U.S. Pat. No. 8,101,661 to Mickle. The use of polymer-agent conjugates is described in R. Satchi et al., “PDEPT: Polymer-Directed Enzyme Prodrug Therapy,” Br. J. Cancer 85: 1070-1076 (2001). The use of prodrugs that are subject to redox activation is described in S. H. van Rijt & P. J. Sadler, “Current Applications and Future Potential for Bioinorganic Chemistry in the Development of Anticancer Drugs,” Drug Discov. Today 14: 1089-1097 (2009).

When the improvement is made by use of a multiple drug system, the multiple drug system can be, but is not limited to, a multiple drug system selected from the group consisting of:

(a) inhibitors of multi-drug resistance;

(b) specific drug resistance inhibitors;

(c) specific inhibitors of selective enzymes;

(d) signal transduction inhibitors;

(e) meisoindigo;

(f) imatinib;

(g) hydroxyurea;

(h) dasatinib;

(i) capecitabine;

(j) nilotinib;

(k) repair inhibition agents; and

(l) topoisomerase inhibitors with non-overlapping side effects.

Multi-drug resistance inhibitors are described in U.S. Pat. No. 6,011,069 to Inomata et al.

Specific drug resistance inhibitors are described in T. Hideshima et al., “The Proteasome Inhibitor PS-341 Inhibits Growth, Induces Apoptosis, and Overcomes Drug Resistance in Human Multiple Myeloma Cells,” Cancer Res. 61: 3071-3076 (2001).

Selective inhibitors of specific enzymes are described in D. Leung et al., “Discovering Potent and Selective Reversible Inhibitors of Enzymes in Complex Proteomes,” Nature Biotechnol. 21: 687-691 (2003).

Repair inhibition is described in N. M. Martin, “DNA Repair Inhibition and Cancer Therapy,” J. Photochem. Photobiol. B 63: 162-170 (2001).

When the improvement is made by biotherapeutic enhancement, the biotherapeutic enhancement can be performed by use in combination as sensitizers/potentiators with a therapeutic agent or technique that can be, but is not limited to, a therapeutic agent or technique selected from the group consisting of:

(a) cytokines;

(b) lymphokines;

(c) therapeutic antibodies;

(d) antisense therapies;

(e) gene therapies;

(f) ribozymes;

(g) RNA interference; and

(h) vaccines.

Antisense therapies are described, for example, in B. Weiss et al., “Antisense RNA Gene Therapy for Studying and Modulating Biological Processes,” Cell. Mol. Life Sci. 55: 334-358 (1999).

Ribozymes are described, for example, in S. Pascolo, “RNA-Based Therapies” in Drug Discovery Handbook (S. C. Gad, ed., Wiley-Interscience, Hoboken, N.J., 2005), ch. 27, pp. 1273-1278.

RNA interference is described, for example, in S. Pascolo, “RNA-Based Therapies” in Drug Discovery Handbook (S. C. Gad, ed., Wiley-Interscience, Hoboken, N.J., 2005), ch. 27, pp. 1278-1283.

As described above, typically, cancer vaccines are based on an immune response to a protein or proteins occurring in cancer cells that does not occur in normal cells. Cancer vaccines include Provenge for metastatic hormone-refractory prostate cancer, Oncophage for kidney cancer, CimaVax-EGF for lung cancer, MOBILAN, Neuvenge for Her2/neu expressing cancers such as breast cancer, colon cancer, bladder cancer, and ovarian cancer, Stimuvax for breast cancer, and others. Cancer vaccines are described in S. Pejawar-Gaddy & O. Finn, (2008), supra.

When the biotherapeutic enhancement is use in combination as sensitizers/potentiators with a therapeutic antibody, the therapeutic antibody can be, but is not limited to, a therapeutic antibody selected from the group consisting of bevacizumab (Avastin), rituximab (Rituxan), trastuzumab (Herceptin), and cetuximab (Erbitux).

When the improvement is made by use of biotherapeutic resistance modulation, the biotherapeutic resistance modulation can be, but is not limited to, use against glioblastoma tumors resistant to a therapeutic agent or technique selected from the group consisting of:

(a) biological response modifiers;

(b) cytokines;

(c) lymphokines;

(d) therapeutic antibodies;

(e) antisense therapies;

(f) gene therapies;

(g) ribozymes;

(h) RNA interference; and vaccines.

When the biotherapeutic resistance modulation is use against tumors resistant to therapeutic antibodies, the therapeutic antibody can be, but is not limited to, a therapeutic antibody selected from the group consisting of bevacizumab (Avastin), rituximab (Rituxan), trastuzumab (Herceptin), and cetuximab (Erbitux).

When the improvement is made by radiation therapy enhancement, the radiation therapy enhancement can be, but is not limited to, a radiation therapy enhancement agent or technique selected from the group consisting of:

(a) hypoxic cell sensitizers;

(b) radiation sensitizers/protectors;

(c) photosensitizers;

(d) radiation repair inhibitors;

(e) thiol depleters;

(f) vaso-targeted agents;

(g) DNA repair inhibitors;

(h) radioactive seeds;

(i) radionuclides;

(j) radiolabeled antibodies; and

(k) brachytherapy.

A substituted hexitol derivative such as dianhydrogalactitol can be used in combination with radiation for the treatment of glioblastoma or other malignancies as described herein.

Hypoxic cell sensitizers are described in C. C. Ling et al., “The Effect of Hypoxic Cell Sensitizers at Different Irradiation Dose Rates,” Radiation Res. 109: 396-406 (1987). Radiation sensitizers are described in T. S. Lawrence, “Radiation Sensitizers and Targeted Therapies,” Oncology 17 (Suppl. 13) 23-28 (2003). Radiation protectors are described in S. B. Vuyyuri et al., “Evaluation of D-Methionine as a Novel Oral Radiation Protector for Prevention of Mucositis,” Clin. Cancer Res. 14: 2161-2170 (2008). Photosensitizers are described in R. R. Allison & C. H. Sibata, “Oncologic Photodynamic Therapy Photosensitizers: A Clinical Review,” Photodiagnosis Photodynamic Ther. 7: 61-75 (2010). Radiation repair inhibitors and DNA repair inhibitors are described in M. Hingorani et al., “Evaluation of Repair of Radiation-Induced DNA Damage Enhances Expression from Replication-Defective Adenoviral Vectors,” Cancer Res. 68: 9771-9778 (2008). Thiol depleters are described in K. D. Held et al., “Postirradiation Sensitization of Mammalian Cells by the Thiol-Depleting Agent Dimethyl Fumarate,” Radiation Res. 127: 75-80 (1991). Vaso-targeted agents are described in A. L. Seynhaeve et al., “Tumor Necrosis Factor α Mediates Homogeneous Distribution of Liposomes in Murine Melanoma that Contributes to a Better Tumor Response,” Cancer Res. 67: 9455-9462 (2007). As described above, radiation therapy is frequently employed for the treatment of glioblastoma, so radiation therapy enhancement is significant for this malignancy.

When the improvement is by use of a novel mechanism of action, the novel mechanism of action can be, but is not limited to, a novel mechanism of action that is a therapeutic interaction with a target or mechanism selected from the group consisting of:

(a) inhibitors of poly-ADP ribose polymerase;

(b) agents that affect vasculature or vasodilation;

(c) oncogenic targeted agents;

(d) signal transduction inhibitors;

(e) EGFR inhibition;

(f) protein kinase C inhibition;

(g) phospholipase C downregulation;

(h) Jun downregulation;

(i) histone genes;

(j) VEGF;

(k) ornithine decarboxylase;

(l) ubiquitin C;

(m) Jun D;

(n) v-Jun;

(o) GPCRs;

(p) protein kinase A;

(q) protein kinases other than protein kinase A;

(r) prostate specific genes;

(s) telomerase;

(t) histone deacetylase; and

(u) tyrosine kinase inhibitors.

EGFR inhibition is described in G. Giaccone & J. A. Rodriguez, “EGFR Inhibitors: What Have We Learned from the Treatment of Lung Cancer,” Nat. Clin. Pract. Oncol. 11: 554-561 (2005). Protein kinase C inhibition is described in H. C. Swannie & S. B. Kaye, “Protein Kinase C Inhibitors,” Curr. Oncol. Rep. 4: 37-46 (2002). Phospholipase C downregulation is described in A. M. Martelli et al., “Phosphoinositide Signaling in Nuclei of Friend Cells: Phospholipase C β Downregulation Is Related to Cell Differentiation,” Cancer Res. 54: 2536-2540 (1994). Downregulation of Jun (specifically, c-Jun) is described in A. A. P. Zada et al., “Downregulation of c-Jun Expression and Cell Cycle Regulatory Molecules in Acute Myeloid Leukemia Cells Upon CD44 Ligation,” Oncogene 22: 2296-2308 (2003). The role of histone genes as a target for therapeutic intervention is described in B. Calabretta et al., “Altered Expression of G1-Specific Genes in Human Malignant Myeloid Cells,” Proc. Natl. Acad. Sci. USA 83: 1495-1498 (1986). The role of VEGF as a target for therapeutic intervention is described in A. Zielke et al., “VEGF-Mediated Angiogenesis of Human Pheochromocytomas Is Associated to Malignancy and Inhibited by anti-VEGF Antibodies in Experimental Tumors,” Surgery 132: 1056-1063 (2002). The role of ornithine decarboxylase as a target for therapeutic intervention is described in J. A. Nilsson et al., “Targeting Ornithine Decarboxylase in Myc-Induced Lymphomagenesis Prevents Tumor Formation,” Cancer Cell 7: 433-444 (2005). The role of ubiquitin C as a target for therapeutic intervention is described in C. Aghajanian et al., “A Phase I Trial of the Novel Proteasome Inhibitor PS341 in Advanced Solid Tumor Malignancies,” Clin. Cancer Res. 8: 2505-2511 (2002). The role of Jun D as a target for therapeutic intervention is described in M. M. Caffarel et al., “JunD Is Involved in the Antiproliferative Effect of Δ⁹-Tetrahydrocannibinol on Human Breast Cancer Cells,” Oncogene 27: 5033-5044 (2008). The role of v-Jun as a target for therapeutic intervention is described in M. Gao et al., “Differential and Antagonistic Effects of v-Jun and c-Jun,” Cancer Res. 56: 4229-4235 (1996). The role of protein kinase A as a target for therapeutic intervention is described in P. C. Gordge et al., “Elevation of Protein Kinase A and Protein Kinase C in Malignant as Compared With Normal Breast Tissue,” Eur. J. Cancer 12: 2120-2126 (1996). The role of telomerase as a target for therapeutic intervention is described in E. K. Parkinson et al., “Telomerase as a Novel and Potentially Selective Target for Cancer Chemotherapy,” Ann. Med. 35: 466-475 (2003). The role of histone deacetylase as a target for therapeutic intervention is described in A. Melnick & J. D. Licht, “Histone Deacetylases as Therapeutic Targets in Hematologic Malignancies,” Curr. Opin. Hematol. 9: 322-332 (2002).

When the improvement is made by use of selective target cell population therapeutics, the use of selective target cell population therapeutics can be, but is not limited to, a use selected from the group consisting of:

(a) use against radiation sensitive cells;

(b) use against radiation resistant cells; and

(c) use against energy depleted cells.

The improvement can also be made by use of dianhydrogalactitol in combination with ionizing radiation.

When the improvement is made by use with an agent to enhance the activity of an alkylating hexitol derivative, the agent to enhance the activity of the alkylating hexitol derivative can be, but is not limited to, an agent selected from the group consisting of:

(a) nicotinamide;

(b) caffeine;

(c) tetandrine; and

(d) berberine.

When the improvement is made by use of an agent that counteracts myelosuppression, the agent that counteracts myelosuppression can be, but is not limited to, a dithiocarbamate.

U.S. Pat. No. 5,035,878 to Borch et al., discloses dithiocarbamates for treatment of myelosuppression; the dithiocarbamates are compounds of the formula R¹R²NCS(S)M or R¹R²NCSS—SC(S)NR³R⁴, wherein R¹, R², R³, and R⁴ are the same or different, and R¹, R², R³, and R⁴ are aliphatic, cycloaliphatic, or heterocycloaliphatic groups that are unsubstituted or substituted by hydroxyl; or wherein one of R¹ and R² and one of R³ and R⁴ can be hydrogen; or wherein R¹, R², R³, and R⁴ taken together with the nitrogen atom upon which the pair of R groups is substituted, can be a 5-membered or 6-membered N-heterocyclic ring which is aliphatic or aliphatic interrupted by a ring oxygen or a second ring nitrogen, and M is hydrogen or one equivalent or a pharmaceutically acceptable cation, in which case the rest of the molecule is negatively charged.

U.S. Pat. No. 5,294,430 to Borch et al., discloses additional dithiocarbamates for treatment of myelosuppression. In general, these are compounds of Formula (D-I):

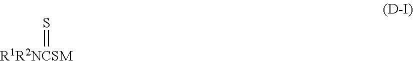

wherein:

(i) R¹ and R² are the same or different C₁-C₆ alkyl groups, C₃-C₆ cycloalkyl groups, or C₅-C₆ heterocycloalkyl groups; or

(ii) one of R¹ and R², but not both, can be H; or

(iii) R¹ and R² taken together with the nitrogen atom can be a 5-membered or 6-membered N-heterocyclic ring which is aliphatic or aliphatic interrupted by a ring oxygen or a second ring nitrogen; and

(iv) M is hydrogen or one equivalent of a pharmaceutically acceptable cation, in which case the rest of the molecule is negatively charged; or

(v) M is a moiety of Formula (D-II):

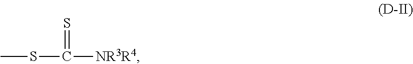

wherein R³ and R⁴ are defined in the same manner as R¹ and R². Where the group defined by Formula (D-I) is an anion, the cation can be an ammonium cation or can be derived from a monovalent or divalent metal such as an alkali metal or an alkaline earth metal, such as Na⁺, K⁺, or Zn⁺². In the case of the dithiocarbamic acids, the group defined by Formula (D-I) is linked to an ionizable hydrogen atom; typically, the hydrogen atom will dissociate at a pH above about 5.0. Among dithiocarbamates that can be used are: N-methyl,N-ethyldithiocarbamates, hexamethylenedithiocarbamic acid, sodium di(3-hydroxyethyl)dithiocarbamate, various dipropyl, dibutyl and diamyl dithiocarbamates, sodium N-methyl,N-cyclobutylmethyl dithiocarbamate, sodium N-allyl-N-cyclopropylmethyldithiocarbamate, cyclohexylamyldithiocarbamates, dibenzyl-dithiocarbamates, sodium dimethylene-dithiocarbamate, various pentamethylene dithiocarbamate salts, sodium pyrrolidine-N-carbodithioate, sodium piperidine-N-carbodithioate, sodium morpholine-N-carbo-dithioate, α-furfuryl dithiocarbamates and imidazoline dithiocarbamates. Another alternative is a compound where R¹ of Formula (D-I) is a hydroxy-substituted or, preferably, a (bis to penta) polyhydroxy-substituted lower alkyl group having up to 6 carbon atoms. For example, R¹ can be HO—CH₂—CHOH—CHOH—CHOH—CHOH—CH₂—. In such compounds, R² can be H or lower alkyl (unsubstituted or substituted with one or more hydroxyl groups). Steric problems can be minimized when R² is H, methyl, or ethyl. Accordingly, a particularly preferred compound of this type is an N-methyl-glucamine dithiocarbamate salt, the most preferred cations of these salts being sodium or potassium. Other preferred dithiocarbamates include the alkali or alkaline earth metal salts wherein the anion is di-n-butyldithiocarbamate, di-n-propyldithiocarbamate, pentamethylenedithiocarbamate, or tetramethylene dithiocarbamate.

When the improvement is made by use with an agent that increases the ability of the substituted hexitol to pass through the blood-brain barrier, the agent that increases the ability of the substituted hexitol to pass through the blood-brain barrier can be, but is not limited to, an agent selected from the group consisting of:

(a) a chimeric peptide of the structure of Formula (D-III):

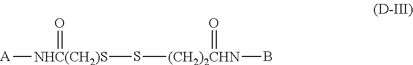

wherein: (A) A is somatostatin, thyrotropin releasing hormone (TRH), vasopressin, alpha interferon, endorphin, muramyl dipeptide or ACTH 4-9 analogue; and (B) B is insulin, IGF-I, IGF-II, transferrin, cationized (basic) albumin or prolactin; or a chimeric peptide of the structure of Formula (D-III) wherein the disulfide conjugating bridge between A and B is replaced with a bridge of Subformula (D-III(a)):

A-NH(CH₂)₂S—S—B (cleavable linkage) (D-III(a)),

wherein the bridge is formed using cysteamine and EDAC as the bridge reagents; or a chimeric peptide of the structure of Formula (D-III) wherein the disulfide conjugating bridge between A and B is replaced with a bridge of Subformula (D-III(b)):

A-NH═CH(CH₂)₃CH═NH—B (non-cleavable linkage) (D-III(b)),

wherein the bridge is formed using glutaraldehyde as the bridge reagent;

(b) a composition comprising either avidin or an avidin fusion protein bonded to a biotinylated substituted hexitol derivative to form an avidin-biotin-agent complex including therein a protein selected from the group consisting of insulin, transferrin, an anti-receptor monoclonal antibody, a cationized protein, and a lectin;

(c) a neutral liposome that is pegylated and incorporates the substituted hexitol derivative, wherein the polyethylene glycol strands are conjugated to at least one transportable peptide or targeting agent;

(d) a humanized murine antibody that binds to the human insulin receptor linked to the substituted hexitol derivative through an avidin-biotin linkage; and

(e) a fusion protein comprising a first segment and a second segment: the first segment comprising a variable region of an antibody that recognizes an antigen on the surface of a cell that after binding to the variable region of the antibody undergoes antibody-receptor-mediated endocytosis, and, optionally, further comprises at least one domain of a constant region of an antibody; and the second segment comprising a protein domain selected from the group consisting of avidin, an avidin mutein, a chemically modified avidin derivative, streptavidin, a streptavidin mutein, and a chemically modified streptavidin derivative, wherein the fusion protein is linked to the substituted hexitol by a covalent link to biotin.

Agents that improve penetration of the blood-brain barrier are disclosed in W. M. Pardridge, “The Blood-Brain Barrier: Bottleneck in Brain Drug Development,” NeuroRx 2: 3-14 (2005).

One class of these agents is disclosed in U.S. Pat. No. 4,801,575 to Pardridge, which discloses chimeric peptides for delivery of agents across the blood-brain barrier. These chimeric peptides include peptides of the general structure of Formula (D-IV):

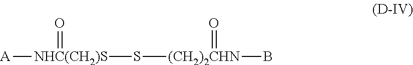

wherein:

(i) A is somatostatin, thyrotropin releasing hormone (TRH), vasopressin, alpha interferon, endorphin, muramyl dipeptide or ACTH 4-9 analogue; and

(ii) B is insulin, IGF-I, IGF-II, transferrin, cationized (basic) albumin or prolactin. In another alternative, the disulfide conjugating bridge between A and B is replaced with a bridge of Subformula (D-IV(a)):

A-NH(CH₂)₂S—S—B (cleavable linkage) (D-IV(a));

the bridge of Subformula (D-III(a)) is formed when cysteamine and EDAC are employed as the bridge reagents. In yet another alternative, the disulfide conjugating bridge between A and B is replaced with a bridge of Subformula (D-IV(b)):

A-NH═CH(CH₂)₃CH═NH—B (non-cleavable linkage) (D-IV(b));

the bridge of Subformula (D-III(b)) is formed when glutaraldehyde is employed as the bridge reagent.

U.S. Pat. No. 6,287,792 to Pardridge et al. discloses methods and compositions for delivery of agents across the blood-brain barrier comprising either avidin or an avidin fusion protein bonded to a biotinylated agent to form an avidin-biotin-agent complex. The avidin fusion protein can include the amino acid sequences of proteins such as insulin or transferrin, an anti-receptor monoclonal antibody, a cationized protein, or a lectin.

U.S. Pat. No. 6,372,250 to Pardridge discloses methods and compositions for delivery of agents across the blood-brain barrier employing liposomes. The liposomes are neutral liposomes. The surface of the neutral liposomes is pegylated. The polyethylene glycol strands are conjugated to transportable peptides or other targeting agents. Suitable targeting agents include insulin, transferrin, insulin-like growth factor, or leptin. Alternatively, the surface of the liposome could be conjugated with 2 different transportable peptides, one peptide targeting an endogenous BBB receptor and the other targeting an endogenous BCM (brain cell plasma membrane) peptide. The latter could be specific for particular cells within the brain, such as neurons, glial cells, pericytes, smooth muscle cells, or microglia. Targeting peptides may be endogenous peptide ligands of the receptors, analogues of the endogenous ligand, or peptidomimetic MAbs that bind the same receptor of the endogenous ligand. Transferrin receptor-specific peptidomimetic monoclonal antibodies can be used as transportable peptides. Monoclonal antibodies to the human insulin receptor can be used as transportable peptides. The conjugation agents which are used to conjugate the blood-barrier targeting agents to the surface of the liposome can be any of the well-known polymeric conjugation agents such as sphingomyelin, polyethylene glycol (PEG) or other organic polymers, with PEG preferred. The liposomes preferably have diameters of less than 200 nanometers. Liposomes having diameters of between 50 and 150 nanometers are preferred. Especially preferred are liposomes or other nanocontainers having external diameters of about 80 nanometers. Suitable types of liposomes are made with neutral phospholipids such as 1-palmitoyl-2-oleoyl-sn-glycerol-3-phosphocholine (POPC), diphosphatidyl phosphocholine, distearoylphosphatidylethanolamine (DSPE), or cholesterol. The transportable peptide is linked to the liposome as follows: A transportable peptide such as insulin or an HIRMAb is thiolated and conjugated to a maleimide group on the tip of a small fraction of the PEG strands; or, surface carboxyl groups on a transportable peptide such as transferrin or a TfRMAb are conjugated to a hydrazide (Hz) moiety on the tip of the PEG strand with a carboxyl activator group such as N-methyl-N′-3(dimethylaminopropyl)carbodiimide hydrochloride (EDAC); a transportable peptide is thiolated and conjugated via a disulfide linker to the liposome that has been reacted with N-succinimidyl 3-(2-pyridylthio)propionate (SPDP); or a transportable peptide is conjugated to the surface of the liposome with avidin-biotin technology, e.g., the transportable peptide is mono-biotinylated and is bound to avidin or streptavidin (SA), which is attached to the surface of the PEG strand.

U.S. Pat. No. 7,388,079 to Pardridge et al. discloses the use of a humanized murine antibody that binds to the human insulin receptor; the humanized murine antibody can be linked to the agent to be delivered through an avidin-biotin linkage.

U.S. Pat. No. 8,124,095 to Pardridge et al. discloses monoclonal antibodies that are capable of binding to an endogenous blood-brain barrier receptor-mediated transport system and are thus capable of serving as a vector for transport of a therapeutic agent across the BBB. The monoclonal antibody can be, for example, an antibody specifically binding the human insulin receptor on the human BBB.

United States Patent Application Publication No. 2005/0085419 by Morrison et al. discloses a fusion protein for delivery of a wide variety of agents to a cell via antibody-receptor-mediated endocytosis comprises a first segment and a second segment: the first segment comprising a variable region of an antibody that recognizes an antigen on the surface of a cell that after binding to the variable region of the antibody undergoes antibody-receptor-mediated endocytosis, and, optionally, further comprises at least one domain of a constant region of an antibody; and the second segment comprising a protein domain selected from the group consisting of avidin, an avidin mutein, a chemically modified avidin derivative, streptavidin, a streptavidin mutein, and a chemically modified streptavidin derivative. Typically, the antigen is a protein. Typically, the protein antigen on the surface of the cell is a receptor such as a transferrin receptor- or an insulin receptor. The invention also includes an antibody construct incorporating the fusion protein that is either a heavy chain or a light chain together with a complementary light chain or heavy chain to form an intact antibody molecule. The therapeutic agent can be a non-protein molecule and can be linked covalently to biotin.

Inhibitors of the enzyme poly-ADP ribose polymerase (PARP) have been developed for multiple indications, especially for treatment of malignancies. Several forms of cancer are more dependent on the activity of PARP than are non-malignant cells.

The enzyme PARP catalyzes the polymerization of poly-ADP ribose chains, typically attached to a single-strand break in cellular DNA. The coenzyme NAD⁺ is required as a substrate for generating ADP-ribose monomers to be polymerized; nicotinamide is the leaving group during polymerization, in contrast to pyrophosphate which is the leaving group during normal DNA or RNA synthesis, which leaves a pyrophosphate as the linking group between adjacent ribose sugars in the chain rather than phosphate as occurs in normal DNA or RNA. The PARP enzyme comprises four domains: a DNA-binding domain, a caspase-cleaved domain, an auto-modification domain, and a catalytic domain. The DNA-binding domain comprises two zinc finger motifs. In the presence of damaged DNA, the DNA-binding domain will bind the DNA and induce a conformational shift. PARP can be inactivated by caspase-3 cleavage, which is a step that occurs in programmed cell death (apoptosis).

Several PARP enzymes are known, including PARP1 and PARP2. Of these two enzymes, PARP1 is responsible for most cellular PARP activity. The binding of PARP1 to single-strand breaks in DNA through the amino-terminal zinc finger motifs recruits XRCC1, DNA ligase III, DNA polymerase β, and a kinase to the nick. This is known as base excision repair (BER). PARP2 has been shown to oligomerize with PARP1, and the oligomerization stimulates catalytic activity. PARP2 is also therefore implicated in BER.

PARP1 inhibitors inhibit the activity of PARP1 and thus inhibit the repair of single-strand breaks in DNA. When such breaks are unrepaired, subsequent DNA replication can induce double-strand breaks. The proteins BRCA1, BRCA2, and PALB2 can repair double-strand breaks in DNA by the error-free homologous recombinational repair (HRR) pathway. In tumors with mutations in the genes BRCA1, BRCA2, or PALB1, these double-strand breaks cannot be efficiently repaired, leading to cell death. Normal cells do not replicate their DNA as frequently as tumor cells, and normal cells that lack mutated BRCA1 or BRCA2 proteins can still repair these double-strand breaks through homologous repair. Therefore, normal cells are less sensitive to the activity of PARP inhibitors than tumor cells.

Some tumor cells that lack the tumor suppressor PTEN may be sensitive to PARP inhibitors because of downregulation of Rad51, a critical homologous recombination component. Tumor cells that are low in oxygen are also sensitive to PARP inhibitors.

PARP inhibitors are also considered potential treatments for other life-threatening diseases, including stroke and myocardial infarction, as well as for long-term neurodegenerative diseases (G. Graziani & C. Szabó, “Clinical Perspectives of PARP Inhibitors,” Pharmacol. Res. 52: 109-118 (2005)).

A number of PARP inhibitors are known in the art. PARP inhibitors include, but are not limited to, iniparib, talazoparib, olaparib, rucaparib, veliparib, CEP-9722 (a prodrug of CEP-8983 (11-methoxy-4,5,6,7-tetrahydro-1H-cyclopenta[a]pyrrolo[3,4-c]carbazole-1,3(2H)-dione), MK 4827 ((S)-2-(4-(piperidin-3-yl)phenyl)-2H-indazole-7-carboxamide), and BGB-290.

U.S. Pat. No. 9,073,893 to Papeo et al., discloses 3-oxo-2,3-dihydro-1H-indazole-4-carboxamide derivatives as PARP inhibitors, including: 3-oxo-2-(piperidin-4-yl)-2,3-dihydro-1H-indazole-4-carboxamide; 2-(1-cyclopentylpiperidin-4-yl)-3-oxo-2,3-dihydro-1H-indazole-4-carboxamide; 2-(1-cyclohexylpiperidin-4-yl)-3-oxo-2,3-dihydro-1H-indazole-4-carboxamide; 2-[1-(4,4-difluorocyclohexyl)piperidin-4-yl]-3-oxo-2,3-dihydro-1H-indazole-4-carboxamide; 2-(1-cyclohexylpiperidin-4-yl)-1-methyl-3-oxo-2,3-dihydro-1H-indazole-4-carboxamide; 2-[1-(4,4-difluorocyclohexyl)piperidin-4-yl]-1-methyl-3-oxo-2,3-dihydro-1-H-indazole-4-carboxamide; 2-(1-cyclopentylpiperidin-4-yl)-1-methyl-3-oxo-2,3-dihydro-1H-indazole-4-carboxamide; 2-(1-methylpiperidin-4-yl)-3-oxo-2,3-dihydro-1H-indazole-4-carboxamide; 1-methyl-3-oxo-2-(piperidin-4-yl)-2,3-dihydro-1H-indazole-4-carboxamide; 1-methyl-2-(1-methylpiperidin-4-yl)-3-oxo-2,3-dihydro-1H-indazole-4-carboxamide; 2-(1-ethylpiperidin-4-yl)-3-oxo-2,3-dihydro-1H-indazole-4-carboxamide; 3-oxo-2-(1-propylpiperidin-4-yl)-2,3-dihydro-1H-indazole-4-carboxamide; 2-(1-ethylpiperidin-4-yl)-1-methyl-3-oxo-2,3-dihydro-1H-indazole-4-carboxamide; 1-methyl-3-oxo-2-[1-(propan-2-yl)piperidin-4-yl]-2,3-dihydro-1H-indazole-4-carboxamide; 3-oxo-2-[1-(propan-2-yl)piperidin-4-yl]-2,3-dihydro-1H-indazole-4-carboxamide; 2-(1-cyclobutylpiperidin-4-yl)-3-oxo-2,3-dihydro-1H-indazole-4-carboxamide; 2-(1-cyclobutylpiperidin-4-yl)-6-fluoro-3-oxo-2,3-dihydro-1H-indazole-4-carboxamide; 2-(1-cyclobutylpiperidin-4-yl)-1-methyl-3-oxo-2,3-dihydro-1H-indazole-4-carboxamide; 2-(1-cyclobutylpiperidin-4-yl)-6-fluoro-1-methyl-3-oxo-2,3-dihydro-1H-indazole-4-carboxamide; 6-fluoro-3-oxo-2-(piperidin-4-yl)-2,3-dihydro-1H-indazole-4-carboxamide; 6-fluoro-1-methyl-3-oxo-2-(piperidin-4-yl)-2,3-dihydro-1H-indazole-4-carboxamide; 2-(1-cyclohexylpiperidin-4-yl)-6-fluoro-1-methyl-3-oxo-2,3-dihydro-1H-indazole-4-carboxamide; 2-(1-cyclohexylpiperidin-4-yl)-6-fluoro-3-oxo-2,3-dihydro-1H-indazole-4-carboxamide; 2-[1-(4,4-difluorocyclohexyl)piperidin-4-yl]-6-fluoro-1-methyl-3-oxo-2,3-dihydro-1H-indazole-4-carboxamide; and 2-[1-(4,4-dichlorocyclohexyl)piperidin-4-yl]-1-methyl-3-oxo-2,3-dihydro-1H-indazole-4-carboxamide.

U.S. Pat. No. 9,062,061 by Honda et al., discloses a PARP inhibitor of Formula (P-I):

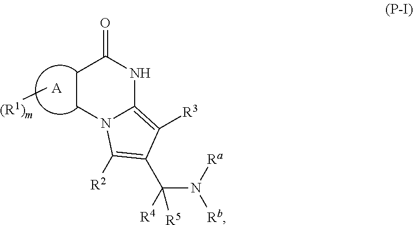

wherein:

(1) R¹ represents a halogen atom, a lower alkyl group, a hydroxy group, a lower alkoxy group, an amino group, a nitro group or a cyano group;

(2) R² and R³ may be the same or different and each represent a hydrogen atom, a halogen atom or a lower alkyl group;

(3) R⁴ and R⁵ may be the same or different and each represent a hydrogen atom, a deuterium atom or a lower alkyl group, or R⁴ and R⁵ may form an oxo group; R^{a} and R^{b} may be the same or different and each represent a hydrogen atom, a lower alkyl group optionally having a substituent or an aryl group optionally having a substituent; R^{a} and R^{b} may bind to each other to form a nitrogen-containing heterocyclic ring which may be substituted by one or plural R^{c};

(4) R^{c} represents a lower alkyl group optionally having a substituent, a lower cycloalkyl group optionally having a substituent, an aryl group optionally having a substituent, a heterocyclic group optionally having a substituent, a hydroxy group, a lower alkoxy group optionally having a substituent, a lower alkylcarbonyl group optionally having a substituent, a lower cycloalkylcarbonyl group optionally having a substituent, a lower alkylaminocarbonyl group optionally having a substituent, a lower cycloalkylaminocarbonyl group optionally having a substituent, a lower alkoxycarbonyl group optionally having a substituent, an amino group, a lower alkylamino group or a carboxyl group;

(5) ring A represents a benzene ring or an unsaturated heteromonocyclic ring; and

(6) m represents 0, 1 or 2.

U.S. Pat. No. 9,062,043 to Chua et al., discloses fused tricyclic PARP inhibitors, including a compound of Formula (P-II):

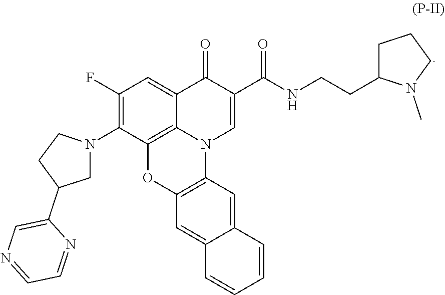 [SMILES: CN1CCCC1CCNC(=O)c1cn2c3c(c(N4CCC(c5cnccn5)C4)c(F)cc3c1=O)Oc1cc3ccccc3cc1-2]

U.S. Pat. No. 9,018,201 to Chu et al., discloses dihydropyridophthalazinone inhibitors of PARP.

U.S. Pat. No. 8,993,594 to Papeo et al., discloses substituted isoquinolin-1(2H)-one derivatives as inhibitors of PARP.

U.S. Pat. No. 8,980,902 to Brown et al., discloses substituted benzamide PARP inhibitors, including: N-ethyl-4-(4-((6-oxo-5,6-dihydropyrido[2,3-e]pyrrolo[1,2-c]pyrimidin-3-yl)methyl)piperazin-1-yl)benzamide; N-methyl-4-(4-((6-oxo-5,6-dihydropyrido[2,3-e]pyrrolo[1,2-c]pyrimidin-3-yl)methyl)piperazin-1-yl)benzamide; N-cyclopropyl-4-(4-((6-oxo-5,6-dihydropyrido[2,3-e]pyrrolo[1,2-c]pyrimidin-3-yl)methyl)piperazin-1-yl)benzamide; 3-chloro-N-methyl-4-(4-((6-oxo-5,6-dihydropyrido[2,3-e]pyrrolo[1,2-c]pyrimidin-3-yl)methyl)piperazin-1-yl)benzamide; 3-chloro-N-ethyl-4-(4((6-oxo-5,6-dihydropyrido[2,3-e]pyrrolo[1,2-c]pyrimidin-3-yl)methyl)piperazin-1-yl)benzamide; N,3-dimethyl-4-(4-((6-oxo-5,6-dihydropyrido[2,3-e]pyrrolo[1,2-c]pyrimidin-3-yl)methyl)piperazin-1-yl)benzamide; N-ethyl-3-methyl-4-(4-((6-oxo-5,6-dihydropyrido[2,3-e]pyrrolo[1,2-c]pyrimidin-3-yl)methyl)piperazin-1-yl)benzamide; 3-fluoro-N-methyl-4-(4-((6-oxo-5,6-dihydropyrido[2,3-e]pyrrolo[1,2-c]pyrimidin-3-yl)methyl)piperazin-1-yl)benzamide; and N-ethyl-3-fluoro-4-(4-((6-oxo-5,6-dihydropyrido[2,3-e]pyrrolo[1,2-c]pyrimidin-3-yl)methyl)piperazin-1-yl)benzamide.

U.S. Pat. No. 8,946,221 to Mevellec et al., discloses phthalazine derivatives as PARP inhibitors.

U.S. Pat. No. 8,894,989 to Xu et al., discloses tetraazaphenalen-3-one derivatives as PARP inhibitors.

U.S. Pat. No. 8,889,866 to Angibaud et al., discloses tetrahydrophenanthridinones and tetrahydrocyclopentaquinolinones as PARP inhibitors.

U.S. Pat. No. 8,883,787 to Xu et al., discloses diazabenzo[de]anthracen-3-one derivatives as PARP inhibitors.

U.S. Pat. No. 8,877,944 to Papeo et al., discloses substituted 3-oxo-2,3-dihydro-1H-isoindole-4-carboxamide derivatives as PARP inhibitors, including 2-[1-(cis-4-methoxycyclohexyl)piperidin-4-yl]-3-oxo-2,3-dihydro-1H-isoindole-4-carboxamide; 2-[1-(trans-4-methoxycyclohexyl)piperidin-4-yl]-3-oxo-2,3-dihydro-1H-isoindole-4-carboxamide; 2-{1-[cis-4-(hydroxymethyl)cyclohexyl]piperidin-4-yl}-3-oxo-2,3-dihydro-1H-isoindole-4-carboxamide; 2-{1-[trans-4-(hydroxymethyl)cyclohexyl]piperidin-4-yl}-3-oxo-2,3-dihydro-1H-isoindole-4-carboxamide; 2-{1-[cis-4-(methoxymethyl)cyclohexyl]piperidin-4-yl}-3-oxo-2,3-dihydro-1H-isoindole-4-carboxamide; 2-{1-[trans-4-(methoxymethyl)cyclohexyl]piperidin-4-yl}-3-oxo-2,3-dihydro-1H-isoindole-4-carboxamide; 2-[1-(4,4-difluorocyclohexyl)piperidin-4-yl]-3-oxo-2,3-dihydro-1H-isoindole-4-carboxamide; 2-[1-(1,4-dioxaspiro[4.5]dec-8-yl)piperidin-4-yl]-3-oxo-2,3-dihydro-1H-isoindole-4-carboxamide; 2-{1-[4-(dimethylamino)benzyl]piperidin-4-yl}-3-oxo-2,3-dihydro-1H-isoindole-4-carboxamide; 2-[1-(4-fluorobenzyl)piperidin-4-yl]-3-oxo-2,3-dihydro-1H-isoindole-4-carboxamide; 2-[1-(2-fluorobenzyl)piperidin-4-yl]-3-oxo-2,3-dihydro-1H-isoindole-4-carboxamide; 2-[1-(3-fluorobenzyl)piperidin-4-yl]-3-oxo-2,3-dihydro-1H-isoindole-4-carboxamide; 2-{1-[(1-methyl-1H-pyrrol-2-yl)methyl]piperidin-4-yl}-3-oxo-2,3-dihydro-1H-isoindole-4-carboxamide; 3-oxo-2-{1-[4-(trifluoromethylbenzyl]piperidin-4-yl}-2,3-dihydro-1H-isoindole-4-carboxamide; 3-oxo-2-[1-(quinolin-2-ylmethyl)piperidin-4-yl]-2,3-dihydro-1H-isoindole-4-carboxamide; 2-[1-(2,4-difluorobenzyl)piperidin-4-yl]-3-oxo-2,3-dihydro-1H-isoindole-4-carboxamide; 2-[1-(3,4-dimethylbenzyl)piperidin-4-yl]-3-oxo-2,3-dihydro-1H-isoindole-4-carboxamide; 2-[1-(2-methylbenzyl)piperidin-4-yl]-3-oxo-2,3-dihydro-1H-isoindole-4-carboxamide; 2-[1-(2-bromobenzyl)piperidin-4-yl]-3-oxo-2,3-dihydro-1H-isoindole-4-carboxamide; 2-[1-(3-bromobenzyl)piperidin-4-yl]-3-oxo-2,3-dihydro-1H-isoindole-4-carboxamide; 2-[1-(4-bromobenzyl)piperidin-4-yl]-3-oxo-2,3-dihydro-1H-isoindole-4-carboxamide; and 3-oxo-2-{1-[3-(trifluoromethyl)benzyl]piperidin-4-yl}-2,3-dihydro-1H-isoindole-4-carboxamide.

U.S. Pat. No. 8,778,966 to Vialard et al., discloses substituted quinolinone derivatives as PARP inhibitors.

U.S. Pat. No. 8,697,736 to Penning et al., discloses 1H-benzimidazole-4-carboxamides as PARP inhibitors.

U.S. Pat. No. 8,669,249 to Brown et al., discloses PARP inhibitors including: 2-methyl-6-((4-phenylpiperidin-1-yl)methyl)-2H-benzo[b][1,4]oxazin-3(4H)-one; 2-methyl-6-((4-phenylpiperazin-1-yl)methyl)-2H-benzo[b][1,4]oxazin-3(4H)-one; 6-((4-(4-fluorophenyl)-5,6-dihydropyridin-1(2H)-yl)methyl)-2-methyl-2H-benzo[b][1,4]oxazin-3(4H)-one; 6-((4-(4-chlorophenyl)-5,6-dihydropyridin-1(2H)-yl)methyl)-2-methyl-2H-benzo[b][1,4]oxazin-3(4H)-one; 2-methyl-6-((4-p-tolylpiperidin-1-yl)methyl)-2H-benzo[b][1,4]oxazin-3(4H)-one; 6-((4-(4-fluorophenyl)piperidin-1-yl)methyl)-2-methyl-2H-benzo[b][1,4]oxazin-3(4H)-one; 6-((4-(4-chlorophenyl)piperidin-1-yl)methyl)-2-methyl-2H-benzo[b][1,4]oxazin-3(4H)-one; 2-methyl-6-((4-(3-phenyl-1,2,4-thiadiazol-5-yl)piperazin-1-yl)methyl)-2H-benzo[b][1,4]oxazin-3(4H)-one; 6-((4-cyclopentylpiperazin-1-yl)methyl)-2-methyl-2H-benzo[b][1,4]oxazin-3-(4H)-one; 6-((4-(1-benzo[d]imidazol-2-yl)piperazin-1-yl)methyl)-2-methyl-2H-benzo[b][1,4]oxazin-3(4H)-one; (S)-2-methyl-6-((4-phenylpiperidin-1-yl)methyl)-2H-benzo[b][1,4]oxazin-3(4H)-one; (R)-2-methyl-6-((4-phenylpiperidin-1-yl)methyl)-2H-benzo[b][1,4]oxazin-3(4H)-one; 6-((4-(1H-benzo[d]imidazol-2-yl)piperidin-1-yl)methyl)-2-methyl-2H-benzo[b][1,4]oxazin-3(4H)-one; 2-methyl-6-((4-(4-nitrophenyl)piperazin-1-yl)methyl)-2H-benzo[b][1,4]oxazin-3(4H)-one; 4-(4((2-methyl-3-oxo-3,4-dihydro-2H-benzo[b][1,4]oxazin-6-yl)methyl)piperazin-1-yl)benzoic acid; 6-((4-cycloheptylpiperazin-1-yl)methyl)-2-methyl-2H-benzo[b][1,4]oxazin-3-(4H)-one; 1,3,7-trimethyl-8-(4((2-methyl-3-oxo-3,4-dihydro-2H-benzo[b][1,4]oxazin-6-yl)methyl)piperazin-1-yl)-1H-purine-2,6(3H,7H)-dione; 6-((4-(4-aminophenyl)piperazin-1-yl)methyl)-2-methyl-2H-benzo[b][1,4]oxazin-3(4H)-one; 6-((4-(6-fluorobenzo[d]isoxazol-3-yl)piperidin-1-yl)methyl)-2-methyl-2H-benzo[b][1,4]oxazin-3(4H)-one; 6-((4-(2-hydroxyphenyl)piperazin-1-yl)methyl)-2-methyl-2H-benzo[b][1,4]oxazin-3(4H)-one; 2-methyl-6-((4-phenyl-5,6-dihydropyridin-1(2H)-yl)methyl)-2H-benzo[b][1,4]oxazin-3(4H)-one; 2-methyl-6-((4-phenyl-5,6-dihydropyridin-1(2H)-yl)methyl)-2H-benzo[b][1,4]oxazin-3(4H)-one; 6-((4-(2-methoxyphenyl)piperazin-1-yl)methyl)-2-methyl-2H-benzo[b][1,4]ox-azin-3(4H)-one; 6-((4-(5-chloropyridin-2-yl)piperazin-1-yl)methyl)-2-methyl-2H-benzo[b][1,4]oxazin-3(4H)-one; (S)-6-((4-(4-chlorophenyl)-5,6-dihydropyridin-1(2H)-yl)methyl)-2-methyl-2H-benzo[b][1,4]oxazin-3(4H)-one; (R)-6-((4-(4-chlorophenyl)-5,6-dihydropyridin-1(2H)-yl)methyl)-2-methyl-2H-benzo[b][1,4]oxazin-3(4H)-one; and 2-methyl-6-((3-oxo-4-phenylpiperazin-1-yl)methyl)-2H-benzo[b][1,4]oxazin-3(4H)-one.

U.S. Pat. No. 8,663,884 to Kennis et al., discloses quinazolinedione derivatives as PARP inhibitors.

U.S. Pat. No. 8,623,872 to Guillemont et al., discloses quinazolinone derivatives as PARP inhibitors.

U.S. Pat. No. 8,546,368 to Penning et al., discloses pyrazoquinolones as PARP inhibitors, including 7,9-dimethyl-1,2,3,4,6,7-hexahydro-5H-pyrazolo[3,4-h]-1,6-naphthyridin-5-one.

U.S. Pat. No. 8,541,417 to Brown et al., discloses PARP inhibitors, including: 3-(hydroxymethyl)pyrido[2,3-e]pyrrolo[1,2-c]pyrimidin-6(5H)-one; N-ethyl-4-(4((6-oxo-5,6-dihydropyrido[2,3-e]pyrrolo[1,2-c]pyrimidin-3-yl)methyl)piperazin-1-yl)benzamide; N-methyl-4-(4((6-oxo-5,6-dihydropyrido[2,3-e]pyrrolo[1,2-c]pyrimidin-3-yl)methyl)piperazin-1-yl)benzamide; N-cyclopropyl-4-(4((6-oxo-5,6-dihydropyrido[2,3-e]pyrrolo[1,2-c]pyrimidin-3-yl)methyl)piperazin-1-yl)benzamide; 3-chloro-N-methyl-4-(4-((6-oxo-5,6-dihydropyrido[2,3-e]pyrrolo[1,2-c]pyrimidin-3-yl)methyl)piperazin-1-yl)benzamide; 3-chloro-N-ethyl-4-(4-((6-oxo-5,6-dihydropyrido[2,3-e]pyrrolo[1,2-c]pyrimidin-3-yl)methyl)piperazin-1-yl)benzamide; N,3-dimethyl-4-(4-((6-oxo-5,6-dihydropyrido[2,3-e]pyrrolo[1,2-c]pyrimidin-3-yl)methyl)piperazin-1-yl)benzamide; N-ethyl-3-methyl-4-(4-((6-oxo-5,6-dihydropyrido[2,3-e]pyrrolo[1,2-c]pyrimidin-3-yl)methyl)piperazin-1-yl)benzamide; 3-fluoro-N-methyl-4-(4-((6-oxo-5,6-dihydropyrido[2,3-e]pyrrolo[1,2-c]pyrimidin-3-yl)methyl)piperazin-1-yl)benzamide; N-ethyl-3-fluoro-4-(4-((6-oxo-5,6-dihydropyrido[2,3-e]pyrrolo[1,2-c]pyrimidin-3-yl)methyl)piperazin-1-yl)benzamide; N-isopropyl-4-(4-((6-oxo-5,6-dihydropyrido[2,3-e]pyrrolo[1,2-c]pyrimidin-3-yl)methyl)piperazin-1-yl)benzamide; N-isopropyl-3-methyl-4-(4-((6-oxo-5,6-dihydropyrido[2,3-e]pyrrolo[1,2-c]pyrimidin-3-yl)methyl)piperazin-1-yl)benzamide; 3-fluoro-N-isopropyl-4-(4-((6-oxo-5,6-dihydropyrido[2,3-e]pyrrolo[1,2-c]pyrimidin-3-yl)methyl)piperazin-1-yl)benzamide; 3-chloro-N-isopropyl-4-(4-((6-oxo-5,6-dihydropyrido[2,3-e]pyrrolo[1,2-c]pyrimidin-3-yl)methyl)piperazin-1-yl)benzamide; 3-bromo-N-isopropyl-4-(4-((6-oxo-5,6-dihydropyrido[2,3-e]pyrrolo[1,2-c]pyrimidin-3-yl)methyl)piperazin-1-yl)benzamide; N-isopropyl-3-methoxy-4-(4-((6-oxo-5,6-dihydropyrido[2,3-e]pyrrolo[1,2-c]pyrimidin-3-yl)methyl)piperazin-1-yl)benzamide; N-isopropyl-3-(methylamino)-4-(4-((6-oxo-5,6-dihydropyrido[2,3-e]pyrrolo[1,2-c]pyrimidin-3-yl)methyl)piperazin-1-yl)benzamide; 3-ethyl-N-isopropyl-4-(4-((6-oxo-5,6-dihydropyrido[2,3-e]pyrrolo[1,2-c]pyrimidin-3-yl)methyl)piperazin-1-yl)benzamide; N-isopropyl-3-(methylthio)-4-(4-((6-oxo-5,6-dihydropyrido[2,3-e]pyrrolo[1,2-c]pyrimidin-3-yl)methyl)piperazin-1-yl)benzamide; 3-bromo-N-methyl-4-(4-((6-oxo-5,6-dihydropyrido[2,3-e]pyrrolo[1,2-c]pyrimidin-3-yl)methyl)piperazin-1-yl)benzamide; 3-methoxy-N-methyl-4-(4-((6-oxo-5,6-dihydropyrido[2,3-e]pyrrolo[1,2-c]pyrimidin-3-yl)methyl)piperazin-1-yl)benzamide; and N-methyl-3-(methylamino)-4-(4-((6-oxo-5,6-dihydropyrido[2,3-e]pyrrolo[1,2-c]pyrimidin-3-yl)methyl)piperazin-1-yl)benzamide.

U.S. Pat. No. 8,541,403 to Chu et al., discloses dihydropyridophthalazinone derivatives as PARP inhibitors, including: 8,9-diphenyl-8,9-dihydro-2H-pyrido[4,3,2-de]phtalazin-3(7H)-one; 8,9bis(4-methylamino)methyl)phenyl-8,9-dihydro-2H-pyrido[4,3,2-de]phtalazin-3(7H)-one; 8,9-di(pyridin-4-yl)-8,9-dihydro-2H-pyrido[4,3,2-de]phthalazin-3(7H)-one; 8,9-di(pyridin-3-yl)-8,9-dihydro-2H-pyrido[4,3,2-de]phthalazin-3(7H)-one; 8,9-di(pyridin-2-yl)-8,9-dihydro-2H-pyrido[4,3,2-de]phthalazin-3(7H)-one; 9-isopropyl-8-phenyl-8,9-dihydro-2H-pyrido[4,3,2-de]phthalazin-3(7H)-one; 9-(4-((methylamino)methyl)phenyl)-8-phenyl-8,9-dihydro-2H-pyrido[4,3,2-de]phthalazin-3(7H)-one; 9-(4-((dimethylamino)methyl)phenyl)-8-phenyl-8,9-dihydro-2H-pyrido[4,3,2-de]phthalazin-3(7H)-one; 9-(3-((methylamino)methyl)phenyl)-8-phenyl-8,9-dihydro-2H-pyrido[4,3,2-de]phthalazin-3(7H)-one; 8-(4((methylamino)methyl)phenyl)-9-phenyl-8,9-dihydro-2Hpyrido[4,3,2-de]phthalazin-3(7H)-one, 8,9-bi(3-((methylamino)methyl)phenyl)-8,9-dihydro)-2H-pyrido[4,3,2-de]phthalazin-3(7H)-one; 9-4-(hydroxymethyl)phenyl)-8-phenyl-8,9-dihydro-2H-pyrido[4,3,2-de]phthalazin-3(7H)-one; 9-(3-(4-isobutyrylpiperazine-1-carbonyl)phenyl)-8-phenyl-8,9-dihydro-2H-pyrido[4,3,2-de]phthalazin-3(7H)-one; 8,9-bis(3-(4-(isobutyrylpiperazine-1-carbonyl)phenyl)-8,9-dihydro-2H-pyrido[4,3,2-de]pthalazin-3(7H)-one; 9-(piperidin-3-yl)-8-(pyridin-3-yl)-8,9-dihydro-2H-pyrido[4,3,2-de]phthalazin-3(7H)-one; 9-(piperidin-4-yl)-8-(pyridin-4-yl)-8,9-dihydro-2H-pyrido[4,3,2-de]phthal-azin-3(7H)-one; 8,9-bis(4-((dimethylamino)methyl)phenyl)-8,9-dihydro-2H-pyrido[4,3,2-de]phthalazin-3(7H)-one; 9-(4-(4-(cyclopropanecarbonyl)piperazine-1-carbonyl)phenyl)-8(4-((methylamino)methyl)phenyl)-8,9-dihydro-2H-pyrido[4,3,2-de]phthalazin-3(7H)-one; and 9-(4-(4-(cyclopropanecarbonyl)piperazine-1-carbonyl)phenyl)-8-(4-((dimethylamino)methyl)phenyl)-8,9-dihydro-2H-pyrido[4,3,2-de]phthalazin-3(7H)-one.

U.S. Pat. No. 8,513,433 to Panicker et al., discloses inhibitors of PARP, including benzyl 2-(4-carbamoyl-1H-benzo[d]imidazol-2-yl)indoline-1-carboxylate; 2-(indolin-2-yl)-1H-benzo[d]imidazole-4-carboxamide; tert-butyl 2-(4-carbamoyl-1H-benzo[d]imidazol-2-yl)-3,4-dihydroquinoline-1(2H)-carboxylate; 2-(1,2,3,4-tetrahydroquinolin-2-yl)-1H-benzo[d]imidazole-4-carboxamide; benzyl 1-(4-carbamoyl-1H-benzo[d]imidazol-2-yl)isoindoline-2-carboxylate; 2-(isoindolin-1-yl)-1H-benzo[d]imidazole-4-carboxamide; benzyl 1-(4-carbamoyl-1H-benzo[d]imidazol-2-yl)-3,4-dihydroisoquinoline-2(1H)-carboxylate; 2-(1,2,3,4-tetrahydroisoquinolin-1-yl)-1H-benzo[d]imidazole-4-carboxamide; benzyl 3-(4-carbamoyl-1H-benzo[d]imidazol-2-yl)-3,4-dihydroisoquinoline-2(1H)-carboxylate; 2-(1,2,3,4-tetrahydroisoquinolin-3-yl)-1H-benzo[d]imidazole-4-carboxamide; benzyl 3-(4-carbamoyl-1H-benzo[d]imidazol-2-yl)-3-methyl-3,4-dihydroisoquinoline-2(1H)-carboxylate; 2-(3-methyl-1,2,3,4-tetrahydroisoquinolin-3-yl)-1H-benzo[d]imidazole-4-carboxamide; tert-butyl 74(tert-butoxycarbonyl)amino)-3-(4-carbamoyl-1H-benzo[d]imidazol-2-yl)-3,4-dihydroisoquinoline-2(1H)-carboxylate; tert-butyl 7-amino-3-(4-carbamoyl-1H-benzo[d]imidazol-2-yl)-3,4-dihydroisoquinoline-2(1H)-carboxylate; tert-butyl (3-(4-carbamoyl-1H-benzo[d]imidazol-2-yl)-1,2,3,4-tetrahydroisoquinolin-7-yl)carbamate; and 2-(7-amino-1,2,3,4-tetrahydroisoquinolin-3-yl)-1H-benzo[d]imidazole-4-carboxamide.

U.S. Pat. No. 8,470,825 to Xu et al., discloses substituted diazabenzo[de]anthracen-3-one compounds as PARP inhibitors.

U.S. Pat. No. 8,420,650 to Wang et al., discloses dihydropyridophthalazinone inhibitors of PARP, including: 8,9-di(pyridin-4-yl)-8,9-dihydro-2H-pyrido[4,3,2-de]phthalazin-3(7H)-one; 8,9-di(pyridin-3-yl)-8,9-dihydro-2H-pyrido[4,3,2-de]phthalazin-3(7H)-one; 8,9-di(pyridin-2-yl)-8,9-dihydro-2H-pyrido[4,3,2-de]phthalazin-3(7H)-one; 5-fluoro-9-(1-methyl-1H-imidazol-2-yl)-8-phenyl-8,9-dihydro-2H-pyrido[4,3,2-de]phthalazin-3(7H)-one; 5-fluoro-8-(4-fluorophenyl)-9-(1-methyl-1H-imidazol-2-yl)-8,9-dihydro-2H-pyrido[4,3,2-de]phthalazin-3(7H)-one; 8-(4-((dimethylamino)methyl)phenyl)-9(1-methyl-1H-imidazol-2-yl)-8,9-dihydro-2H-pyrido[4,3,2-de]phthalazin-3(7H)-one; 9-(1-isopropyl-1H-imidazol-5-yl)-8-phenyl-8,9-dihydro-2H-pyrido[4,3,2-de]phthalazin-3(7H)-one; 9-(4-methyl-1H-imidazol-2-yl)-8-phenyl-8,9-dihydro-2H-pyrido[4,3,2-de]phthalazin-3(7H)-one; 8-phenyl-9-(thiazol-5-yl)-8,9-dihydro-2H-pyrido[4,3,2-de]phthalazin-3(7H)-one; 9-(furan-3-yl)-8-phenyl-8,9-dihydro-2H-pyrido[4,3,2-de]phthalazin-3(7H)-one; 9-(1-methyl-1H-imidazol-2-yl)-8-phenyl-8,9-dihydro-2H-pyrido[4,3,2-de]phthalazin-3(7H)-one; 8,9-bis(1-methyl-1H-imidazol-2-yl)-8,9-dihydro-2H-pyrido[4,3,2-de]phthalazin-3(7H)-one; 9-(1H-imidazol-2-yl)-8-phenyl-8,9-dihydro-2H-pyrido[4,3,2-de]phthalazin-3(7H)-one; 9-(1-ethyl-1H-imidazol-2-yl)-8-phenyl-8,9-dihydro-2H-pyrido[4,3,2-de]phthalazin-3(7H)-one; 8-phenyl-9-(1-propyl-1H-imidazol-2-yl)-8,9-dihydro-2H-pyrido[4,3,2-de]phthalazin-3(7H)-one; 9-(1-methyl-1H-imidazol-5-yl)-8-phenyl-8,9-dihydro-2H-pyrido[4,3,2-de]phthalazin-3(7H)-one; 8-(4-fluorophenyl)-9-(1-methyl-1H-imidazol-2-yl)-8,9-dihydro-2H-pyrido[4,3,2-de]phthalazin-3(7H)-one; 9-(1-methyl-1H-1,2,4-triazol-5-yl)-8-phenyl-8,9-dihydro-2H-pyrido[4,3,2-de]phthalazin-3(7H)-one; and 8-(4-((dimethylamino)methyl)phenyl)-9-(1-methyl-1H-1,2,4-triazol-5-yl)-8,9-dihydro-2H-pyrido[4,3,2-de]phthalazin-3(7H)-one.

U.S. Pat. No. 8,362,030 to Ingenito et al., discloses tricyclic PARP inhibitors, including: N-methyl[4-(6-oxo-3,4,5,6-tetrahydro-2H-azepino[5,4,3-cd]indazol-2-yl)phenyl]methanaminium trifluoroacetate; N,N-dimethyl[4-(6-oxo-3,4,5,6-tetrahydro-2H-azepino[5,4,3-cd]indazol-2-yl)phenyl]methanaminium trifluoroacetate; N²,N²-dimethyl-N-[4-(1-oxo-1,2,3,4-tetrahydroazepino[3,4,5-hi]indolizin-5-yl)phenyl]glycinamide; 3-[4-(8-fluoro-6-oxo-3,4,5,6-tetrahydro-2H-azepino[5,4,3-cd]indazol-2-yl)phenyl]piperidinium trifluoroacetate; 8-fluoro-2-{4-[(3R)-piperidin-3-yl]phenyl}-2,3,4,5-tetrahydro-6H-azepino[5,4,3-cd]indazol-6-one; 8-fluoro-2-{4-(3S)-piperidin-3-yl]phenyl}-2,3,4,5-tetrahydro-6H-azepino[5,4,3-cd]indazol-6-one; 2-[4-(6-oxo-3,4,5,6-tetrahydro-2H-azepino[5,4,3-cd]indazol-2-yl)benzyl]-2,7-diazoniaspiro[4.5]decane bis(trifluoroacetate); [4-(8-fluoro-6-oxo-3,4,5,6-tetrahydro-2H-azepino[5,4,3-cd]indazol-2-yl)phenyl]-N,N-dimethylmethanaminium trifluoroacetate; 5-phenyl-3,4-dihydroazepino[3,4,5-hi]indolizin-1(2H)-one; ethyl 4-(1-oxo-1,2,3,4-tetrahydroazepino[3,4,5-hi]indolizin-5-yl)benzoate; 5-(4-nitrophenyl)-3,4-dihydroazepino[3,4,5-hi]indolizin-1(2H)-one; 5-[4-(hydroxymethyl)phenyl]-3,4-dihydroazepino[3,4,5-hi]indolizin-1(2H)-one; N-[4-(1-oxo-1,2,3,4-tetrahydroazepino[3,4,5-hi]indolizin-5-yl)phenyl]nicotinamide; N-[4-(1-oxo-1,2,3,4-tetrahydroazepino[3,4,5-hi]indolizin-5-yl)phenyl]pyridine-2-carboxamide; and N-[4-(1-oxo-1,2,3,4-tetrahydroazepino[3,4,5-hi]indolizin-5-yl)phenyl]-2-pyrrolidin-1-ylacetamide.

U.S. Pat. No. 8,354,413 to Jones et al., discloses quinolin-4-one and 4-oxodihydrocinnoline derivatives as PARP inhibitors, including: 1-[3-(8-aza-1-azoniaspiro[4.5]dec-8-ylcarbonyl)-4-fluorobenzyl]-4-oxo-1,4-dihydroquinolinium bis(trifluoroacetate); 1-[4-fluoro-3-({4-[2-(4-fluorobenzyl)prolyl]piperazin-1-yl}carbonyl)benzyl]quinolin-4(1H)-one; 1-[3-(8-aza-1-azoniaspiro[4.5]dec-8-ylcarbonyl)-4-fluorobenzyl]-4-oxo-1,4-dihydrocinnolin-1-ium bis(trifluoroacetate); 1-[3-(1,4-diazepan-1-ylcarbonyl)-4-fluorobenzyl]quinolin-4(1H)-one; 1-{4-fluoro-3-[(4-propionylpiperazin-1-yl)carbonyl]benzyl}quinolin-4(1H)-one; 1-(4-fluoro-3-{[3-(trifluoromethyl)-5,6-dihydro[1,2,4]triazolo[4,3-a]pyrazin-7(8H)-yl]carbonyl}benzyl)quinolin-4(1H)-one; 1-[3-(1,8-diazaspiro[4.5]dec-8-ylcarbonyl)-4-fluorobenzyl]quinolin-4(1H)-one; 1-[4-fluoro-3-(piperazin-1-ylcarbonyl)benzyl]quinolin-4(1H)-one; 1-[3-(2,6-diazaspiro[3.5]non-2-ylcarbonyl)-4-fluorobenzyl]quinolin-4(1H)-one; 1-[3-(2,5-diazabicyclo[2.2.2]oct-2-ylcarbonyl)-4-fluorobenzyl]quinolin-4(1H)-one; 1-(4-fluoro-3-{[4-(2-methylprolyl)piperazin-1-yl]carbonyl}benzyl)quinolin-4(1H)-one; 1-(4-fluoro-3-{[4-(3,3,3-trifluoro-N,N-dimethylalanyl)piperazin-1-yl]carbonyl}benzyl)quinolin-4(1H)-one; (2R)-2-[(4-{2-fluoro-5-[(4-oxoquinolin-1(4H)-yl)methyl]benzoyl}piperazin-1-yl)carbonyl]-2-methylazetidinium trifluoroacetate; 1-{4-fluoro-3-[(4-propionylpiperazin-1-yl)carbonyl]benzyl}-4-oxo-1,4-dihydrocinnolin-1-ium trifluoroacetate; 1-{3-[(3-ethyl-5,6-dihydro[1,2,4]triazolo[4,3-a]pyrazin-2-ium-7(8H)-yl)carbonyl]-4-fluorobenzyl}-4-oxo-1,4-dihydrocinnolin-1-ium bis(trifluoroacetate); 1-(4-fluoro-3-{[3-(trifluoromethyl)-5,6-dihydro[1,2,4]triazolo[4,3-a]pyrazin-7(8H)-yl]carbonyl}benzyl)-4-oxo-1,4-dihydrocinnolin-1-ium trifluoroacetate; and 8-fluoro-1-(4-fluoro-3-{[3-(trifluoromethyl)-5,6-dihydro[1,2,4]triazolo[4,3-a]pyrazin-7(8H)-yl]carbonyl}benzyl)-4-oxo-1,4-dihydrocinnolin-1-ium trifluoroacetate.

U.S. Pat. No. 8,268,827 to Branca et al., discloses pyridazinone derivatives as PARP inhibitors, including: 6-{4-fluoro-3-[(3-oxo-4-phenylpiperazin-1-yl)carbonyl]benzyl}-4,5-dimethyl-3-oxo-2,3-dihydropyridazin-1-ium trifluoroacetate; 6-{3-[(4-cyclohexyl-3-oxopiperazin-1-yl)carbonyl]-4-fluorobenzyl}-4,5-dimethyl-3-oxo-2,3-dihydropyridazin-1-ium trifluoroacetate; 6-{3-[(4-cyclopentyl-3-oxopiperazin-1-yl)carbonyl]-4-fluorobenzyl}-4,5-dimethylpyridazin-3(2H)-one; 6-{4-fluoro-3-[(3-oxo-4-phenylpiperazin-1-yl)carbonyl]benzyl}-4,5-dimethylpyridazin-3(2H)-one hydrochloride; 4-ethyl-6-{4-fluoro-3-[(3-oxo-4-phenylpiperazin-1-yl)carbonyl]benzyl}pyridazin-3(2H)-one trifluoroacetate; 6-{3-[(4-cyclohexyl-3-oxopiperazin-1-yl)carbonyl]-4-fluorobenzyl}-4-ethylpyridazin-3(2H)-one trifluoroacetate; 3-{4-fluoro-3-[(4-methyl-3-oxopiperazin-1-yl)carbonyl]benzyl}-4,5-dimethyl-6-oxo-1,6-dihydropyridazin-1-ium trifluoroacetate; 3-(4-fluoro-3-{[4-(4-fluorobenzyl)-3-oxopiperazin-1-yl]carbonyl}benzyl)-4,5-dimethyl-6-oxo-1,6-dihydropyridazin-1-ium trifluoroacetate; 6-(3-{[4-(2-chlorophenyl)-3-oxopiperazin-1-yl]carbonyl}-4-fluorobenzyl)-4,5-dimethyl-3-oxo-2,3-dihydropyridazin-1-ium trifluoroacetate; 6-(3-{[4-(3-chloro-4-fluorophenyl)-3-oxopiperazin-1-yl]carbonyl}-4-fluoro-benzyl)-4,5-dimethyl-3-oxo-2,3-dihydropyridazin-1-ium trifluoroacetate; 6-(3-{[4-(3,4-difluorophenyl)-3-oxopiperazin-1-yl]carbonyl}-4-fluorobenzyl)-4,5-dimethyl-3-oxo-2,3-dihydropyridazin-1-ium trifluoroacetate; and 6-(3-{[4-(3,5-difluorophenyl)-3-oxopiperazin-1-yl]carbonyl}-4-fluorobenzyl)-4,5-dimethyl-3-oxo-2,3-dihydropyridazin-1-ium trifluoroacetate.

U.S. Pat. No. 8,217,070 to Zhu et al., discloses 2-substituted-1H-benzimidazole-4-carboxamides as PARP inhibitors, including: 2-(1-aminocyclopropyl)-1H-benzimidazole-4-carboxamide; 2-[1-(isopropylamino)cyclopropyl]-1H-benzimidazole-4-carboxamide; 2-[1-(cyclobutylamino)cyclopropyl]-1H-benzimidazole-4-carboxamide; 2-{1-[(3,5-dimethylbenzyl)amino]cyclopropyl}-1H-benzimidazole-4-carboxamide; 2-{1-[(pyridin-4-ylmethyl)amino]cyclopropyl}-1H-benzimidazole-4-carboxamide; 2-[1-(dipropylamino)cyclopropyl]-1H-benzimidazole-4-carboxamide; 2-{1-[bis(cyclopropylmethy)amino]cyclopropyl}-1H-benzimidazole-4-carboxamide; 2-(1-aminocyclobutyl)-1H-benzimidazole-4-carboxamide; 2-(1-(propylamino)cyclobutyl]-1H-benzimidazole-4-carboxamide; 2-{1-[(cyclopropylmethyl)amino]cyclobutyl}-1H-benzimidazole-4-carboxamide; 2-[1-(isopropylamino)cyclobutyl]-1H-benzimidazole-4-carboxamide; 2-[1-(dipropylamino)cyclobutyl]-1H-benzimidazole-4-carboxamide; 2-(1-(dibutylamino)cyclobutyl]-1H-benzimidazole-4-carboxamide; 2-(1-aminocyclohexyl)-1H-benzimidazole-4-carboxamide; 9H-fluoren-9-ylmethyl 4-[4-(aminocarbonyl)-1H-benzimidazol-2-yl]piperidin-4-ylcarbamate; benzyl 4-amino-4-[4-(aminocarbonyl)-1H-benzimidazol-2-yl]piperidine-1-carboxylate; [2-(4-amino-piperidin-4-yl]-1H-benzoimidazole-4-carboxylic acid amide; 2-(2-amino-1,2,3,4-tetrahydronaphthalen-2-yl)-1H-benzimidazole-4-carboxamide; and 2-(2-amino-2,3-dihydro-1H-inden-2-yl)-1H-benzimidazole-4-carboxamide.

U.S. Pat. No. 8,188,103 to Van der Aa et al., discloses substituted 2-alkyl quinazolinone derivatives as PARP inhibitors.

U.S. Pat. No. 8,173,682 to Weintraub et al., discloses 2,3,5-substituted pyridone derivatives as PARP inhibitors, including: 5-(5-ethyl-2-methyl-6-oxo-1,6-dihydro-pyridin-3-yl)-thiophene-2-sulfonic acid [3-(3-hydroxy-pyrrolidin-1-yl)-propyl]-amide hydrochloride; 5-(5-ethyl-2-methyl-6-oxo-1,6-dihydropyridin-3-yl)thiophene-2-sulfonic acid [2-(1-methylpyrrolidin-2-yl)ethyl]amide hydrochloride; 5-(5-ethyl-2-methyl-6-oxo-1,6-dihydro-pyridin-3-yl)-thiophene-2-sulfonic acid [3-(3,3-difluoro-pyrrolidin-1-yl)-propyl]-amide hydrochloride; 5-[5-ethyl-2-methyl-6-oxo-1,6-dihydropyridin-3-yl]thiophene-2-sulfonic acid [3-(2-oxopyrrolidin-1-yl)propyl]amide; 5-[5-ethyl-2-methyl-6-oxo-1,6-dihydropyridin-3-yl]thiophene-2-sulfonic acid methyl (1-methylpyrrolidin-3-yl)amide hydrochloride; 3-ethyl-5-[5-(3-hydroxypyrrolidine-1-sulfonyl)thiophen-2-yl]-6-methyl-1H-pyridin-2-one; 5-(5-ethyl-2-methyl-6-oxo-1,6-dihydropyridin-3-yl)thiophene-2-sulfonic acid (2-pyrrolidin-1-yl)ethylamide hydrochloride; 5-(5-ethyl-2-methyl-6-oxo-1,6-dihydropyridin-3-yl)-thiophene-2-sulfonic acid (1-ethyl-pyrrolidin-2-ylmethyl)amide hydrochloride; 3-ethyl-6-methyl-5-[5-((S)-2-phenylaminomethylpyrrolidine-1-sulfonyl)-thiophen-2-yl]-1H-pyridin-2-one hydrochloride; 5-(5-ethyl-2-methyl-6-oxo-1,6-dihydro-pyridin-3-yl)-thiophene-2-sulfonic acid [3-(2R-hydroxymethyl-pyrrolidin-1-yl)-propyl]-amide hydrochloride; 5-(5-ethyl-2-methyl-6-oxo-1,6-dihydro-pyridin-3-yl)-thiophene-2-sulfonic acid [2-(2R-hydroxymethyl-pyrrolidin-1-yl)-ethyl]-amide hydrochloride; 5-(5-ethyl-2-methyl-6-oxo-1,6-dihydro-pyridin-3-yl)-thiophene-2-sulfonic acid [2-(3,3-difluoro-pyrrolidin-1-yl)-ethyl]-amide; and 1-{2-[5-(5-ethyl-2-methyl-6-oxo-1,6-dihydro-pyridin-3-yl)-thiophene-2-sul-fonylamino]-ethyl}-pyrrolidine-2-carboxylic acid.

U.S. Pat. No. 8,129,382 to Kalish et al., discloses PARP inhibitors of Formula (P-III)

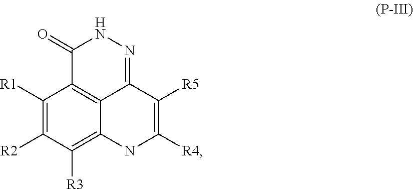

wherein:

(1) R1 is H, halogen, alkoxy, or lower alkyl;

(2) R2 is H, halogen, alkoxy, or lower alkyl;

(3) R3 is independently H, amino, hydroxy, —N—N, halogen-substituted amino, —O-alkyl, —O-aryl, or an optionally substituted alkyl, alkenyl, alkynyl, cycloalkyl, heterocycloalkyl, aryl, heteroaryl, —COR8, where R8 is H, —OH an optionally substituted alkyl, alkenyl, alkynyl, cycloalkyl, heterocycloalkyl, aryl or heteroaryl, or —OR6 or —NR6R7 where R6 and R7 are each independently hydrogen or an optionally substituted alkyl, alkenyl, alkynyl, cycloalkyl, heterocycloalkyl, aryl or heteroaryl;

(4) R4 is independently H, amino, hydroxy, —N—N, —CO—N—N, halogen-substituted amino, —O-alkyl, —O-aryl, or an optionally substituted alkyl, alkenyl, alkynyl, cycloalkyl, heterocycloalkyl, aryl, heteroaryl, —COR8, where R8 is H, —OH an optionally substituted alkyl, alkenyl, alkynyl, cycloalkyl, heterocycloalkyl, aryl or heteroaryl, or —OR6 or —NR6R7 where R6 and R7 are each independently hydrogen or an optionally substituted alkyl, alkenyl, alkynyl, cycloalkyl, heterocycloalkyl, aryl or heteroaryl; and

(5) R5 is independently H, amino, hydroxy, —N—N, —CO—N—N, halogen-substituted amino, —O-alkyl, —O-aryl, or an optionally substituted alkyl, alkenyl, alkynyl, cycloalkyl, heterocycloalkyl, aryl, heteroaryl, —COR8, where R8 is H, —OH an optionally substituted alkyl, alkenyl, alkynyl, cycloalkyl, heterocycloalkyl, aryl or heteroaryl, or —OR6 or —NR6R7 where R6 and R7 are each independently hydrogen or an optionally substituted alkyl, alkenyl, alkynyl, cycloalkyl, heterocycloalkyl, aryl or heteroaryl.

U.S. Pat. No. 8,088,760 to Chu et al., discloses benzoxazole carboxamide inhibitors of PARP, including: 2-(4-((methylamino)methyl)phenyl)benzo[d]oxazole-4-carboxamide; 2-(2-methylpyrrolidin-2-yl)benzo[d]oxazole-4-carboxamide; 2-(4-((methylamino)methyl)phenyl)benzo[d]oxazole-7-carboxamide; 2-(2-methylpyrrolidin-2-yl)benzo[d]oxazole-7-carboxamide; 2-(pyrrolidin-2-yl)benzo[d]oxazole-4-carboxamide; 2-(Pyrrolidin-2-yl)benzo[d]oxazole-7-carboxamide; 2-(7-azabicyclo[2.2.1]heptan-1-yl)benzo[d]oxazole-4-carboxamide; 2-(7-azabicyclo[2.2.1]heptan-1-yl)benzo[d]oxazole-7-carboxamide; 2-(2-methyl-7-azabicyclo[2.2.1]heptan-1-yl)benzo[d]oxazole-4-carboxamide; 2-(2-methyl-7-azabicyclo[2.2.1]heptan-1-yl)benzo[d]oxazole-7-carboxamide; 2-(2-azabicyclo[2.1.1]hexan-1-yl)benzo[d]oxazole-4-carboxamide; 2-(2-azabicyclo[2.1.1]hexan-1-yl)benzo[d]oxazole-7-carboxamide; 2-(6-azabicyclo[3.2.1]octan-5-yl)benzo[d]oxazole-4-carboxamide; 2-(6-azabicyclo[3.2.1]octan-5-yl)benzo[d]oxazole-7-carboxamide; 2-((1S,5R)-6-azabicyclo[3.2.1]octan-5-yl)benzo[d]oxazole-4-carboxamide; 2-((1S,5R)-6-azabicyclo[3.2.1]octan-5-yl)benzo[d]oxazole-7-carboxamide; 2-((1R,5S)-6-azabicyclo[3.2.1]octan-5-yl)benzo[d]oxazole-4-carboxamide; 2-((1R,5S)-6-azabicyclo[3.2.1]octan-5-yl)benzo[d]oxazole-7-carboxamide; 2-(2-benzyl-2-azabicyclo[2.2.2]octan-1-yl)benzo[d]oxazole-4-carboxamide; 2-(2-benzyl-2-azabicyclo[2.2.2]octan-1-yl)benzo[d]oxazole-7-carboxamide; 2-(2-azabicyclo[2.2.2]octan-1-yl)benzo[d]oxazole-4-carboxamide; 2-(2-azabicyclo[2.2.2]octan-1-yl)benzo[d]oxazole-7-carboxamide; 2-(4-azaspiro[2.4]heptan-5-yl)benzo[d]oxazole-4-carboxamide; 2-(4-azaspiro[2.4]heptan-5-yl)benzo[d]oxazole-7-carboxamide; 2-((1R,4S)-2-methyl-2-azabicyclo[2.2.1]heptan-1-yl)benzo[d]oxazole-4-carboxamide; and 2-((1R,4S)-2-methyl-2-azabicyclo[2.2.1]heptan-1-yl)benzo[d]oxazole-7-carboxamide.

U.S. Pat. No. 8,071,623 to Jones et al., discloses amide-substituted indazoles as PARP inhibitors, including: 2-(4-piperidin-3-ylphenyl)-2H-indazole-7-carboxamide; 2-{4-[(3R)-piperidin-3-yl]phenyl}-2H-indazole-7-carboxamide; 2-{4-[(3S)-piperidin-3-yl]phenyl}-2H-indazole-7-carboxamide; 5-fluoro-2-(4-piperidin-3-ylphenyl)-2H-indazole-7-carboxamide; 5-fluoro-2-{4-[(3S)-piperidin-3-yl]phenyl}-2H-indazole-7-carboxamide; 5-fluoro-2-{4-[(3R)-piperidin-3-yl]phenyl}-2H-indazole-7-carboxamide; 5-fluoro-2-(3-fluoro-4-piperidin-3-ylphenyl)-2H-indazole-7-carboxamide; 5-fluoro-2-{3-fluoro-4-[(3R)-piperidin-3-yl]phenyl}-2H-indazole-7-carboxamide; and 5-fluoro-2-{3-fluoro-4-[(3S)-piperidin-3-yl]phenyl}-2H-indazole-7-carboxamide.

U.S. Pat. No. 8,058,275 to Xu et al., discloses diazabenzo[de]anthracen-3-one compounds as PARP inhibitors.

U.S. Pat. No. 8,012,976 to Wang et al., discloses dihydropyridophthalazinone compounds as PARP inhibitprs, including 5-fluoro-8-(4-fluorophenyl)-9-(1-methyl-1H-1,2,4-triazol-5-yl)-8,9-dihydro-2H-pyrido[4,3,2-de]phthalazin-3(7H)-one.

U.S. Pat. No. 8,008,491 to Jiang et al., discloses substituted aza-indole derivatives as PARP inhibitors, including: 1-phenyl-2-(piperazin-1-yl)-1,3-dihydropyrrolo[2,3-b]pyridine-3-carboxald-ehyde, 1-phenyl-2-(piperazin-1-yl)-1H-pyrrolo[2,3-c]pyridine-3-carboxaldehyde, 2-[1,4]diazepan-1-yl-1-phenyl-1H-pyrrolo[2,3-b]pyridine-3-carbaldehyde trifluoroacetic acid salt, and 2-piperazin-1-yl-1-pyridin-3-yl-1H-pyrrolo[2,3-b]pyridine-3-carbaldehyde bis-trifluoroacetic acid salt.

U.S. Pat. No. 7,999,117 to Giranda et al., discloses 1H-benzimidazole-4-carboxamides as PARP inhibitors, including: 6-fluoro-2-[4-((S)-2-hydroxymethylpyrrolidin-1-ylmethyl)phenyl]-1H-benzimidazole-4-carboxamide; 6-fluoro-2-[4-(2-trifluoromethylpyrrolidin-1-ylmethyl)phenyl]-1H-benzimidazole-4-carboxamide; 6-fluoro-2-[4-((R)-2-hydroxymethylpyrrolidin-1-ylmethyl)phenyl]-1H-benzimidazole-4-carboxamide; 2-[4-((S)-2-hydroxymethylpyrrolidin-1-ylmethyl)phenyl]-1H-benzimidazole-4-carboxamide; 2-[4-(2-trifluoromethylpyrrolidin-1-ylmethyl)phenyl]-1H-benzimidazole-4-carboxamide; 2-[4-((R)-2-hydroxymethylpyrrolidin-1-ylmethyl)phenyl]-1H-benzimidazole-4-carboxamide; 6-chloro-2-[4-(2-trifluoromethylpyrrolidin-1-ylmethyl)phenyl]-1H-benzimidazole-4-carboxamide; 6-chloro-2-[4-((S)-2-hydroxymethylpyrrolidin-1-ylmethyl)phenyl]-1H-benzimidazole-4-carboxamide; 6-chloro-2-[4-((R)-2-hydroxymethylpyrrolidin-1-ylmethyl)phenyl]-1H-benzimidazole-4-carboxamide; 2-[2-fluoro-44(S)-2-hydroxymethylpyrrolidin-1-ylmethyl)phenyl]-1H-benzimidazole-4-carboxamide; 2-{4-[(3-aminopyrrolidin-1-yl)methyl]phenyl}-1H-benzimidazole-4-carboxamide; and 2-(4-{[3-(dimethylamino)pyrrolidin-1-yl]methyl}phenyl)-1H-benzimidazole-4-carboxamide.

U.S. Pat. No. 7,994,182 to Sumegi et al., discloses quinazolinone derivatives as PARP inhibitors of Formula (P-IV):

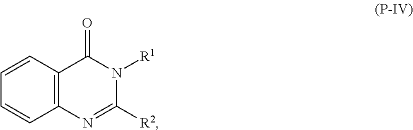

wherein:

(1) R1 is hydrogen or a moiety of Subformula (P-IV(a)):

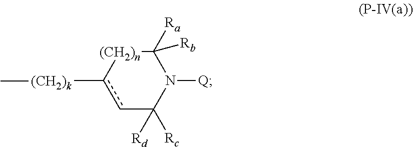

(2) k is 1, 2, 3, or 4;

(3) n is 0 or 1;

(4) Q is an oxyl group or hydrogen;

(5) Rₐ and R_{b} are independently hydrogen or C₁-C₆ alkyl;

(6) R_{b} and R_{d} are independently C₁-C₆ alkyl;

(7) the broken line in the six-membered ring is an optional valence bond (the bond is either a single or a double bond);

(8) R² is either:

(8a) if R¹ is other than hydrogen, hydrogen or C₁-C₆ alkyl;

(8b) if R1 is hydrogen, a group of Subformula (P-IV(b)), Subformula (P-IV(c)), or Subformula (P-IV(d)):

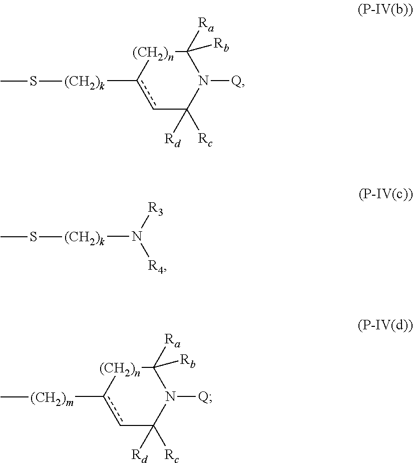

wherein:

(9) in Subformula (P-IV(b)), k, n, Rₐ, R_{b}, R_{c}, R_{d}, and the broken line are as defined above in (2), (3), (5), (6), and (7);

(10) in Subformula (P-IV(c)), k is 1, 2, or 3, and R³ and R⁴ are independently C₁-C₆ alkyl;

(11) or together with the attached nitrogen form a group of Subformula (P-IV(e)), wherein p is 0 or 1 and R_{a′}, R_{b′}, R_{c′}, and R_{d′}, are independently hydrogen or C₁-C₆ alkyl;

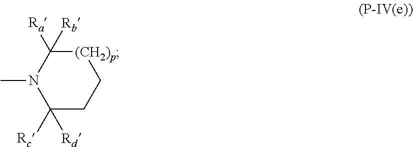

and

(11) in Subformula (P-IV(d), n, Rₐ, R_{b}, R_{c}, R_{d}, and the broken line are as defined above in (3), (5), (6), and (7).

U.S. Pat. No. 7,834,015 to Jones et al., discloses pyrrolo[1,2-a] pyrazin-1(2H)-one and pyrrolo[1,2-d][1,2,4]triazin-1(2H)-one derivatives as PARP inhibitors.

U.S. Pat. No. 7,825,129 to Pellicciari et al., discloses thieno[2,3-c]quinolones as PARP inhibitors, including compounds of Formula (P-V):

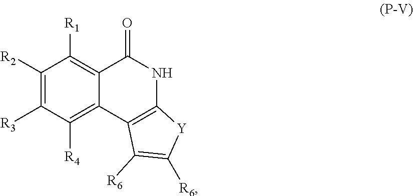

wherein:

(1) Y is selected from sulfur, nitrogen, and oxygen;

(2) R₁, R₂, R₃, R₄, R₅ and R₆ are the same or different, and each represent hydrogen, hydroxy, OR₇, COOR₇, carboxy, amino, NHR₇ or halogen, or R₅ and R₆ taken together form a fused non-aromatic 5- or 6-membered carbocylic ring; and

(3) R₇ is C₁-C₆ alkyl, C₂-C₆ alkenyl or C₃-C₇ cycloalkyl optionally substituted with one or more group selected from hydroxyl, C₁-C₄ alkoxy, carboxy, C₁-C₆ alkoxycarbonyl, amino, C₁-C₆ mono-alkylamino, C₁-C₆ di-alkylamino and halogen.

U.S. Pat. No. 7,820,668 to Xu et al., discloses diazabenzo[de]anthracen-3-one compounds as PARP inhibitors.

U.S. Pat. No. 7,732,491 to Sherman et al., discloses 4-iodo-3-nitrobenzamide as a PARP inhibitor.

U.S. Pat. No. 7,728,026 to Zhu et al., discloses 2-substituted 1H-benzimidazole-4-carboxamides as PARP inhibitors, including 2-(1-amino-1-methylethyl)-1H-benzimidazole-4-carboxamide; 2-[1-methyl-1-(propylamino)ethyl]-1H-benzimidazole-4-carboxamide; 2-[1-(butylamino)-1-methylethyl]-1H-benzimidazole-4-carboxamide; 2-{1-methyl-1-[(2-phenylethyl)amino]ethyl}-1H-benzimidazole-4-carboxamide; 2-[1-(isopropylamino)-1-methylethyl]-1H-benzimidazole-4-carboxamide; 2-{1-[(cyclopropylmethyl)amino]-1-methylethyl}-1H-benzimidazole-4-carboxamide; 2-[1-(cyclobutylamino)-1-methylethyl]-1H-benzimidazole-4-carboxamide; 2-[1-(cyclopentylamino)-1-methylethyl]-1H-benzimidazole-4-carboxamide; 2-(1-{[(cyclopentylamino)carbonyl]amino}-1-methylethyl)-1H-benzimidazole-4-carboxamide; 2-(1-{[(ethylamino)carbonyl]amino}-1-methylethyl)-1H-benzimidazole-4-carboxamide; 2-(1-{[(dimethylamino)sulfonyl]amino}-1-methylethyl)-1H-benzimidazole-4-carboxamide; 2-(3-amino-1-methylpropyl)-1H-benzimidazole-4-carboxamide; 2-[3-(cyclopentylamino)-1-methylpropyl]-1H-benzimidazole-4-carboxamide; 2-[3-(cyclohexylamino)-1-methylpropyl]-1H-benzimidazole-4-carboxamide; 2-(1-aminoethyl)-1H-benzimidazole-4-carboxamide; 2-[1-(propylamino)ethyl]-1H-benzimidazole-4-carboxamide; 2-[1-(butylamino)ethyl]-1H-benzimidazole-4-carboxamide; 2-{1-[(cyclopropylmethyl)amino]ethyl}-1H-benzimidazole-4-carboxamide; 2-[1-(isobutylamino)ethyl]-1H-benzimidazole-4-carboxamide; 2-[1-(isopropylamino)ethyl]-1H-benzimidazole-4-carboxamide; 2-[1-(cyclopentylamino)ethyl]-1H-benzimidazole-4-carboxamide; 2-[1-(cyclohexylamino)ethyl]-1H-benzimidazole-4-carboxamide; 2-{1-[(2-phenylethyl)amino]ethyl}-1H-benzimidazole-4-carboxamide; 2-[1-(dipropylamino)ethyl]-1H-benzimidazole-4-carboxamide; 2-{1-[butyl(pentyl)amino]ethyl}-1H-benzimidazole-4-carboxamide; 2-{1-[bis(cyclopropylmethyl)amino]ethyl}-1H-benzimidazole-4-carboxamide; 2-(1-{[(dimethylamino)sulfonyl]amino}ethyl)-1H-benzimidazole-4-carboxamide; 2-(1-aminopropyl)-1H-benzimidazole-4-carboxamide; 2-[(1R)-1-(dim ethylamino)-2-phenylethyl]-1H-benzimidazole-4-carboxamide; and 2-(1-amino-1-methylethyl)-5-chloro-1H-benzimidazole-7-carboxamide.

U.S. Pat. No. 7,595,406 to Penning et al., discloses substituted 1H-benzimidazole-4-carboxamides as PARP inhibitors, including 2-{4-[1-(cyclohexylmethylamino)ethyl]phenyl}-1H-benzimidazole-4-carboxamide; 2-[4-(1-cyclobutylaminoethyl)phenyl]-1H-benzimidazole-4-carboxamide; 2-[3-(2-cyclopropylaminoethyl)phenyl]-1H-benzimidazole-4-carboxamide; 2-(4-cyclopropylaminomethylphenyl)-1H-benzimidazole-4-carboxamide; 2-(4-cyclobutylaminomethylphenyl)-1H-benzimidazole-4-carboxamide; 2-(4-cyclopentylaminomethylphenyl)-1H-benzimidazole-4-carboxamide; 6-chloro-2-{4-[(1,2,3,4-tetrahydronaphthalen-1-ylamino)methyl]phenyl}-1H-benzimidazole-4-carboxamide; 2-(4-cyclopropylaminomethylphenyl)-6-fluoro-1H-benzimidazole-4-carboxamide; 2-(4-cyclobutylaminomethylphenyl)-6-fluoro-1H-benzimidazole-4-carboxamide; 2-(4-cyclopentylaminomethylphenyl)-6-fluoro-1H-benzimidazole-4-carboxamide; 2-[4-(2-cyclopropylaminoethyl)phenyl]-1H-benzimidazole-4-carboxamide; 2-[4-(2-cyclobutylaminoethyl)phenyl]-1H-benzimidazole-4-carboxamide; 2-(4-cyclopropylaminomethyl-2-fluorophenyl)-1H-benzimidazole-4-carboxamide; 2-[4-(1-cyclopropylaminoethyl)phenyl]-1H-benzimidazole-4-carboxamide; 2-(4-cyclobutylaminomethyl-2-fluorophenyl]-1H-benzimidazole-4-carboxamide; 2-(4-cyclohexylaminomethyl-2-fluorophenyl)-1H-benzimidazole-4-carboxamide; and 2-(4-cyclopentylaminomethyl-2-fluorophenyl)-1H-benzimidazole-4-carboxamide.

U.S. Pat. No. 7,550,603 to Zhu et al., discloses 1H-benzimidazole-4-carboxamides substituted with a quaternary carbon at the 2-position as PARP inhibitors, including 2-(2-methylpyrrolidin-2-yl)-1H-benzimidazole-4-carboxamide; 2-[(2R)-2-methylpyrrolidin-2-yl]-1H-benzimidazole-4-carboxamide; 2-[(2S)-2-methylpyrrolidin-2-yl]-1H-benzimidazole-4-carboxamide; 2-(1,2-dimethylpyrrolidin-2-yl)-1H-benzimidazole-4-carboxamide; 2-(1-ethyl-2-methylpyrrolidin-2-yl)-1H-benzimidazole-4-carboxamide; 2-(2-methyl-1-propylpyrrolidin-2-yl)-1H-benzimidazole-4-carboxamide; 2-(1-isopropyl-2-methylpyrrolidin-2-yl)-1H-benzimidazole-4-carboxamide; 2-(1-cyclobutyl-2-methylpyrrolidin-2-yl)-1H-benzimidazole-4-carboxamide; 2-(3-methylpyrrolidin-3-yl)-1H-benzimidazole-4-carboxamide; 2-[3-methyl-1-propylpyrrolidin-3-yl)-1H-benzimidazole-4-carboxamide; 2-(1-(cyclopropylmethyl)-3-methylpyrrolidin-3-yl]-1H-benzimidazole-4-carb-oxamide; 2-(1-isobutyl-3-methylpyrrolidin-3-yl)-1H-benzimidazole-4-carboxamide; 2-(1-isopropyl-3-methylpyrrolidin-3-yl)-1H-benzimidazole-4-carboxamide; 2-(1-cyclobutyl-3-methylpyrrolidin-3-yl)-1H-benzimidazole-4-carboxamide; 2-(1-cyclopentyl-3-methylpyrrolidin-3-yl)-1H-benzimidazole-4-carboxamide; 2-(1-cyclohexyl-3-methylpyrrolidin-3-yl)-1H-benzimidazole-4-carboxamide; 2-[3-methyl-1-tetrahydro-2H-pyran-4-ylpyrrolidin-3-yl]-1H-benzimidazole-4-carboxamide; 2-[3-methyl-1-(pyridin4-ylmethyl)pyrrolidin-3-yl]-1H-benzimidazole-4-carboxamide; 2-[3-methyl-1-(2-phenylethyl)pyrrolidin-3-yl]-1H-benzimidazole-4-carboxamide; 2-[3-methyl-1-(1-methyl-3-phenylpropyl)pyrrolidin-3-yl]-1H-benzimidazole-4-carboxamide; 2-(2-methylazetidin-2-yl)-1H-benzimidazole-4-carboxamide; 2-(1-isopropyl-2-methylazetidin-2-yl)-1H-benzimidazole-4-carboxamide; 2-(1-cyclobutyl-2-methylazetidin-2-yl)-1H-benzimidazole-4-carboxamide; 2-(1-cyclopentyl-2-methylazetidin-2-yl)-1H-benzimidazole-4-carboxamide; 2-(1-cyclohexyl-2-methylazetidin-2-yl)-1H-benzimidazole-4-carboxamide; 2-(3-methylazetidin-3-yl)-1H-benzimidazole-4-carboxamide; 2-[3-methyl-1-propylazetidin-3-yl)-1H-benzimidazole-4-carboxamide; 2-[1-(cyclopropylmethyl)-3-methylazetidin-3-yl]-1H-benzimidazole-4-carboxamide; 2-(1-isobutyl-3-methylazetidin-3-yl)-1H-benzimidazole-4-carboxamide; 2-(1-cyclobutyl-3-methylazetidin-3-yl)-1H-benzimidazole-4-carboxamide; 2-(1-cyclopentyl-3-methylazetidin-3-yl)-1H-benzimidazole-4-carboxamide; 2-(1-cyclohexyl-3-methylazetidin-3-yl)-1H-benzimidazole-4-carboxamide; 2-(3-methyl-1-tetrahydro-2H-pyran-4-ylazetidin-3-yl)-1H-benzimidazole-4-carboxamide; 2-{1-[(dimethylamino)sulfonyl]-3-methylazetidin-3-yl}-1H-benzimidazole-4-carboxamide; and 2-(2-methylpiperidin-2-yl)-1H-benzimidazole-4-carboxamide.

U.S. Pat. No. 7,405,300 to Jiang et al., discloses substituted indoles as PARP inhibitors, including 2-(piperazin-1-yl)-1-(3-nitrophenyl)-1H-indole-3-carboxaldehyde; 2-(piperazin-1-yl)-1-(4-methoxyphenyl)-1H-indole-3-carboxaldehyde; 2-(piperazin-1-yl)-1-(4-tert-butylphenyl)-1H-indole-3-carboxaldehyde; 2-(piperazin-1-yl)-1-(4-bromophenyl)-1H-indole-3-carboxaldehyde; 2-(piperazin-1-yl)-1-(4-chlorophenyl)-1H-indole-3-carboxaldehyde; 2-(piperazin-1-yl)-1-(3-chloro-4-fluorophenyl)-1H-indole-3-carboxaldehyde, 2-(piperazin-1-yl)-1-(3-methoxyphenyl)-1H-indole-3-carboxaldehyde; 2-(piperazin-1-yl)-1-(4-thiomethylphenyl)-1H-indole-3-carboxaldehyde; 2-(piperazin-1-yl)-1-(3-fluorophenyl)-1H-indole-3-carboxaldehyde; 2-(piperazin-1-yl)-1-(3-methylphenyl)-1H-indole-3-carboxaldehyde; 1-(4-tert-butylphenyl)-2-piperazin-1-yl-1H-indole-3-carboxaldehyde, 1-(4-tert-butylphenyl)-2-piperidin-1-yl-1H-indole-3-carboxaldehyde; 1-(3-formylphenyl)-2-(piperazin-2-yl)-1H-indole-3-carboxaldehyde; 1-(biphenyl-4-yl)-2-(piperazin-1-yl)-1H-indole-3-carboxaldehyde hydrochloride; 1-(4-ethylphenyl)-2-(piperazin-1-yl)-1H-indole-3-carboxaldehyde hydrochloride; and 1-(3-bromophenyl)-2-piperazin-1-yl-1H-indole-3-carboxaldehyde.

U.S. Pat. No. 7,087,637 to Grandel et al., discloses indole derivatives as PARP inhibitors, including: 2-(4(4-n-propyl-piperazin-1-yl)-phenyl)-1H-indol-4-carboxamide; 2-(4-piperazin-1-yl-phenyl)-1H-indol-4-carboxamide; 244(4-Isopropyl-piperazin-1-yl)-phenyl)-1H-indol-4-carboxamide; 2-(4(4-benzyl-piperazin-1-yl)-phenyl)-1H-indol-4-carboxamide; 2-(4(4-n-butyl-piperazin-1-yl)-phenyl)-1H-indol-4-carboxamide; 2-(4(4-ethyl-piperazin-1-yl)-phenyl)-1H-indol-4-carboxamide; 2-(4-(2-N,N-dimethylamino-eth-1-yloxy)-phenyl)-1H-indol-4-carboxamide; 2-(4-(2-pyrrolidinl-yl-eth-1-yloxy)-phenyl)-1H-indol-4-carboxamide; 2-(4-(2-piperidin-yl-eth-1-yloxy)-phenyl)-1H-indol-4-carboxamide; 2-(4-(2-piperazin-1-yl-eth-1-yloxy)-phenyl)-1H-indol-4-carboxamide; 2-(4-(2-(4-methyl-piperazin-1-yl)-eth-1-yloxy)-phenyl)-1H-indol-4-carboxamide; 2-(4-(2-(4-propyl-piperazin-1-yl)-eth-1-yloxy)-phenyl)-1H-indol-4-carboxamide; 2-(4-(2-(4-ethyl-piperazin-1-yl)-eth-1-yloxy)-phenyl)-1H-indol-4-carboxamide; and 2-(4-(2-(4-benzyl-piperazin-1-yl)-eth-1-yloxy)-phenyl)-1H-indol-4-carboxamide.

U.S. Pat. No. 7,041,675 to Lubisch et al., discloses substituted pyridine carboxamides as PARP inhibitors, including 2-phenylimidazo[1,2-a]pyridine-8-carboxamide; 2-(4-nitrophenyl)imidazo[1,2-a]pyridine-8-carboxamide; 2-(4-aminophenyl)imidazo[1,2-a]pyridine-8-carboxamide; 2-(2-benzothienyl)imidazo[1,2-a]pyridine-8-carboxamide; 2-(4-bromophenyl)-imidazo[1,2-a]pyridine-8-carboxamide; and 2-(4-imidazol-1-ylphenyl)imidazo[1,2-a]pyridine-8-carboxamide.

U.S. Pat. No. 6,924,284 to Beaton et al., discloses substituted bicyclic aryl PARP inhibitors, including: N-[3-(4-oxo-3,4-dihydro-phthalazin-1-ylamino)-propyl]-3-[3-(1H-pyrrol-2-yl)-[1,2,4]oxadiaol-5-yl]propionamide; N-[3-(4-oxo-3,4-dihydro-phthalazin-1-ylamino)-propyl]-3-(3-thiophen-3-yl-[1,2,4]oxadiazol-5-yl)propionamide; 3-(3-furan-2-yl-[1,2,4]oxadiazol-5-yl)-N-[3-(4-oxo-3,4-dihydro-phthalazin-1-ylamino)-propyl]-propionamide; N-[3-(4-oxo-3,4-dihydro-phthalazin-1-ylamino)-propyl]-3-(3-thiophen-2-yl-[1,2,4]oxadiazol-5-yl)-propionamide; 3-[3-(2-methyl-thiophen-3-yl)-[1,2,4]oxadiazol-5-yl]-N-[3-(4-oxo-3,4-dihydro-phthalazin-1-ylamino)-propyl]-propionamide; 3-[3-(3,5-dihydroxy-phenyl)-[1,2,4]oxadiazol-5-yl]-N-[3-(4-oxo-3,4-dihydro-phthalazin-1-ylamino)-propyl]-propionamide; 3-[3-(3-hydroxy-phenyl)-[1,2,4]oxadiazol-5-yl]-N-[3-(4-oxo-3,4-dihydro-phthalazin-1-ylamino)-propyl]-propionamide; and 3-[3-(5-amino-3H-imidazol-4-yl)-[1,2,4]oxadiazol-5-yl]-N-[3-(4-oxo-3,4-dihydro-phthalazin-1-ylamino)-propyl]-propionamide.

U.S. Pat. No. 6,635,642 to Jackson et al., discloses phthalazinone derivatives as PARP inhibitors, including 4-(3-nitro-4-(piperidin-1-yl)phenyl-phthalazin-1(2H)-one; 4-(4-(dimethylamino)-3-nitrophenyl)-phthalazin-1(2H)-one; 4-(3-amino-4-(dimethylamino)phenyl)-phthalazin-1(2H)-one; 4-(4-phenylpiperazin-1-yl)-phthalazin-1(2H)-one; and 4-(4-(4-chlorophenyl)-piperazin-1-yl)-phthalazin-1(2H)-one.

U.S. Pat. No. 6,448,271 to Lubisch et al., discloses substituted benzimidazoles as PARP inhibitors, including 2-(piperidin-4-yl)benzimidazole-4-carboxamide dihydrochloride; 2-(N-acetylpiperidin-4-yl)benzimidazole-4-carboxamide; 2-(N-propylpiperidin-4-yl)benzimidazole-4-carboxamide; 2-piperidin-3-yl)benzimidazole-4-carboxamide dihydrochloride; 2-(N—(O-t-butoxycarbonyl)piperidin-3-yl)benzimidazole-4-carboxamide; 2-(N-benzylpiperidin-3-yl)benzimidazole-4-carboxamide; 2-(N-methylpiperidin-3-yl)benzimidazole-4-carboxamide dihydrochloride; 2-piperazin-4-yl-benzimidazole-4-carboxamide; 2-(N-propylpiperidin-3-yl)benzimidazole-4-carboxamide dihydrochloride; 2-(N-(3-phenylprop-1-yl)-piperidin-3-yl)benzimidazole-4-carboxamide dihydrochloride; 2-(N-benzoylpiperidin-3-yl)benzimidazole-4-carboxamide; 2-(N-benzylpiperidin-4-yl)benzimidazole-4-carboxamide dihydrochloride; 2-(1-(1-methylpiperidin-4-yl)piperidin-4-yl)benzimidazole-4-carboxamide trihydrochloride; 2-(N-n-pentylpiperidin-4-yl)benzimidazole-4-carboxamide; 2-(N-isobut-1-yl-piperidin-4-yl)benzimidazole-4-carboxamide; 2-(N-n-butylpiperidin-4-yl)benzimidazole-4-carboxamide hydrochloride; 2-(N-(3-methyl-but-1-yl)piperidin-4-yl)benzimidazole-4-carboxamide hydrochloride; 2-(1,4-dimethylpiperazin-2-yl)benzimidazole-4-carboxamide dihydrochloride; 2-piperazin-2-yl-benzimidazole-4-carboxamide dihydrochloride; 2-(N-isopropylpiperidin-4-yl)benzimidazole-4-carboxamide hydrochloride; 2-(4-(2-ethyl-prop-1-yl)piperidin-4-yl)benzimidazole-4-carboxamide; 2-(1,4-dibenzylpiperazin-2-yl)benzimidazole-4-carboxamide dihydrochloride; and 2-(N-benzylpiperidin-4-yl)-1-(1-benzylpiperidin-4-ylcarbonyl)benzimidazole-4-carboxamide.

U.S. Pat. No. 6,426,415 to Jackson et al., discloses alkoxy-substituted PARP inhibitors, including 1-(benzyloxy)-5-methylphthalazine; 1-(methoxy)-5-methyl-phthalazine; 1-(ethoxy)-5-methylphthalazine; 1-(propoxy)-5-methylphthalazine; 1-(butoxy)-5-methyl-phthalazine; 1-(methoxy)-5-hydroxyphthalazine; 1-(ethoxy)-5-hydroxyphthalazine; 1-(propoxyoxy)-5-hydroxy-phthalazine; 1-(butoxy)-5-hydroxyphthalazine; 1-(benzyloxy)-5-methylisoquinoline; 1-(methoxy)-5-methyl-isoquinoline; 1-(ethoxy)-5-methylisoquinoline; 1-(propoxy)-5-methylisoquinoline; 1-(butoxy)-5-methylisoquinoline; 1-(ethoxy)-5-hydroxyisoquinoline; 1-(propoxy)-5-hydroxyisoquinoline; and 1-(butoxy)-5-hydroxy-isoquinoline.

U.S. Pat. No. 6,395,749 to Li et al., discloses substituted carboxamides as PARP inhibitors, including 5-carbamoylquinoline-4-carboxylic acid.

U.S. Pat. No. 6,387,902 to Zhang et al., discloses substituted phenazines as PARP inhibitors, including compounds of Formula (P-VI):

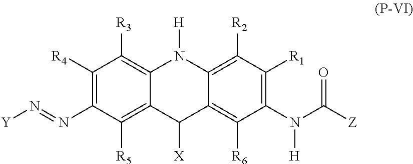

wherein:

(1) R₁-R₉ and Z are independently hydrogen, hydroxy, halo, haloalkyl, thiocarbonyl, cyano, nitro, amino, imino, alkylamino, aminoalkyl, sulfhydryl, thioalkyl, alkylthio, sulfonyl, alkylsulfonyl, C₁-C₉ straight or branched chain alkyl, C₂-C₉ straight or branched chain alkenyl, C₂-C₉ straight or branched chain alkynyl, C₁-C₆ straight or branched chain alkoxy, C₂-C₆ straight or branched chain alkenoxy, C₂-C₆ straight or branched chain alkynoxy, aryl, carbocycle, heterocycle, aralkyl, alkylaryl, alkylaryloxy, aryloxy, aralkyloxy, aralkylsulfonyl, aralkylamino, arylamino, arylazo, arylthio, or aralkylthio; or

(2) Z is a moiety of Subformula (P-VI(a))

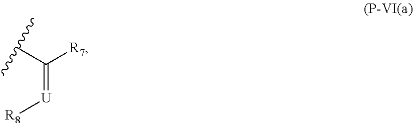

wherein U is C or N; R₇ and R₈ are as defined in (1); and X and Y are independently aryl, carbocycle, or heterocycle.

U.S. Pat. No. 6,380,211 to Jackson et al., discloses alkoxy-substituted PARP inhibitors, including 1-(methoxy)-5-methylisoquinoline, 1-(ethoxy)-5-methyl-isoquinoline, 1-(propoxy)-5-methylisoquinoline, 1-(butoxy)-5-methylisoquinoline, 1-(ethoxy)-5-hydroxy-isoquinoline, 1-(propoxy)-5-hydroxyisoquinoline, 1-(butoxy)-5-hydroxyisoquinoline, 1-(benzyloxy)-5-methylphthalazine and 1-(benzyloxy)-5-methylisoquinoline.

U.S. Pat. No. 6,358,975 to Eliasson et al., discloses PARP inhibitors, including 6(5H)-phenanthridinone, 2-nitro-6(5H)-phenanthridinone, 4-hydroxyquinazoline, 2-methyl-4(3H)-quinazoline, 2-mercapto-4(3H)-quinazoline, benzoyleneurea, 6-amino-1,2-benzopyrone, trp-P-1(3-amino-1,4-dimethyl-5H-pyrido[4,3-b]indole), juglone, luminol, 1(2H)-phthalazinone, phthalhydrazide, and chlorothenoxazin.

U.S. Pat. No. 6,235,748 to Li et al., discloses oxo-substituted compounds containing at least one ring nitrogen as PARP inhibitors.

U.S. Pat. No. 6,201,020 to Zhang et al., discloses ortho-diphenol compounds as PARP inhibitors, including compounds of Formula (P-VII):

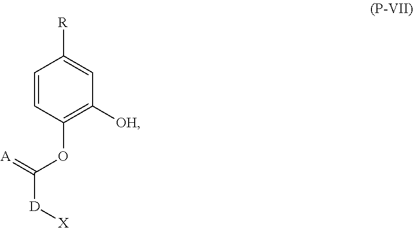

wherein:

(1) A is O or S;

(2) R is C₁-C₁₀ straight or branched chain alkyl, C₂-C₁₀ straight or branched chain alkenyl, C₂-C₁₀ straight or branched chain alkynyl, aryl, heteroaryl, carbocycle, or heterocycle;

(3) D is a bond, or a C₁-C₃ straight or branched chain alkyl, C₂-C₃ straight or branched chain alkenyl, C₂-C₃ straight or branched chain alkynyl, wherein any of the carbon atoms of said alkyl, alkenyl, or alkynyl of D are optionally replaced with oxygen, nitrogen, or sulfur; and

(4) X is aryl, heteroaryl, carbocycle, or heterocycle.

U.S. Pat. No. 5,756,510 to Griffin et al., discloses benzamide analogs that are PARP inhibitors, including: 3-benzyloxybenzamide; 3-(4-methoxybenzyloxy)benzamide; 3-(4-nitrobenzyloxy)benzamide; 3-(4-azidobenzyloxy)benzamide; 3-(4-bromobenzyloxy)benzamide; 3-(4-fluorobenzyloxy)benzamide; 3-(4-aminobenzyloxy)benzamide; 3-(3-nitrobenzyloxy)benzamide; 3-(3,4-methylenedioxyphenylmethyloxy)benzamide; 3-(piperonyloxy)benzamide; 3-(N-acetyl-4-aminobenzyloxy)benzamide; 3-(4-trifluoromethylbenzyloxy)benzamide; 3-(4-cyanobenzyloxy)benzamide; 3-(4-carboxymethylbenzyloxy)benzamide; 3-(2-nitrobenzyloxy)benzamide; 3-(4-carboxybenzyloxy)benzamide; 3-(8-adenos-9-yloctyloxy)benzamide; 3-[5-(6-chloropurin-9-yl)pentyloxy]benzamide; 3-(5-adenos-9-ylpentyloxy)benzamide; 3-[8-(6-chloropurin-9-yl)octyloxy]benzamide; 3-[12-(6-chloropurin-9-yl)dodecyloxy]benzamide; and 3-(12-adenos-9-yldodecyloxy)benzamide.

United States Patent Application Publication No. 2015/0175617 by Zhou et al., discloses fused tetra or penta-cyclic dihydrodiazepinocarbazolones as PARP inhibitors, including: 2,3,5,10-tetrahydro-[1,2]diazepino[3,4:5,6-def]carbazol-6(1H)-one; 5,6,7,8-tetrahydro-4H-4,9,10-triazaindeno[2,1,7-kla]heptalen-11(10H)-one; 2-methyl-2,3,5,10-tetrahydro-[1,2]diazepino[3,4:5,6-def]carbazol-6(1H)-one; 3,3-dimethyl-2,3,5,10-tetrahydro-[1,2]diazepino[3,4:5,6-def]carbazol-6(1H)-one; 2-phenyl-2,3,5,10-tetrahydro-[1,2]diazepino[3,4:5,6-def]carbazol-6(1H)-one; and 2-isopropyl-2,3,5,10-tetrahydro-[1,2]diazepino[3,4:5,6-def]carbazol-6(1H)-one.

United States Patent Application Publication No. 2015/0152118 by Jana et al., discloses tetrahydroquinazolinone derivatives as PARP inhibitors, including: 2′-(3-(4-(4-fluorophenyl)piperazin-1-yl)propyl)-6′,7′-dihydro-3′H-spiro[cyclopropane-1,8′-quinazolin]-4′(5′H)-one; 2′-(3-(4-(4-chlorophenyl)piperazin-1-yl)propyl)-6′,7′-dihydro-3′H-spiro[cyclopropane-1,8′-quinazolin]-4′(5′H)-one; 2′-(3-(4-phenyl-5,6-dihydropyridin-1(2H)-yl)propyl)-6′,7′-dihydro-3′H-spiro[cyclopropane-1,8′-quinazolin]-4′(5′H)-one; 2′-(3-(3-(4-fluorophenyl)-3,8-diazabicyclo[3.2.1]octan-8-yl)propyl)-4a′,5′,6′,7′-tetrahydro-3′H-spiro[cyclopropane-1,8′-quinazolin]-4′(8a′H)-one; 2′-(3-(4-(4-fluorophenyl)piperazin-1-yl)propyl)-7′,8′-dihydro-3′H-spiro[cyclopropane-1,6′-quinazolin]-4′(5′H)-one; 2′-(3-(4-(4-chlorophenyl)piperazin-1-yl)propyl)-7′,8′-dihydro-3′H-spiro[c yclopropane-1,6′-quinazolin]-4′(5′H)-one; 2′-(3-(3-(4-fluorophenyl)-3,8-diazabicyclo[3.2.1]octan-8-yl)propyl)-7′,8′-dihydro-3′H-spiro[cyclopropane-1,6′-quinazolin]-4′(5′H)-one; 2′-(3-(8-(4-fluorophenyl)-3,8-diazabicyclo[3.2.1]octan-3-yl)propyl)-7′,8′-dihydro-3′H-spiro[cyclopropane-1,6′-quinazolin]-4′(5′H)-one; 2′-(3-(4-(4-fluorophenyl)-5,6-dihydropyridin-1(2H)-yl)propyl)-7′,8′-dihydro-3′H-spiro[cyclopropane-1,6′-quinazolin]-4′(5′H)-one; and 2′-(3-(4-(3,4-dichlorophenyl)piperazin-1-yl)propyl)-7′,8′-dihydro-3′H-spiro[cyclopropane-1,6′-quinazolin]-4′(5′H).

United States Patent Application Publication No. 2015/0031652 by Gangloff et al., discloses substituted 1,2,3,4-tetrahydropyrido[2,3-b]pyrazines as PARP inhibitors, including (S)-3-((4-(4-chlorophenyl)piperazin-1-yl)methyl)-6a,7,8,9-tetrahydropyrido[3,2-e]pyrrolo[1,2-a]pyrazin-6(5H)-one; (S)-3-((4-(4-chlorophenyl)-5,6-dihydropyridin-1(2H)-yl)methyl)-6a,7,8,9-tetrahydropyrido[3,2-e]pyrrolo[1,2-a]pyrazin-6(5H)-one; (S)-3-((4-(4-chlorophenyl)piperidin-1-yl)methyl)-6a,7,8,9-tetrahydropyrido[3,2-e]pyrrolo[1,2-a]pyrazin-6(5H)-one; (S)-4-(4-((6-oxo-5,6,6a,7,8,9-hexahydropyrido[3,2-e]pyrrolo[1,2-a]pyrazin-3-yl)methyl)piperazin-1-yl)benzonitrile; (5)-6-(4-((6-oxo-5,6,6a,7,8,9-hexahydropyrido[3,2-e]pyrrolo[1,2-a]pyrazin-3-yl)methyl)piperazin-1-yl)nicotinonitrile; (S)—N-methyl-6-(4-((6-oxo-5,6,6a,7,8,9-hexahydropyrido[3,2-e]pyrrolo[1,2-a]pyrazin-3-yl)methyl)piperazin-1-yl)nicotinamide; (S)-ethyl 6-(4-((6-oxo-5,6,6a,7,8,9-hexahydropyrido[3,2-e]pyrrolo[1,2-a]pyrazin-3-yl)methyl)piperazin-1-yl)nicotinate; (S)-6-(4-((6-oxo-5,6,6a,7,8,9-hexahydropyrido[3,2-e]pyrrolo[1,2-a]pyrazin-3-yl)methyl)piperazin-1-yl)nicotinic acid; (S)—N-ethyl-6-(4-((6-oxo-5,6,6a,7,8,9-hexahydropyrido[3,2-e]pyrrolo[1,2-a]pyrazin-3-yl)methyl)piperazin-1-yl)nicotinamide; (S)—N-cyclopropyl-6-(4-((6-oxo-5,6,6a,7,8,9-hexahydropyrido[3,2-e]pyrrolo[1,2-a]pyrazin-3-yl)methyl)piperazin-1-yl)nicotinamide; (S)—N-isopropyl-6-(4((6-oxo-5,6,6a,7,8,9-hexahydropyrido[3,2-e]pyrrolo[1,2-a]pyrazin-3-yl)methyl)piperazin-1-yl)nicotinamide; (S)—N-ethyl-4-(4((6-oxo-5,6,6a,7,8,9-hexahydropyrido[3,2-e]pyrrolo[1,2-a]pyrazin-3-yl)methyl)piperazin-1-yl)benzamide; (S)-ethyl 4-(4-((6-oxo-5,6,6a,7,8,9-hexahydropyrido[3,2-e]pyrrolo[1,2-a]pyrazin-3-yl)methyl)piperazin-1-yl)benzoate; (S)-4-(4-((6-oxo-5,6,6a,7,8,9-hexahydropyrido[3,2-e]pyrrolo[1,2-a]pyrazin-3-yl)methyl)piperazin-1-yl)benzoic acid; (S)—N-methyl-4-(4((6-oxo-5,6,6a,7,8,9-hexahydropyrido[3,2-e]pyrrolo[1,2-a]pyrazin-3-yl)methyl)piperazin-1-yl)benzamide; (S)—N-isopropyl-4-(4((6-oxo-5,6,6a,7,8,9-hexahydropyrido[3,2-e]pyrrolo[1,2-a]pyrazin-3-yl)methyl)piperazin-1-yl)benzamide; (S)-N-cyclopropyl-4-(4((6-oxo-5,6,6a,7,8,9-hexahydropyrido[3,2-e]pyrrolo[1,2-a]pyrazin-3-yl)methyl)piperazin-1-yl)benzamide; (S)-3-fluoro-4-(4-((6-oxo-5,6,6a,7,8,9-hexahydropyrido[3,2-e]pyrrolo[1,2-a]pyrazin-3-yl)methyl)piperazin-1-yl)benzonitrile; and (S)-3-((4-(2,4-difluorophenyl)piperazin-1-yl)methyl)-6a,7,8,9-tetrahydropyrido[3,2-e]pyrrolo[1,2-a]pyrazin-6(5H)-one.

United States Patent Application Publication No. 2015/0025071 by Buchstaller et al., discloses tetrahydroquinazolinone derivatives as PARP inhibitors, including: 2-[4-(4-methoxy-phenyl)-piperazin-1-yl]-5,6,7,8-tetrahydro-3H-quinazolin-4-one; 2-[4-(3-fluorophenyl)piperazin-1-yl]-5,6,7,8-tetrahydro-3H-quinazolin-4-one; 2-[4-(4-fluorophenyl)piperazin-1-yl]-5,6,7,8-tetrahydro-3H-quinazolin-4-one; 2-[4-(3-methoxyphenyl)piperazin-1-yl]-5,6,7,8-tetrahydro-3H-quinazolin-4-one; 2-[4-(4-chlorophenyl)piperazin-1-yl]-5,6,7,8-tetrahydro-3H-quinazolin-4-one; 2-[4-(2-chlorophenyl)piperazin-1-yl]-5,6,7,8-tetrahydro-3H-quinazolin-4-one; 2-(4-trifluoromethylpiperidin-1-yl)-5,6,7,8-tetrahydro-3H-quinazolin-4-one; 2-[4-(3-chlorophenyl)piperazin-1-yl]-5,6,7,8-tetrahydro-3H-quinazolin-4-one; 2-[4-(2-methoxyphenyl)piperazin-1-yl]-5,6,7,8-tetrahydro-3H-quinazolin-4-one; 2-(4-tert-butylpiperazin-1-yl)-5,6,7,8-tetrahydro-3H-quinazolin-4-one; 2-[4-(4-methoxyphenyl)-3-oxopiperazin-1-yl]-5,6,7,8-tetrahydro-3H-quinazolin-4-one; 2-[4-(piperidine-1-carbonyl)piperazin-1-yl]-5,6,7,8-tetrahydro-3H-quinazolin-4-one; 2-[4-(6-hydroxypyridin-2-yl)piperazin-1-yl]-5,6,7,8-tetrahydro-3H-quinazolin-4-one; 2-(4-benzoylpiperazin-1-yl)-5,6,7,8-tetrahydro-3H-quinazolin-4-one; N-pyridin-2-yl-2-[4-(4-oxo-3,4,5,6,7,8-hexahydroquinazolin-2-yl)piperazin-1-yl]acetamide; 2-(4-acetylpiperazin-1-yl)-5,6,7,8-tetrahydro-3H-quinazolin-4-one; 2-[4-(morpholine-4-carbonyl)piperazin-1-yl]-5,6,7,8-tetrahydro-3H-quinazolin-4-one; 2-[4-(3-aminopropanoyl)piperazin-1-yl]-5,6,7,8-tetrahydro-3H-quinazolin-4-one; 2-[4-(4-oxo-5,6,7,8-tetrahydro-3H-quinazolin-2-yl)piperazin-1-yl]pyridine-3-carboxamide; 2-[4-(4-oxo-3,4,5,6,7,8-hexahydroquinazolin-2-yl)piperazin-1yl]-N-pyridin-3-ylacetamide; 2-[4-(2,2-dimethylpropanoyl)piperazin-1-yl]-5,6,7,8-tetrahydro-3H-quinazolin-4-one; 2-[4-(2-hydroxyethyl)piperazin-1-yl]-5,6,7,8-tetrahydro-3H-quinazolin-4-one; 2-[4-(2-(2-pyridyl)ethyl]piperazin-1-yl]-5,6,7,8-tetrahydro-3H-quinazolin-4-one; 2-[4-(piperidine-2-carbonyl)piperazin-1-yl]-5,6,7,8-tetrahydro-3H-quinazolin-4-one; 4-(4-oxo-5,6,7,8-tetrahydro-3H-quinazolin-2-yl)piperazine-2-carboxamide; 2-[3-(hydroxymethyl)piperazin-1-yl]-5,6,7,8-tetrahydro-3H-quinazolin-4-one; (2R)-1-(4-oxo-5,6,7,8-tetrahydro-3H-quinazolin-2-yl)piperazine-2-carboxamide; and 2-[(2R)-2-(hydroxymethyl)piperazin-1-yl]-5,6,7,8-tetrahydro-3H-quinazolin-4-one.

United States Patent Application Publication No. 2015/0018356 by Zhou et al., discloses fused tetra- or pentacyclic pyridophthalazinones as PARP inhibitors.

United States Patent Application Publication No. 2014/0336192 to Papeo et al., discloses substituted 3-phenyl-isoquinolin-1(2H)-one derivatives as PARP inhibitors, including: 4-(2-amino-ethoxy)-3-(4-bromo-phenyl)-7-fluoro-2H-isoquinolin-1-one; 4-(2-amino-ethoxy)-7-fluoro-3-(3-trifluoromethyl-phenyl)-2H-isoquinolin-1-one; 4-(2-amino-ethoxy)-7-fluoro-3-(4-morpholin-4-yl-phenyl)-2H-isoquinolin-1-one; 4-(2-amino-ethoxy)-3-(3-bromo-4-morpholin-4-yl-phenyl)-7-fluoro-2H-isoquinolin-1-one; 4-(2-amino-ethoxy)-3-(3-bromo-phenyl)-7-fluoro-2H-isoquinolin-1-one; 4-[4-(2-amino-ethoxy)-7-fluoro-1-oxo-1,2-dihydro-isoquinolin-3-yl]-benzonitrile; 4-(2-aminoethoxy)-7-fluoro-3-(4-pyrrolidin-1-yl-phenyl)-2H-isoquinolin-1-one; 4-(2-amino-ethoxy)-3-(4-chloro-phenyl)-7-fluoro-2H-isoquinolin-1-one; 4-(2-amino-ethoxy)-7-fluoro-3-(4-methanesulfonyl-phenyl)-2H-isoquinolin-1-one; 4-(2-amino-ethoxy)-7-fluoro-3-(4-fluoro-phenyl)-2H-isoquinolin-1-one; 3-[4-(2-amino-ethoxy)-7-fluoro-1-oxo-1,2-dihydro-isoquinolin-3-yl]-benzonitrile; 4-(2-amino-ethoxy)-3-(4-bromo-phenyl)-7,8-difluoro-2H-isoquinolin-1-one; 4-(2-amino-ethoxy)-3-(4-chloro-3-methyl-phenyl)-7-fluoro-2H-isoquinolin-1-one; 4-(2-amino-ethoxy)-3-(3,4-dichloro-phenyl)-7-fluoro-2H-isoquinolin-1-one; 4-(2-amino-ethoxy)-3-(3,4-difluoro-phenyl)-7-fluoro-2H-isoquinolin-1-one; 5-[4-(2-amino-ethoxy)-7-fluoro-1-oxo-1,2-dihydro-isoquinolin-3-yl]-2-morpholin-4-yl-benzonitrile; 5-[4-(2-amino-ethoxy)-7-fluoro-1-oxo-1,2-dihydro-isoquinolin-3-yl]-2-pyrrolidin-1-yl-benzonitrile; 4-(2-amino-ethoxy)-3-(3-bromo-4-pyrrolidin-1-yl-phenyl)-7-fluoro-2H-isoquinolin-1-one; 4-(2-amino-ethoxy)-3-(2,3-dihydro-benzo[1,4]dioxin-6-yl)-7-fluoro-2H-isoquinolin-1-one; 4-(2-amino-ethoxy)-3-benzo[1,3]dioxol-5-yl-7-fluoro-2H-isoquinolin-1-one; 4-(2-amino-ethoxy)-7-fluoro-3-(3-fluoro-4-methoxy-phenyl)-2H-isoquinolin-1-one; 4-(2-amino-ethoxy)-7-fluoro-3-(4-trifluoromethoxy-phenyl)-2H-isoquinolin-1-one; and 4-(2-amino-ethoxy)-3-(3,4-dihydro-2H-benzo[b][1,4]dioxepin-7-yl)-7-fluoro-2H-isoquinolin-1-one.

United States Patent Application Publication No. 2014/0235675 by Papeo et al., discloses 3-oxo-2,3-dihydro-1H-indazole-4-carboxamide derivatives as PARP inhibitors, including: 3-oxo-2-(piperidin-4-yl)-2,3-dihydro-1H-indazole-4-carboxamide; 2-(1-cyclopentylpiperidin-4-yl)-3-oxo-2,3-dihydro-1H-indazole-4-carboxamide; 2-(1-cyclohexylpiperidin-4-yl)-3-oxo-2,3-dihydro-1H-indazole-4-carboxamide; 2-[1-(4,4-difluorocyclohexyl)piperidin-4-yl]-3-oxo-2,3-dihydro-1H-indazole-4-carboxamide; 2-(1-cyclohexylpiperidin-4-yl)-1-methyl-3-oxo-2,3-dihydro-1H-indazole-4-carboxamide; 2-[1-(4,4-difluorocyclohexyl)piperidin-4-yl]-1-methyl-3-oxo-2,3-dihydro-1H-indazole-4-carboxamide; 2-(1-cyclopentylpiperidin-4-yl)-1-methyl-3-oxo-2,3-dihydro-1H-indazole-4-carboxamide; 2-(1-methylpiperidin-4-yl)-3-oxo-2,3-dihydro-1H-indazole-4-carboxamide; 1-methyl-3-oxo-2-(piperidin-4-yl)-2,3-dihydro-1H-indazole-4-carboxamide; 1-methyl-2-(1-methylpiperidin-4-yl)-3-oxo-2,3-dihydro-1H-indazole-4-carboxamide; 2-(1-ethylpiperidin-4-yl)-3-oxo-2,3-dihydro-1H-indazole-4-carboxamide; 3-oxo-2-(1-propylpiperidin-4-yl)-2,3-dihydro-1H-indazole-4-carboxamide; 2-(1-ethylpiperidin-4-yl)-1-methyl-3-oxo-2,3-dihydro-1H-indazole-4-carboxamide; 1-methyl-3-oxo-2-[1-(propan-2-yl)piperidin-4-yl]-2,3-dihydro-1H-indazole-4-carboxamide; 3-oxo-2-[1-(propan-2-yl)piperidin-4-yl]-2,3-dihydro-1H-indazole-4-carboxamide; 2-(1-cyclobutylpiperidin-4-yl)-3-oxo-2,3-dihydro-1H-indazole-4-carboxamide; 2-(1-cyclobutylpiperidin-4-yl)-6-fluoro-3-oxo-2,3-dihydro-1H-indazole-4-carboxamide; 2-(1-cyclobutylpiperidin-4-yl)-1-methyl-3-oxo-2,3-dihydro-1H-indazole-4-carboxamide; 2-(1-cyclobutylpiperidin-4-yl)-6-fluoro-1-methyl-3-oxo-2,3-dihydro-1H-indazole-4-carboxamide; 6-fluoro-3-oxo-2-(piperidin-4-yl)-2,3-dihydro-1H-indazole-4-carboxamide; 6-fluoro-1-methyl-3-oxo-2-(piperidin-4-yl)-2,3-dihydro-1H-indazole-4-carboxamide; 2-(1-cyclohexylpiperidin-4-yl)-6-fluoro-1-methyl-3-oxo-2,3-dihydro-1H-indazole-4-carboxamide; 2-(1-cyclohexylpiperidin-4-yl)-6-fluoro-3-oxo-2,3-dihydro-1H-indazole-4-carboxamide; and 2-[1-(4,4-difluorocyclohexyl)piperidin-4-yl]-6-fluoro-1-methyl-3-oxo-2,3-dihydro-1H-indazole-4-carboxamide.

United States Patent Application Publication No. 2014/0023642 by Cai et al., discloses 1-(arylmethyl)quinazoline-2,4(1H,3H)-diones as PARP inhibitors, including: 1-(3-methoxycarbonylbenzyl)quinazoline-2,4(1H,3H)-dione; 1-(3-carboxybenzyl)quinazoline-2,4(1H,3H)-dione; 1-(3-(4-(pyridin-2-yl)piperazine-1-carbonyl)benzyl)quinazoline-2,4(1H,3H)-dione; 1-(3-(4-(pyrimidin-2-yl)piperazine-1-carbonyl)benzyl)quinazoline-2,4(1H,3H)-dione; 1-(3-(4-cyclohexylpiperazine-1-carbon yl)benzyl)quinazoline-2,4(1H,3H)-dione; 1-(3-(4-([1,2,4]triazolo[4,3-b]pyridazin-6-yl)piperazine-1-carbonyl)benzyl)quinazoline-2,4(1H,3H)-dione; 1-(3-(4-ethylpiperazine-1-carbonyl)benzyl)quinazoline-2,4(1H,3H)-dione; 1-(3-(4-benzoylpiperazine-1-carbonyl)benzyl)quinazoline-2,4(1H,3H)-dione; 1-(3-(4-(4-fluorobenzoyl)piperazine-1-carbonyl)benzyl)quinazoline-2,4(1H,3H)-dione; 1-(3-(4-(4-chlorobenzoyl)piperazine-1-carbonyl)benzyl)quinazoline-2,4(1H,3H)-dione; 1-(3-(4-(4-bromobenzoyl)piperazine-1-carbonyl)benzyl)quinazoline-2,4(1H,3H)-dione; 1-(3-(4-(4-methoxybenzoyl)piperazine-1-carbonyl)benzyl)quinazoline-2,4(1H,3H)-dione; 1-(3-(4-(tetrahydro-2H-pyran-4-yl)carbonylpiperazine-1-carbonyl)benzyl)quinazoline-2,4(1H,3H)-dione; 1-(3-(4-(cyclopentylcarbonyl)piperazine-1-carbonyl)benzyl)quinazoline-2,4-(1H,3H)-dione; 1-(3-(4-(cyclopropylcarbonyl)piperazine-1-carbonyl)benzyl)quinazoline-2,4-(1H,3H)-dione; 1-(3-(4-(ethylsulfonyl)piperazine-1-carbonyl)benzyl)quinazoline-2,4(1H,3H)-dione; 1-(3-(4-acetylpiperazine-1-carbonyl)benzyl)quinazoline-2,4(1H,3H)-dione; 1-(3-(4-phenylpiperidine-1-carbonyl)benzyl)quinazoline-2,4(1H,3H)-dione; 1-(3-(4-phenylpiperazine-1-carbonyl)benzyl)quinazoline-2,4(1H,3H)-dione; and 1-(3-(4-(pyrazin-2-yl)piperazine-1-carbonyl)benzyl)quinazoline-2,4(1H,3H)-dione.

United States Patent Application Publication No. 2013/0225647 by Donawho et al., discloses PARP inhibitors of Formula (P-VIII):

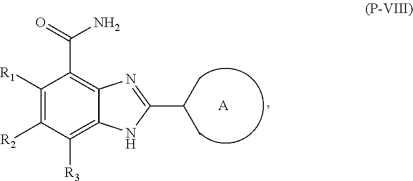

wherein:

(1) R₁, R₂, and R₃ are independently selected from the group consisting of hydrogen, alkenyl, alkoxy, alkoxycarbonyl, alkyl, alkynyl, cyano, haloalkoxy, haloalkyl, halogen, hydroxy, hydroxyalkyl, nitro, NR_{A}R_{B}, and (NR_{A}R_{B})carbonyl;

(2) A is a nonaromatic 4, 5, 6, 7, or 8-membered ring that contains 1 or 2 nitrogen atoms and, optionally, one sulfur or oxygen atom, wherein the nonaromatic ring is optionally substituted with 1, 2, or 3 substituents selected from the group consisting of alkenyl, alkoxy, alkoxyalkyl, alkoxycarbonyl, alkoxycarbonylalkyl, alkyl, alkynyl, aryl, arylalkyl, cycloalkyl, cycloalkylalkyl, cyano, haloalkoxy, haloalkyl, halogen, heterocycle, heterocyclealkyl, heteroaryl, heteroarylalkyl, hydroxy, hydroxyalkyl, nitro, NR_{C}R_{D}, (NRcRo)alkyl, (NR_{C}R_{D})carbonyl, (NR_{C}R_{D})carbonylalkyl, and (NR_{C}R_{D})sulfonyl; and;

(3) R_{A}, R_{B}, R_{C}, and R_{D} are independently selected from the group consisting of hydrogen, alkyl, and alkycarbonyl.

United States Patent Application Publication No. 2013/0129841 by Ciavolella et al., disclosed PARP inhibitors including 2-[1-(4,4-difluorocyclohexyl)piperidin-4-yl]-3-oxo-2,3-dihydro-1H-isoindole-4-carboxamide; 2-(1-(4,4-difluorocyclohexy)piperidin-4-yl]-6-fluoro-3-oxo-2,3-dihydro-1H-isoindole-4-carboxamide; 6-fluoro-3-oxo-2-[1-(4-oxocyclohexy)piperidin-4-yl]-2,3-dihydro-1H-isoindole-4-carboxamide, and 2-[1-(4,4-dichlorocyclohexyl)piperidin-4-yl]-6-fluoro-3-oxo-2,3-dihydro-1-H-isoindole-4 carboxamide.

The gene PTEN encodes a protein, PTEN, that acts as a tumor suppressor. The protein encoded by PTEN is a phosphatidylinositol-3,4,5-trisphosphate 3-phosphatase. It contains a tensin-like domain as well as a catalytic domain similar to that of the dual specificity protein tyrosine phosphatases. Unlike most of the protein tyrosine phosphatases, this protein preferentially dephosphorylates phosphoinositide substrates. It negatively regulates intracellular levels of phosphatidylinositol-3,4,5-trisphosphate in cells and functions as a tumor suppressor by negatively regulating the Akt/PKB signaling pathway. The PTEN gene is frequently mutated, lost, or its expression downregulated in cancer. As such, the loss or inactivation of PTEN function is increasingly viewed as a target for therapeutic intervention (L. M. Dillon & T. W. Miller, “Therapeutic Targeting of Cancers with Loss of PTEN Function,” Curr. Drug Targets 15: 65-79 (2014)). PTEN deficiency can be caused by inherited germ line mutations, somatic mutations, epigenetic and transcriptional silencing, post-translational modifications, and protein-protein interactions.

The germ line mutations can include, but are not limited to, mutations in Exon 5 encoding the PTEN phosphatase domain. Other mutations have been shown to occur in the PTEN promoter or in splice donor and acceptor sites.

Missense, nonsense, insertion, and deletion mutations occur throughout PTEN and contribute to loss of PTEN expression and/or function. Although the distribution of these mutations is generally sporadic and such mutations can occur throughout the genome, there are a number of mutational hotspots, including Arg130, Arg173, and Arg233.

PTEN loss of function can also result from epigenetic and transcriptional silencing. Several studies have shown that CpG islands in the PTEN promoter are hypermethylated in cancer, leading to silencing of PTEN transcription. Transcription of PTEN can be repressed by the epigenetic repressor complex Mi-2/NuRD that contains a chromatin-remodeling ATPase and a histone deacetylase (HDAC). This repression occurs when the transcription factor Sal-Like Protein 4 (SALL4) binds to the PTEN promoter and recruits Mi-2/NuRD. PTEN transcription can also be repressed by the transcription factors NF-κB, c-JUN, and BM1. The tumor suppressor p53, which can act as a transcription factor, promotes PTEN expression; therefore, loss of function for p53 can have the effect of reducing PTEN expression. The ubiquitous transcription factor Specificity Protein 1 (Sp1) can also inhibit PTEN expression: acetylated Sp1 binds to the PTEN promoter and recruits HDAC1 to repress PTEN transcription. Accordingly, Sp1 overexpression upregulated PI3K pathway activation (assessed by AKT phosphorylation), and promoted migration and invasion of human salivary adenoid cystic cancer cells. MicroRNAs (miRNAs), have been shown to repress translation of PTEN mRNA by interacting with the 3′ untranslated region. In particular, the miRNA miR-21 represses PTEN expression in many cancer subtypes and metabolic diseases; this miRNA may also repress PTEN expression by increasing the expression of other miRNAs that are known to repress the expression of PTEN. The transcription factor transforming growth factor beta (TGF-β), which inhibits PTEN expression in some models, upregulates miR-21 expression. Post-translational modifications including phosphorylation, acetylation, oxidation, and ubiquitylation have been shown to cause loss of PTEN function. The phosphatase activity of PTEN can be inhibited by phosphorylation of several serine and threonine resides in its C-terminal tail. This phosphorylation may be driven by the activity of the kinase CK2. While such phosphorylation stabilizes PTEN, it reduces PTEN localization to the plasma membrane, thereby limiting its interaction with PIP₃. PTEN can be also inhibited by oxidation and acetylation. PTEN contains a residue characteristic of protein tyrosine phosphatases termed a catalytic cysteine nucleophile which is prone to oxidation at Cys124; Cys124 can form a disulfide bond with Cys71, inhibiting the catalytic activity of PTEN. Additionally, PTEN is subject to acetylation at residues Lys125-128, which also inhibits the catalytic activity of PTEN. PTEN monoubiquitination at Lys13 and Lys289 promotes its nuclear localization and suppresses its phosphatase activity.

Several proteins have been shown to interact with PTEN to repress its tumor suppressive functions. Parkinson Protein 7 (PARK7, DJ-1) binds PTEN under conditions of oxidative stress, and this interaction is associated with increased AKT activation and poor clinical outcome in different cancer subtypes. PIP₃-dependent Rac Exchange Factor 2a (P-REX2a), Shank-Interacting Protein-Like 1 (SIPL1) and α-Mannosidase 2C1 (MAN2C1) have also been shown to bind PTEN and inhibit its phosphatase activity, leading to increased activation of AKT. Other proteins can stabilize PTEN, but mutations in these proteins can, therefore, reduce the activity of PTEN and promote tumorigenesis. The membrane-localized proteins E-cadherin and MAGI-2, which are lost in some cancers, promote PTEN stability. The p85 subunit of PI3K binds PTEN to promote stability. The genes encoding p85 isoforms (PIK3R1, PIK3R2) are frequently mutated in endometrial cancer, and some mutations destabilize PTEN and promote PI3K pathway activation.

There is also an interaction between PTEN and p53 that may repress or promote tumorigenesis. Nuclear PTEN binds p53 in a phosphatase-independent manner to promote p53 stabilization, thus promoting PTEN transcription. PTEN complexes with p300/CBP acetyltransferase to promote p53 acetylation in response to DNA damage, and p53 acetylation enhances PTEN-p53 interaction. Therefore, in cells expressing wild-type p53, PTEN inhibits cell proliferation and increases apoptosis. In contrast, PTEN promotes proliferation and suppresses apoptosis in cells expressing mutant p53.

Among agents that are potentially useful for countering loss of PTEN function are temsirolimus, everolimus, and other inhibitors of the AKT/mTOR pathway, including AZD6482 ((R)-2-(1-(7-methyl-2-morpholino-4-oxo-4H-pyrido[1,2-a]pyrimidin-9-yl)ethylamino)benzoic acid), which is a PI3K/p110β inhibitor, MK-2206 (8-(4-(1-aminocyclobutyl)phenyl)-9-phenyl-[1,2,4]triazolo[3,44][1,6]naphthyridin-3(2H)-one), which is an allosteric AKT inhibitor, and 17-AAG ([(3S,5S,6R,7S,8E,10R,11S,12E,14E)-21-(allylamino)-6-hydroxy-5,11-dimethoxy-3,7,9,15-tetramethyl-16,20,22-trioxo-17-azabicyclo[16.3.1]docosa-8,12,14,18,21-pentaen-10-yl] carbamate), which is a Hsp90 chaperonin inhibitor that induces degradation of many proteins including HER2 and AKT.

Loss of or inhibition of PTEN can drive resistance to a large range of anti-neoplastic therapies.

A large number of drugs have been proposed for use in the treatment of PTEN-deficient malignancies. These drugs include: (1) buparlisib; (2) XL-147 (N-[3-(2,1,3-benzothiadiazol-5-ylamino)quinoxalin-2-yl]-4-methylbenzenesulfonamide); (3) PX-866 ((1E,4S,4aR,5R,6aS,9aR)-5-(acetyloxy)-1-[(di-2-propen-1-ylamino)methylene]-4,4a,5,6,6a,8,9,9a-octahydro-11-hydroxy-4-(methoxymethyl)-4a,6a-dimethyl-cyclopenta[5,6]naphtho[1,2-c]pyran-2,7,10(1H)-trione); (4) pictilisib dimethanesulfonate; (5) copanlisib; (6) CH5132799 (5-(7-(methylsulfonyl)-2-morpholino-6,7-dihydro-5H-pyrrolo[2,3-d]pyrimidin-4-yl)pyrimidin-2-amine); (7) GDC-0084; (8) SZTK474 (2-(difluoromethyl)-1-(4,6-dimorpholino-1,3,5-triazin-2-yl)-1H-benzo[d]imidazole); (9) GDC-0032 (2-methyl-2-[4-[2-(5-methyl-2-propan-2-yl-1,2,4-triazol-3-yl)-5,6-dihydroimidazo[1,2-d][1,4]benzoxazepin-9-yl]pyrazol-1-yl]propanamide); (10) alpelisib; (11) MLN1117 (6-(2aminobenzo[d]oxazol-5-yl(1,2-a]pyridine-3-yl(morpholinomethanone); (12) GSK2636771 (2-methyl-1-[[2-methyl-3-(trifluoromethyl)phenyl]methyl]-6-(4-morpholinyl)-1H-benzimidazole-4-carboxylic acid; (13) rigosertib; (14) CUDC-097 (N-hydroxy-2-(((2-(6-methoxypyridin-3-yl)-4-morpholinothieno[3,2-d]pyrimidin-6-yl)methyl)(methyl)amino)pyrimidine-5-carboxamide); (15) gedatolisib; (16) dactolisib; (17) BGT226 (8-(6-methoxypyridin-3-yl)-3-methyl-1-(4-(piperazin-1-yl)-3-(trifluoromethyl)phenyl)-1H-imidazo[4,5-c]quinolin-2(3H)-one maleic acid); (18) apitolisib; (19) voxtalisib; (20) SF1126 ((85,145,175)-14-(carboxymethyl)-8-(3-guanidinopropyl)-17-(hydroxymethyl)-3,6,9,12,15-pentaoxo-1-(4-(4-oxo-8-phenyl-4H-chromen-2-yl)morpholino-4-ium)-2-oxa-7,10,13,16-tetraazaoctadecan-18-oate); (21) LY3023414; (22) everolimus; (23) temsirolimus; (24) ridaforolimus; (25) MLN0128 (3-(2-aminobenzo[d]oxazol-5-yl)-1-isopropyl-1H-pyrazolo[3,4-d]pyrimidin-4-am ine); (26) AZD-2014 (3-(2,4-bis((S)-3-methylmorpholino)pyrido[2,3-d]pyrimidin-7-yl)-N-methylbenzamide); (27) CC-223; (28) AZD-5313 (5-[(4-bromo-2-chlorophenyl)amino]-4-fluoro-N-(2-hydroxyethoxy)-1-methyl-1H-benzimidazole-6-carboxamide); (29) LY2780301; (30) ipatasertib; (31) afuresertib; (32) MK-2206 (8-(4-(1-aminocyclobutyl)phenyl)-9-phenyl-[1,2,4]triazolo[3,44][1,6]naphthyridin-3(2H)-one); (33) olaparib; (34) veliparib; (35) iniparib; (36) rucaparib; (37) CEP-9722 (11-methoxy-4,5,6,7-tetrahydro-1H-cyclopenta[a]pyrrolo[3,4-c]carbazole-1,3(2H)dione; (38) E7016 (10-((4-Hydroxypiperidin-1-yl)methyl)chromeno[4,3,2-de]phthalazin-3(2H)-one); and (39) E7449 (9-isoindolin-2ylmethyl-1,2-dihydro-3H-pyridazino[3,4,5-de]quinazolin-3-one). Other therapeutic agents are known in the art.

U.S. Pat. No. 8,933,070 to Pan et al., discloses treatment of malignancies characterized by a PTEN gene mutation by administration of a PLK4 antagonist.

United States Patent Application Publication No. 2015/0159161 by Krieg et al., discloses single-stranded oligonucleotides for enhancing or activating expression of PTEN, such as oligonucleotides having a sequence 5′-X—Y—Z, wherein X is any nucleotide, Y is a nucleotide sequence of 6 nucleotides in length that is not a seed sequence of a human microRNA, and Z is a nucleotide sequence of 1-23 nucleotides in length, wherein the single stranded oligonucleotide is complementary with at least 8 consecutive nucleotides of a PRC2-associated region of a PTEN gene.

United States Patent Application Publication No. 2014/0378525 by Ashworth et al., discloses the use of inhibitors of mitotic kinases for the treatment of cancers that are characterized by mutated or deficient PTEN. The mitotic kinases include AURKA, TTK, CDK4, PLK4, BUB1B, PLK1, CDC2, PLK3, and AURKB. The inhibitors can be small molecule inhibitors, such as small molecule inhibitors of TTK, such as AZ3146 (9-cyclopentyl-2-(2-methoxy-4-(1-methylpiperidin-4-yloxy)phenylamino)-7-methyl-7H-purin-8(9H)-one) or CCT132774. Antibodies, peptide fragments, antisense nucleic acids, or interfering RNA molecules can also be used.

United States Patent Application Publication No. 2014/0112917 by Yu et al., discloses use of an ErbB2-targeting agent such as trastuzumab, LY294002, Wortmannin, demethoxyviridin, Perifosine, SAR245408 (XL147), BKM120, BEZ235, GS-1101 (CAL-101), PX-866, IPI-145, and BAY 80-6946 when PTEN transcription is reduced or when at least one allele of PTEN is lost.

United States Patent Application Publication No. 2012/0165340 by Furnari et al., discloses that phosphorylation of the Y240 residue in PTEN is associated with poorer prognosis and increased resistance to antineoplastic agents. The resistance can be driven by src family kinase-mediated phosphorylation of Y240.

United States Patent Application Publication No. 2012/0107299 by Morishita et al., discloses a protein, NDRG2, that inhibits phosphorylation of residues T382, T383, and S380 of PTEN or induces dephosphorylation of these amino acid residues of PTEN; the activity of this protein can be used to inhibit the activation of the PI3K/Akt pathway.

United States Patent Application Publication No. 2011/0189169 by Abounader et al., discloses administration of an agonist for PTEN together with an inhibitor of hepatocyte growth factor (HGF). The agonist for PTEN can be an mTOR inhibitor (see WO 00/00388), which can be rapamycin, sirolimus, temsirolimus, everolimus, monoclonal antibodies, zinc fingers, or other agonists (see 2007/0280918).

United States Patent Application Publication No. 2010/0286141 by Durden, is relevant for the following description of the PTEN protein. The crystal structure of PTEN, solved in 1999, revealed that the 403 amino acid protein comprises three domains of known function. These are the N terminal catalytic domain (residues 1-185), the C2 domain (residues 186-349) that participates in membrane binding and catalysis and the C terminal tail region (residues 350-403). Each of these domains provide suitable targets for the rational design of therapeutic agents which modulate PTEN activity. Particularly preferred regions are the N terminal and C2 domains, specifically regions including certain unique residues within and adjacent to the P loop, the WPD loop and the TI loop. It is these residues that participate in specific PIP3 substrate recognition and catalysis thereof. Another suitable region includes the C terminal tail which participates in PTEN regulatory and degradation in vivo. Small peptide molecules corresponding to these regions may be used to advantage in the design of therapeutic agents which effectively modulate the activity of PTEN, PI-3 kinase cascades, AKT cascades, as well as p53-mediated transcription and cell death. PTEN is phosphorylated on tyrosine, serine and threonine residues. Agents which affect the phosphorylation state of the protein will also be screened as those small molecules which affect phosphorylation of PTEN should also modulate PTEN interactions with other proteins. The DLDLTYIYP motif (residues 22-30; SEQ ID NO: 1) at the extreme N terminus of PTEN contains an YXXP motif (SEQ ID NO: 2), a possible docking site for adapter proteins like crk and crkl via SH2 interactions. Another motif, YFSPN (SEQ ID NO: 3) in the C terminus has been identified as the binding site for crk and crkl. The YLVLTL motif (SEQ ID NO: 4) in the extreme C terminus is a site for SH2 interactions with Shc or SHP-1. The YSYL motif (SEQ ID NO: 5), which contains a tyrosine at position 178, is 100% conserved from Drosophila to man. Other tyrosine phosphorylated motifs include: YRNNIDD (SEQ ID NO: 6), Y at position 46, a sequence present in the catalytic domain identified as a binding site for Grb2 via its SH2 domain.

United States Patent Application Publication No. 2009/0019558 by Song et al., discloses agents that modulate the activity of PGD to restore function of PTEN.

Substituted hexitol derivatives as described above can be used together with other DNA-damaging anti-neoplastic agents. DNA-damaging anti-neoplastic agents are disclosed in K. Cheung-Ong et al., “DNA-Damaging Agents in Cancer Chemotherapy: Serendipity and Chemical Biology,” Chem. Biol. 20: 648-659 (2013). DNA-damaging anti-neoplastic agents are also disclosed in the following patents or published patent applications, all of which are incorporated herein by this reference: U.S. Pat. No. 9,097,722 to Yu; U.S. Pat. No. 9,096,602 to Everitt et al.; U.S. Pat. No. 8,840,898 to Goldmakher; U.S. Pat. No. 8,735,590 to Adejare et al.; U.S. Pat. No. 8,415,357 to Kawabe et al.; U.S. Pat. No. 8,476,025 to Clifford; U.S. Pat. No. 7,902,165 to Kim; U.S. Pat. No. 7,875,586 to Kovbasnjuk et al.; U.S. Pat. No. 7,652,042 to Kawabe et al.; U.S. Pat. No. 7,465,542 to Chu et al.; U.S. Pat. No. 7,070,968 to Kufe et al.; United States Patent Application Publication No. 2011/0028422 by Aloyz et al.; and United States Patent Application Publication No. 2007/0032502 by Mallams et al. DNA-damaging anti-neoplastic agents can act by a variety of mechanisms, including modification of DNA bases such as by alkylation, intercalation into the DNA structure, formation of crosslinks in DNA, prevention of unwinding or replication of DNA to induce double-strand breaks, incorporation into DNA in place of normal nucleosides, and other mechanisms. DNA-damaging anti-neoplastic agents include, but are not limited to: cisplatin, carboplatin, oxaliplatin, picoplatin, nedaplatin, satraplatin, tetraplatin, doxorubicin, daunorubicin, methotrexate, 5-fluorouracil, gemcitabine, podophyllotoxin, etoposide, teniposide, cyclophosphamide, chlorambucil, melphalan, carmustine, lomustine, estramustine, semustine, bendamustine, prednamustine, uramustine, chlornaphazine, dacarbazine, altretamine, temozolomide, mitomycin C, streptozotocin, chlorozotocin, capecitabine, floxuridine, 6-mercaptopurine, 8-azaguanine, azathiopurine, 5-ethynyluracil, thioguanine, fludarabine, cytarabine, cladribine, 2-fluoro-arabinosyl-adenine, aminopterin, pemetrexed, ralitrexed, camptothecin, epirubicin, idarubicin, methylnitronitrosoguanidine, topotecan, irinotecan, mechlorethamine, ifosfamide, trofosfamide, busulfan, procarbazine, mitoxantrone, actinomycin, calicheamicin, Tegafur (R,S-1-(tetrahydro-2-furanyl)-5-fluorouracil), 2′,2′-difluoro-2′-deoxycytidine, bischloroethylsulfide, thiotepa, aziridinylbenzoquinone, BCNU, CCNU, 4-methyl CCNU, ACNU, rebeccamycin, bleomycin, pepleomycin, ethylmethanesulfonate, methylmethanesulfonate, dimethylnitrosamine, dimethyl sulfate, and N′-[2-[2-(4-methoxypheny)ethenyl]-4-quinazolinyl]-N,N-dimethyl-1,3-propanediamine dihydrochloride.

Additional mechanistic findings with respect to the activity of dianhydrogalactitol provide methods for the use of dianhydrogalactitol to prevent cell division in cancer cells and thus optimize the use of agents such as topoisomerase inhibitors to inhibit cancer cell replication and division and promote apoptosis. The findings suggest that dianhydrogalactitol at any point in the cell cycle can introduce DNA crosslinks. These crosslinks cannot be sufficiently removed before the cell moves into the S-phase of the cell cycle. In the S-phase, the cell attempts to duplicate the DNA, and thus needs to separate the two DNA strands of each DNA molecule to initiate the duplication process. However, when DNA helicase attempts to separate the DNA strands, the persistent DNA crosslink induced by dianhydrogalactitol leads to a DNA double-strand break (DSB). This double strand break needs to be repaired before the cell can move on in the cell cycle, and the cell is thus stalled in the S-phase while it tries to repair the break. The activated repair system is called homologous recombination repair and these findings demonstrate continuous activation of proteins in this repair system. These findings also indicate that the cells are unable to repair the break and are thus stalled in S-phase for an extended period of time, before eventually being directed to apoptosis. In particular, these findings enable us to predict which combination therapies would be useful for the treatment of the malignancies recited above, including, but not limited to, glioblastoma, NSCLC, and ovarian cancer.

Topoisomerase inhibitors used in cancer therapy, such as, but not limited to, doxorubicin, target the topoisomerase-DNA complex when the cell is in S-phase. This means that these inhibitors have to be present in the cell until the cell actually reaches the S-phase in order to act efficiently. Moreover, the half-life of these topoisomerase inhibitors is short and they have significant side effects, including dose-limiting cardiotoxicity, so their use is limited. However, if dianhydrogalactitol could stall the malignant cells in the S-phase, and the data presented herein suggests that the cells are stalled in the S-phase for at least 72 hours, the topoisomerase inhibitor would be able to attack all the malignant cells in a short period of time. This would diminish the exposure of the cells to the topoisomerase inhibitor and the ensuing side effects, while maximizing the effect of the topoisomerase inhibitor in killing malignant cells.

Accordingly, one aspect of the present invention is a method for use of a combination of dianhydrogalactitol or a derivative or analog thereof together with a topoisomerase inhibitor such that the topoisomerase inhibitor can be administered at a lower concentration or for a shorter period of administration than would be required if the dianhydrogalactitol or the derivative or analog thereof were not administered. Typically, the method for use of the combination reduces side effects associated with administration of the topoisomerase inhibitor.

The topoisomerase inhibitor can be selected from the group consisting of topoisomerase I inhibitors, topoisomerase II inhibitors, and topoisomerase inhibitors having both topoisomerase I inhibitory activity and topoisomerase II inhibitor activity. Topoisomerase inhibitors include the following inhibitors of Type I topoisomerase: irinotecan, topotecan, camptothecin, (S)-10-hydroxycamptothecin, SN-38 ((4S)-4,11-diethyl-4,9-dihydroxy-1H-pyrano[3′,4′:6,7]indolizino[1,2-b]quinoline-3,14(4H,12H)-dione), β-lapachone, and lamellarin. Topoisomerase inhibitors include the following inhibitors of Type II topoisomerase: etoposide, teniposide, doxorubicin, idarubicin, epirubicin, daunorubicin, pirarubicin, mitoxantrone, amsacrine, ellipticines, aurintricarboxylic acid, ICRF 193 (4-[2-(3,5-dioxo-1-piperazinyl)-1-methylpropyl]piperazine-2,6-dione), amonafide, voreloxin, and HU-331 (3-hydroxy-2-[(1R)-6-isopropenyl-3-methyl-cyclohex-2-en-1-yl]-5-pentyl-1,4-benzoquinone). There are additional agents, typically derived from plants, that have activity against both topoisomerase I and topoisomerase II. These agents include epigallocatechin-3-gallate (EGCG), genistein, quercetin, ellagic acid, and resveratrol.

Still other topoisomerase inhibitors are known in the art. These include but are not limited to the following: U.S. Pat. No. 5,272,146 to Haugwitz et al. discloses 1,2-dihydroellipticines with topoisomerase inhibitory activity as well as other ellipticines. These compounds include:

(a) compounds of Formula (T-I):

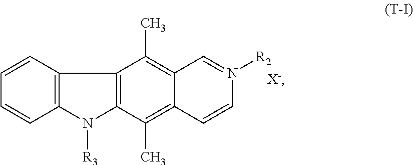

wherein:

(1) R₁ represents a hydrogen atom, a hydroxyl group, an alkoxyl group having 1 to 4 carbon atoms, or an acyloxy group having 2 to 7 carbon atoms;

(2) R₂ represents an aldose residue, a deoxyaldose residue, an N-acylaminoaldose residue, an aldohexuronic amide residue, an aldohexuronic acid residue, an acylated aldose residue, an acylated deoxyaldose residue, an acylated N-acylamino aldose residue, an acylated aldohexuronic amide residue, an acylated aldohexuronic acid residue, an acylated aldohexuronic acid ester residue, an arylalkylated aldose residue, an arylalkylated N-acylaminoaldose residue, an arylalkylated aldohexuronic amide residue, an arylalkylated aldohexuronic acid residue, an arylalkylated aldohexuronic acid ester residue;

(3) R₃ represents a hydrogen atom, a linear, branched, cyclic, or cyclic-linear alkyl group having 1 to 5 carbon atoms;

(4) X⁻ represents a pharmaceutically acceptable inorganic or organic acid anion;

and

(5) the bond represented by N⁺—R₃ in the general formula represents a glycoside bond between a nitrogen atom in the 2-position of the ellipticine and a carbon atom in the 1-position of the sugar;

(b) compounds of Formula (T-II):

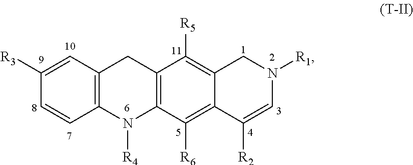

wherein:

(1) R₁ is alkyl having 1 to about 5 carbon atoms, benzyl, alkenyl having 2 to about 5 carbon atoms, alkyloxyalkyl wherein the alkyl portion has 1 to about 5 carbon atoms, hydroxyalkyl having 1 to about 5 carbon atoms, cyanoalkyl having 1 to about 5 carbon atoms, dialkylaminoalkyl wherein each alkyl has 1 to about 5 carbon atoms, glycosyl residue derived from threose, ribose, arabinose, xylose, glucose, mannose, galactose, or acetyl derivatives thereof, acids or alkyl esters selected from the group consisting of —R₇—COOH and —R₇—COOR₈ wherein R₇ is an alkyl having 1 to about 4 carbon atoms and R₈ is an alkyl having 1 to about 5 carbon atoms;

(2) R₂ is hydrogen or formyl;

(3) R₃ is hydrogen, hydroxy, alkyl having 1 to about 5 carbon atoms, alkoxy having 1 to about 5 carbon atoms, phenoxy, benzyloxy, acyloxy, benzoyloxy, fluorine, chlorine, bromine, alkylamino or dialkylamino wherein each alkyl portion has 1 to about 5 carbon atoms or acyloxy having 1 to about 5 carbon atoms;

(4) R₄ is hydrogen, alkyl having 1 to about 5 carbon atoms, formyl, dialkylaminoalkyl wherein each alkyl portion has 1 to about 5 carbon atoms, morpholino N-alkyl or piperidine N-alkyl, wherein the alkyl has 1 to about 5 carbon atoms; and (5) R₅ and R₆ are the same or different and are hydrogen or methyl;

(c) 9-hydroxy-2-methyl-ellipticinium acetate; (d) 9-hydroxy-2-ethyl-ellipticinium acetate; (e) 9-hydroxy-2-hydroxyethyl-ellipticinium acetate; (f) 9-hydroxy-2-hydroxypropyl-ellipticinium acetate; (g) 9-hydroxy-2-dihydroxypropyl-ellipticinium acetate; (h) 9-hydroxy-2(3-diethylamino-ethyl)-ellipticinium acetate; (i) 9-hydroxy-2(3-diisopropylamino-ethyl)-ellipticinium acetate; (j) 9-hydroxy-2(3-piperidino-ethyl)-ellipticinium acetate; (k) 9-methoxy-2-methyl-ellipticinium acetate; (I) 9-acetoxy-2-methyl-ellipticinium acetate; (m) 9-acetoxy-2-ethyl-ellipticinium acetate; (n) 9-benzyloxy-2-methyl-ellipticinium acetate; (o) 9-benzyloxy-2-ethyl-ellipticinium acetate; (p) 9-hydroxy-2;6-dimethyl-ellipticinium acetate; (q) 9-hydroxy-6-methyl-2-ethyl-ellipticinium acetate; (r) 9-hydroxy-6-methyl-2-hydroxyethyl-ellipticinium acetate; (s) 9-hydroxy-2;6-diethyl-ellipticinium acetate; (t) 9-hydroxy-6-ethyl-2-hydroxyethyl-ellipticinium acetate; (u) 9-ethoxy-2;6-diethyl-ellipticinium acetate; (v) 9-ethoxy-6-ethyl-2-(beta-hydroxy-ethyl)-ellipticinium acetate; (w) 9-benzoyloxy-2;6-dimethyl-ellipticinium acetate; and (x) 9-benzoyloxy-6-methyl-2-ethyl-ellipticinium acetate; U.S. Pat. No. 6,509,344 to Cushman et al. discloses indenoisoquinolines as topoisomerase I inhibitors, including compounds of Formula (T-III):

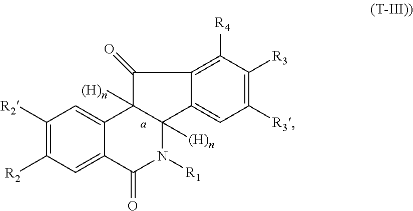

wherein:

(1) R₁ is hydrogen, formyl, phenyl, phenyl substituted with C₁-C₆ alkoxy or C₁-C₆ alkyl, or R₁ is a group —(CH₂)ₘZ, wherein m is 1-6 and Z is selected from the group consisting of hydrogen, hydroxy, carboxy, formyl, C₁-C₆ alkyl, carbo-(C₁-C₆ alkoxy), C₂-C₆ alkenyl, phenyl, C₁-C₆ alkylamino, and C₁-C₆ hydroxyalkylamino;

(2) R₂, R₂′ and R₄ are independently selected from the group consisting of hydrogen, C₁-C₆ alkyl, C2-C₆ alkenyl, C₁-C₆ alkoxy, phenoxy and benzyloxy, or R₂ and R₂′ taken together form a group of the formula —OCH₂O—;

(3) R₃ and R₃′ are independently selected from the group consisting of hydrogen, C₁-C₆ alkyl, C₁-C₆ alkoxy, C₂-C₆ alkenyl, phenoxy, and benzyloxy, or R₃ and R₃′ taken together form a group of the formula —OCH₂O—;

(4) n is 1 or 0, and

(5) bond a is a single bond when n is, and bond a is a double bond when n is 0; provided that when R₂, R₂′, R₄, R₃ and R₃′ are hydrogen, Z is not C₁-C₆ hydroxyalkylamino; and further provided that when R₁ is methyl, R₃ and R_{3′} are independently selected from the group consisting of hydrogen, C₁-C₆ alkyl, C₁-C₆ alkoxy, C₂-C₆ alkenyl, phenoxy, and benzyloxy;

and compounds of Formula (T-IV):

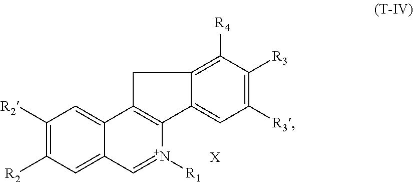

wherein:

(1) R₁ is phenyl or phenyl substituted with C₁-C₆ alkoxy or C₁-C₆ alkyl, or R₁ is a group —(CH₂)ₘZ wherein m is 1-6 and Z is selected from the group consisting of hydrogen, hydroxy, carboxy, formyl, C₁-C₆ alkyl, carbo-(C₁-C₆ alkoxy), C₂-C₆ alkenyl, phenyl, C₁-C₆ alkylamino, and C₁-C₆ hydroxyalkylamino, provided that when Z is hydrogen, m is 2-6;

(2) R₂, R₂′ and R₄ are independently selected from the group consisting of hydrogen, C₁-C₆ alkyl, C₂-C₆ alkenyl, C₁-C₆ alkoxy, phenoxy and benzyloxy, or R₂ and R₂′ taken together form a group of the formula —OCH₂O—;

(3) R₃ and R₃′ are independently selected from the group consisting of hydrogen, C₁-C₆ alkyl, C₁-C₆ alkoxy, C₂-C₆ alkenyl, phenoxy, and benzyloxy, or R₃ and R₃′ taken together form a group of the formula —OCH₂O—; and

(4) X is a pharmaceutically acceptable anion;

particularly preferred topoisomerase inhibitors include 6-(3-carboxy-1-propyl)-5,6-dihydro-5,11-dioxo-11H-indeno[1,2-c]isoquinoline, 6-ethyl-2,3-dimethoxy-8,9-(methylenedioxy)-11H-indeno[1,2-c]isoquinolinium chloride; 6-allyl-5,6-dihydro-2,3-dimethoxy-8,9-(methylenedioxy)-5,11-dioxo-11H-indeno[1,2-c]isoquinoline; and 5,6-dihydro-6-(4-hydoxybut-1-yl)-2,3-dimethoxy-8,9-methylenedioxy-5,11-dioxo-(11H)-indeno[1,2-c]isoquinoline; U.S. Pat. No. 7,312,228 to Cushman et al. discloses benzoisoindoloquinolones and C-11-substituted indenoisoquinolinones that are topoisomerase I inhibitors; U.S. Pat. No. 7,495,100 to Cushman et al. discloses indenoisoquinolines and dihydroindenoisoquinolines with one or more electron-withdrawing substituents that are topoisomerase I inhibitors; U.S. Pat. No. 7,781,445 to Cushman et al. discloses indenoisoquinolinium compounds as topoisomerase I inhibitors; U.S. Pat. No. 8,053,442 to Cushman et al. discloses N-substituted indenoisoquinolines as topoisomerase I inhibitors; U.S. Pat. No. 8,686,146 to Cushman et al. discloses azaindenoisoquinolines as topoisomerase I inhibitors; U.S. Pat. No. 8,912,213 to Cushman et al. discloses N-substituted indenoisoquinolines that have dual activities as topoisomerase I inhibitors and inhibitors of tyrosyl-DNA phosphodiesterase I; U.S. Pat. No. 9,073,920 to Cushman et al. discloses substituted dibenzonaphthyridines as topoisomerase I inhibitors; and U.S. Pat. No. 9,206,193 to Cushman et al. discloses substituted norindenoisoquinoline compounds as topoisomerase I inhibitors.

Additionally, because of the activity of dianhydrogalactitol or a derivative or analog thereof with respect to both the induction of cessation of the cell cycle at the S-phase and the induction of double-strand breaks in DNA, the following additional agents can be used in combination with dianhydrogalactitol or a derivative or analog thereof.

One alternative for such a combination is the use of dianhydrogalactitol or a derivative or analog thereof with an inhibitor of ATM. ATM inhibitors are disclosed in U.S. Pat. No. 7,642,254 to Hummersone et al., including compounds of Formula (ATM-I):

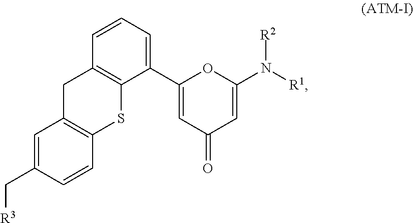

wherein:

(1) R¹ and R² together form, along with the nitrogen atom to which they are attached, an optionally substituted nitrogen containing heterocyclic ring having from 4 to 8 ring atoms; and

(2) R³ is selected from hydroxy and —NR^{N1}R^{N2}, where R^{N1} and R^{N2} are independently selected from hydrogen, optionally substituted C₁-C₇ alkyl groups, optionally substituted C₃-C₂₀ heterocyclyl groups and optionally substituted C₃-C₂₀ aryl groups, or together form, along with the nitrogen atom to which they are attached, an optionally substituted nitrogen containing heterocyclic ring having from 4 to 8 ring atoms.

Additional ATM inhibitors are disclosed in U.S. Pat. No. 7,429,660 by Smith et al. These include compounds of Formula (ATM-II):

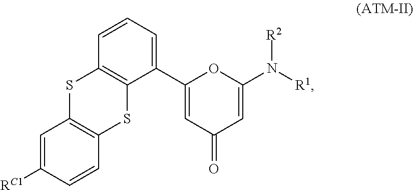

wherein:

(1) R¹ and R² together form, along with the nitrogen atom to which they are attached, an optionally substituted heterocyclic ring having from 4 to 8 ring atoms;

(2) R^{C1} is —NR³R⁴, where R³ and R⁴ are independently selected from hydrogen, optionally substituted C₁-C₇ alkyl groups, optionally substituted C₃-C₂₀ heterocyclyl groups and optionally substituted C₅-C₂₀ aryl groups, or together form, along with the nitrogen atom to which they are attached, an optionally substituted heterocyclic ring having from 4 to 8 ring atoms, or R^{C1} is of Formula (ATM-II(a)):

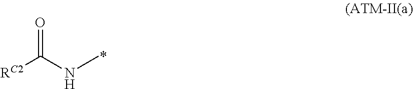

wherein R^{C2} is selected from an optionally substituted C₁-C₇ alkyl group, an optionally substituted C₃-C₂₀ heterocyclyl group, an optionally substituted C₅-C₂₀ aryl group, an ester group, an ether group and an amino group.

Another alternative for such a combination is the use of dianhydrogalactitol or a derivative or analog thereof with an inhibitor of ATR kinase. ATR inhibitors are disclosed in U.S. Pat. No. 9,096,584 to Charrier (pyridine compounds); U.S. Pat. No. 9,062,008 to Charrier et al. (pyrazine and pyridine compounds); U.S. Pat. No. 8,969,360 to Charrier et al. (pyrazolopyrimidine carboxamide compounds); U.S. Pat. No. 8,969,356 to Charrier et al. (pyrazine and pyridine compounds); U.S. Pat. No. 8,962,631 to Charrier et al. (pyrazine compounds); U.S. Pat. No. 8,957,078 to Brenchley et al. (pyrazolopyrimidine carboxylate compounds); U.S. Pat. No. 8,912,198 to Charrier et al. (aminopyrazine compounds); U.S. Pat. No. 8,877,759 to Charier et al. (aminopyrazine compounds); U.S. Pat. No. 8,846,918 to Charrier et al. (aminopyrazine compounds); U.S. Pat. No. 8,846,917 to Charrier et al. (pyrazinyl carbamate compounds); U.S. Pat. No. 8,846,686 to Charrier et al. (aminopyrazine compounds); U.S. Pat. No. 8,841,450 to Charrier et al. (aminopyrazine compounds); U.S. Pat. No. 8,841,449 to Charrier et al. (piperidinyl carboxylate compounds); U.S. Pat. No. 8,841,337 to Charrier et al. (aminopyrazine compounds); U.S. Pat. No. 8,841,308 to Charrier et al. U.S. Pat. No. 8,841,308 (pyrazine-2-amines); U.S. Pat. No. 8,822,469 to MacCormick et al. (pyrrolo[2,3-b]pyrazines); U.S. Pat. No. 8,765,751 to Charrier et al. (pyrazine-2-amines); U.S. Pat. No. 8,623,869 to Charrier et al. (pyrrolopyrazine compounds); and U.S. Pat. No. 8,410,122 to Charrier et al. (imidazolylpyrazine compounds).

The following additional proteins or factors are known to participate in homologous recombination.

Mre11 (MRE11A in humans) is encoded by MRE11 A and forms part of a complex with Rad50 and Nbs1; the protein has 3′ to 5′ exonuclease activity and endonuclease activity (J. H. Petrini et al., “Isolation and Characterization of the Human MRE11 Homologue,” Genomics 29: 80-86 (1996)).

Rad50 is encoded by RAD50 in humans and forms a complex with MRE11 and NBS; this MRN complex binds to broken DNA ends and displays numerous enzymatic activities that are associated with double-strand-break repair, including homologous recombination (E. Kinoshita et al., “RAD50, an SMC Family Member with Multiple Roles in Break Repair: How Does ATP Affect Function,” Chromosome Res. 17: 277-288 (2009)).

NBS1 or nibrin is another protein involved in the complex and involved in DNA repair (R. Varon et al., “Nibrin, a novel DNA Double-Strand Break Repair protein, Is Mutated in Nijmegen Breakage Syndrome,” Cell 93: 467-476 (1998)).

CtIP (choline transporter-like protein) is encoded by SLC44A1 and is also involved in DNA repair (Z. Yuan et al., “Genomic Organization, Promoter Activity, and Expression of the Human Choline Transporter-Like Protein 1,” Physiol. Genomics 26: 76-90 (2007)).

RPA (replication protein A) binds to single-stranded DNA during early stages of homologous recombination (Y. Zhu et al., “Functions of Human Replication Protein A (RPA): From DNA Replication to DNA Damage and Stress Responses,” J. Cell. Physiol. 208: 267-273 (2006)). The protein is a heterotrimer.

RAD51 is encoded by RAD51. This protein is involved in strand base-pairing and assists in repair of double-strand breaks (A. Shinohara et al., “Rad51 Protein Involved in Repair and Recombination in S. cerevisiae Is a RecA-Like Protein,” Cell 69: 457-470 (1992)).

Other proteins or factors involved in DNA repair via homologous recombination include HRP2 and LEDGF.

Therefore, an additional aspect of the present invention is use of an inhibitor of one or more of these proteins or factors involved in DNA repair via homologous recombination in combination with dianhydrogalactitol or a derivative or analog thereof.

As stated below in the Example, the phosphorylated histone variant H2A.X (γH2A.X) is an indicator of activation of the DNA damage pathways. Therefore, analysis of the quantity of γH2A.X can be used to determine the extent of the DNA damage response and can be used either to determine dosage of dianhydrogalactitol or a derivative or analog thereof or to determine sensitivity or resistance of the cells to dianhydrogalactitol or a derivative or analog thereof.

Similarly, analysis of phosphorylation of the serine 1981 amino acid residue in the ATM protein or of the serine 33 amino acid residue in the RPA32 protein can be used to determine the degree of activation of the DNA repair pathways. Therefore, analysis of the degree of phosphorylation of ATM (S1981) or RPA32 (S33) can be used to determine the extent of the DNA damage response and can be used either to determine dosage of dianhydrogalactitol or a derivative or analog thereof or to determine sensitivity or resistance of the cells to dianhydrogalactitol or a derivative or analog thereof.

Another aspect of the present invention is the use of substituted hexitol derivatives as described above for leptomeningeal disease. Leptomeningeal carcinomatosis (LC) is a complication of cancer in which the disease spreads to the meninges surrounding the brain and spinal cord. LC occurs in approximately 5% of people with cancer and is usually terminal. If left untreated, median survival is 4-6 weeks; if treated, median survival is 2-3 months. Meningeal symptoms are the first manifestations in some patients (pain and seizures are the most common presenting complaints) and can include the following: headaches, which are usually associated with nausea, vomiting, or light-headedness; gait difficulties from weakness or ataxia; memory problems; incontinence; or sensory abnormalities. LC is generally considered difficult to treat and generally incurable. The standard therapies are (1) radiation therapy to symptomatic sites and regions where imaging has demonstrated bulk disease and (2) intrathecal chemotherapy. Radiation palliates local symptoms, relieves CSF flow obstruction, and treats areas such as nerve-root sleeves, Virchow-Robin spaces, and the interior of bulky lesions that chemotherapy does not reach. Intrathecal chemotherapy treats subclinical leptomeningeal deposits and tumor cells floating in the CSF, preventing further seeding. Cytarabine (Ara-C), methotrexate (MTX), and thiotepa are three agents routinely administered. Supportive care for patients includes analgesia with opioids, anticonvulsants for seizures, antidepressants, and anxiolytics. Attention problems and somnolence from whole-brain radiation can be treated with psychostimulants or modafinil.

U.S. Pat. No. 9,066,979 to Li et al., discloses quinazoline inhibitors of activating mutant forms of EGFR, including 4-[(3-chloro-2-fluorophenyl)amino]-7-methoxyquinazolin-6-yl (2R)-2,4-dimethylpiperazine-1-carboxylate for treating LC.

U.S. Pat. No. 9,017,938 to Zollo, discloses the use of microRNA 199b-5p in anti-cancer therapy for medulloblastoma and LC.

U.S. Pat. No. 9,005,900 to Ring et al., discloses methotrexate as therapy for LC.

U.S. Pat. No. 9,000,179 to Priebe et al., discloses the use of interleukin-2 as therapy for leptomeningeal cancer, together with pyridine STAT3 and STAT5 modulators.

U.S. Pat. No. 8,993,758 to Natarajan et al., discloses substituted quinoxalines for inhibiting IKKβ and the NFκB and mTOR pathways for treating leptomeningeal cancer.

U.S. Pat. No. 8,986,690 to Nykjaer et al., discloses rituximab for treating leptomeningeal cancer.

U.S. Pat. No. 8,834,921 to Kim et al., discloses cytarabine for treating leptomeningeal cancer.

U.S. Pat. No. 8,377,985 to Kun et al., discloses irinotecan for treating leptomeningeal cancer.

U.S. Pat. No. 8,202,860 to Stendel et al., discloses methylol transfer agents such as taurolidine or taurultam for treating leptomeningeal cancer.

U.S. Pat. No. 7,422,741 to Alitalo et al., discloses VEGFR-3 fusion proteins for treatment of leptomeningeal cancer.

U.S. Pat. No. 6,815,441 to Stendel et al., discloses the use of reaction products of taurultam with glucose for treatment of leptomeningeal cancer.

U.S. Pat. No. 6,251,886 to Friedman, discloses the use of temozolomide for treatment of leptomeningeal cancer; the temozolomide can be in a microcrystalline suspension.

U.S. Pat. No. 5,407,925 to Bigner et al., discloses use of 4-hydroperoxycyclophosphamide for treatment of leptomeningeal cancer.

U.S. Pat. No. 4,590,001 to Stjernholm, discloses the use of platinum-transferrin for treatment of leptomeningeal cancer.

United States Patent Application Publication No. 2015/0157744 by Johnson et al., discloses use of phenylbenzothiazole, stilbene, biphenylalkyne, or pyridine derivatives for treatment of leptomeningeal cancer.

United States Patent Application Publication No. 2014/0271540 by Stogniew et al., discloses use of 7-benzyl-10-(2-methylbenzyl)-2,6,7,8,9,10-hexahydroimidazo[1,2-a]pyrido[4,3-d]pyrimidin-5(3H)-one for treatment of leptomeningeal cancer.

United States Patent Application Publication No. 2013/0274281 by Bradley et al., discloses use of 4-iodo-3-nitrobenzamide and irinotecan for treatment of leptomeningeal cancer.

United States Patent Application Publication No. 2013/0129675 by Priebe et al., discloses administration of interferon-α or interferon-β in combination with a STAT3 inhibitor for treatment of leptomeningeal cancer.

United States Patent Application Publication No. 2013/0122056 by Zhang et al., discloses nanoparticles and multi-drug conjugates with stimuli-sensitive linkers for treatment of leptomeningeal cancer.

United States Patent Application Publication No. 2012/0269867 by Jimenez et al., discloses treatment with coenzyme Q10 for leptomeningeal cancer.

United States Patent Application Publication No. 2012/0171118 by Papisov, discloses conjugates for administration of therapeutic agents to leptomeningeal tissue for treatment of leptomeningeal cancer.

United States Patent Application Publication No. 2012/0149888 by Srivastava et al., discloses use of arabino-2′-O-methyl nucleosides and their phosphoramidites; the nucleosides, phosphates, and triphosphates as therapeutic agents for treatment of leptomeningeal cancer.

United States Patent Application Publication No. 2010/0285046 by Tumer et al., discloses ricin mutants for treatment of malignancies, including leptomeningeal cancer.

United States Patent Application Publication No. 2010/0048542 by Stendel et al., discloses use of taurolidine, taurultam, a combination of taurolidine and taurultam, methylol taurinamide, methylol-taurultam, an aminoglycan of taurultam, or taurultam-glucose for treatment of leptomeningeal cancer.

United States Patent Application Publication No. 2009/0258832 by Power et al., discloses use of interferon-3 to treat leptomeningeal metastases.

United States Patent Application Publication No. 2009/0005398 by Dar, discloses use of benzimidazole thiophene compounds for treatment of leptomeningeal cancer.

United States Patent Application Publication No. 2003/0082229 by Anderson et al., discloses use of chlorambucil for treatment of leptomeningeal cancer.

United States Patent Application Publication No. 2002/0128228 by Hwu et al., discloses use of thalidomide and temozolomide to treat leptomeningeal cancer. Other analogs of thalidomide, such as lenalidomide, can be used.

United States Patent Application Publication No. 2002/0009444 by Grillo-Lopez et al., discloses use of intrathecal rituximab for treatment of leptomeningeal cancer.

The dianhydrogalactitol or derivative or analog thereof can also be used together with a topoisomerase inhibitor or an agent inhibiting the activity of one or more proteins involved in DNA repair as described above for the treatment of leptomeningeal cancer.

Another aspect of the present invention is a composition to improve the efficacy and/or reduce the side effects of suboptimally administered drug therapy employing a substituted hexitol as described above for the treatment of glioblastoma, non-small-cell lung carcinoma, or ovarian cancer comprising an alternative selected from the group consisting of:

(i) a therapeutically effective quantity of a modified hexitol derivative or a derivative, analog, or prodrug of a hexitol derivative or a modified hexitol derivative, wherein the modified hexitol derivative or the derivative, analog or prodrug of the modified hexitol derivative possesses increased therapeutic efficacy or reduced side effects for treatment of glioblastoma, non-small-cell lung carcinoma, or ovarian cancer as compared with an unmodified hexitol derivative;

(ii) a composition comprising:
(a) a therapeutically effective quantity of a hexitol derivative, a modified hexitol derivative, or a derivative, analog, or prodrug of a hexitol derivative or a modified hexitol derivative; and
(b) at least one additional therapeutic agent, therapeutic agent subject to chemosensitization, therapeutic agent subject to chemopotentiation, diluent, excipient, solvent system, drug delivery system, agent for counteracting myelosuppression, or agent for increasing the ability of the hexitol derivative, the modified hexitol derivative, or the derivative, analog, or prodrug of the hexitol derivative or the modified hexitol derivative to pass through the blood-brain barrier, wherein the composition possesses increased therapeutic efficacy or reduced side effects for treatment of glioblastoma, non-small-cell lung carcinoma, or ovarian cancer as compared with an unmodified hexitol derivative;

(iii) a therapeutically effective quantity of a hexitol derivative, a modified hexitol derivative, or a derivative, analog, or prodrug of a hexitol derivative or a modified hexitol derivative that is incorporated into a dosage form, wherein a hexitol derivative, a modified hexitol derivative, or a derivative, analog, or prodrug of a hexitol derivative or a modified hexitol derivative incorporated into the dosage form possesses increased therapeutic efficacy or reduced side effects for treatment of glioblastoma, non-small-cell lung carcinoma, or ovarian cancer as compared with an unmodified hexitol derivative;

(iv) a therapeutically effective quantity of a hexitol derivative, a modified hexitol derivative, or a derivative, analog, or prodrug of an hexitol derivative or a modified hexitol derivative that is incorporated into a dosage kit and packaging, wherein a hexitol derivative, a modified hexitol derivative, or a derivative, analog, or prodrug of a hexitol derivative or a modified hexitol derivative incorporated into the dosage kit and packaging possesses increased therapeutic efficacy or reduced side effects for treatment of glioblastoma, non-small-cell lung carcinoma, or ovarian cancer as compared with an unmodified hexitol derivative; and

(v) a therapeutically effective quantity of a hexitol derivative, a modified hexitol derivative, or a derivative, analog, or prodrug of a hexitol derivative or a modified hexitol derivative that is subjected to a bulk drug product improvement, wherein the hexitol derivative, the modified hexitol derivative, or the derivative, analog, or prodrug of the hexitol derivative or the modified hexitol derivative subject to the bulk drug product improvement possesses increased therapeutic efficacy or reduced side effects for treatment of glioblastoma, non-small-cell lung carcinoma, or ovarian cancer as compared with an unmodified alkylating hexitol derivative.

As described above, the alkylating hexitol derivative can be, but is not limited to, dianhydrogalactitol, a derivative or analog of dianhydrogalactitol, diacetyldianhydrogalactitol, or a derivative or analog of diacetyldianhydrogalactitol.

In one alternative, the pharmaceutical composition is formulated to exert a cytotoxic effect against cancer stem cells.

In one alternative, the composition comprises a drug combination comprising:

(i) an alkylating hexitol derivative, a modified alkylating hexitol derivative, or a derivative, analog, or prodrug of an alkylating hexitol derivative or a modified alkylating hexitol derivative; and

(ii) an additional therapeutic agent selected from the group consisting of:
(a) topoisomerase inhibitors;
(b) fraudulent nucleosides;
(c) fraudulent nucleotides;
(d) thymidylate synthetase inhibitors;
(e) signal transduction inhibitors;
(f) cisplatin or platinum analogs;
(g) alkylating agents;
(h) anti-tubulin agents;
(i) antimetabolites;
(j) berberine;
(k) apigenin;
(l) amonafide;
(m) vinca alkaloids;
(n) 5-fluorouracil;
(o) curcumin;
(p) NF-κB inhibitors;
(q) rosmarinic acid;
(r) mitoguazone;
(s) tetrandrine;
(t) an ATM inhibitor; and
(u) an ATR inhibitor.

In these alternatives, when the additional therapeutic agent is an alkylating agent, the alkylating agent can be, but is not limited to, an alkylating agent selected from the group consisting of BCNU, BCNU wafers, CCNU, bendamustine (Treanda), and temozolimide (Temodar). In another alternative, the drug composition comprises one or more additional agents that are described above with respect to methods according to the present invention employing drug combinations. In drug combinations according to the present invention, both the alkylating hexitol derivative and the additional agent are present in a therapeutically effective quantity. More than one additional agent can be present in a drug combination according to the present invention, subject to the condition that the at least one additional agent does not interact deleteriously with either the alkylating hexitol derivative present in the composition or other additional agent or agents present in the composition. For example, and not by way of limitation, the composition can comprise, as additional agents: (i) a topoisomerase inhibitor; and (ii) an inhibitor of CHK1 kinase or CHK2 kinase.

Topoisomerase inhibitors, ATM inhibitors, and ATR inhibitors suitable for use in compositions according to the present invention are as described above.

In another alternative, the composition comprises:

(i) an alkylating hexitol derivative, a modified alkylating hexitol derivative, or a derivative, analog, or prodrug of an alkylating hexitol derivative or a modified alkylating hexitol derivative; and

(ii) a therapeutic agent subject to chemosensitization selected from the group consisting of:
(a) topoisomerase inhibitors;
(b) fraudulent nucleosides;
(c) fraudulent nucleotides;
(d) thymidylate synthetase inhibitors;
(e) signal transduction inhibitors;
(f) cisplatin or platinum analogs;
(g) alkylating agents;
(h) anti-tubulin agents;
(i) antimetabolites;
(j) berberine;
(k) apigenin;
(l) colchicine or an analog of colchicine;
(m) genistein;
(n) etoposide;
(o) cytarabine;
(p) camptothecin;
(q) vinca alkaloids;
(r) 5-fluorouracil;
(s) curcumin;
(t) NF-κB inhibitors;
(u) rosmarinic acid; and
(v) mitoguazone;
   wherein the alkylating hexitol derivative, a modified alkylating hexitol derivative, or a derivative, analog, or prodrug of an alkylating hexitol derivative or a modified alkylating hexitol derivative acts as a chemosensitizer.

In still another alternative, the composition comprises:

(i) an alkylating hexitol derivative, a modified alkylating hexitol derivative, or a derivative, analog, or prodrug of an alkylating hexitol derivative or a modified alkylating hexitol derivative; and

(ii) a therapeutic agent subject to chemopotentiation selected from the group consisting of:
(a) fraudulent nucleosides;
(b) fraudulent nucleotides;
(c) thymidylate synthetase inhibitors;
(d) signal transduction inhibitors;
(e) cisplatin or platinum analogs;
(f) alkylating agents;
(g) anti-tubulin agents;
(h) antimetabolites;
(i) berberine;
(j) apigenin;
(k) colchicine or analogs of colchicine;
(l) genistein;
(m) etoposide;
(n) cytarabine;
(o) camptothecins;
(p) vinca alkaloids;
(q) topoisomerase inhibitors;
(r) 5-fluorouracil;
(s) curcumin;
(t) NF-κB inhibitors;
(u) rosmarinic acid;
(v) mitoguazone; and
(w) a biotherapeutic;
   wherein the alkylating hexitol derivative, a modified alkylating hexitol derivative, or a derivative, analog, or prodrug of an alkylating hexitol derivative or a modified alkylating hexitol derivative acts as a chemopotentiator.

In these alternatives, wherein the additional therapeutic agent is a biotherapeutic, the biotherapeutic can be, but is not limited to, a biotherapeutic selected from the group consisting of Avastin, Herceptin, Rituxan, and Erbitux.

In yet another alternative, the alkylating hexitol derivative, a modified alkylating hexitol derivative, or a derivative, analog, or prodrug of the alkylating hexitol derivative or the modified alkylating hexitol derivative of the composition is subjected to a bulk drug product improvement, wherein the bulk drug product improvement is selected from the group consisting of:

(a) salt formation;

(b) preparation as a homogeneous crystal structure;

(c) preparation as a pure isomer;

(d) increased purity;

(e) preparation with lower residual solvent content; and

(f) preparation with lower residual heavy metal content.

In still another alternative, the composition comprises an alkylating hexitol derivative, a modified alkylating hexitol derivative, or a derivative, analog, or prodrug of an alkylating hexitol derivative or a modified alkylating hexitol derivative and a diluent, wherein the diluent is selected from the group consisting of:

(a) an emulsion;

(b) dimethylsulfoxide (DMSO);

(c) N-methylformamide (NMF)

(d) dimethylformamide (DMF)

(e) dimethylacetamide (DMA);

(f) ethanol;

(g) benzyl alcohol;

(h) dextrose-containing water for injection;

(i) Cremophor;

(j) cyclodextrins; and

(k) PEG.

In still another alternative, the composition comprises an alkylating hexitol derivative, a modified alkylating hexitol derivative, or a derivative, analog, or prodrug of an alkylating hexitol derivative or a modified alkylating hexitol derivative and a solvent system, wherein the solvent system is selected from the group consisting of:

(a) an emulsion;

(b) DMSO;

(c) NMF;

(d) DMF;

(e) DMA;

(f) ethanol;

(g) benzyl alcohol;

(h) dextrose-containing water for injection;

(i) Cremophor;

(j) PEG; and

(k) salt systems.

In yet another alternative, the composition comprises an alkylating hexitol derivative, a modified alkylating hexitol derivative, or a derivative, analog, or prodrug of an alkylating hexitol derivative or a modified alkylating hexitol derivative and an excipient, wherein the excipient is selected from the group consisting of:

(a) mannitol;

(b) albumin;

(c) EDTA;

(d) sodium bisulfite;

(e) benzyl alcohol;

(f) carbonate buffers;

(g) phosphate buffers;

(h) PEG;

(i) vitamin A;

(j) vitamin D;

(k) vitamin E;

(l) esterase inhibitors;

(m) cytochrome P450 inhibitors;

(n) multi-drug resistance (MDR) inhibitors;

(o) organic resins;

(p) detergents;

(q) perillyl alcohol or an analog thereof; and

(r) activators of channel-forming receptors.

In still another alternative, the alkylating hexitol derivative, modified alkylating hexitol derivative, or derivative, analog, or prodrug of the alkylating hexitol derivative or modified alkylating hexitol derivative is incorporated into a dosage form selected from the group consisting of:

(a) tablets;

(b) capsules;

(c) topical gels;

(d) topical creams;

(e) patches;

(f) suppositories;

(g) lyophilized dosage fills;

(h) immediate-release formulations;

(i) slow-release formulations;

(j) controlled-release formulations; and

(k) liquid in capsules.

In still another alternative, the alkylating hexitol derivative, modified alkylating hexitol derivative, or derivative, analog, or prodrug of an alkylating hexitol derivative or modified alkylating hexitol derivative is incorporated into a dosage kit and packaging selected from the group consisting of amber vials to protect from light and stoppers with specialized coatings to improve shelf-life stability.

In still another alternative, the composition comprises: (i) an alkylating hexitol derivative, modified alkylating hexitol derivative, or derivative, analog, or prodrug of an alkylating hexitol derivative or modified alkylating hexitol derivative; and (ii) a drug delivery system, wherein the drug delivery system is selected from the group consisting of:

(a) oral dosage forms;

(b) nanocrystals;

(c) nanoparticles;

(d) cosolvents;

(e) slurries;

(f) syrups;

(g) bioerodible polymers;

(h) liposomes;

(i) slow-release injectable gels;

(j) microspheres; and

(k) targeting compositions with epidermal growth factor receptor-binding peptides.

In still another alternative of a composition according to the present invention, the therapeutic agent is a modified alkylating hexitol derivative, and the modification is selected from the group consisting of:

(a) alteration of side chains to increase or decrease lipophilicity;

(b) addition of an additional chemical functionality to alter a property selected from the group consisting of reactivity, electron affinity, and binding capacity; and

(c) alteration of salt form.

In still another alternative of a composition according to the present invention, the therapeutic agent is an alkylating hexitol derivative, modified alkylating hexitol derivative, or derivative or analog of an alkylating hexitol derivative or modified alkylating hexitol derivative and the therapeutic agent is present in the composition in a drug conjugate form, wherein the drug conjugate form is a drug conjugate form selected from the group consisting of:

(a) a polymer system;

(b) polylactides;

(c) polyglycolides;

(d) amino acids;

(e) peptides;

(f) multivalent linkers;

(g) immunoglobulins;

(h) cyclodextrin polymers;

(i) modified transferrin;

(j) hydrophobic or hydrophobic-hydrophilic polymers;

(k) conjugates with a phosphonoformic acid partial ester;

(l) conjugates with a cell-binding agent incorporating a charged cross-linker; and

(m) conjugates with β-glucuronides through a linker.

In still another alternative of a composition according to the present invention, the therapeutic agent is an alkylating hexitol derivative, modified alkylating hexitol derivative, or derivative or analog of an alkylating hexitol derivative or modified alkylating hexitol derivative and the therapeutic agent is in the form of a prodrug system, wherein the prodrug system is selected from the group consisting of:

(a) enzyme sensitive esters;

(b) dimers;

(c) Schiff bases;

(d) pyridoxal complexes;

(e) caffeine complexes;

(f) nitric oxide-releasing prodrugs;

(g) prodrugs with fibroblast activation protein α-cleavable oligopeptides;

(h) products of reaction with an acetylating or carbamylating agent;

(i) hexanoate conjugates;

(j) polymer-agent conjugates; and

(k) prodrugs that are subject to redox activation.

In still another alternative of a composition according to the present invention, the therapeutic agent is an alkylating hexitol derivative, modified alkylating hexitol derivative, or derivative, analog, or prodrug of an alkylating hexitol derivative or modified alkylating hexitol derivative and the composition further comprises at least one additional therapeutic agent to form a multiple drug system, wherein at least one additional therapeutic agent is selected from the group consisting of:

(a) an inhibitor of multi-drug resistance;

(b) a specific drug resistance inhibitor;

(c) a specific inhibitor of a selective enzyme;

(d) a signal transduction inhibitor;

(e) an inhibitor of a repair enzyme; and

(f) a topoisomerase inhibitor with non-overlapping side effects.

In still another alternative of a composition according to the present invention, the therapeutic agent is an alkylating hexitol derivative, modified alkylating hexitol derivative, or derivative, analog, or prodrug of an alkylating hexitol derivative or modified alkylating hexitol derivative and the composition further comprises an agent for counteracting myelosuppression. Typically, the agent that counteracts myelosuppression is a dithiocarbamate.

In still another alternative of a composition according to the present invention, the therapeutic agent is an alkylating hexitol derivative, modified alkylating hexitol derivative, or derivative, analog, or prodrug of an alkylating hexitol derivative or modified alkylating hexitol derivative and the composition further comprises an agent that increases the ability of the substituted hexitol to pass through the blood-brain barrier, wherein the agent that increases the ability of the substituted hexitol to pass through the blood-brain barrier is selected from the group consisting of:

(a) a chimeric peptide of the structure of Formula (D-III):

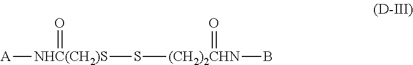

wherein: (A) A is somatostatin, thyrotropin releasing hormone (TRH), vasopressin, alpha interferon, endorphin, muramyl dipeptide or ACTH 4-9 analogue; and (B) B is insulin, IGF-I, IGF-II, transferrin, cationized (basic) albumin or prolactin; or a chimeric peptide of the structure of Formula (D-III) wherein the disulfide conjugating bridge between A and B is replaced with a bridge of Subformula (D-III(a)):

A-NH(CH₂)₂S—S—B (cleavable linkage) (D-III(a)),

wherein the bridge is formed using cysteamine and EDAC as the bridge reagents; or a chimeric peptide of the structure of Formula (D-III) wherein the disulfide conjugating bridge between A and B is replaced with a bridge of Subformula (D-III(b)):

A-NH═CH(CH₂)₃CH═NH—B (non-cleavable linkage) (D-III(b)),

wherein the bridge is formed using glutaraldehyde as the bridge reagent;

(b) a composition comprising either avidin or an avidin fusion protein bonded to a biotinylated substituted hexitol derivative to form an avidin-biotin-agent complex including therein a protein selected from the group consisting of insulin, transferrin, an anti-receptor monoclonal antibody, a cationized protein, and a lectin;

(c) a neutral liposome that is pegylated and incorporates the substituted hexitol derivative, wherein the polyethylene glycol strands are conjugated to at least one transportable peptide or targeting agent;

(d) a humanized murine antibody that binds to the human insulin receptor linked to the substituted hexitol derivative through an avidin-biotin linkage; and

(e) a fusion protein comprising a first segment and a second segment: the first segment comprising a variable region of an antibody that recognizes an antigen on the surface of a cell that after binding to the variable region of the antibody undergoes antibody-receptor-mediated endocytosis, and, optionally, further comprises at least one domain of a constant region of an antibody; and the second segment comprising a protein domain selected from the group consisting of avidin, an avidin mutein, a chemically modified avidin derivative, streptavidin, a streptavidin mutein, and a chemically modified streptavidin derivative, wherein the fusion protein is linked to the substituted hexitol by a covalent link to biotin.

In one alternative, when the alkylating hexitol derivative is dianhydrogalactitol, the composition is formulated for administration of dianhydrogalactitol by dosing once daily for three consecutive days every 21 days.

Compositions according to the present invention can be formulated for the treatment of leptomeningeal carcinomatosis; such compositions can include the various alternatives described above. When the composition is formulated for treatment of leptomeningeal carcinomatosis, the composition can further comprise a therapeutically effective quantity of an additional therapeutic agent for treatment of leptomeningeal carcinomatosis, including, but not limited to, cytarabine, methotrexate, thiotepa, 4-[(3-chloro-2-fluorophenyl)amino]-7-methoxyquinazolin-6-yl (2R)-2,4-dimethylpiperazine-1-carboxylate, microRNA 199b-5p, interleukin-2, a pyridine STAT3/STAT5 modulator, a substituted quinoxaline inhibitor of inhibiting IKKβ and the NFκB and mTOR pathways, rituximab, irinotecan, taurolidine, taurultam, VEGFR-3 fusion proteins, a reaction product of taurultam with glucose, temozolomide, 4-hydroperoxycyclophosphamide, platinum-transferrin, phenylbenzothiazole, stilbene, biphenylalkyne, pyridine derivatives, 7-benzyl-10-(2-methylbenzyl)-2,6,7,8,9,10-hexahydroimidazo[1,2-a]pyrido[4,3-d]pyrimidin-5(3H)-one, 4-iodo-3-nitrobenzamide, interferon-α, interferon-β, a STAT3 inhibitor, coenzyme Q10, arabino-2′-O-methyl nucleosides and derivatives thereof, ricin mutants, methylol taurinamide, methylol-taurultam, an aminoglycan of taurultam, benzimidazole thiophene compounds, chlorambucil, temozolomide, thalidomide, and lenalidomide.

When a pharmaceutical composition according to the present invention includes a prodrug, prodrugs and active metabolites of a compound may be identified using routine techniques known in the art. See, e.g., Bertolini et al., J. Med. Chem., 40, 2011-2016 (1997); Shan et al., J. Pharm. Sci., 86 (7), 765-767; Bagshawe, Drug Dev. Res., 34, 220-230 (1995); Bodor, Advances in Drug Res., 13, 224-331 (1984); Bundgaard, Design of Prodrugs (Elsevier Press 1985); Larsen, Design and Application of Prodrugs, Drug Design and Development (Krogsgaard-Larsen et al., eds., Harwood Academic Publishers, 1991); Dear et al., J. Chromatogr. B, 748, 281-293 (2000); Spraul et al., J. Pharmaceutical & Biomedical Analysis, 10, 601-605 (1992); and Prox et al., Xenobiol., 3, 103-112 (1992).

When the pharmacologically active compound in a pharmaceutical composition according to the present invention possesses a sufficiently acidic, a sufficiently basic, or both a sufficiently acidic and a sufficiently basic functional group, these group or groups can accordingly react with any of a number of inorganic or organic bases, and inorganic and organic acids, to form a pharmaceutically acceptable salt. Exemplary pharmaceutically acceptable salts include those salts prepared by reaction of the pharmacologically active compound with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, 213rabic213, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, p-hydroxybutyrates, glycolates, tartrates, methane-sulfonates, propanesulfonates, naphthalene-1-sulfonates, naphthalene-2-sulfonates, and mandelates. If the pharmacologically active compound has one or more basic functional groups, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like. If the pharmacologically active compound has one or more acidic functional groups, the desired pharmaceutically acceptable salt may be prepared by any suitable method available in the art, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

In the case of agents that are solids, it is understood by those skilled in the art that the inventive compounds and salts may exist in different crystal or polymorphic forms, all of which are intended to be within the scope of the present invention and specified formulas.

The amount of a given pharmacologically active agent, such as dianhydrogalactitol or an analog or derivative of dianhydrogalactitol as described above, that is included in a unit dose of a pharmaceutical composition according to the present invention will vary depending upon factors such as the particular compound, disease condition and its severity, the identity (e.g., weight) of the subject in need of treatment, but can nevertheless be routinely determined by one skilled in the art. Typically, such pharmaceutical compositions include a therapeutically effective quantity of the pharmacologically active agent and an inert pharmaceutically acceptable carrier or diluent. Typically, these compositions are prepared in unit dosage form appropriate for the chosen route of administration, such as oral administration or parenteral administration. A pharmacologically active agent as described above can be administered in conventional dosage form prepared by combining a therapeutically effective amount of such a pharmacologically active agent as an active ingredient with appropriate pharmaceutical carriers or diluents according to conventional procedures. These procedures may involve mixing, granulating and compressing or dissolving the ingredients as appropriate to the desired preparation. The pharmaceutical carrier employed may be either a solid or liquid. Exemplary of solid carriers are lactose, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time-delay or time-release material known in the art, such as glyceryl monostearate or glyceryl distearate alone or with a wax, ethylcellulose, hydroxypropylmethylcellulose, methylmethacrylate and the like.

A variety of pharmaceutical forms can be employed. Thus, if a solid carrier is used, the preparation can be tableted, placed in a hard gelatin capsule in powder or pellet form or in the form of a troche or lozenge. The amount of solid carrier may vary, but generally will be from about 25 mg to about 1 g. If a liquid carrier is used, the preparation will be in the form of syrup, emulsion, soft gelatin capsule, sterile injectable solution or suspension in an ampoule or vial or non-aqueous liquid suspension.

To obtain a stable water-soluble dose form, a pharmaceutically acceptable salt of a pharmacologically active agent as described above is dissolved in an aqueous solution of an organic or inorganic acid, such as 0.3 M solution of succinic acid or citric acid. If a soluble salt form is not available, the agent may be dissolved in a suitable cosolvent or combinations of cosolvents. Examples of suitable cosolvents include, but are not limited to, alcohol, propylene glycol, polyethylene glycol 300, polysorbate 80, glycerin and the like in concentrations ranging from 0-60% of the total volume. In an exemplary embodiment, a compound of Formula I is dissolved in DMSO and diluted with water. The composition may also be in the form of a solution of a salt form of the active ingredient in an appropriate aqueous vehicle such as water or isotonic saline or dextrose solution.

It will be appreciated that the actual dosages of the agents used in the compositions of this invention will vary according to the particular complex being used, the particular composition formulated, the mode of administration and the particular site, host and disease and/or condition being treated. Actual dosage levels of the active ingredients in the pharmaceutical compositions of the present invention can be varied so as to obtain an amount of the active ingredient which is effective to achieve the desired therapeutic response for a particular subject, composition, and mode of administration, without being toxic to the subject. The selected dosage level depends upon a variety of pharmacokinetic factors including the activity of the particular therapeutic agent, the route of administration, the time of administration, the rate of excretion of the particular compound being employed, the severity of the condition, other health considerations affecting the subject, and the status of liver and kidney function of the subject. It also depends on the duration of the treatment, other drugs, compounds and/or materials used in combination with the particular therapeutic agent employed, as well as the age, weight, condition, general health and prior medical history of the subject being treated, and like factors. Methods for determining optimal dosages are described in the art, e.g., Remington: The Science and Practice of Pharmacy, Mack Publishing Co., 20^{th} ed., 2000. Optimal dosages for a given set of conditions can be ascertained by those skilled in the art using conventional dosage-determination tests in view of the experimental data for an agent. For oral administration, an exemplary daily dose generally employed is from about 0.001 to about 3000 mg/kg of body weight, with courses of treatment repeated at appropriate intervals. In some embodiments, the daily dose is from about 1 to 3000 mg/kg of body weight.

Typical daily doses in a patient may be anywhere between about 500 mg to about 3000 mg, given once or twice daily, e.g., 3000 mg can be given twice daily for a total dose of 6000 mg. In one embodiment, the dose is between about 1000 to about 3000 mg. In another embodiment, the dose is between about 1500 to about 2800 mg. In other embodiments, the dose is between about 2000 to about 3000 mg.

Plasma concentrations in the subjects may be between about 100 μM to about 1000 μM. In some embodiments, the plasma concentration may be between about 200 μM to about 800 μM. In other embodiments, the concentration is about 300 μM to about 600 μM. In still other embodiments the plasma concentration may be between about 400 to about 800 μM. Administration of prodrugs is typically dosed at weight levels, which are chemically equivalent to the weight levels of the fully active form.

The compositions of the invention may be manufactured using techniques generally known for preparing pharmaceutical compositions, e.g., by conventional techniques such as mixing, dissolving, granulating, dragee-making, levitating, emulsifying, encapsulating, entrapping or lyophilizing. Pharmaceutical compositions may be formulated in a conventional manner using one or more physiologically acceptable carriers, which may be selected from excipients and auxiliaries that facilitate processing of the active compounds into preparations, which can be used pharmaceutically.

Proper formulation is dependent upon the route of administration chosen. For injection, the agents of the invention may be formulated into aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers known in the art. Such carriers enable the compounds of the invention to be formulated as tablets, pills, dragees, capsules, liquids, gels, syrups, slurries, solutions, suspensions and the like, for oral ingestion by a patient to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum 218rabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

Pharmaceutical formulations for parenteral administration can include aqueous solutions or suspensions. Suitable lipophilic solvents or vehicles include fatty oils such as sesame oil or synthetic fatty acid esters, such as ethyl oleate or triglycerides. Aqueous injection suspensions may contain substances which increase the viscosity of the suspension, such as sodium carboxymethyl cellulose, sorbitol, or dextran. Optionally, the suspension may also contain suitable stabilizers or modulators which increase the solubility or dispersibility of the composition to allow for the preparation of highly concentrated solutions, or can contain suspending or dispersing agents. Pharmaceutical preparations for oral use can be obtained by combining the pharmacologically active agent with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating modulators may be added, such as the cross-linked polyvinyl pyrrolidone, agar, or alginic acid or a salt thereof such as sodium alginate.

Other ingredients such as stabilizers, for example, antioxidants such as sodium citrate, ascorbyl palmitate, propyl gallate, reducing agents, ascorbic acid, vitamin E, sodium bisulfite, butylated hydroxytoluene, BHA, acetylcysteine, monothioglycerol, phenyl-α-naphthylamine, or lecithin can be used. Also, chelators such as EDTA can be used. Other ingredients that are conventional in the area of pharmaceutical compositions and formulations, such as lubricants in tablets or pills, coloring agents, or flavoring agents, can be used. Also, conventional pharmaceutical excipients or carriers can be used. The pharmaceutical excipients can include, but are not necessarily limited to, calcium carbonate, calcium phosphate, various sugars or types of starch, cellulose derivatives, gelatin, vegetable oils, polyethylene glycols and physiologically compatible solvents. Other pharmaceutical excipients are well known in the art. Exemplary pharmaceutically acceptable carriers include, but are not limited to, any and/or all of solvents, including aqueous and non-aqueous solvents, dispersion media, coatings, antibacterial and/or antifungal agents, isotonic and/or absorption delaying agents, and/or the like. The use of such media and/or agents for pharmaceutically active substances is well known in the art. Except insofar as any conventional medium, carrier, or agent is incompatible with the active ingredient or ingredients, its use in a composition according to the present invention is contemplated. Supplementary active ingredients can also be incorporated into the compositions, particularly as described above. For administration of any of the compounds used in the present invention, preparations should meet sterility, pyrogenicity, general safety, and purity standards as required by the FDA Office of Biologics Standards or by other regulatory organizations regulating drugs.

For administration intranasally or by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of gelatin for use in an inhaler or insufflator and the like may be formulated containing a powder mix of the compound and a suitable powder base such as lactose or starch.

The compounds may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents, which increase the solubility of the compounds to allow for the preparation of highly concentrated solutions.

Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides.

In addition to the formulations described above, the compounds may also be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt.

An exemplary pharmaceutical carrier for hydrophobic compounds is a cosolvent system comprising benzyl alcohol, a nonpolar surfactant, a water-miscible organic polymer, and an aqueous phase. The cosolvent system may be a VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) contains VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid- or gel-phase carriers or excipients. Examples of such carriers or excipients include calcium carbonate, calcium phosphate, sugars, starches, cellulose derivatives, gelatin, and polymers such as polyethylene glycols.

A pharmaceutical composition can be administered by a variety of methods known in the art. The routes and/or modes of administration vary depending upon the desired results. Depending on the route of administration, the pharmacologically active agent may be coated in a material to protect the targeting composition or other therapeutic agent from the action of acids and other compounds that may inactivate the agent. Conventional pharmaceutical practice can be employed to provide suitable formulations or compositions for the administration of such pharmaceutical compositions to subjects. Any appropriate route of administration can be employed, for example, but not limited to, intravenous, parenteral, intraperitoneal, intravenous, transcutaneous, subcutaneous, intramuscular, intraurethral, or oral administration. Depending on the severity of the malignancy or other disease, disorder, or condition to be treated, as well as other conditions affecting the subject to be treated, either systemic or localized delivery of the pharmaceutical composition can be used in the course of treatment. The pharmaceutical composition as described above can be administered together with additional therapeutic agents intended to treat a particular disease or condition, which may be the same disease or condition that the pharmaceutical composition is intended to treat, which may be a related disease or condition, or which even may be an unrelated disease or condition.

Dianhydrogalactitol, and pharmaceutical compositions comprising dianhydrogalactitol, are typically administered orally or intravenously.

Pharmaceutical compositions according to the present invention can be prepared in accordance with methods well known and routinely practiced in the art. See, e.g., Remington: The Science and Practice of Pharmacy, Mack Publishing Co., 20^{th} ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Formulations for parenteral administration may, for example, contain excipients, sterile water, or saline, polyalkylene glycols such as polyethylene glycol, oils of vegetable origin, or hydrogenated naphthalenes. Biocompatible, biodegradable lactide polymers, lactide/glycolide copolymers, or polyoxyethylene-polyoxypropylene copolymers may be used to control the release of the compounds. Other potentially useful parenteral delivery systems for molecules of the invention include ethylene-vinyl acetate copolymer particles, osmotic pumps, and implantable infusion systems. Formulations for inhalation may contain excipients, for example, lactose, or may be aqueous solutions containing, e.g., polyoxyethylene-9-lauryl ether, glycocholate and deoxycholate, or can be oily solutions for administration or gels.

Pharmaceutical compositions according to the present invention are usually administered to the subjects on multiple occasions. Intervals between single dosages can be weekly, monthly or yearly. Intervals can also be irregular as indicated by therapeutic response or other parameters well known in the art. Alternatively, the pharmaceutical composition can be administered as a sustained release formulation, in which case less frequent administration is required. Dosage and frequency vary depending on the half-life in the subject of the pharmacologically active agent included in a pharmaceutical composition. The dosage and frequency of administration can vary depending on whether the treatment is prophylactic or therapeutic. In prophylactic applications, a relatively low dosage is administered at relatively infrequent intervals over a long period of time. Some subjects may continue to receive treatment for the rest of their lives. In therapeutic applications, a relatively high dosage at relatively short intervals is sometimes required until progression of the disease is reduced or terminated, and preferably until the subject shows partial or complete amelioration of symptoms of disease. Thereafter, the subject can be administered a prophylactic regime.

For the purposes of the present application, treatment can be monitored by observing one or more of the improving symptoms associated with the disease, disorder, or condition being treated, or by observing one or more of the improving clinical parameters associated with the disease, disorder, or condition being treated, as described above.

Sustained-release formulations or controlled-release formulations are well-known in the art. For example, the sustained-release or controlled-release formulation can be (1) an oral matrix sustained-release or controlled-release formulation; (2) an oral multilayered sustained-release or controlled-release tablet formulation; (3) an oral multiparticulate sustained-release or controlled-release formulation; (4) an oral osmotic sustained-release or controlled-release formulation; (5) an oral chewable sustained-release or controlled-release formulation; or (6) a dermal sustained-release or controlled-release patch formulation.

The pharmacokinetic principles of controlled drug delivery are described, for example, in B. M. Silber et al., “Pharmacokinetic/Pharmacodynamic Basis of Controlled Drug Delivery” in Controlled Drug Delivery: Fundamentals and Applications (J. R. Robinson & V. H. L. Lee, eds, 2d ed., Marcel Dekker, New York, 1987), ch. 5, pp. 213-251.

One of ordinary skill in the art can readily prepare formulations for controlled release or sustained release comprising a pharmacologically active agent according to the present invention by modifying the formulations described above, such as according to principles disclosed in V. H. K. Li et al, “Influence of Drug Properties and Routes of Drug Administration on the Design of Sustained and Controlled Release Systems” in Controlled Drug Delivery: Fundamentals and Applications (J. R. Robinson & V. H. L. Lee, eds, 2d ed., Marcel Dekker, New York, 1987), ch. 1, pp. 3-94. This process of preparation typically takes into account physicochemical properties of the pharmacologically active agent, such as aqueous solubility, partition coefficient, molecular size, stability, and nonspecific binding to proteins and other biological macromolecules. This process of preparation also takes into account biological factors, such as absorption, distribution, metabolism, duration of action, the possible existence of side effects, and margin of safety, for the pharmacologically active agent. Accordingly, one of ordinary skill in the art could modify the formulations into a formulation having the desirable properties described above for a particular application.

U.S. Pat. No. 6,573,292 by Nardella, U.S. Pat. No. 6,921,722 by Nardella, U.S. Pat. No. 7,314,886 to Chao et al., and U.S. Pat. No. 7,446,122 by Chao et al., which disclose methods of use of various pharmacologically active agents and pharmaceutical compositions in treating a number of diseases and conditions, including cancer, and methods of determining the therapeutic effectiveness of such pharmacologically active agents and pharmaceutical compositions, are all incorporated herein by this reference.

Typically, the therapeutically effective quantity of dianhydrogalactitol is about 40 mg/m². The therapeutically effective quantity of diacetyldianhydrogalactitol is similar taking into account differences in molecular weight. Other dosages can be employed, including up to 50 mg/m² for dianhydrogalactitol. Higher dosages may also be used, particularly when steps are taken to prevent myelosuppression.

Typically, the dianhydrogalactitol is administered by a route selected from the group consisting of intravenous and oral. Preferably, the dianhydrogalactitol is administered intravenously. Similar routes can be used for diacetyldianhydrogalactitol.

The method can further comprise the step of administering a therapeutically effective dose of ionizing radiation.

In another alternative, the method can further comprise the steps of, subsequent to the administration of the initial dose of the dianhydrogalactitol or the derivative or analog thereof: (1) determining the quantity of a protein associated with the activation of the DNA repair pathway to determine the extent of the activation of the DNA repair pathway; and (2) adjusting the dose of the dianhydrogalactitol or the derivative or analog thereof in response to the extent of the DNA repair pathway. The activation of the DNA repair pathway is a measure of the degree of DNA damage induced by the administration of the dianhydrogalactitol or the derivative or analog thereof. The protein associated with the activation of the DNA repair pathway can be, but is not limited to, phosphorylated ATM, phosphorylated RPA32, or γH2A.X. The ATM protein is a serine/threonine protein kinase that is recruited and activated by DNA double-strand breaks. It phosphorylates several key proteins that initiate activation of the DNA damage checkpoint, leading to cell cycle arrest, DNA repair or apoptosis. Several of these targets, including p53, CHK2, BRCA1, NBS1 and γH2A.X are tumor suppressors. ATM is a 350-kDa protein with 3056 amino acid residues. Upon DNA damage, ATM autophosphorylates at Ser¹⁹⁸¹. Therefore, the quantity of phosphorylated ATM is an indicator of DNA damage. RPA32 is another protein that is involved in DNA repair and is phosphorylated in response to DNA damage, specifically the accumulation of single-stranded DNA to which Replication Protein A is bound; RPA32 is a subunit of Replication Protein A. This phosphorylation can be carried out by ataxia telangiectasia mutated (ATM), ataxia telangiectasia and Rad3 related (ATR), and DNA-dependent protein kinase catalytic subunit (DNA-PKcs), a kinase related to ATM and ATR (S. Liu et al., “Distinct Roles for DNA-PK, ATM, and ATR in RPA Phosphorylation and Checkpoint Activation in Response to Replication Stress,” Nucl. Acids Res. doi:10.1093/nar/gks849 (2012). The γH2A.X protein is a phosphorylated histone in which phosphorylation occurs at Ser¹³⁹ as the result of DNA damage, particularly double-strand breaks. Therefore, the quantity of these modified proteins is an indicator of the activation of the DNA repair pathway.

Another aspect of the present invention is a kit comprising, separately packaged, two or more different doses of a hexitol derivative as described above for treatment of a malignancy. Typically, the hexitol derivative is dianhydrogalactitol or diacetyldianhydrogalactitol. When the alkylating hexitol derivative is dianhydrogalactitol, the kit can comprise, but is not limited to, the following combinations of doses: (i) 1.5 mg/m² and 3.0 mg/m²; (ii) 1.5 mg/m², 3.0 mg/m², and 5.0 mg/m²; (iii) 1.5 mg/m², 3.0 mg/m², 5.0 mg/m², and 10 mg/m²; (iv) 1.5 mg/m², 3.0 mg/m², 5.0 mg/m², 10 mg/m², and 15 mg/m²; (v) 10 mg/m²; (iv) 1.5 mg/m², 3.0 mg/m², 5.0 mg/m², 10 mg/m², 15 mg/m², and 20 mg/m²; (vi) 1.5 mg/m², 3.0 mg/m², 5.0 mg/m², 10 mg/m², 15 mg/m², 20 mg/m², and 25 mg/m²; (vii) 1.5 mg/m², 3.0 mg/m², 5.0 mg/m², 10 mg/m², 15 mg/m², 20 mg/m², 25 mg/m², and 30 mg/m²; (viii) 1.5 mg/m², 3.0 mg/m², 5.0 mg/m², 10 mg/m², 15 mg/m², 20 mg/m², 25 mg/m², 30 mg/m², and 40 mg/m²; and (ix) 1.5 mg/m², 3.0 mg/m², 5.0 mg/m², 10 mg/m², 15 mg/m², 20 mg/m², 25 mg/m², 30 mg/m², 40 mg/m², and 50 mg/m². Other combinations of doses including two or more of these alternative doses are also possible. The hexitol derivative can be in the form of a pharmaceutical composition. The doses can be assembled into a blister pack as is conventionally used for packaging of pharmaceutical doses. The kit can further comprise instructions for use. The kit can further include, separately packaged, doses of: (i) a topoisomerase inhibitor; (ii) an inhibitor of ATM; or (iii) an inhibitor of ATR.

Accordingly, one aspect of the present invention is a method of treating a malignancy selected from the group consisting of glioblastoma, non-small-cell lung carcinoma, and ovarian cancer by the induction of double-strand breaks in the DNA of tumor cells by administration of a therapeutically effective quantity of a substituted hexitol derivative according to the present invention as described above.

The method of treating the malignancy selected from the group consisting of glioblastoma, non-small-cell lung carcinoma, and ovarian cancer can employ one of the methods to improve the efficacy and/or reduce the side effects of the administration of the substituted hexitol described above.

The method of treating the malignancy selected from the group consisting of glioblastoma, non-small-cell lung carcinoma, and ovarian cancer can employ a composition according to the present invention as described above.

The method of treating the malignancy selected from the group consisting of glioblastoma, non-small-cell lung carcinoma, and ovarian cancer can further comprise administration of a therapeutically effective quantity of an inhibitor of PARP.

The method of treating the malignancy selected from the group consisting of glioblastoma, non-small-cell lung carcinoma, and ovarian cancer can further comprise administration of a therapeutically effective quantity of an agent that counters loss of PTEN function as described above.

The method of treating the malignancy selected from the group consisting of glioblastoma, non-small-cell lung carcinoma, and ovarian cancer can further comprise administration of a therapeutically effective quantity of an additional DNA-damaging agent as described above. The method of treating the malignancy selected from the group consisting of glioblastoma, non-small-cell lung carcinoma, and ovarian cancer can further comprise administration of a therapeutically effective quantity of an agent modulating at least one of the following pathways: γH2AX, p-RPA32 (S4/8, S33), ATR, ATM, Rad51, CtIP, BRCA1, and LEDGF. As described above, the method can further comprise the steps of, subsequent to the administration of the initial dose of the dianhydrogalactitol or the derivative or analog thereof: (1) determining the quantity of a protein associated with the activation of the DNA repair pathway to determine the extent of the activation of the DNA repair pathway; and (2) adjusting the dose of the dianhydrogalactitol or the derivative or analog thereof in response to the extent of the DNA repair pathway. The activation of the DNA repair pathway is a measure of the degree of DNA damage induced by the administration of the dianhydrogalactitol or the derivative or analog thereof. The protein associated with the activation of the DNA repair pathway can be, but is not limited to, phosphorylated ATM, phosphorylated RPA32, or γH2A.X. The method can further comprise the administration of a therapeutically effective quantity of a topoisomerase inhibitor as described above. The method can also further comprise the administration of a therapeutically effective quantity of an inhibitor of ATM or an inhibitor of ATR as described above.

Also, as described above, substituted hexitol derivatives according to the present invention can be used for treatment of leptomeningeal carcinomatosis (LC). Therefore, another aspect of the present invention is a method of treating leptomeningeal carcinomatosis (LC) by the induction of double-strand breaks in the DNA of tumor cells by administration of a therapeutically effective quantity of a substituted hexitol derivative according to the present invention as described above.

The method of treating LC can employ one of the methods to improve the efficacy and/or reduce the side effects of the administration of the substituted hexitol described above.

The method of treating LC can employ a composition according to the present invention as described above. Such a composition can be formulated for the treatment of LC as described above. The method of treating LC can further comprise administration of a therapeutically effective quantity of an additional agent for treatment of LC.

The method of treating LC can further comprise administration of a therapeutically effective quantity of an inhibitor of PARP.

The method of treating LC can further comprise administration of a therapeutically effective quantity of an agent that counters loss of PTEN function as described above.

The method of treating LC can further comprise administration of a therapeutically effective quantity of an additional DNA-damaging agent as described above.

The method of treating LC can further comprise administration of a therapeutically effective quantity of a topoisomerase inhibitor as described above.

The method of treating LC can further comprise administration of a therapeutically effective quantity of an an inhibitor of ATM or an inhibitor of ATR.

The invention is illustrated by the following Example. This Example is included for illustrative purposes only, and is not intended to limit the invention.

### Example

Dianhydrogalactitol (VAL-083) is a bi-functional alkylating agent causing N⁷-guanine methylation and interstrand DNA crosslinks. Preclinical and clinical trial data suggest antineoplastic effect of VAL-083 in various cancers. However, the detailed molecular mechanisms mediating VAL-083 sensitivity or resistance in cancer is still unclear. The results of this Example are intended to investigate the signaling events responsible for VAL-083 activity against cancer.

Nine cancer cell lines were evaluated by cell proliferation assay for VAL-083 sensitivity. Relatively resistant cell lines (PC3 and H2122) and relatively sensitive cells (LNCaP and H1792) were chosen to investigate DNA damage response induced by VAL-083. VAL-083 treatment led to cell cycle arrest at S and G₂ phase. The data also showed increased phosphorylation of histone variant H2A.X (γH2A.X) due to DNA damage response to VAL-083-induced double strand breaks. Alterations in DNA damage repair signaling pathways may be responsible for the sensitivity or resistance to VAL-083 in different cancer cells.

VAL-083 is a bi-functional alkylating agent causing N⁷-guanine methylation and interstrand DNA crosslinks. However, the detailed mechanism of the anti-neoplastic activity of VAL-083 remains to be elucidated.

VAL-083 generates DNA double-strand breaks (DSBs). If left unrepaired, such DSBs may have severe consequences, such as genomic instability, chromosome aberrations, or cell death.

It is hypothesized that VAL-083 cytotoxicity is due to the activation of the DNA-damage response. The anti-neoplastic effect of VAL-083 is dependent on the ability of cancer cells to repair the VAL-083-induced DNA damage. Alterations in the DNA damage repair signaling pathway lead to VAL-083 sensitivity or resistance in tumor cells.

Methods

Cell Culture:

All cell lines were maintained at 37° C. in 5% CO₂ atmosphere. PC3 and A549 cells were cultured in Dulbecco's modified Eagle's medium (DMEM) supplemented with 10% fetal bovine serum. H2122, H1792, H23, and LNCaP cells were cultured in RPMI1640 with 10% fetal bovine serum.

Crystal Violet Assay:

Following 72 h of different concentrations of VAL-083 treatment, cells were fixed in 1% glutaraldehyde for 5 min. After rinsing with distilled water, cells were incubated with 0.1% crystal violet solution dye for 10 min. Cells were then gently washed with distilled water and air-dried. The crystals on the plate were dissolved in Sorenson's solution before reading absorbance at 560 nm wave length in a microplate reader. Cell growth is expressed as percentage compared to untreated cells.

Cell Cycle Analysis Using Propidium Iodide (PI) Staining:

Cell cycle distribution was evaluated based on DNA content using PI staining. Serum starvation synchronized cells were treated with 5 μM VAL-083 for 1 h, 4 h, 19 h, 24 h, 44 h, and 49 h. Cells were then trypsinized, washed in PBS, and centrifuged at 1000 rpm for 5 min. Cell pellets were fixed in 70% ethanol overnight at 4° C. After washing, cells were incubated with 500 μl PI solution in PBS containing 50 μg/ml PI, 100 μg/mIRNase A, and 0.05% Triton X-100 for 40 min at 37° C. in the dark. Thereafter, cells were washed and resuspended in PBS. DNA content was analyzed by flow cytometry and histograms were made using FlowJo software. Untreated cells were included as control.

Western Blotting:

Cells were lysed in EBC buffer (50 mM Tris-HCl, pH 8.0, 120 mM NaCl, 1% NP-40, and 1 mM EDTA) supplemented with phosphatase inhibitor and protease inhibitor. Cellular proteins were separated by SDS-PAGE and transferred onto PVDF membrane. After incubation with blocking buffer for 1 h, the membranes were incubated with designated primary antibodies overnight at 4° C. Then, Membranes were washed three times for 10 min and incubated with horseradish peroxidase-conjugated anti-mouse or anti-rabbit antibodies for 1-2 h. Membranes were washed with TBST three times and developed with ECL system (Pierce) according to the manufacturer's instruction. The following primary antibodies were used for immunoblotting: γH2A.X (Cell Signaling Technology, 2577); H2A.X (Abcam, ab11175); phospho-ATM (S1981) (Rockland Antibodies and Assays, 200-301-400); ATM (Cell Signaling Technology, 2873); GAPDH (Cell Signaling Technology, 5174); phospho-RPA32 (S33) (Bethyl Laboratories, A300-246A); phospho-CHK1 (S345) (Cell Signalling Technology, 2348), phospho-CHK2 (T68) (Cell Signaling Technology, 2661), Cyclin A2 (Abcam, ab16726), RPA32 (Abcam, ab2175).

Immunofluorescence (IF):

Cells were grown on glass coverslips for at least 16 h before serum starvation for 24 h. Synchronized cells were treated with VAL-083 for 1 h followed by washout and incubation with complete medium for another 24 h. Subsequently, cells were fixed for 30 min with 4% paraformaldehyde in PBS at room temperature. Then, cells were washed three times with PBS and permeabilized for 20 min with 0.5% Triton X-100 in PBS. After washing with PBS for three times and blocking with 3% BSA in PBS for 1 h at room temperature, cells were incubated overnight at 4° C. with primary antibodies diluted in fresh blocking solution. Next, cells were washed three times with PBS and incubated with secondary antibodies for 1 h at room temperature. After washing with PBS for three times, coverslips were mounted with Vectashield mounting medium (with DAPI). Antibodies used in IF staining were γH2A.X (Cell Signaling Technology, 2577); Cyclin A2 (Abcam, ab16726); donkey anti-rabbit Alexa-Fluor 594 (Life Technologies, A21207) and donkey anti-mouse Alexa-Fluor 488 (Life Technologies, A21202). Images were acquired using a Zeiss AxioObserver.

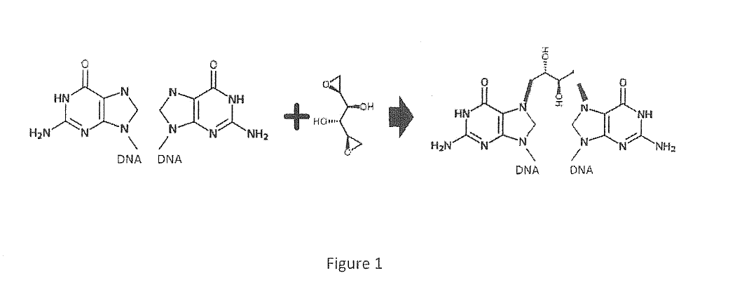

FIG. 1 is a diagram showing the activity of dianhydrogalactitol in inducing N⁷-guanine inter-strand DNA crosslinking.

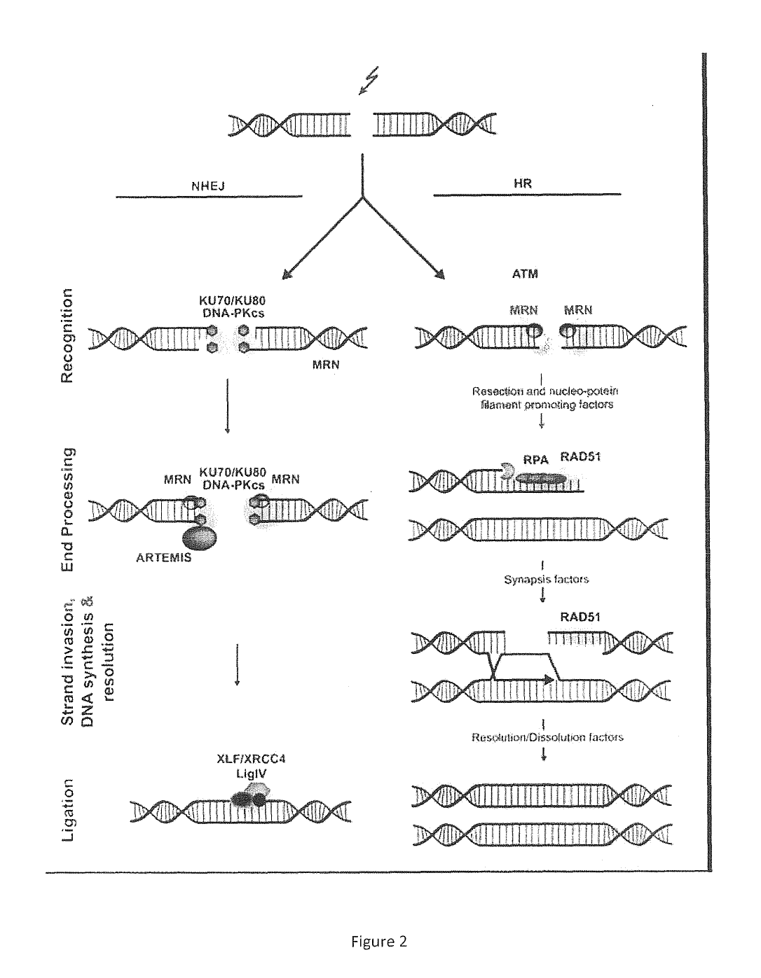

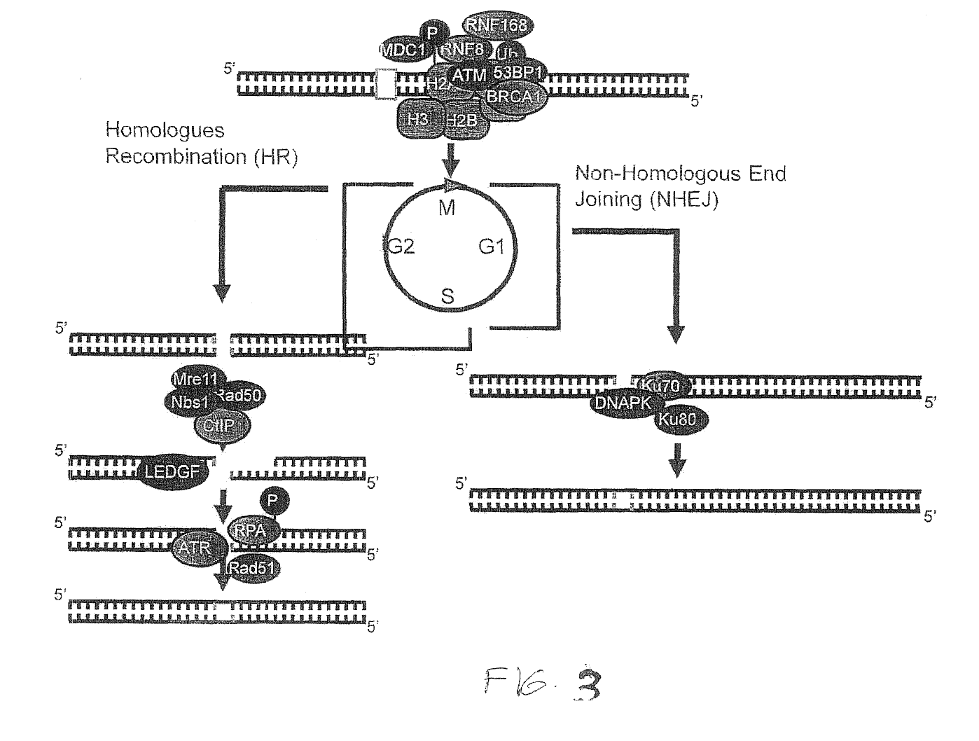

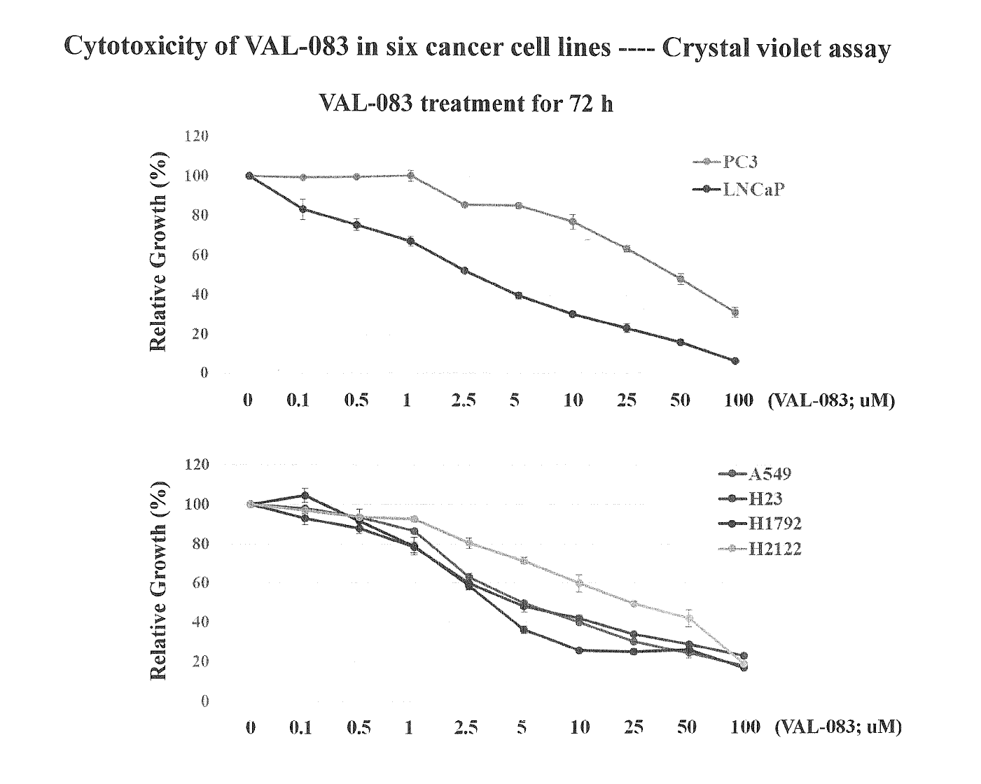

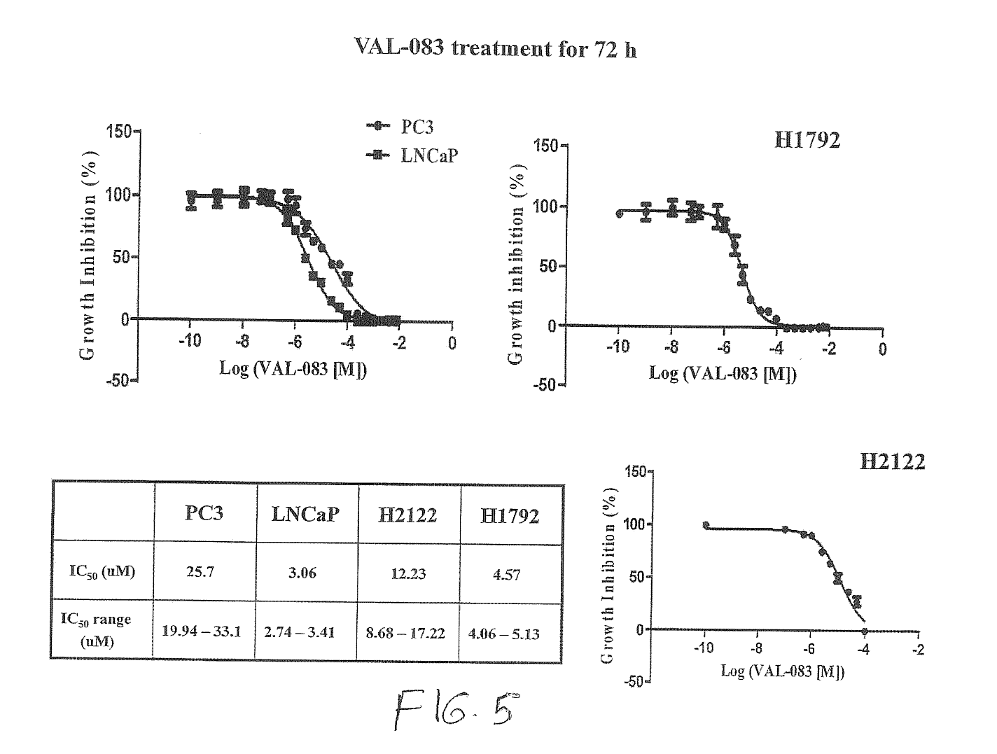

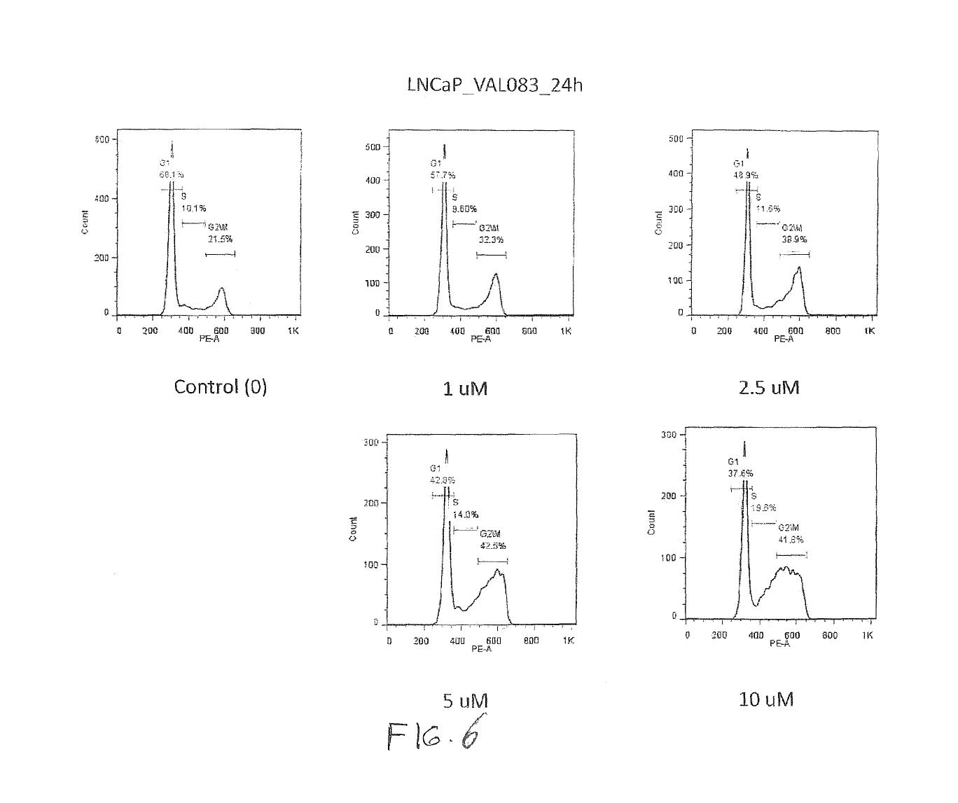

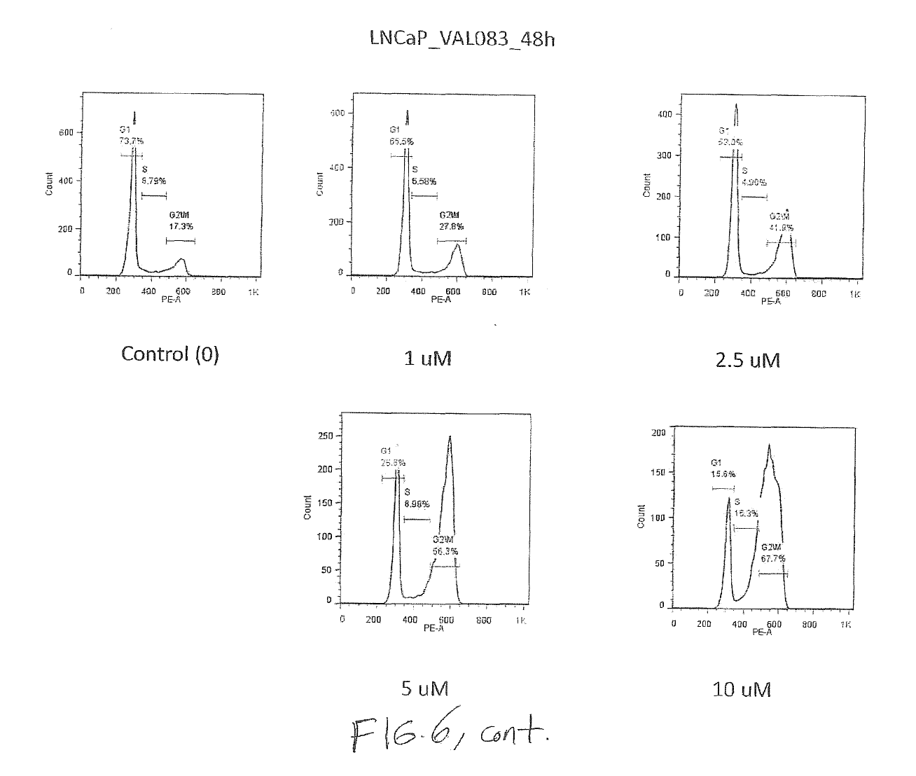

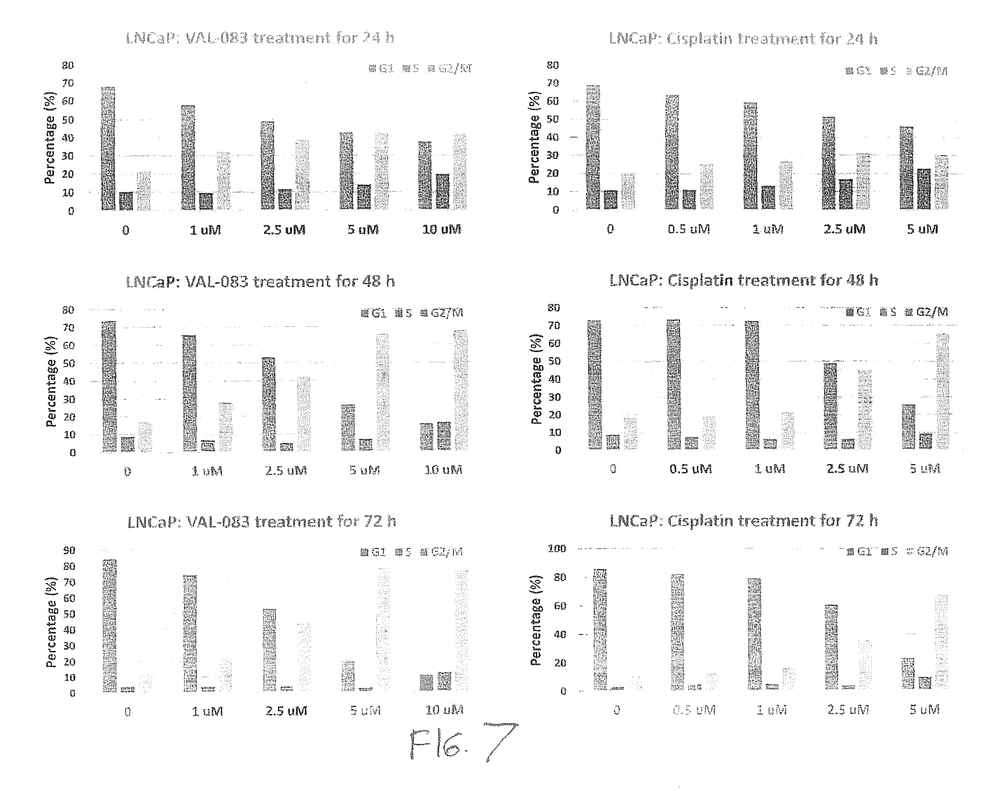

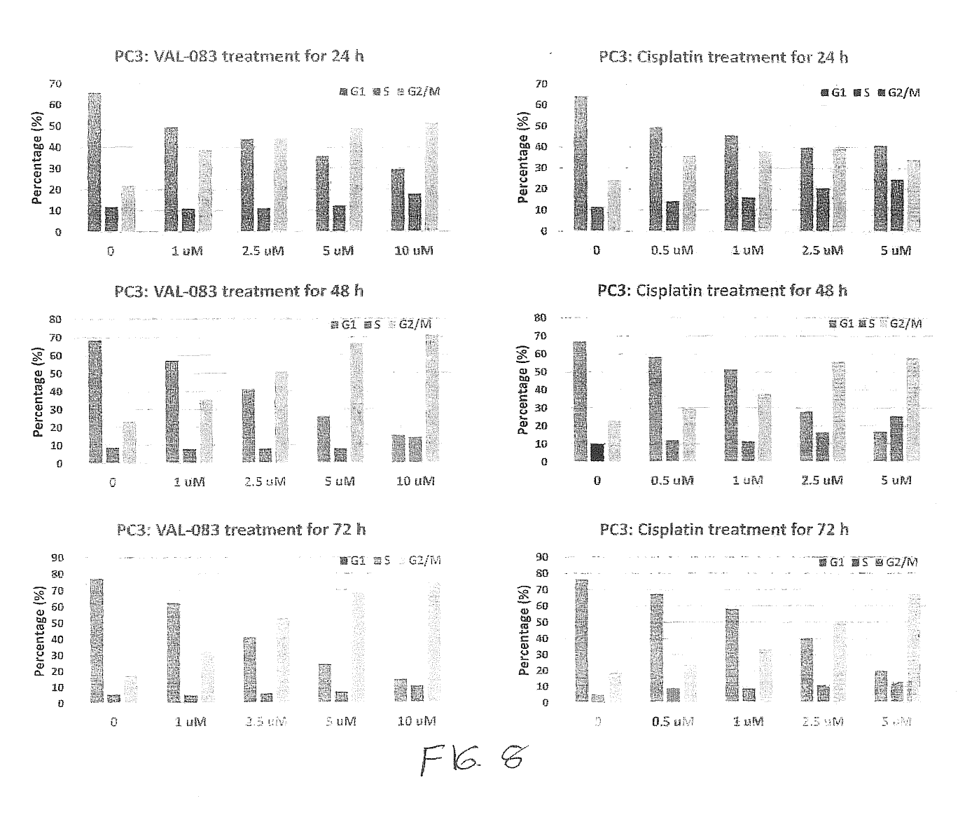

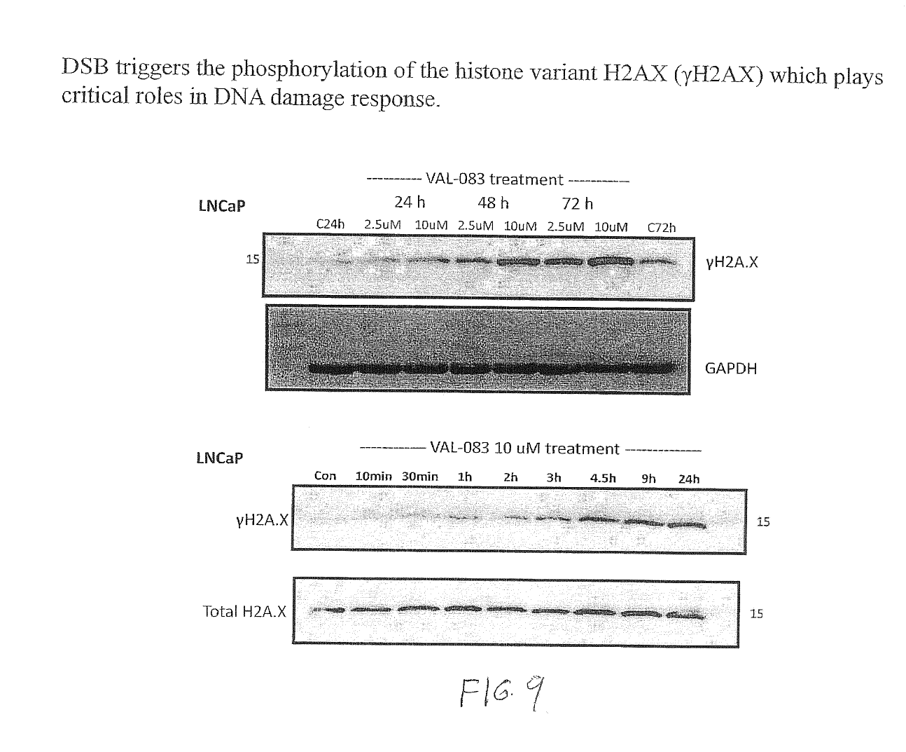

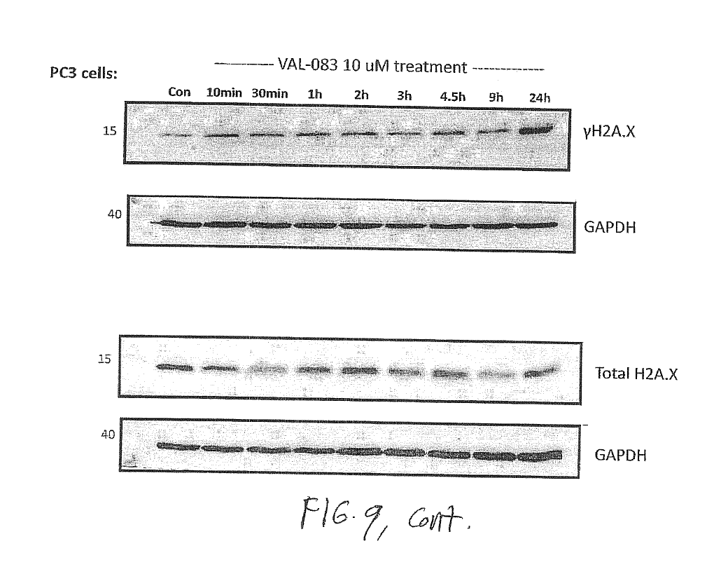

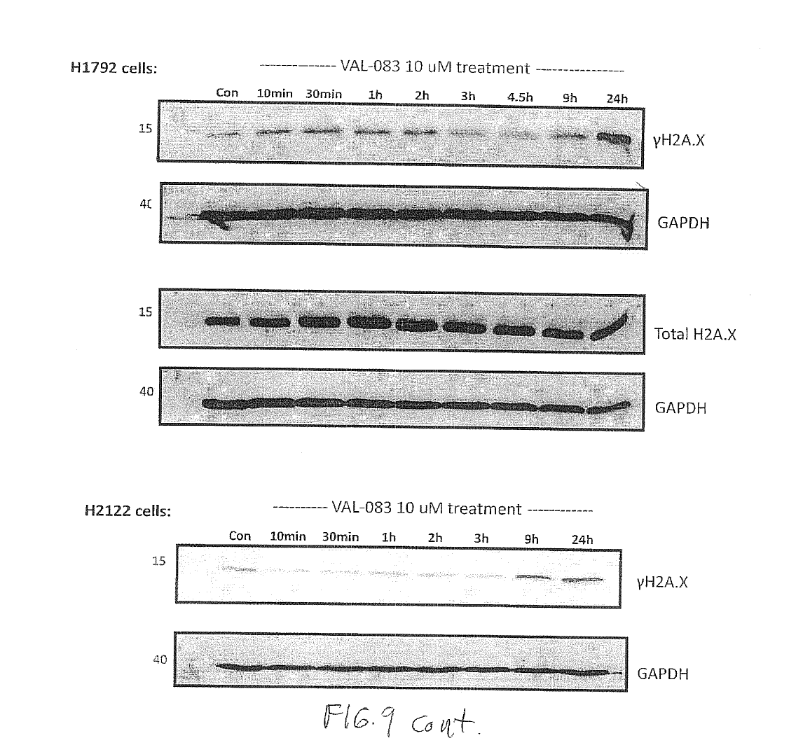

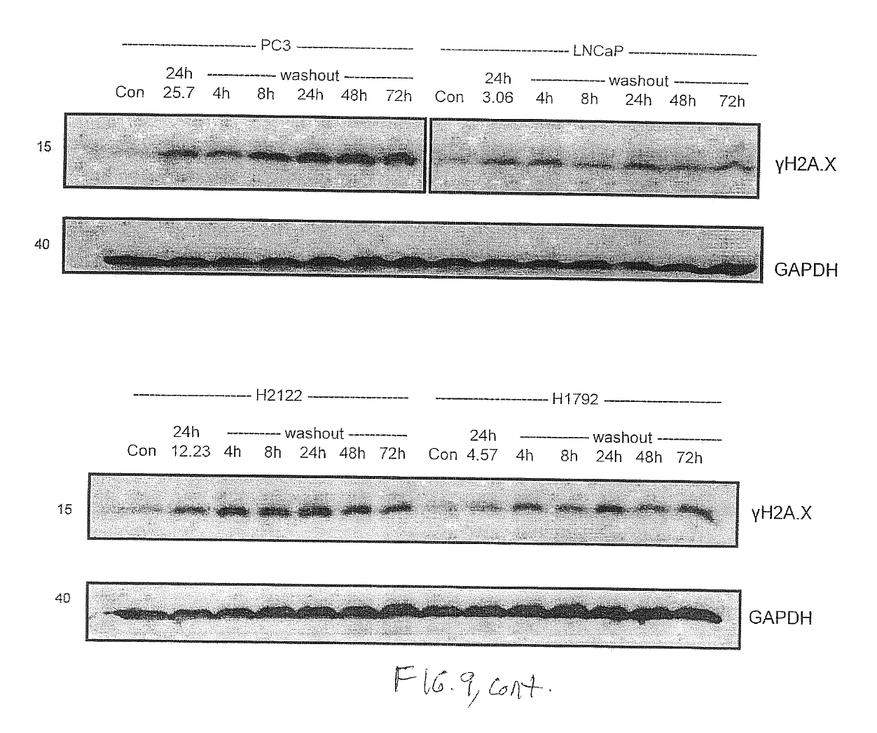

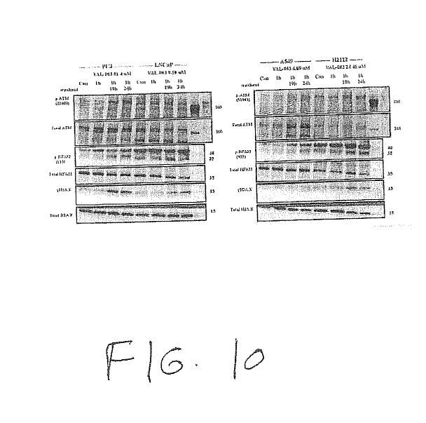

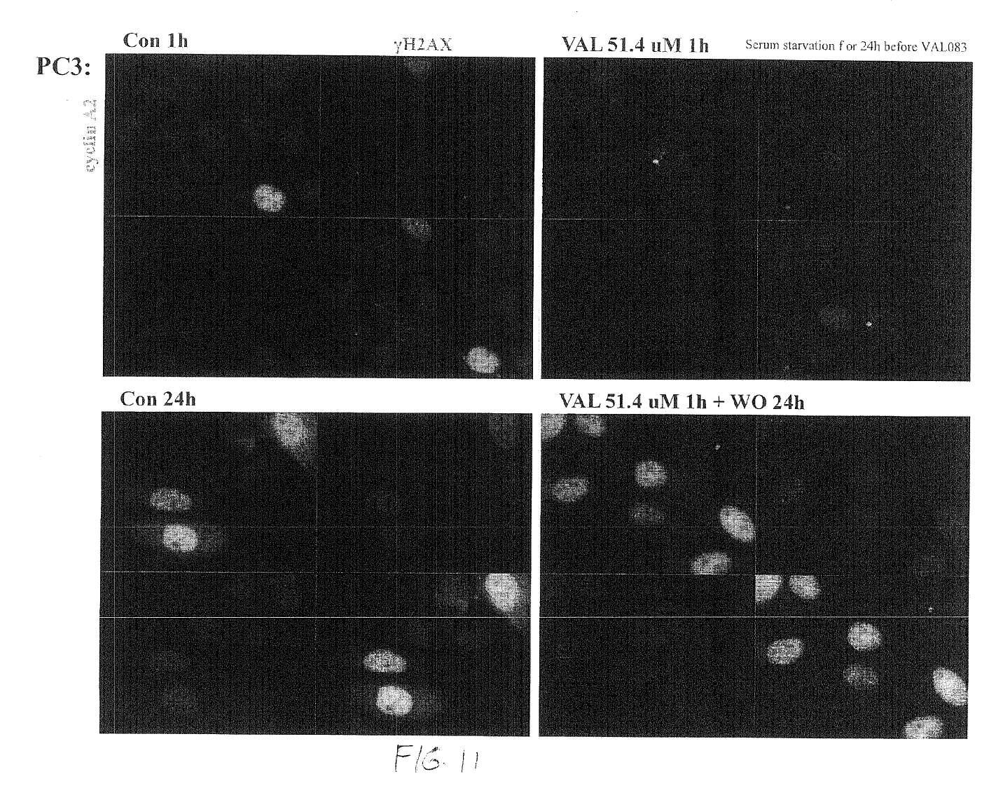

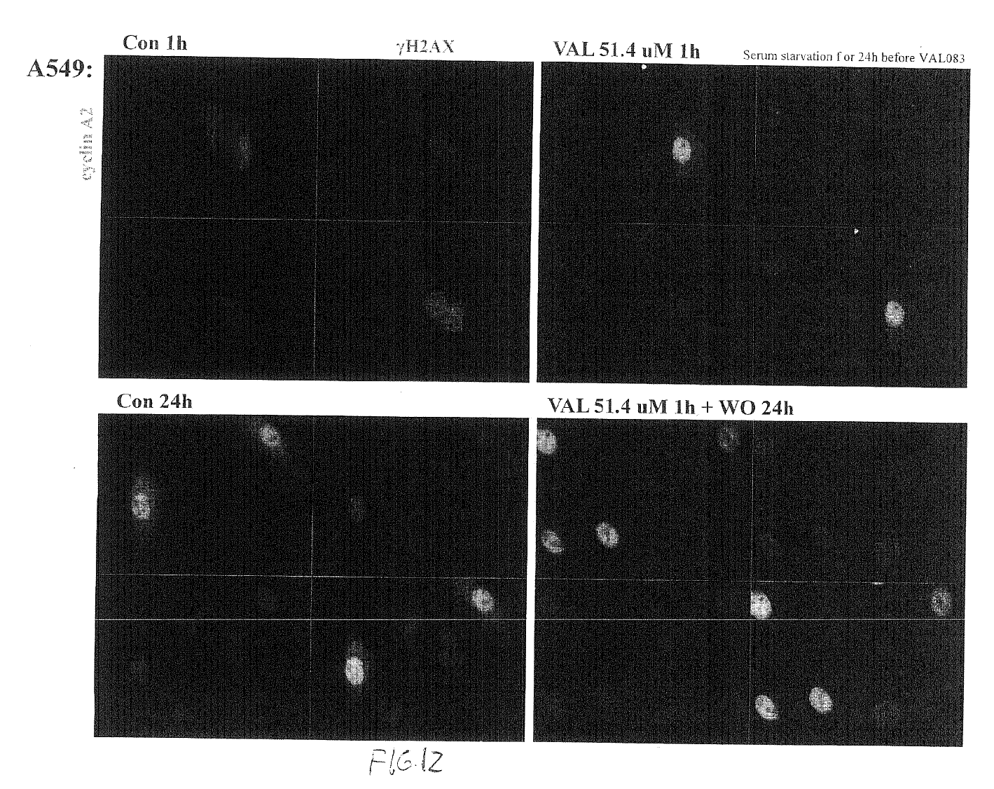

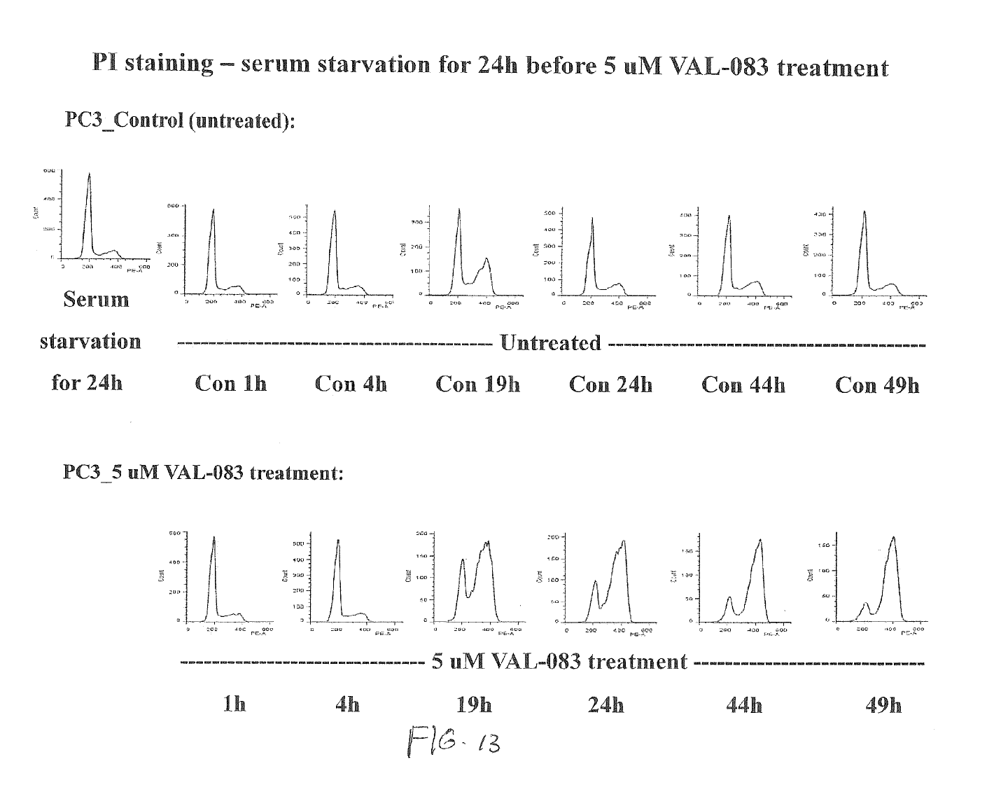

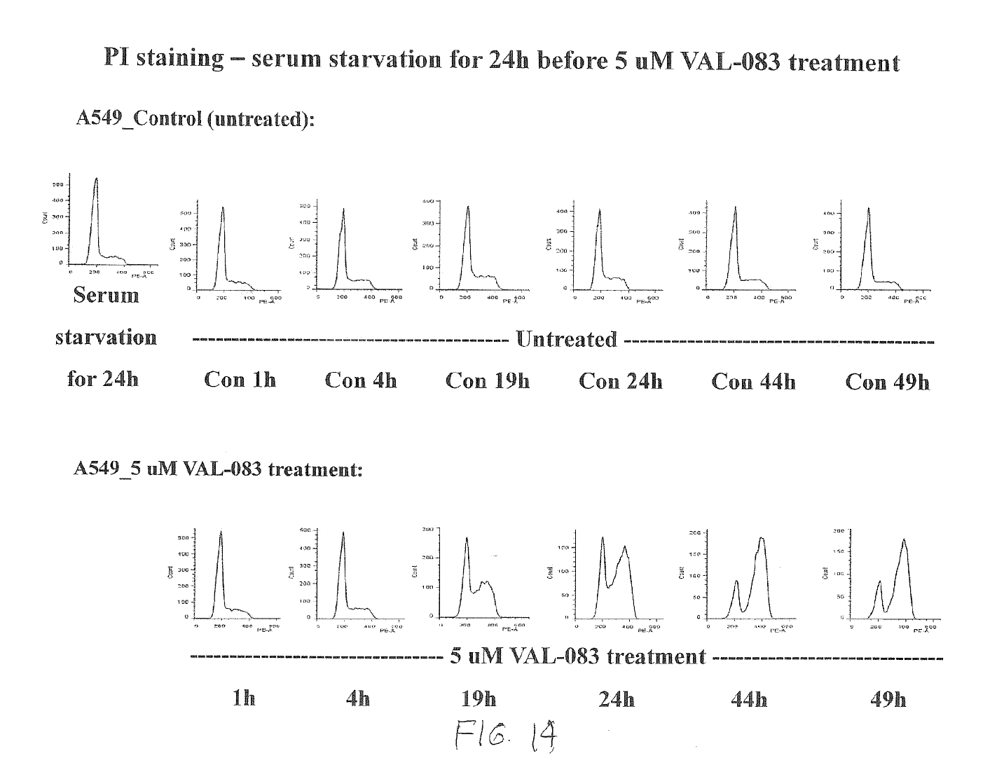

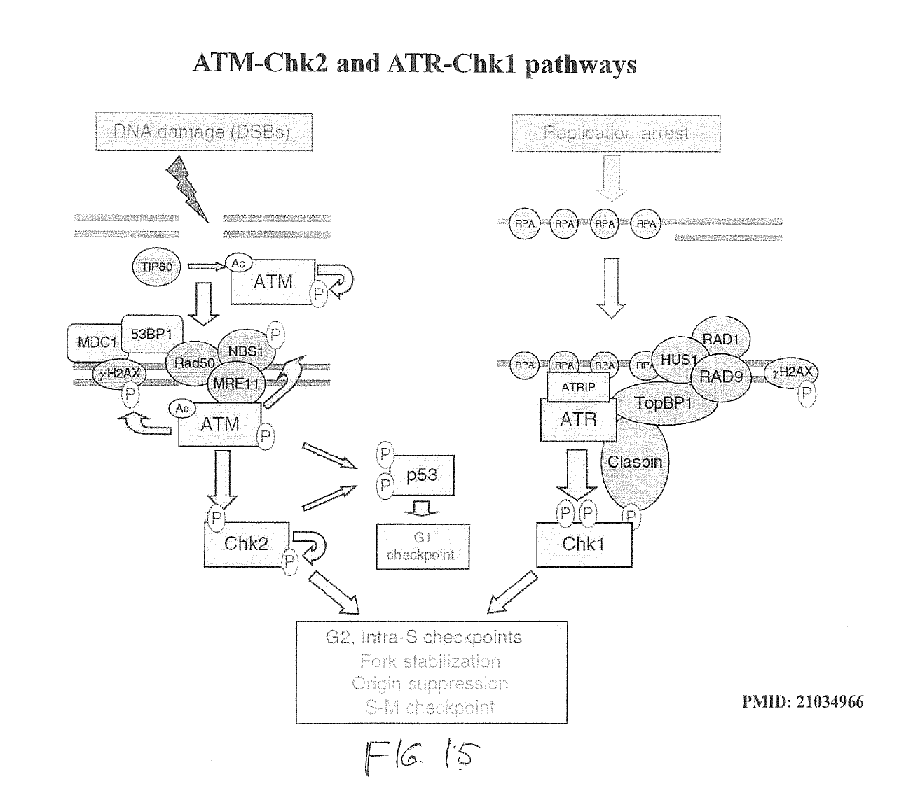

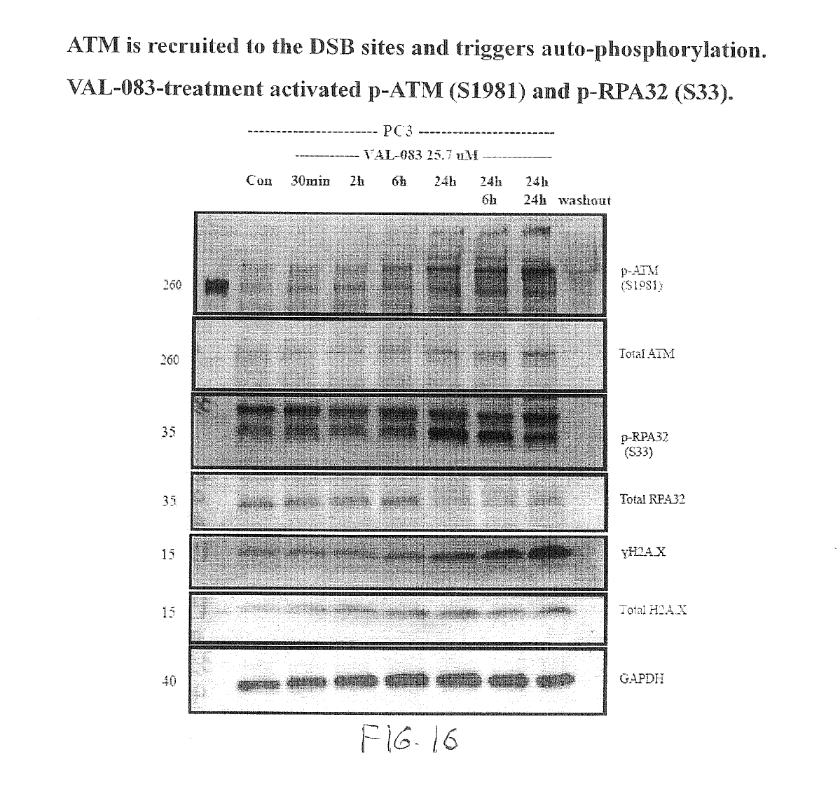

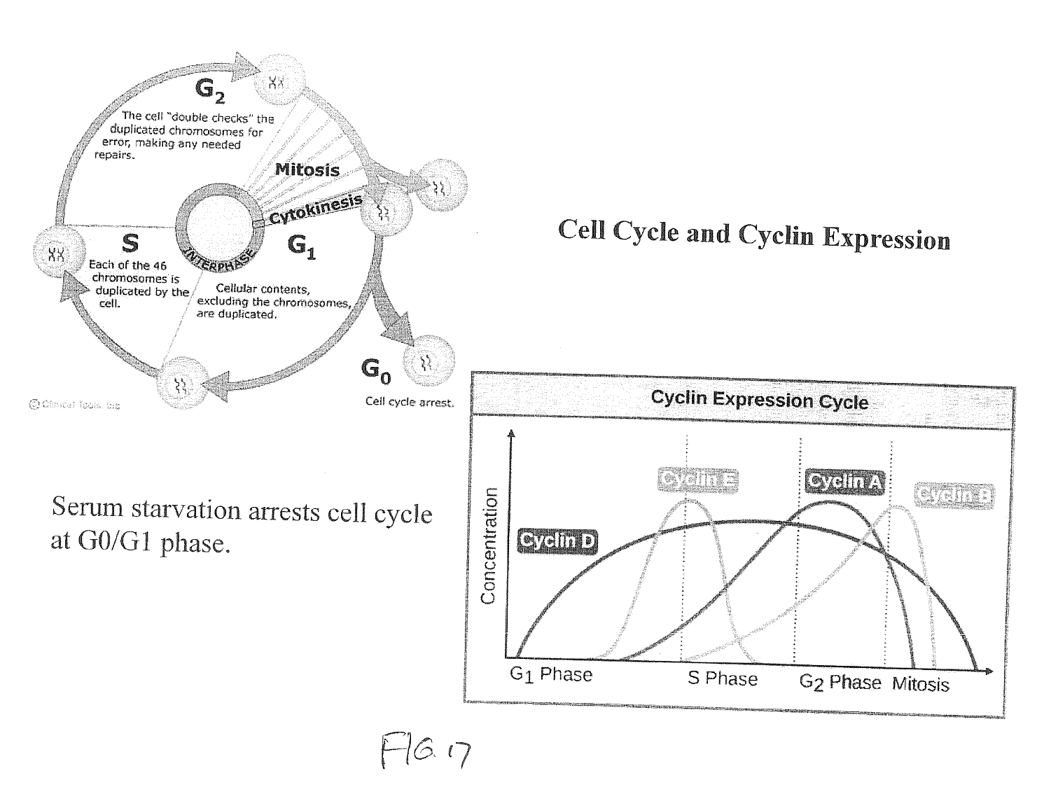

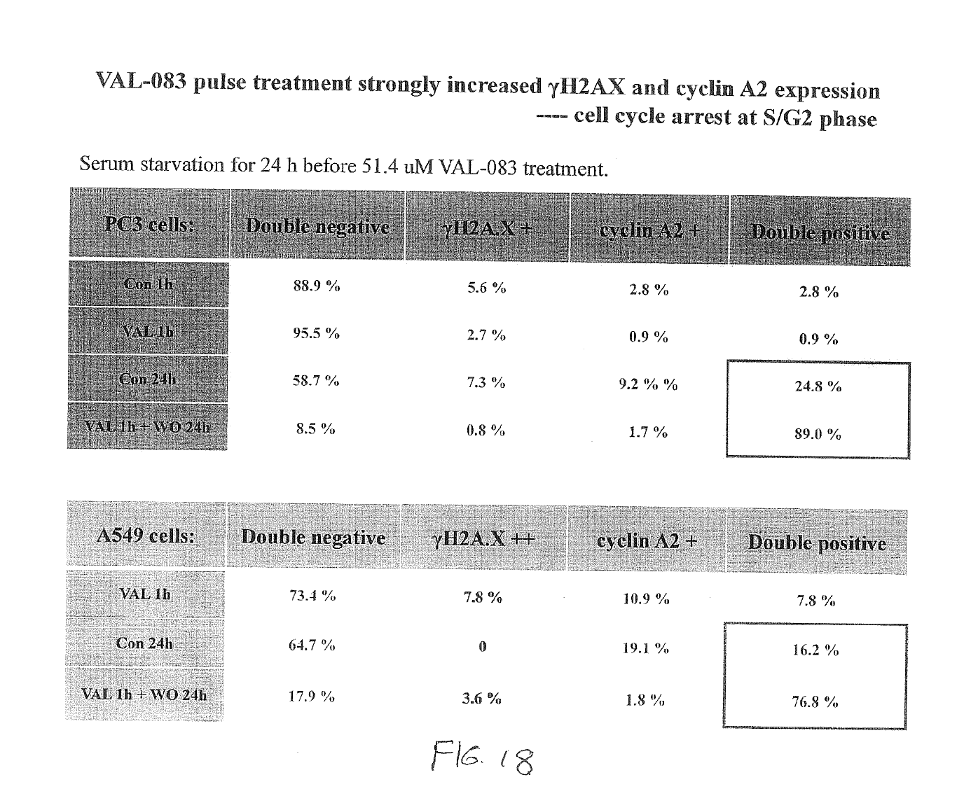

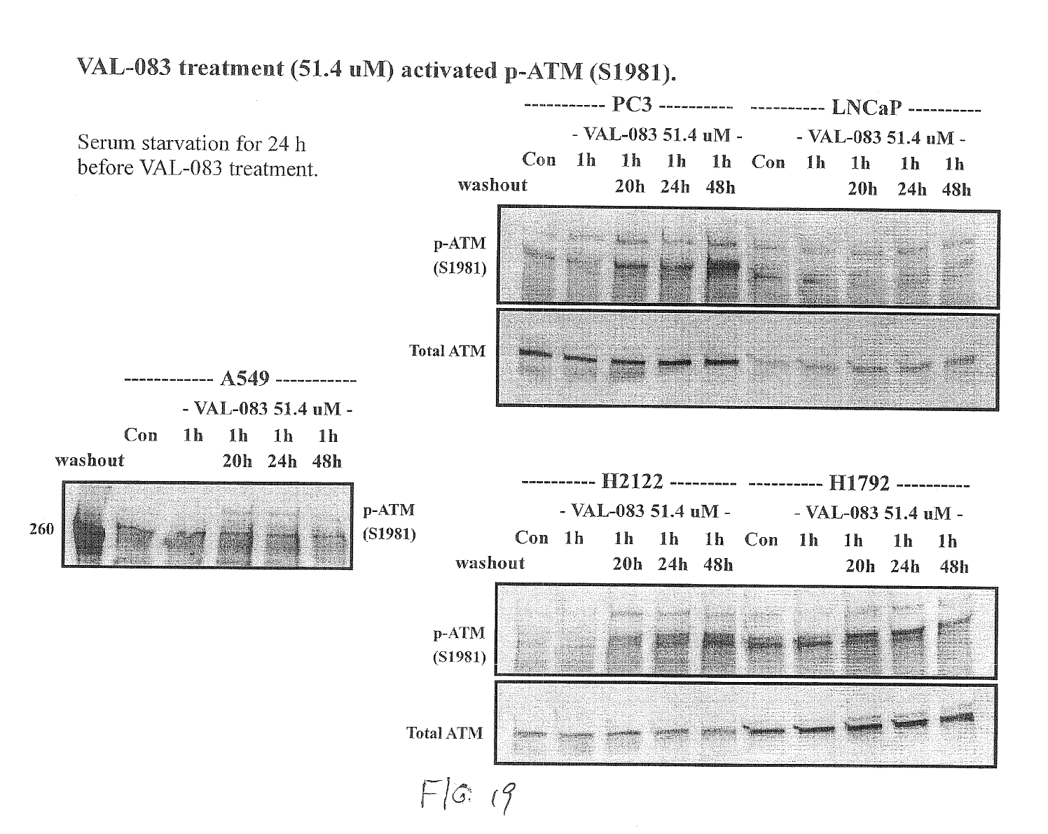

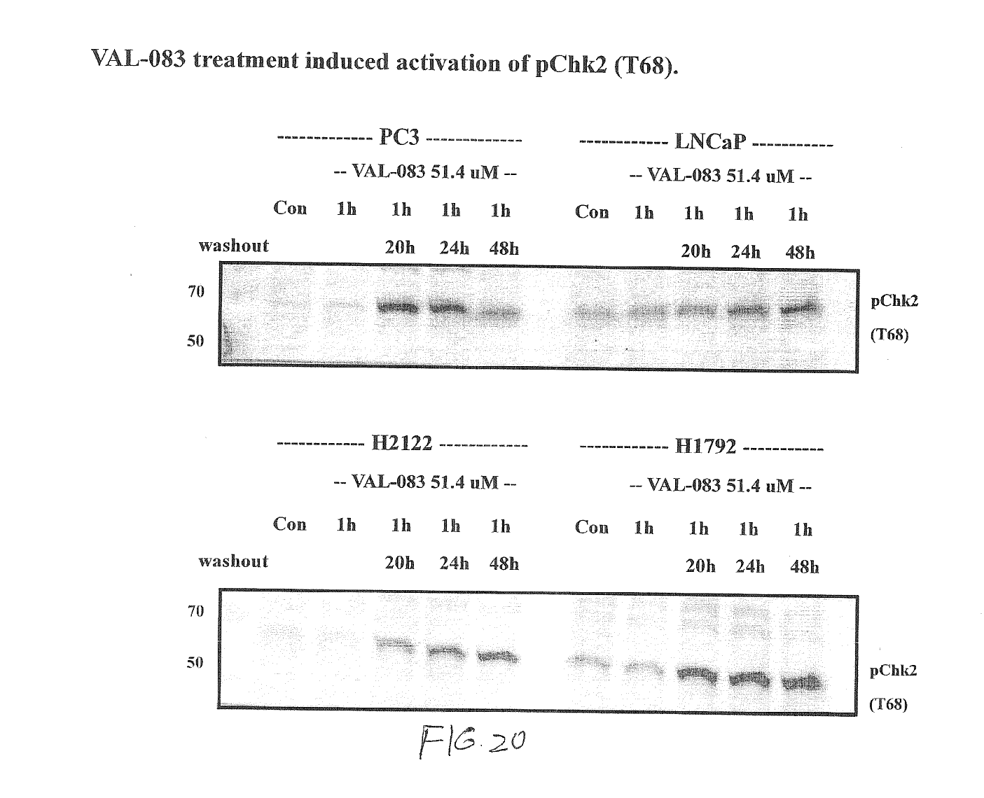

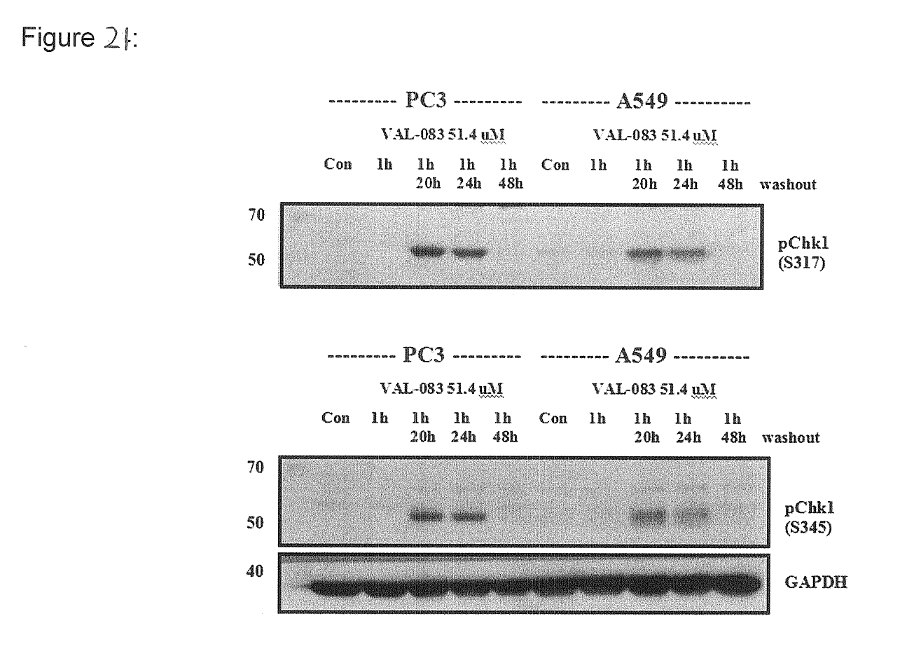

FIG. 2 is a diagram showing DNA damage repair signaling pathways. (FIG. 2 and FIG. 3 are both showing the two most common DNA DSB mechanisms. Maybe only use FIG. 3.)

FIG. 3 is a diagram showing the two most common DNA double-strand break repair mechanisms in mammalian cells; homologous recombination (HR) and non-homologous end joining (NHEJ).

FIG. 4 is a diagram showing crystal violet assay for viability following administration of VAL-083 for 72 hours in six human cell lines: prostate cancer cell lines PC3 and LNCaP in the top panel; NSCLC cell lines A549, H23, H1792, and H2122 in the bottom panel. (Please use FIG. 4 as attached in the email because “Growth Inhibition” should be “Relative Growth”.)

(FIG. 5 and FIG. 15 are the same. I think we can only use FIG. 15.) FIG. 15 is a diagram with graphs showing the effect of VAL-083 treatment for 72 hours at various concentrations on cell growth for PC3, LNCaP, A549, H1792, and H2122, showing IC₅₀ for VAL-083 in these cell lines.

FIG. 6 shows cell cycle analyses for LNCaP treated with 1 μM, 2.5 μM, 5 μM, and 10 μM of VAL-083 for 24 hours or 48 hours, and a control with no treatment, showing the proportions of the cells in G1, S, and G2/M phase.

FIG. 7 shows cell cycle analysis for LNCaP treated with 1 μM, 2.5 μM, 5 μM, and 10 μM of VAL-083 or cisplatin for 24 hours, 48 hours, or 72 hours, together with controls with no treatment, showing the proportions of the cells in G1, S, and G2/M phase.

FIG. 8 shows cell cycle analysis for PC3 treated with 1 μM, 2.5 μM, 5 μM, and 10 μM of VAL-083 or cisplatin for 24 hours, 48 hours, or 72 hours, together with controls with no treatment, showing the proportions of the cells in G1, S, and G2/M phase.

FIG. 9 shows that VAL-083 treatment induces DNA double strand breaks (DSB) in LNCaP cells, PC3 cells, H1792 cells, and H2122 cells. DSB triggers the phosphorylation of the histone variant H2AX (γH2AX) which plays critical roles in DNA damage response, and the accumulation of γH2AX in LNCaP cells, PC3 cells, H1792 cells, and H2122 cells after VAL-083 treatment is shown in Western blots. GAPDH is shown as a loading control. FIG. 9 shows that γH2AX is detectable at around 24 hours and lasted for 48-72 hours after removal of VAL-083 from the cell culture medium.

FIG. 10 shows that VAL-083 treatment activated DNA damage signaling pathways as demonstrated by expression of phospho-ATM (S1981) and phospho-RPA32 (S33), especially in PC3 and H2122 cells. In the left panel, results for PC3 cells (VAL-083 at 51.4 μM) and LNCaP cells (VAL-083 at 9.18 μM) are shown. In the right panel, results for A549 cells (VAL-083 at 6.89 μM) and H2122 cells (VAL-083 at 24.46 μM) are shown. For each cell line, a control is shown, and results are shown (Western blots) for 1 hour of treatment, 1 hour of treatment followed by a 19-hour washout, and 1 hour of treatment followed by a 24-hour washout, respectively. Results are shown for each time point for p-ATM (S1981), total ATM, p-RPA32 (33), total RPA32, γH2A.X, and total H2A.X. (FIG. 10 is the same as FIG. 19.)

FIG. 11 shows the results of immunofluorescent staining after VAL-083 treatment in PC3 cells (left panel) and A549 cells (right panel). The results show increased γH2A.X and late S/G2 phase cell cycle arrest after VAL-083 treatment in PC3 and A549 cells. VAL-083 was administered at 51.4 μM for 1 hour. In each panel, the results, in a clockwise direction, are shown for untreated cells at 1 hour, VAL-083 treatment for 1 hour, VAL-083 treatment for 1 hour followed by a 24-hour washout (WO), and untreated cells for 24 hours. Cyclin A2 is also shown.

FIG. 12 shows VAL-083 induced activation of γH2AX at around 24 hours of treatment. (FIG. 12 is the same as FIG. 9.)

FIG. 13 shows PI staining and shows that VAL-083 treatment led to cell cycle arrest at S/G2 phase in non-synchronized LNCaP cells.

FIG. 14 shows PI staining and shows that 5 μM VAL-083 treatment caused S/G2 cell cycle arrest in serum starvation synchronized PC3 and A549 cells.

FIG. 15 shows IC₅₀ analysis by crystal violet assay after VAL-083 treatment for 72 hours. (FIG. 15 is the same as FIG. 5.)

FIG. 16 shows the persistence of γH2AX activation for 24-72 hours after VAL083 treatment (IC₅₀) for 24 hours. (FIG. 16 has been showed before in FIG. 9.)

FIG. 17 shows the ATM-Chk2 and ATR-Chk1 pathways. (DA. Gillespie et al., “The ATM-Chk2 and ATR-Chk1 pathways in DNA damage signaling and cancer.” Adv Cancer Res 108: 73-112 (2010).)

FIG. 18 shows that ATM is recruited to the DSB sites and triggers autophosphorylation. There is also increased expression of pRPA32 (S33) and γH2A.X.

FIG. 19 shows that VAL-083 treatment for 1 hour activated p-ATM (S1981) and p-RPA32 (S33). (FIG. 19 is the same as FIG. 10.)

FIG. 20 shows the cell cycle and its association with cyclin expression.

FIG. 21 shows that VAL-083 pulse treatment strongly increased γH2AX and cyclin A2 expression with cell cycle arrest at S/G2 phase.

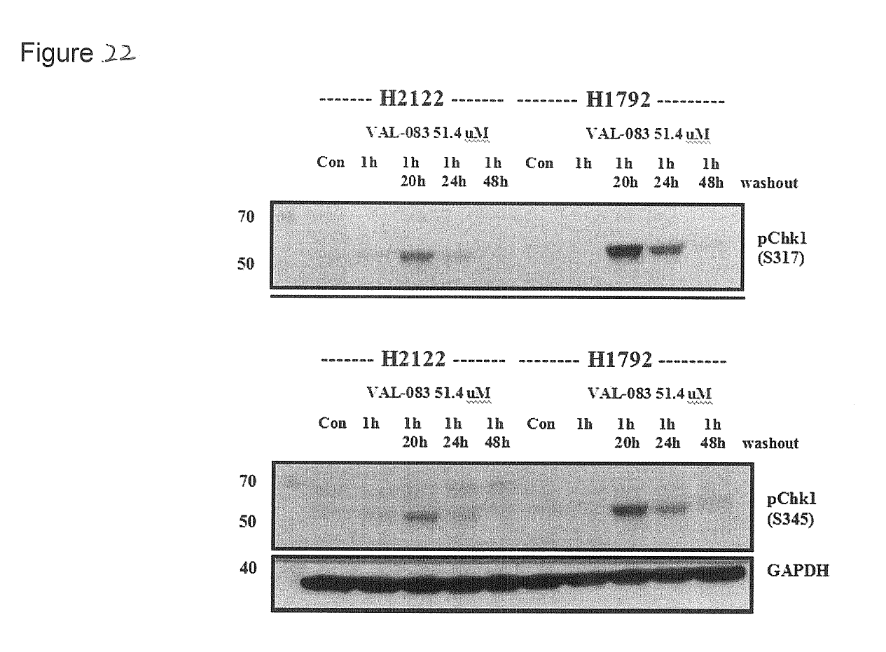

FIG. 22 shows that VAL-083 pulse treatment (51.4 μM for 1 h) activated p-ATM (S1981) in serum starvation synchronized cells.

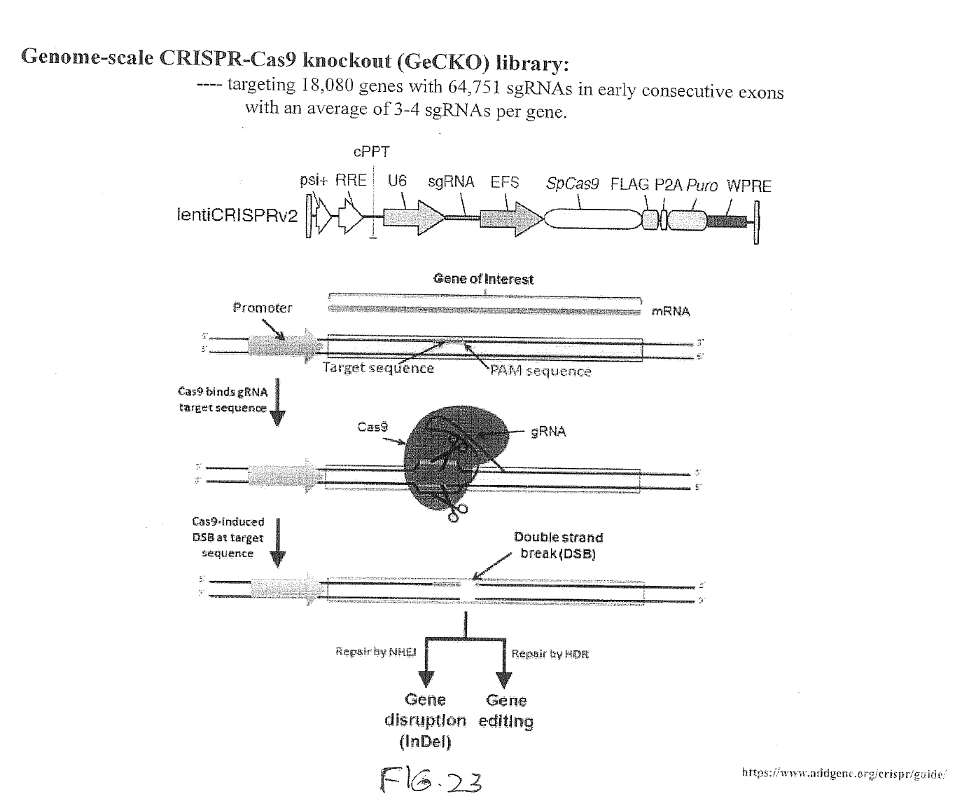

FIG. 23 shows that VAL-083 pulse treatment (51.4 μM for 1 h) induced activation of pChk2 (T68), which is the downstream effector of activated ATM kinase.

(Please use the new FIG. 24 in the attachment of email.) FIG. 24 shows activation of pChk1 (S317 and S345) with VAL-083 pulse treatment (51.4 μM for 1 h).

FIG. 25 depicts a genome-scale CRISPR-Cas9 knockout (GeCKO) library (O. Shalem et al., “Genome-Scale CRISPR-Cas9 Knockout Screening in Human Cells,” Science 343: 84-87 (2014)).

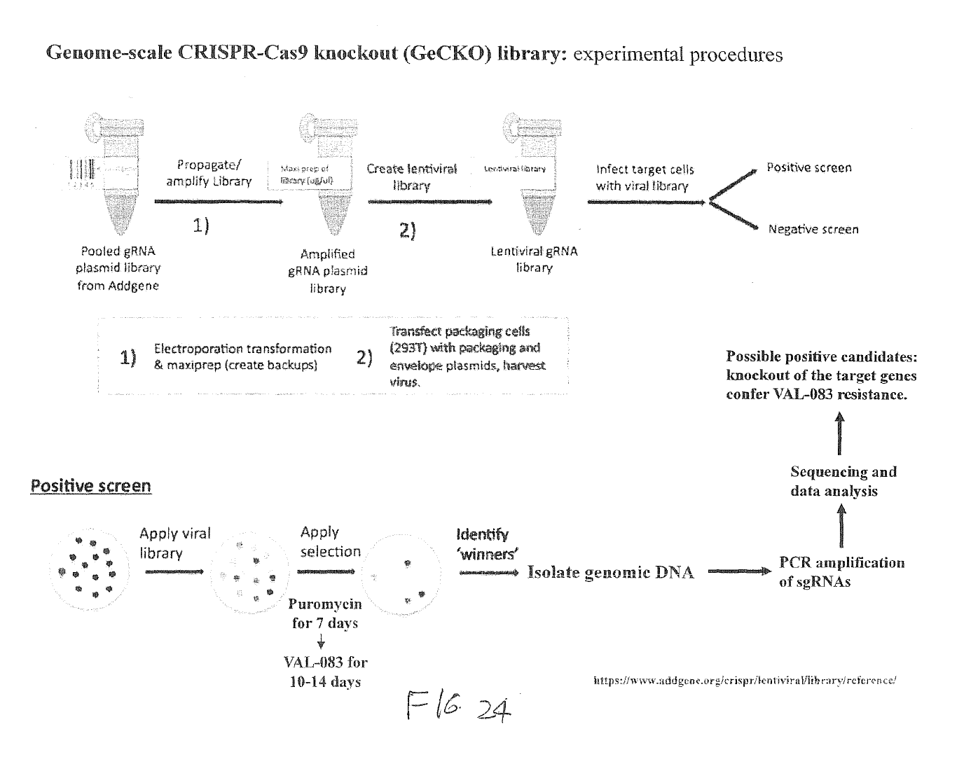

FIG. 26 depicts the experimental procedures for developing the genome-scale CRISPR-Cas9 knockout (GeCKO) library of FIG. 25.

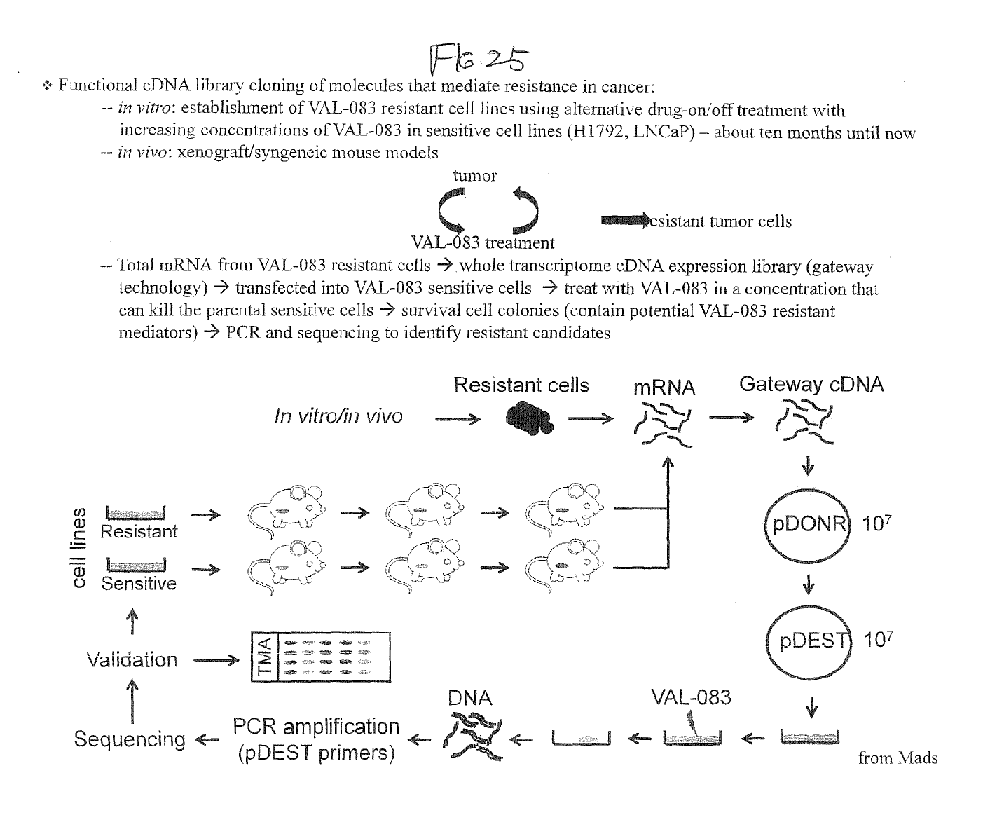

FIG. 27 depicts functional cDNA library cloning and proposed experiments.

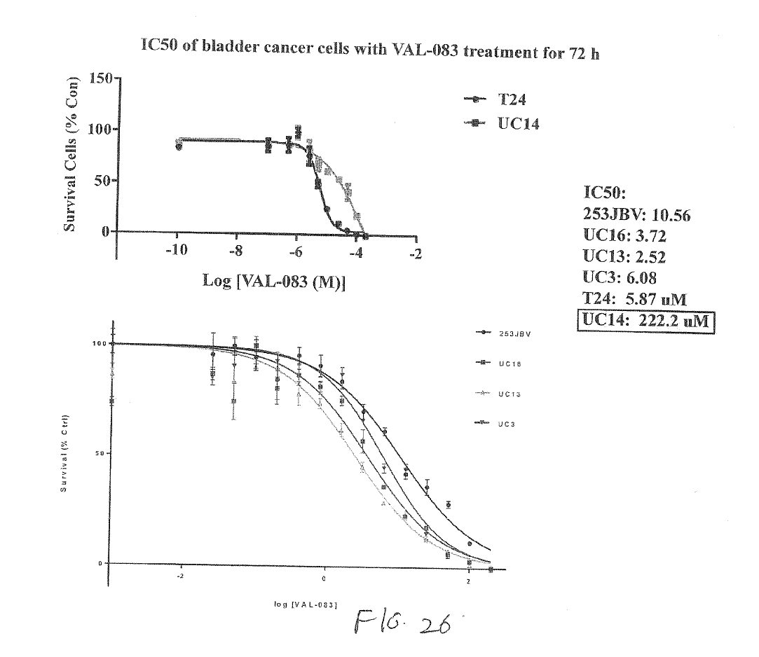

FIG. 28 is a graph showing the IC₅₀ of several bladder cancer cell lines with treatment by dianhydrogalactitol (VAL-083) for 72 hours, including 253JBV, UC16, UC13, UC3, T24, and UC14.

It is known that a number of signaling pathways are involved in response to DNA damage, particularly DNA damage characterized by the generation of double-strand breaks. Results obtained by immunochemistry such as by use of the Western blot technique show that the following pathways or molecules are involved: γH2AX, p-RPA32 (S4/8, S33), ATR, ATM, Rad51, CtIP, and BRCA1. Results obtained by the use of siRNA knockdown techniques show that the following pathways or molecules are involved: LEDGF, CtIP, BRCA1, and Rad51. Modulation of one or more of these pathways can be useful for the prevention or reduction of resistance to anti-neoplastic agents, including, but not limited to, substituted hexitol derivatives.

Methods according to the present invention possess industrial applicability for the preparation of a medicament for the treatment of a number of diseases and conditions in subjects, including the malignancies of the central nervous system. Compositions according to the present invention possess industrial applicability as pharmaceutical compositions.

The method claims of the present invention provide specific method steps that are more than general applications of laws of nature and require that those practicing the method steps employ steps other than those conventionally known in the art, in addition to the specific applications of laws of nature recited or implied in the claims, and thus confine the scope of the claims to the specific applications recited therein. In some contexts, these claims are directed to new ways of using an existing drug.

The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein.

In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. The disclosures of all articles and references, including patents and patent publications, are incorporated herein by reference.

What is claimed is:

1. A method for the treatment of a malignancy selected from the group consisting of glioblastoma, non-small-cell lung carcinoma (NSCLC), and ovarian cancer by the induction of double-strand breaks in the DNA of tumor cells by administration of a therapeutically effective quantity of a substituted hexitol derivative selected from the group consisting of dianhydrogalactitol, a derivative or analog of dianhydrogalactitol, diacetyldianhydrogalactitol, and a derivative or analog of diacetyldianhydrogalactitol.

2. The method of claim 1 wherein the malignancy is glioblastoma.

3. The method of claim 1 wherein the malignancy is NSCLC.

4. The method of claim 1 wherein the malignancy is ovarian cancer.

5. The method of claim 1 wherein the substituted hexitol derivative is selected from the group consisting of dianhydrogalactitol and a derivative or analog of dianhydrogalactitol.

6. The method of claim 5 wherein the substituted hexitol derivative selected from the group consisting of dianhydrogalactitol and a derivative or analog of dianhydrogalactitol is dianhydrogalactitol.

7. The method of claim 5 wherein the substituted hexitol derivative selected from the group consisting of dianhydrogalactitol and a derivative or analog of dianhydrogalactitol is a derivative or analog of dianhydrogalactitol.

8. The method of claim 7 wherein the derivative or analog of dianhydrogalactitol is a derivative of dianhydrogalactitol that is selected from the group consisting of: (i) a derivative of dianhydrogalactitol that has one or both of the hydrogens of the two hydroxyl groups of dianhydrogalactitol replaced with lower alkyl; (ii) a derivative of dianhydrogalactitol that has one or more of the hydrogens attached to the two epoxide rings replaced with lower alkyl; (iii) a derivative of dianhydrogalactitol that has one or both of the methyl groups present in dianhydrogalactitol and that are attached to the same carbons that bear the hydroxyl groups replaced with C₂-C₆ lower alkyl; and (iv) a derivative of dianhydrogalactitol that has one or both of the methyl groups present in dianhydrogalactitol and that are attached to the same carbons that bear the hydroxyl groups substituted with a halo group by replacing a hydrogen of the methyl group with a halo group.

9. The method of claim 1 wherein the hexitol derivative is selected from the group consisting of diacetyldianhydrogalactitol and a derivative or analog of diacetyldianhydrogalactitol.

10. The method of claim 9 wherein the hexitol derivative selected from the group consisting of diacetyldianhydrogalactitol and a derivative or analog of diacetyldianhydrogalactitol is diacetyldianhydrogalactitol.

11. The method of claim 9 wherein the hexitol derivative is selected from the group consisting of diacetyldianhydrogalactitol and a derivative or analog of diacetyldianhydrogalactitol is a derivative or analog of diacetyldianhydrogalactitol.

12. The method of claim 11 wherein the derivative or analog of diacetyldianhydrogalactitol is a derivative of diacetyldianhydrogalactitol that is selected from the group consisting of: (i) a derivative of diacetyldianhydrogalactitol that has one or both of the methyl groups that are part of the acetyl moieties replaced with C₂-C₆ lower alkyl; (ii) a derivative of diacetyldianhydrogalactitol that has one or both of the hydrogens attached to the epoxide ring replaced with lower alkyl; (iii) a derivative of diacetyldianhydrogalactitol that has one or both of the methyl groups attached to the same carbons that bear the acetyl groups replaced with C₂-C₆ lower alkyl; and (iv) a derivative of diacetyldianhydrogalactitol that has one or both of the methyl groups that are attached to the same carbons that bear the hydroxyl groups substituted with a halo group by replacing a hydrogen of the methyl group with a halo group.

13. The method of claim 1 wherein the method comprises the steps of:
(a) identifying at least one factor or parameter associated with the efficacy and/or occurrence of side effects of the administration of the substituted hexitol derivative for treatment of glioblastoma, NSCLC, or ovarian cancer; and
(b) modifying the factor or parameter to improve the efficacy and/or reduce the side effects of the administration of the substituted hexitol derivative for treatment of glioblastoma, NSCLC, or ovarian cancer.

14. The method of claim 13 wherein the factor or parameter is selected from the group consisting of:
(a) dose modification;
(b) route of administration;
(c) schedule of administration;
(d) indications for use;
(e) selection of disease stage;
(f) other indications;
(g) patient selection;
(h) patient/disease phenotype;
(i) patient/disease genotype;
(j) pre/post-treatment preparation
(k) toxicity management;
(l) pharmacokinetic/pharmacodynamic monitoring;
(m) drug combinations;
(n) chemosensitization;
(o) chemopotentiation;
(p) post-treatment patient management;
(q) alternative medicine/therapeutic support;
(r) bulk drug product improvements;
(s) diluent systems;
(t) solvent systems;
(u) excipients;
(v) dosage forms;
(w) dosage kits and packaging;
(x) drug delivery systems;
(y) drug conjugate forms;
(z) compound analogs;
(aa) prodrugs;
(ab) multiple drug systems;
(ac) biotherapeutic enhancement;
(ad) biotherapeutic resistance modulation;
(ae) radiation therapy enhancement;
(af) novel mechanisms of action;
(ag) selective target cell population therapeutics;
(ah) use with ionizing radiation;
(ai) use with an agent enhancing its activity;
(aj) use with an agent that counteracts myelosuppression; and
(ak) use with an agent that increases the ability of the substituted hexitol to pass through the blood-brain barrier.

15. The method of claim 14 wherein the factor or parameter is dose modification, and wherein the dose modification is a dose modification selected from the group consisting of:
(a) continuous i.v. infusion for hours to days;
(b) biweekly administration;
(c) doses greater than 5 mg/m²/day;
(d) progressive escalation of dosing from 1 mg/m²/day based on patient tolerance;
(e) use of caffeine to modulate metabolism;
(f) use of isoniazid to modulate metabolism;
(g) selected and intermittent boosting of dosage administration;
(h) administration of single and multiple doses escalating from 5 mg/m²/day via bolus;
(i) oral dosages of below 30 mg/m²;
oral dosages of above 130 mg/m²;
(k) oral dosages up to 40 mg/m² for 3 days and then a nadir/recovery period of 18-21 days;
(l) dosing at a lower level for an extended period;
(m) dosing at a higher level;
(n) dosing with a nadir/recovery period longer than 21 days;
(o) dosing at a level to achieve a concentration of the substituted hexitol derivative such as dianhydrogalactitol in the cerebrospinal fluid (CSF) of equal to or greater than 5 μM;
(p) dosing at a level to achieve a cytotoxic concentration in the CSF;
(q) the use of a substituted hexitol derivative such as dianhydrogalactitol as a single cytotoxic agent;
(r) administration on a 33-day cycle with a cumulative dose of about 9 mg/m²;
(s) administration on a 33-day cycle with a cumulative dose of about 10 mg/m²;
(t) administration on a 33-day cycle with a cumulative dose of about 20 mg/m²;
(u) administration on a 33-day cycle with a cumulative dose of about 40 mg/m²;
(v) administration on a 33-day cycle with a cumulative dose of about 80 mg/m²;
(w) administration on a 33-day cycle with a cumulative dose of about 160 mg/m²;
(x) administration on a 33-day cycle with a cumulative dose of about 240 mg/m²;
(y) administration so that the plasma half-life is about 1-2 hours;
(z) administration so that the Cₘₐₓ is <200 ng/ml; and
(aa) administration so that the substituted hexitol derivative has a half-life of >20 hours in the cerebrospinal fluid.

16. The method of claim 15 wherein the substituted hexitol derivative is dianhydrogalactitol.

17. The method of claim 14 wherein the factor or parameter is route of administration, and the route of administration is selected from the group consisting of:
(a) topical administration;
(b) oral administration;
(c) slow release oral delivery;
(d) intrathecal administration;
(e) intraarterial administration;
(f) continuous infusion;
(g) intermittent infusion;
(h) intravenous administration, such as intravenous administration for 30 minutes;
(i) administration through a longer infusion;
(j) administration through IV push; and
(k) administration to maximize the concentration of the substituted hexitol derivative in the CSF.

18. The method of claim 17 wherein the substituted hexitol derivative is dianhydrogalactitol.

19. The method of claim 14 wherein the factor or parameter is schedule of administration, and wherein the schedule of administration is selected from the group consisting of:
(a) daily administration;
(b) weekly administration;
(c) weekly administration for three weeks;
(d) biweekly administration;
(e) biweekly administration for three weeks with a 1-2 week rest period;
(f) intermittent boost dose administration;
(g) daily administration for one week for multiple weeks; and
(h) administration on days 1, 2, and 3 of a 33-day cycle.

20. The method of claim 19 wherein the substituted hexitol derivative is dianhydrogalactitol.

21. The method of claim 14 wherein the factor or parameter is selection of disease stage, and wherein the selection of disease stage is a selection of disease stage selected from the group consisting of:
(a) use in an appropriate disease stage for glioblastoma, NSCLC, or ovarian cancer;
(b) use with an angiogenesis inhibitor to prevent or limit metastatic spread;
(c) use for newly diagnosed disease;
(d) use for recurrent disease;
(e) use for resistant or refractory disease; and
(f) use for childhood glioblastoma.

22. The method of claim 21 wherein the substituted hexitol derivative is dianhydrogalactitol.

23. The method of claim 14 wherein the factor or parameter is patient selection and wherein the patient selection is a patient selection carried out by a criterion selected from the group consisting of:
(a) selecting patients with a disease condition characterized by a high level of a metabolic enzyme selected from the group consisting of histone deacetylase and ornithine decarboxylase;
(b) selecting patients with a low or high susceptibility to a condition selected from the group consisting of thrombocytopenia and neutropenia;
(c) selecting patients intolerant of GI toxicities;
(d) selecting patients characterized by over- or under-expression of a gene selected from the group consisting of c-Jun, a GPCR, a signal transduction protein, VEGF, a prostate-specific gene, and a protein kinase.
(e) selecting patients characterized by carrying extra copies of the EGFR gene for glioblastoma, NSCLC, or ovarian cancer;
(f) selecting patients characterized by mutations in at least one gene selected from the group consisting of TP53, PDGFRA, IDH1, and NF1 for glioblastoma, NSCLC, or ovarian cancer;
(g) selecting patients characterized by methylation or lack of methylation of the promoter of the MGMT gene;
(h) selecting patients characterized by the existence of an IDH1 mutation;
(i) selecting patients characterized by the presence of IDH1 wild-type gene;
(j) selecting patients characterized by the presence of 1p/19q co-deletion;
(k) selecting patients characterized by the absence of an 1p/19q co-deletion;
(l) selecting patients characterized by an unmethylated promoter region of MGMT (O⁶-methylguanine methyltransferase);
(m) selecting patients characterized by a methylated promoter region of MGMT;
(n) selecting patients characterized by a high expression of MGMT;
(o) selecting patients characterized by a low expression of MGMT; and
(p) selecting patients characterized by a mutation in EGFR.

24. The method of claim 23 wherein the substituted hexitol derivative is dianhydrogalactitol.

25. The method of claim 14 wherein the factor or parameter is analysis of patient or disease phenotype and wherein the analysis of patient or disease phenotype is a method of analysis of patient or disease phenotype carried out by a method selected from the group consisting of:
(a) use of a diagnostic tool, a diagnostic technique, a diagnostic kit, or a diagnostic assay to confirm a patient's particular phenotype;
(b) use of a method for measurement of a marker selected from the group consisting of histone deacetylase, ornithine decarboxylase, VEGF, a protein that is a gene product of jun, and a protein kinase;
(c) surrogate compound dosing; and
(d) low dose pre-testing for enzymatic status.

26. The method of claim 25 wherein the substituted hexitol derivative is dianhydrogalactitol.

27. The method of claim 14 wherein the factor or parameter is analysis of patient or disease genotype and wherein the analysis of patient or disease genotype is a method of analysis of patient or disease genotype carried out by a method selected from the group consisting of:
(a) use of a diagnostic tool, a diagnostic technique, a diagnostic kit, or a diagnostic assay to confirm a patient's particular genotype;
(b) use of a gene chip;
(c) use of gene expression analysis;
(d) use of single nucleotide polymorphism (SNP) analysis;
(e) measurement of the level of a metabolite or a metabolic enzyme;
(f) determination of mutation of PDGFRA gene;
(g) determination of mutation of IDH1 gene; (This is the same as (k)) below.)
(h) determination of mutation of NF1 gene;
(i) determination of copy number of the EGFR gene;
(j) determination of status of methylation of promoter of MGMT gene; (This is the same as (o) and (p) below.)
(k) determination of the existence of an IDH1 mutation;
(l) determination of the existence of IDH1 wild-type;
(m) determination of the existence of a 1p/19q co-deletion;
(n) determination of the absence of a 1p/19q co-deletion;
(o) determination of the existence of an unmethylated promoter region of the MGMT gene;
(p) determination of the existence of a methylated promoter region of the MGMT gene;
(q) determination of the existence of high expression of MGMT; and
(r) determination of the existence of low expression of MGMT.

28. The method of claim 27 wherein the substituted hexitol derivative is dianhydrogalactitol.

29. The method of claim 14 wherein the factor or parameter is pre/post treatment preparation and wherein the pre/post-treatment preparation is a method of pre/post treatment preparation selected from the group consisting of:
(a) the use of colchicine or an analog thereof;
(b) the use of a diuretic;
(c) the use of a uricosuric;
(d) the use of uricase;
(e) the non-oral use of nicotinamide;
(f) the use of a sustained-release form of nicotinamide;
(g) the use of an inhibitor of poly-ADP ribose polymerase;
(h) the use of caffeine;
(i) the use of leucovorin rescue;
(j) infection control; and
(k) the use of an anti-hypertensive agent.

30. The method of claim 29 wherein the substituted hexitol derivative is dianhydrogalactitol.

31. The method of claim 14 wherein the factor or parameter is toxicity management, and wherein the toxicity management is a method of toxicity management selected from the group consisting of:
(a) the use of colchicine or an analog thereof;
(b) the use of a diuretic;
(c) the use of a uricosuric;
(d) the use of uricase;
(e) the non-oral use of nicotinamide;
(f) the use of a sustained-release form of nicotinamide;
(g) the use of an inhibitor of poly-ADP ribose polymerase;
(h) the use of caffeine;
(i) the use of leucovorin rescue;
(j) the use of sustained-release allopurinol;
(k) the non-oral use of allopurinol;
(l) the use of bone marrow transplants;
(m) the use of a blood cell stimulant;
(n) the use of blood or platelet infusions;
(o) the administration of an agent selected from the group consisting of filgrastim, G-CSF, and GM-CSF;
(p) the application of a pain management technique;
(q) the administration of an anti-inflammatory agent;
(r) the administration of fluids;
(s) the administration of a corticosteroid;
(t) the administration of an insulin control medication;
(u) the administration of an antipyretic;
(v) the administration of an anti-nausea treatment;
(w) the administration of an anti-diarrheal treatment;
(x) the administration of N-acetylcysteine; and
(y) the administration of an antihistamine.

32. The method of claim 31 wherein the substituted hexitol derivative is dianhydrogalactitol.

33. The method of claim 14 wherein the factor or parameter is pharmacokinetic/pharmacodynamic monitoring, and wherein the pharmacokinetic/pharmacodynamic monitoring is a method selected from the group consisting of:
(a) multiple determinations of blood plasma levels; and
(b) multiple determinations of at least one metabolite in blood or urine.

34. The method of claim 33 wherein the substituted hexitol derivative is dianhydrogalactitol.

35. The method of claim 14 wherein the factor or parameter is drug combination, and wherein the drug combination is a drug combination selected from the group consisting of:
(a) use with fraudulent nucleosides;
(b) use with fraudulent nucleotides;
(c) use with thymidylate synthetase inhibitors;
(d) use with signal transduction inhibitors;
(e) use with cisplatin or platinum analogs;
(f) use with alkylating agents;
(g) use with anti-tubulin agents;
(h) use with antimetabolites;
(i) use with berberine;
(j) use with apigenin;
(k) use with colchicine or an analog thereof;
(l) use with genistein;
(m) use with etoposide;
(n) use with cytarabine;
(o) use with camptothecins;
(p) use with vinca alkaloids;
(q) use with topoisomerase inhibitors;
(r) use with 5-fluorouracil;
(s) use with curcumin;
(t) use with NF-κB inhibitors;
(u) use with rosmarinic acid;
(v) use with mitoguazone;
(w) use with meisoindigo;
(x) use with imatinib;
(y) use with dasatinib;
(z) use with nilotinib;
(aa) use with epigenetic modulators;
(ab) use with transcription factor inhibitors;
(ac) use with taxol;
(ad) use with homoharringtonine;
(ae) use with pyridoxal;
(af) use with spirogermanium;
(ag) use with caffeine;
(ah) use with nicotinamide;
(ai) use with methylglyoxal bisguanylhydrazone;
(aj) use with Rho kinase inhibitors;
(ak) use with 1,2,4-benzotriazine oxides;
(al) use with an alkylglycerol;
(am) use with an inhibitor of a Mer, Ax1, or Tyro-3 receptor kinase;
(an) use with an inhibitor of ATR kinase;
(ao) use with a modulator of Fms kinase, Kit kinase, MAP4K4 kinase, TrkA kinase, or TrkB kinase;
(ap) use with endoxifen;
(aq) use with a mTOR inhibitor;
(ar) use with an inhibitor of Mnk1a kinase, Mkn1b kinase, Mnk2a kinase, or Mnk2b kinase;
(as) use with a modulator of pyruvate kinase M2;
(at) use with a modulator of phosphoinositide 3-kinases;
(au) use with a cysteine protease inhibitor;
(av) use with phenformin;
(aw) use with Sindbis virus-based vectors;
(ax) use with peptidomimetics that act as mimetics of Smac and inhibit IAPs to promote apoptosis;
(ay) use with a Raf kinase inhibitor;
(az) use with a nuclear transport modulator;
(ba) use with an acid ceramidase inhibitor and a choline kinase inhibitor;
(bb) use with tyrosine kinase inhibitors;
(bc) use with anti-CS1 antibodies;
(bd) use with inhibitors of protein kinase CK2;
(be) use with anti-guanylyl cyclase C (GCC) antibodies;
(bf) use with histone deacetylase inhibitors;
(bg) use with cannabinoids;
(bh) use with glucagon-like peptide-1 (GLP-1) receptor agonists;
(bi) use with inhibitors of Bcl-2 or Bcl-xL;
(bj) use with Stat3 pathway inhibitors;
(bk) use with inhibitors of polo-like kinase 1 (Plk1);
(bl) use with GBPAR1 activators;
(bm) use with modulators of serine-threonine protein kinase and poly(ADP-ribose) polymerase (PARP) activity;
(bn) use with taxanes;
(bo) use with inhibitors of dihydrofolate reductase;
(bp) use with inhibitors of aromatase;
(bq) use with benzimidazole-based anti-neoplastic agents;
(br) use with an O6-methylguanine-DNA-methyltransferase (MGMT) inhibitor;
(bs) use with CCR9 inhibitors;
(bt) use with acid sphingomyelinase inhibitors;
(bu) use with peptidomimetic macrocycles;
(bv) use with cholanic acid amides;
(bw) use with substituted oxazaphosphorines;
(bx) use with anti-TWEAK receptor antibodies;
(by) use with an ErbB3 binding protein;
(bz) use with a glutathione S-transferase-activated anti-neoplastic compound;
(ca) use with substituted phosphorodiamidates;
(cb) use with inhibitors of MEKK protein kinase;
(cd) use with COX-2 inhibitors;
(ce) use with cimetidine and a cysteine derivative;
(cf) use with anti-IL-6 receptor antibody;
(cg) use with an antioxidant;
(ch) use with an isoxazole inhibitor of tubulin polymerization;
(ci) use with PARP inhibitors;
(cj) use with Aurora protein kinase inhibitors;
(ck) use with peptides binding to prostate-specific membrane antigen;
(cl) use with CD19 binding agents;
(cm) use with benzodiazepines;
(cn) use with Toll-like receptor (TLR) agonists;
(co) use with bridged bicyclic sulfamides;
(cp) use with inhibitors of epidermal growth factor receptor kinase;
(cq) use with a ribonuclease of the T2 family having actin-binding activity;
(cr) use with myrsinoic acid A or an analog thereof;
(cs) use with inhibitors of a cyclin-dependent kinase;
(ct) use with inhibitors of the interaction between p53 and MDM2;
(cu) use with inhibitors of the receptor tyrosine kinase MET;
(cv) use with largazole or largazole analogs;
(cw) use with inhibitors of AKT protein kinase;
(cx) use with 2′-fluoro-5-methyl-β-L-arabinofuranosyluridine or L-deoxythymidine;
(cy) use with HSP90 modulators;
(cz) use with inhibitors of JAK kinases;
(da) use with inhibitors of PDK1 protein kinase;
(db) use with PDE4 inhibitors;
(de) use with inhibitors of proto-oncogene c-Met tyrosine kinase;
(df) use with inhibitors of indoleamine 2,3-dioxygenase;
(dg) use with agents that inhibit expression of ATDC (TRIM29);
(dh) use with proteomimetic inhibitors of the interaction of nuclear receptor with coactivator peptides;
(di) use with antagonists of XIAP family proteins;
(dj) use with tumor-targeted superantigens;
(dk) use with inhibitors of Pim kinases;
(dl) use with inhibitors of CHK1 or CHK2 kinases;
(dm) use with inhibitors of angiopoietin-like 4 protein;
(dn) use with Smo antagonists;
(do) use with nicotinic acetylcholine receptor antagonists;
(dp) use with farnesyl protein transferase inhibitors;
(dq) use with adenosine A3 receptor antagonists;
(dr) use with a cancer vaccine;
(ds) use with a JAK2 inhibitor; and
(dt) use with a Src inhibitor.

36. The method of claim 35 wherein the substituted hexitol derivative is dianhydrogalactitol.

37. The method of claim 14 wherein the factor or parameter is chemosensitization, and wherein the chemosensitization is use of a substituted hexitol derivative as a chemosensitizer in combination with an agent selected from the group consisting of:
(a) topoisomerase inhibitors; (This is the same as (s) below.)
(b) fraudulent nucleosides;
(c) fraudulent nucleotides;
(d) thymidylate synthetase inhibitors;
(e) signal transduction inhibitors;
(f) cisplatin or platinum analogs;
(g) alkylating agents;
(h) anti-tubulin agents;
(i) antimetabolites;
(j) berberine;
(k) apigenin;
(l) amonafide;
(m) colchicine or analogs;
(n) genistein;
(o) etoposide;
(p) cytarabine;
(q) camptothecins;
(r) vinca alkaloids;
(s) topoisomerase inhibitors;
(t) 5-fluorouracil;
(u) curcumin;
(v) NF-κB inhibitors;
(w) rosmarinic acid;
(x) mitoguazone;
(y) tetrandrine;
(z) a tyrosine kinase inhibitor;
(aa) an inhibitor of EGFR; and
(ab) an inhibitor of PARP.

38. The method of claim 37 wherein the substituted hexitol derivative is dianhydrogalactitol.

39. The method of claim 14 wherein the factor or parameter is chemopotentiation, and wherein the chemopotentiation is use of a substituted hexitol derivative as a chemopotentiator in combination with an agent selected from the group consisting of:
(a) topoisomerase inhibitors;
(b) fraudulent nucleosides;
(c) fraudulent nucleotides;
(d) thymidylate synthetase inhibitors;
(e) signal transduction inhibitors;
(f) cisplatin or platinum analogs;
(g) alkylating agents;
(h) anti-tubulin agents;
(i) antimetabolites;
(j) berberine;
(k) apigenin;
(l) amonafide;
(m) colchicine or analogs;
(n) genistein;
(o) etoposide;
(p) cytarabine;
(q) camptothecins;
(r) vinca alkaloids;
(s) 5-fluorouracil;
(t) curcumin;
(u) NF-κB inhibitors;
(v) rosmarinic acid;
(w) mitoguazone;
(x) tetrandrine;
(y) a tyrosine kinase inhibitor;
(z) an inhibitor of EGFR; and
(aa) an inhibitor of PARP.

40. The method of claim 39 wherein the substituted hexitol derivative is dianhydrogalactitol.

41. The method of claim 14 wherein the factor or parameter is post-treatment management, and wherein the post-treatment management is a method selected from the group consisting of:
(a) a therapy associated with pain management;
(b) administration of an anti-emetic;
(c) an anti-nausea therapy;
(d) administration of an anti-inflammatory agent;
(e) administration of an anti-pyretic agent; and
(f) administration of an immune stimulant.

42. The method of claim 41 wherein the substituted hexitol derivative is dianhydrogalactitol.

43. The method of claim 14 wherein the factor or parameter is alternative medicine/post-treatment support, and wherein the alternative medicine/post-treatment support is a herbal medication created either synthetically or through extraction, wherein the herbal medication created either synthetically or through extraction is selected from the group consisting of:
(a) a NF-κB inhibitor;
(b) a natural anti-inflammatory;
(c) an immunostimulant;
(d) an antimicrobial; and
(e) a flavonoid, isoflavone, or flavone.

44. The method of claim 43 wherein the substituted hexitol derivative is dianhydrogalactitol.

45. The method of claim 14 wherein the factor or parameter is use of a bulk drug product improvement, and wherein the bulk drug product improvement is selected from the group consisting of:
(a) salt formation;
(b) preparation as a homogeneous crystal structure;
(c) preparation as a pure isomer;
(d) increased purity;
(e) preparation with lower residual solvent content; and
(f) preparation with lower residual heavy metal content.

46. The method of claim 45 wherein the substituted hexitol derivative is dianhydrogalactitol.

47. The method of claim 14 wherein the factor or parameter is use of a diluent, and the diluent is selected from the group consisting of:
(a) an emulsion;
(b) dimethylsulfoxide (DMSO);
(c) N-methylformamide (NMF)
(d) DMF;
(e) ethanol;
(f) benzyl alcohol;
(g) dextrose-containing water for injection;
(h) Cremophor;
(i) cyclodextrin; and
(j) PEG.

48. The method of claim 47 wherein the substituted hexitol derivative is dianhydrogalactitol.

49. The method of claim 14 wherein the factor or parameter is use of a solvent system, and the solvent system is selected from the group consisting of:
(a) an emulsion;
(b) dimethylsulfoxide (DMSO);
(c) N-methylformamide (NMF)
(d) DMF;
(e) ethanol;
(f) benzyl alcohol;
(g) dextrose-containing water for injection;
(h) Cremophor;
(i) cyclodextrin; and
(j) PEG.

50. The method of claim 49 wherein the substituted hexitol derivative is dianhydrogalactitol.

51. The method of claim 14 wherein the factor or parameter is use of an excipient, and the excipient is selected from the group consisting of:
(a) mannitol;
(b) albumin;
(c) EDTA;
(d) sodium bisulfite;
(e) benzyl alcohol;
(f) carbonate buffers;
(g) phosphate buffers;
(h) PEG;
(i) vitamin A;
(j) vitamin D;
(k) vitamin E;
(l) esterase inhibitors;
(m) cytochrome P450 inhibitors;
(n) multi-drug resistance (MDR) inhibitors;
(o) organic resins;
(p) detergents;
(q) perillyl alcohol or an analog thereof; and
(r) activators of channel-forming receptors.

52. The method of claim 51 wherein the substituted hexitol derivative is dianhydrogalactitol.

53. The method of claim 14 wherein the factor or parameter is use of a dosage form, and the dosage form is selected from the group consisting of:
(a) tablets;
(b) capsules;
(c) topical gels;
(d) topical creams;
(e) patches;
(f) suppositories;
(g) lyophilized dosage fills;
(h) immediate-release formulations;
(i) slow-release formulations;
(j) controlled-release formulations; and
(k) liquid in capsules.

54. The method of claim 53 wherein the substituted hexitol derivative is dianhydrogalactitol.

55. The method of claim 14 wherein the factor or parameter is use of a drug delivery system, and the drug delivery system is selected from the group consisting of:
(a) oral dosage forms;
(b) nanocrystals;
(c) nanoparticles;
(d) cosolvents;
(e) slurries;
(f) syrups;
(g) bioerodible polymers;
(h) liposomes;
(i) slow-release injectable gels;
(j) microspheres; and
(k) targeting compositions with epidermal growth factor receptor-binding peptides.

56. The method of claim 55 wherein the substituted hexitol derivative is dianhydrogalactitol.

57. The method of claim 14 wherein the factor or parameter is use of a drug conjugate form, and the drug conjugate form is selected from the group consisting of:
(a) a polymer system;
(b) polylactides;
(c) polyglycolides;
(d) amino acids;
(e) peptides;
(f) multivalent linkers;
(g) immunoglobulins;
(h) cyclodextrin polymers;
(i) modified transferrin;
(j) hydrophobic or hydrophobic-hydrophilic polymers;
(k) conjugates with a phosphonoformic acid partial ester;
(l) conjugates with a cell-binding agent incorporating a charged cross-linker; and
(m) conjugates with β-glucuronides through a linker.

58. The method of claim 57 wherein the substituted hexitol derivative is dianhydrogalactitol.

59. The method of claim 14 wherein the factor or parameter is use of a prodrug system, and the prodrug system is selected from the group consisting of:
(a) the use of enzyme sensitive esters;
(b) the use of dimers;
(c) the use of Schiff bases;
(d) the use of pyridoxal complexes;
(e) the use of caffeine complexes; and
(f) the use of nitric oxide-releasing prodrugs;
(g) the use of prodrugs with fibroblast activation protein α-cleavable oligopeptides;
(h) the use of prodrugs that are products of reaction with an acetylating or carbamylating agent;
(i) the use of prodrugs that are hexanoate conjugates;
(j) the use of prodrugs that are polymer-agent conjugates; and
(k) the use of prodrugs that are subject to redox activation.

60. The method of claim 59 wherein the substituted hexitol derivative is dianhydrogalactitol.

61. The method of claim 14 wherein the factor or parameter is use of a multiple drug system, and the multiple drug system is selected from the group consisting of:
(a) inhibitors of multi-drug resistance;
(b) specific drug resistance inhibitors;
(c) specific inhibitors of selective enzymes;
(d) signal transduction inhibitors;
(e) meisoindigo;
(f) imatinib;
(g) hydroxyurea;
(h) dasatinib;
(i) capecitabine;
(j) nilotinib;
(k) repair inhibition agents; and
(l) topoisomerase inhibitors with non-overlapping side effects.

62. The method of claim 61 wherein the substituted hexitol derivative is dianhydrogalactitol.

63. The method of claim 14 wherein the factor or parameter is biotherapeutic enhancement, and wherein the biotherapeutic enhancement is performed by use in combination as sensitizers/potentiators with a therapeutic agent or technique selected from the group consisting of:
(a) biological response modifiers;
(b) cytokines;
(c) lymphokines;
(d) therapeutic antibodies;
(e) antisense therapies;
(f) gene therapies;
(g) ribozymes;
(h) RNA interference; and
(i) vaccines.

64. The method of claim 63 wherein the substituted hexitol derivative is dianhydrogalactitol.

65. The method of claim 14 wherein the factor or parameter is biotherapeutic resistance modulation, and wherein the biotherapeutic resistance modulation is use against a malignancy resistant to a therapeutic agent or technique selected from the group consisting of:
(a) biological response modifiers;
(b) cytokines;
(c) lymphokines;
(d) therapeutic antibodies;
(e) antisense therapies;
(f) gene therapies;
(g) ribozymes;
(h) RNA interference; and
(i) vaccines.

66. The method of claim 65 wherein the substituted hexitol derivative is dianhydrogalactitol.

67. The method of claim 14 wherein the factor or parameter is radiation therapy enhancement, and wherein the radiation therapy enhancement is a radiation therapy enhancement agent or technique selected from the group consisting of:
(a) use with hypoxic cell sensitizers;
(b) use with radiation sensitizers/protectors;
(c) use with photosensitizers;
(d) use with radiation repair inhibitors;
(e) use with thiol depleting agents;
(f) use with vaso-targeted agents;
(g) use with DNA repair inhibitors;
(h) use with radioactive seeds;
(i) use with radionuclides;
(j) use with radiolabeled antibodies; and
(k) use with brachytherapy.

68. The method of claim 67 wherein the substituted hexitol derivative is dianhydrogalactitol.

69. The method of claim 14 wherein the factor or parameter is use of a novel mechanism of action, and wherein the novel mechanism of action is a novel mechanism of action that is a therapeutic interaction with a target or mechanism selected from the group consisting of:
(a) inhibitors of poly-ADP ribose polymerase;
(b) agents that affect vasculature or vasodilation;
(c) oncogenic targeted agents;
(d) signal transduction inhibitors;
(e) EGFR inhibition;
(f) protein kinase C inhibition;
(g) phospholipase C downregulation;
(h) Jun downregulation;
(i) histone genes;
(j) VEGF;
(k) ornithine decarboxylase;
(l) ubiquitin C;
(m) Jun D;
(n) v-Jun;
(o) GPCRs;
(p) protein kinase A;
(q) protein kinases other than protein kinase A;
(r) prostate specific genes;
(s) telomerase;
(t) histone deacetylase; and
(u) tyrosine kinase inhibitors.

70. The method of claim 69 wherein the substituted hexitol derivative is dianhydrogalactitol.

71. The method of claim 14 wherein the factor or parameter is use of selective target cell population therapeutics, and wherein the use of selective target cell population therapeutics is a use selected from the group consisting of:
(a) use against radiation sensitive cells;
(b) use against radiation resistant cells; and
(c) use against energy depleted cells.

72. The method of claim 71 wherein the substituted hexitol derivative is dianhydrogalactitol.

73. The method of claim 14 wherein the factor or parameter is use with an agent to enhance the activity of an alkylating hexitol derivative, and wherein the agent to enhance the activity of the alkylating hexitol derivative is an agent selected from the group consisting of:
(a) nicotinamide;
(b) caffeine;
(c) tetandrine; and
(d) berberine.

74. The method of claim 73 wherein the substituted hexitol derivative is dianhydrogalactitol.

75. The method of claim 14 wherein the factor or parameter is use with an agent to counteract myelosuppression and wherein the agent to counteract myelosuppression is a dithiocarbamate.

76. The method of claim 75 wherein the substituted hexitol derivative is dianhydrogalactitol.

77. The method of claim 14 wherein the factor or parameter is use with an agent that increases the ability of the substituted hexitol to pass through the blood-brain barrier, and wherein the agent that increases the ability of the substituted hexitol to pass through the blood-brain barrier is selected from the group consisting of:
(a) a chimeric peptide of the structure of Formula (D-III): 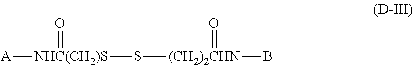
wherein: (A) A is somatostatin, thyrotropin releasing hormone (TRH), vasopressin, alpha interferon, endorphin, muramyl dipeptide or ACTH 4-9 analogue; and (B) B is insulin, IGF-I, IGF-II, transferrin, cationized (basic) albumin or prolactin; or a chimeric peptide of the structure of Formula (D-III) wherein the disulfide conjugating bridge between A and B is replaced with a bridge of Subformula (D-III(a)):
A-NH(CH₂)₂S—S—B (cleavable linkage) (D-III(a)),
wherein the bridge is formed using cysteamine and EDAC as the bridge reagents; or a chimeric peptide of the structure of Formula (D-III) wherein the disulfide conjugating bridge between A and B is replaced with a bridge of Subformula (D-III(b)):
A-NH═CH(CH₂)₃CH═NH—B (non-cleavable linkage) (D-III(b)),
wherein the bridge is formed using glutaraldehyde as the bridge reagent;
(b) a composition comprising either avidin or an avidin fusion protein bonded to a biotinylated substituted hexitol derivative to form an avidin-biotin-agent complex including therein a protein selected from the group consisting of insulin, transferrin, an anti-receptor monoclonal antibody, a cationized protein, and a lectin;
(c) a neutral liposome that is pegylated and incorporates the substituted hexitol derivative, wherein the polyethylene glycol strands are conjugated to at least one transportable peptide or targeting agent;
(d) a humanized murine antibody that binds to the human insulin receptor linked to the substituted hexitol derivative through an avidin-biotin linkage; and
(e) a fusion protein comprising a first segment and a second segment: the first segment comprising a variable region of an antibody that recognizes an antigen on the surface of a cell that after binding to the variable region of the antibody undergoes antibody-receptor-mediated endocytosis, and, optionally, further comprises at least one domain of a constant region of an antibody; and the second segment comprising a protein domain selected from the group consisting of avidin, an avidin mutein, a chemically modified avidin derivative, streptavidin, a streptavidin mutein, and a chemically modified streptavidin derivative, wherein the fusion protein is linked to the substituted hexitol by a covalent link to biotin.

78. The method of claim 77 wherein the substituted hexitol derivative is dianhydrogalactitol.

79. The method of claim 1 wherein the method further comprises administration of a therapeutically effective quantity of a PARP inhibitor.

80. The method of claim 79 wherein the PARP inhibitor is selected from the group consisting of iniparib, talazoparib, olaparib, rucaparib, veliparib, CEP-9722 (a prodrug of CEP-8983 (11-methoxy-4,5,6,7-tetrahydro-1H-cyclopenta[a]pyrrolo[3,4-c]carbazole-1,3(2H)-dione), MK 4827 ((S)-2-(4-(piperidin-3-yl)phenyl)-2H-indazole-7-carboxamide), and BGB-290.

81. The method of claim 79 wherein the substituted hexitol derivative is dianhydrogalactitol.

82. The method of claim 1 wherein the method further comprises administration of a therapeutically effective quantity of an agent that counters loss of PTEN function.

83. The method of claim 82 wherein the agent that counters loss of PTEN function is selected from the group consisting of temsirolimus, everolimus, AZD6482 ((R)-2-(1-(7-methyl-2-morpholino-4-oxo-4H-pyrido[1,2-a]pyrimidin-9-yl)ethylamino)benzoic acid), MK-2206 (8-(4-(1-aminocyclobutyl)phenyl)-9-phenyl-[1,2,4]triazolo[3,44][1,6]naphthyridin-3(2H)-one), and 17-AAG ([(3S,5S,6R,7S,8E,10R,11S,12E,14E)-21-(allylamino)-6-hydroxy-5,11-dimethoxy-3,7,9,15-tetramethyl-16,20,22-trioxo-17-azabicyclo[16.3.1]docosa-8,12,14,18,21-pentaen-10-yl] carbamate).

84. The method of claim 82 wherein the substituted hexitol derivative is dianhydrogalactitol.

85. The method of claim 1 wherein the method further comprises administration of a therapeutically effective quantity of an additional anti-neoplastic agent that damages DNA.

86. The method of claim 85 wherein the additional anti-neoplastic agent that damages DNA is selected from the group consisting of cisplatin, carboplatin, oxaliplatin, picoplatin, nedaplatin, satraplatin, tetraplatin, doxorubicin, daunorubicin, methotrexate, 5-fluorouracil, gemcitabine, podophyllotoxin, etoposide, teniposide, cyclophosphamide, chlorambucil, melphalan, carmustine, lomustine, estramustine, semustine, bendamustine, prednamustine, uramustine, chlornaphazine, dacarbazine, altretamine, temozolomide, mitomycin C, streptozotocin, chlorozotocin, capecitabine, floxuridine, 6-mercaptopurine, 8-azaguanine, azathiopurine, 5-ethynyluracil, thioguanine, fludarabine, cytarabine, cladribine, 2-fluoro-arabinosyl-adenine, aminopterin, pemetrexed, ralitrexed, camptothecin, epirubicin, idarubicin, methylnitronitrosoguanidine, topotecan, irinotecan, mechlorethamine, ifosfamide, trofosfamide, busulfan, procarbazine, mitoxantrone, actinomycin, calicheamicin, Tegafur (R,S-1-(tetrahydro-2-furanyl)-5-fluorouracil), 2′,2′-difluoro-2′-deoxycytidine, bischloroethylsulfide, thiotepa, aziridinylbenzoquinone, BCNU, CCNU, 4-methyl CCNU, ACNU, rebeccamycin, bleomycin, pepleomycin, ethylmethanesulfonate, methylmethanesulfonate, dimethylnitrosamine, dimethyl sulfate, and N′-[2-[2-(4-methoxyphenyl)ethenyl]-4-quinazolinyl]-N, N-dimethyl-1,3-propanediamine dihydrochloride.

87. The method of claim 85 wherein the substituted hexitol derivative is dianhydrogalactitol.

88. The method of claim 1 wherein the method further comprises administration of a therapeutically effective quantity of an agent modulating at least one of the following pathway mediators: γH2AX, p-RPA32 (S4/8, S33), ATR, ATM, Rad51, CtIP, BRCA1, and LEDGF.

89. The method of claim 1 wherein the method further comprises:
(i) administration of a therapeutically effective quantity of a topoisomerase inhibitor; and
(ii) administration of a therapeutically effective quantity of an inhibitor of CHK1 kinase or CHK2 kinase.

90. The method of claim 89 wherein the substituted hexitol derivative is dianhydrogalactitol.

91. The method of claim 89 wherein the topoisomerase inhibitor is selected from the group consisting of a topoisomerase I inhibitor, a topoisomerase II inhibitor, and an agent having both topoisomerase I and topoisomerase II activity.

92. The method of claim 1 wherein the method further comprises, subsequent to the administration of an initial dose of the substituted hexitol derivative selected from the group consisting of dianhydrogalactitol, a derivative or analog of dianhydrogalactitol, diacetyldianhydrogalactitol, and a derivative or analog of diacetyldianhydrogalactitol: the steps of: (1) determining the quantity of a protein associated with the activation of the DNA repair pathway to determine the extent of the activation of the DNA repair pathway; and (2) adjusting the dose of the substituted hexitol derivative selected from the group consisting of dianhydrogalactitol, a derivative or analog of dianhydrogalactitol, diacetyldianhydrogalactitol, and a derivative or analog of diacetyldianhydrogalactitol in response to the extent of the DNA repair pathway.

93. The method of claim 92 wherein the protein associated with the activation of the DNA repair pathway is selected from the group consisting of phosphorylated ATM, phosphorylated RPA32, and γH2A.X.

94. The method of claim 92 wherein the substituted hexitol derivative selected from the group consisting of dianhydrogalactitol, a derivative or analog of dianhydrogalactitol, diacetyldianhydrogalactitol, and a derivative or analog of diacetyldianhydrogalactitol is dianhydrogalactitol.

95. A composition to improve the efficacy and/or reduce the side effects of suboptimally administered drug therapy employing a substituted hexitol derivative for the treatment of glioblastoma, NSCLC, or ovarian cancer comprising an alternative selected from the group consisting of:
(i) a therapeutically effective quantity of a modified substituted hexitol derivative or a derivative, analog, or prodrug of a substituted hexitol derivative or a modified substituted hexitol derivative, wherein the modified substituted hexitol derivative or the derivative, analog or prodrug of the substituted hexitol derivative or modified substituted hexitol derivative possesses increased therapeutic efficacy or reduced side effects for treatment of glioblastoma, NSCLC, or ovarian cancer as compared with an unmodified substituted hexitol derivative;
(ii) a composition comprising:
(a) a therapeutically effective quantity of a substituted hexitol derivative, a modified substituted hexitol derivative, or a derivative, analog, or prodrug of a substituted hexitol derivative or a modified substituted hexitol derivative; and
(b) at least one additional therapeutic agent, therapeutic agent subject to chemosensitization, therapeutic agent subject to chemopotentiation, diluent, excipient, solvent system, drug delivery system, agent to counteract myelosuppression, or agent that increases the ability of the substituted hexitol to pass through the blood-brain barrier, wherein the composition possesses increased therapeutic efficacy or reduced side effects for treatment of glioblastoma, NSCLC, or ovarian cancer as compared with an unmodified substituted hexitol derivative;
(iii) a therapeutically effective quantity of a substituted hexitol derivative, a modified substituted hexitol derivative or a derivative, analog, or prodrug of a substituted hexitol derivative or a modified substituted hexitol derivative that is incorporated into a dosage form, wherein the substituted hexitol derivative, the modified substituted hexitol derivative or the derivative, analog, or prodrug of a substituted hexitol derivative or a modified substituted hexitol derivative incorporated into the dosage form possesses increased therapeutic efficacy or reduced side effects for treatment of glioblastoma, NSCLC, or ovarian cancer as compared with an unmodified substituted hexitol derivative;
(iv) a therapeutically effective quantity of a substituted hexitol derivative, a modified substituted hexitol derivative or a derivative, analog, or prodrug of a substituted hexitol derivative or a modified substituted hexitol derivative that is incorporated into a dosage kit and packaging, wherein the substituted hexitol derivative, the modified substituted hexitol derivative or the derivative, analog, or prodrug of a substituted hexitol derivative or a modified substituted hexitol derivative incorporated into the dosage kit and packaging possesses increased therapeutic efficacy or reduced side effects for treatment of glioblastoma, NSCLC, or ovarian cancer as compared with an unmodified substituted hexitol derivative; and
(v) a therapeutically effective quantity of a substituted hexitol derivative, a modified substituted hexitol derivative or a derivative, analog, or prodrug of a substituted hexitol derivative or a modified substituted hexitol derivative that is subjected to a bulk drug product improvement, wherein substituted hexitol derivative, a modified substituted hexitol derivative or a derivative, analog, or prodrug of a substituted hexitol derivative or a modified substituted hexitol derivative subjected to the bulk drug product improvement possesses increased therapeutic efficacy or reduced side effects for treatment of glioblastoma, NSCLC, or ovarian cancer as compared with an unmodified substituted hexitol derivative.

96. The composition of claim 95 wherein the substituted hexitol derivative is selected from the group consisting of dianhydrogalactitol and a derivative or analog of dianhydrogalactitol.

97. The composition of claim 96 wherein the substituted hexitol derivative selected from the group consisting of dianhydrogalactitol and a derivative or analog of dianhydrogalactitol is dianhydrogalactitol.

98. The composition of claim 96 wherein the substituted hexitol derivative selected from the group consisting of dianhydrogalactitol and a derivative or analog of dianhydrogalactitol is a derivative or analog of dianhydrogalactitol.

99. The composition of claim 98 wherein the derivative or analog of dianhydrogalactitol is a derivative of dianhydrogalactitol that is selected from the group consisting of: (i) a derivative of dianhydrogalactitol that has one or both of the hydrogens of the two hydroxyl groups of dianhydrogalactitol replaced with lower alkyl; (ii) a derivative of dianhydrogalactitol that has one or more of the hydrogens attached to the two epoxide rings replaced with lower alkyl; (iii) a derivative of dianhydrogalactitol that has one or both of the methyl groups present in dianhydrogalactitol and that are attached to the same carbons that bear the hydroxyl groups replaced with C₂-C₆ lower alkyl; and (iv) a derivative of dianhydrogalactitol that has one or both of the methyl groups present in dianhydrogalactitol and that are attached to the same carbons that bear the hydroxyl groups substituted with a halo group by replacing a hydrogen of the methyl group with a halo group.

100. The composition of claim 95 wherein the hexitol derivative is selected from the group consisting of diacetyldianhydrogalactitol and a derivative or analog of diacetyldianhydrogalactitol.

101. The composition of claim 100 wherein the hexitol derivative selected from the group consisting of diacetyldianhydrogalactitol and a derivative or analog of diacetyldianhydrogalactitol is diacetyldianhydrogalactitol.

102. The composition of claim 101 wherein the hexitol derivative is selected from the group consisting of diacetyldianhydrogalactitol and a derivative or analog of diacetyldianhydrogalactitol is a derivative or analog of diacetyldianhydrogalactitol.

103. The composition of claim 102 wherein the derivative or analog of diacetyldianhydrogalactitol is a derivative of diacetyldianhydrogalactitol that is selected from the group consisting of: (i) a derivative of diacetyldianhydrogalactitol that has one or both of the methyl groups that are part of the acetyl moieties replaced with C₂-C₆ lower alkyl; (ii) a derivative of diacetyldianhydrogalactitol that has one or both of the hydrogens attached to the epoxide ring replaced with lower alkyl; (iii) a derivative of diacetyldianhydrogalactitol that has one or both of the methyl groups attached to the same carbons that bear the acetyl groups replaced with C₂-C₆ lower alkyl; and (iv) a derivative of diacetyldianhydrogalactitol that has one or both of the methyl groups that are attached to the same carbons that bear the hydroxyl groups substituted with a halo group by replacing a hydrogen of the methyl group with a halo group.

104. The composition of claim 95 wherein the composition possesses increased therapeutic efficacy or reduced side effects for treatment of glioblastoma.

105. The composition of claim 95 wherein the composition possesses increased therapeutic efficacy or reduced side effects for treatment of NSCLC.

106. The composition of claim 95 wherein the composition possesses increased therapeutic efficacy or reduced side effects for treatment of ovarian cancer.

107. The composition of claim 95 wherein the composition is formulated to exert a cytotoxic effect against cancer stem cells.

108. The composition of claim 95 wherein the composition comprises a drug combination comprising:
(i) an alkylating hexitol derivative, a modified alkylating hexitol derivative, or a derivative, analog, or prodrug of an alkylating hexitol derivative or a modified alkylating hexitol derivative; and
(ii) an additional therapeutic agent selected from the group consisting of:
(a) topoisomerase inhibitors;
(b) fraudulent nucleosides;
(c) fraudulent nucleotides;
(d) thymidylate synthetase inhibitors;
(e) signal transduction inhibitors;
(f) cisplatin or platinum analogs;
(g) alkylating agents;
(h) anti-tubulin agents;
(i) antimetabolites;
(j) berberine;
(k) apigenin;
(l) amonafide;
(m) vinca alkaloids;
(n) 5-fluorouracil;
(o) curcumin;
(p) NF-κB inhibitors;
(q) rosmarinic acid;
(r) mitoguazone; and
(s) tetrandrine.

109. The composition of claim 98 wherein the composition comprises, as additional therapeutic agents: (i) a topoisomerase inhibitor; and (ii) an inhibitor of CHK1 kinase or CHK2 kinase.

110. The composition of claim 95 wherein the composition comprises:
(i) an alkylating hexitol derivative, a modified alkylating hexitol derivative, or a derivative, analog, or prodrug of an alkylating hexitol derivative or a modified alkylating hexitol derivative; and
(ii) a therapeutic agent subject to chemosensitization selected from the group consisting of:
(a) topoisomerase inhibitors;
(b) fraudulent nucleosides;
(c) fraudulent nucleotides;
(d) thymidylate synthetase inhibitors;
(e) signal transduction inhibitors;
(f) cisplatin or platinum analogs;
(g) alkylating agents;
(h) anti-tubulin agents;
(i) antimetabolites;
(j) berberine;
(k) apigenin;
(l) colchicine or an analog of colchicine;
(m) genistein;
(n) etoposide;
(o) cytarabine;
(p) camptothecin;
(q) vinca alkaloids;
(r) 5-fluorouracil;
(s) curcumin;
(t) NF-κB inhibitors;
(u) rosmarinic acid; and
(v) mitoguazone;
wherein the alkylating hexitol derivative, a modified alkylating hexitol derivative, or a derivative, analog, or prodrug of an alkylating hexitol derivative or a modified alkylating hexitol derivative acts as a chemosensitizer.

111. The composition of claim 95 wherein the composition comprises:
(i) an alkylating hexitol derivative, a modified alkylating hexitol derivative, or a derivative, analog, or prodrug of an alkylating hexitol derivative or a modified alkylating hexitol derivative; and
(ii) a therapeutic agent subject to chemopotentiation selected from the group consisting of:
(a) fraudulent nucleosides;
(b) fraudulent nucleotides;
(c) thymidylate synthetase inhibitors;
(d) signal transduction inhibitors;
(e) cisplatin or platinum analogs;
(f) alkylating agents;
(g) anti-tubulin agents;
(h) antimetabolites;
(i) berberine;
(j) apigenin;
(k) colchicine or analogs of colchicine;
(l) genistein;
(m) etoposide;
(n) cytarabine;
(o) camptothecins;
(p) vinca alkaloids;
(q) topoisomerase inhibitors;
(r) 5-fluorouracil;
(s) curcumin;
(t) NF-κB inhibitors;
(u) rosmarinic acid;
(v) mitoguazone; and
(w) a biotherapeutic;
wherein the alkylating hexitol derivative, a modified alkylating hexitol derivative, or a derivative, analog, or prodrug of an alkylating hexitol derivative or a modified alkylating hexitol derivative acts as a chemopotentiator.

112. The composition of claim 95 wherein the alkylating hexitol derivative, a modified alkylating hexitol derivative, or a derivative, analog, or prodrug of the alkylating hexitol derivative or the modified alkylating hexitol derivative of the composition is subjected to a bulk drug product improvement, wherein the bulk drug product improvement is selected from the group consisting of:
(a) salt formation;
(b) preparation as a homogeneous crystal structure;
(c) preparation as a pure isomer;
(d) increased purity;
(e) preparation with lower residual solvent content; and
(f) preparation with lower residual heavy metal content.

113. The composition of claim 95 wherein the composition comprises an alkylating hexitol derivative, a modified alkylating hexitol derivative, or a derivative, analog, or prodrug of an alkylating hexitol derivative or a modified alkylating hexitol derivative and a diluent, wherein the diluent is selected from the group consisting of:
(a) an emulsion;
(b) dimethylsulfoxide (DMSO);
(c) N-methylformamide (NMF)
(d) dimethylformamide (DMF)
(e) dimethylacetamide (DMA);
(f) ethanol;
(g) benzyl alcohol;
(h) dextrose-containing water for injection;
(i) Cremophor;
(j) cyclodextrins; and
(k) PEG.

114. The composition of claim 95 wherein the composition comprises an alkylating hexitol derivative, a modified alkylating hexitol derivative, or a derivative, analog, or prodrug of an alkylating hexitol derivative or a modified alkylating hexitol derivative and a solvent system, wherein the solvent system is selected from the group consisting of:
(a) an emulsion;
(b) DMSO;
(c) NMF;
(d) DMF;
(e) DMA;
(f) ethanol;
(g) benzyl alcohol;
(h) dextrose-containing water for injection;
(i) Cremophor;
(j) PEG; and
(k) salt systems.

115. The composition of claim 95 wherein the composition comprises an alkylating hexitol derivative, a modified alkylating hexitol derivative, or a derivative, analog, or prodrug of an alkylating hexitol derivative or a modified alkylating hexitol derivative and an excipient, wherein the excipient is selected from the group consisting of:
(a) mannitol;
(b) albumin;
(c) EDTA;
(d) sodium bisulfite;
(e) benzyl alcohol;
(f) carbonate buffers;
(g) phosphate buffers;
(h) PEG;
(i) vitamin A;
(j) vitamin D;
(k) vitamin E;
(l) esterase inhibitors;
(m) cytochrome P450 inhibitors;
(n) multi-drug resistance (MDR) inhibitors;
(o) organic resins;
(p) detergents;
(q) perillyl alcohol or an analog thereof; and
(r) activators of channel-forming receptors.

116. The composition of claim 95 wherein the alkylating hexitol derivative, modified alkylating hexitol derivative, or derivative, analog, or prodrug of the alkylating hexitol derivative or modified alkylating hexitol derivative is incorporated into a dosage form selected from the group consisting of:
(a) tablets;
(b) capsules;
(c) topical gels;
(d) topical creams;
(e) patches;
(f) suppositories;
(g) lyophilized dosage fills;
(h) immediate-release formulations;
(i) slow-release formulations;
(j) controlled-release formulations; and
(k) liquid in capsules.

117. The composition of claim 95 wherein the composition comprises: (i) an alkylating hexitol derivative, modified alkylating hexitol derivative, or derivative, analog, or prodrug of an alkylating hexitol derivative or modified alkylating hexitol derivative; and (ii) a drug delivery system, wherein the drug delivery system is selected from the group consisting of:
(a) oral dosage forms;
(b) nanocrystals;
(c) nanoparticles;
(d) cosolvents;
(e) slurries;
(f) syrups;
(g) bioerodible polymers;
(h) liposomes;
(i) slow-release injectable gels;
(j) microspheres; and
(k) targeting compositions with epidermal growth factor receptor-binding peptides.

118. The composition of claim 95 wherein the therapeutic agent is an alkylating hexitol derivative, modified alkylating hexitol derivative, or derivative or analog of an alkylating hexitol derivative or modified alkylating hexitol derivative and the therapeutic agent is present in the composition in a drug conjugate form, wherein the drug conjugate form is a drug conjugate form selected from the group consisting of:
(a) a polymer system;
(b) polylactides;
(c) polyglycolides;
(d) amino acids;
(e) peptides;
(f) multivalent linkers;
(g) immunoglobulins;
(h) cyclodextrin polymers;
(i) modified transferrin;
(i) hydrophobic or hydrophobic-hydrophilic polymers;
(k) conjugates with a phosphonoformic acid partial ester;
(l) conjugates with a cell-binding agent incorporating a charged cross-linker; and
(m) conjugates with β-glucuronides through a linker.

119. The composition of claim 95 wherein the therapeutic agent is an alkylating hexitol derivative, modified alkylating hexitol derivative, or derivative or analog of an alkylating hexitol derivative or modified alkylating hexitol derivative and the therapeutic agent is in the form of a prodrug system, wherein the prodrug system is selected from the group consisting of:
(a) enzyme sensitive esters;
(b) dimers;
(c) Schiff bases;
(d) pyridoxal complexes;
(e) caffeine complexes;
(f) nitric oxide-releasing prodrugs;
(g) prodrugs with fibroblast activation protein α-cleavable oligopeptides;
(h) products of reaction with an acylating or carbamylating agent;
(i) hexanoate conjugates;
(j) polymer-agent conjugates; and
(k) prodrugs that are subject to redox activation.

120. The composition of claim 95 wherein the therapeutic agent is an alkylating hexitol derivative, modified alkylating hexitol derivative, or derivative, analog, or prodrug of an alkylating hexitol derivative or modified alkylating hexitol derivative and the composition further comprises at least one additional therapeutic agent to form a multiple drug system, wherein the at least one additional therapeutic agent is selected from the group consisting of:
(a) an inhibitor of multi-drug resistance;
(b) a specific drug resistance inhibitor;
(c) a specific inhibitor of a selective enzyme;
(d) a signal transduction inhibitor;
(e) an inhibitor of a repair enzyme; and
(f) a topoisomerase inhibitor with non-overlapping side effects.

121. The composition of claim 95 wherein the therapeutic agent is an alkylating hexitol derivative, modified alkylating hexitol derivative, or derivative, analog, or prodrug of an alkylating hexitol derivative or modified alkylating hexitol derivative and the composition further comprises an agent for counteracting myelosuppression, wherein the agents for counteracting myelosuppression is a dithiocarbamate.

122. The composition of claim 95 wherein the therapeutic agent is an alkylating hexitol derivative, modified alkylating hexitol derivative, or derivative, analog, or prodrug of an alkylating hexitol derivative or modified alkylating hexitol derivative and the composition further comprises an agent that increases the ability of the substituted hexitol to pass through the blood-brain barrier, wherein the agent that increases the ability of the substituted hexitol to pass through the blood-brain barrier is selected from the group consisting of:
(a) a chimeric peptide of the structure of Formula (D-III): 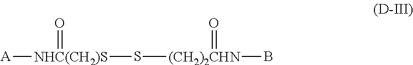
wherein: (A) A is somatostatin, thyrotropin releasing hormone (TRH), vasopressin, alpha interferon, endorphin, muramyl dipeptide or ACTH 4-9 analogue; and (B) B is insulin, IGF-I, IGF-II, transferrin, cationized (basic) albumin or prolactin; or a chimeric peptide of the structure of Formula (D-III) wherein the disulfide conjugating bridge between A and B is replaced with a bridge of Subformula (D-III(a)):
A-NH(CH₂)₂S—S—B (cleavable linkage) (D-III(a)),
wherein the bridge is formed using cysteamine and EDAC as the bridge reagents; or a chimeric peptide of the structure of Formula (D-III) wherein the disulfide conjugating bridge between A and B is replaced with a bridge of Subformula (D-III(b)):
A-NH═CH(CH₂)₃CH═NH—B (non-cleavable linkage) (D-III(b)),
wherein the bridge is formed using glutaraldehyde as the bridge reagent;
(b) a composition comprising either avidin or an avidin fusion protein bonded to a biotinylated substituted hexitol derivative to form an avidin-biotin-agent complex including therein a protein selected from the group consisting of insulin, transferrin, an anti-receptor monoclonal antibody, a cationized protein, and a lectin;
(c) a neutral liposome that is pegylated and incorporates the substituted hexitol derivative, wherein the polyethylene glycol strands are conjugated to at least one transportable peptide or targeting agent;
(d) a humanized murine antibody that binds to the human insulin receptor linked to the substituted hexitol derivative through an avidin-biotin linkage; and
(e) a fusion protein comprising a first segment and a second segment: the first segment comprising a variable region of an antibody that recognizes an antigen on the surface of a cell that after binding to the variable region of the antibody undergoes antibody-receptor-mediated endocytosis, and, optionally, further comprises at least one domain of a constant region of an antibody; and the second segment comprising a protein domain selected from the group consisting of avidin, an avidin mutein, a chemically modified avidin derivative, streptavidin, a streptavidin mutein, and a chemically modified streptavidin derivative, wherein the fusion protein is linked to the substituted hexitol by a covalent link to biotin.

123. The composition of claim 97 wherein the composition is formulated for administration of dianhydrogalactitol by dosing once daily for three consecutive days every 21 days.

124. A method for the treatment of leptomeningeal carcinomatosis (LC) by the induction of double-strand breaks in the DNA of tumor cells by administration of a therapeutically effective quantity of a substituted hexitol derivative selected from the group consisting of dianhydrogalactitol, a derivative or analog of dianhydrogalactitol, diacetyldianhydrogalactitol, and a derivative or analog of diacetyldianhydrogalactitol.

125. The method of claim 124 wherein the substituted hexitol derivative is selected from the group consisting of dianhydrogalactitol and a derivative or analog of dianhydrogalactitol.

126. The method of claim 125 wherein the substituted hexitol derivative selected from the group consisting of dianhydrogalactitol and a derivative or analog of dianhydrogalactitol is dianhydrogalactitol.

127. The method of claim 124 wherein the substituted hexitol derivative selected from the group consisting of dianhydrogalactitol and a derivative or analog of dianhydrogalactitol is a derivative or analog of dianhydrogalactitol.

128. The method of claim 127 wherein the derivative or analog of dianhydrogalactitol is a derivative of dianhydrogalactitol that is selected from the group consisting of: (i) a derivative of dianhydrogalactitol that has one or both of the hydrogens of the two hydroxyl groups of dianhydrogalactitol replaced with lower alkyl; (ii) a derivative of dianhydrogalactitol that has one or more of the hydrogens attached to the two epoxide rings replaced with lower alkyl; (iii) a derivative of dianhydrogalactitol that has one or both of the methyl groups present in dianhydrogalactitol and that are attached to the same carbons that bear the hydroxyl groups replaced with C₂-C₆ lower alkyl; and (iv) a derivative of dianhydrogalactitol that has one or both of the methyl groups present in dianhydrogalactitol and that are attached to the same carbons that bear the hydroxyl groups substituted with a halo group by replacing a hydrogen of the methyl group with a halo group.

129. The method of claim 124 wherein the hexitol derivative is selected from the group consisting of diacetyldianhydrogalactitol and a derivative or analog of diacetyldianhydrogalactitol.

130. The method of claim 129 wherein the hexitol derivative selected from the group consisting of diacetyldianhydrogalactitol and a derivative or analog of diacetyldianhydrogalactitol is diacetyldianhydrogalactitol.

131. The method of claim 129 wherein the hexitol derivative selected from the group consisting of diacetyldianhydrogalactitol and a derivative or analog of diacetyldianhydrogalactitol is a derivative or analog of diacetyldianhydrogalactitol.

132. The method of claim 131 wherein the derivative or analog of diacetyldianhydrogalactitol is a derivative of diacetyldianhydrogalactitol that is selected from the group consisting of: (i) a derivative of diacetyldianhydrogalactitol that has one or both of the methyl groups that are part of the acetyl moieties replaced with C₂-C₆ lower alkyl; (ii) a derivative of diacetyldianhydrogalactitol that has one or both of the hydrogens attached to the epoxide ring replaced with lower alkyl; (iii) a derivative of diacetyldianhydrogalactitol that has one or both of the methyl groups attached to the same carbons that bear the acetyl groups replaced with C₂-C₆ lower alkyl; and (iv) a derivative of diacetyldianhydrogalactitol that has one or both of the methyl groups that are attached to the same carbons that bear the hydroxyl groups substituted with a halo group by replacing a hydrogen of the methyl group with a halo group.

133. The method of claim 124 wherein the method comprises the steps of:
(a) identifying at least one factor or parameter associated with the efficacy and/or occurrence of side effects of the administration of the substituted hexitol derivative for treatment of leptomeningeal carcinomatosis (LC); and
(b) modifying the factor or parameter to improve the efficacy and/or reduce the side effects of the administration of the substituted hexitol derivative for treatment of LC.

134. The method of claim 133 wherein the factor or parameter is selected from the group consisting of:
(a) dose modification;
(b) route of administration;
(c) schedule of administration;
(d) indications for use;
(e) selection of disease stage;
(f) other indications;
(g) patient selection;
(h) patient/disease phenotype;
(i) patient/disease genotype;
(j) pre/post-treatment preparation
(k) toxicity management;
(l) pharmacokinetic/pharmacodynamic monitoring;
(m) drug combinations;
(n) chemosensitization;
(o) chemopotentiation;
(p) post-treatment patient management;
(q) alternative medicine/therapeutic support;
(r) bulk drug product improvements;
(s) diluent systems;
(t) solvent systems;
(u) excipients;
(v) dosage forms;
(w) dosage kits and packaging;
(x) drug delivery systems;
(y) drug conjugate forms;
(z) compound analogs;
(aa) prodrugs;
(ab) multiple drug systems;
(ac) biotherapeutic enhancement;
(ad) biotherapeutic resistance modulation;
(ae) radiation therapy enhancement;
(af) novel mechanisms of action;
(ag) selective target cell population therapeutics;
(ah) use with ionizing radiation;
(ai) use with an agent enhancing its activity;
(aj) use with an agent that counteracts myelosuppression; and
(ak) use with an agent that increases the ability of the substituted hexitol to pass through the blood-brain barrier.

135. The method of claim 124 wherein the method further comprises administration of a therapeutically effective quantity of a PARP inhibitor.

136. The method of claim 135 wherein the PARP inhibitor is selected from the group consisting of iniparib, talazoparib, olaparib, rucaparib, veliparib, CEP-9722 (a prodrug of CEP-8983 (11-methoxy-4,5,6,7-tetrahydro-1H-cyclopenta[a]pyrrolo[3,4-c]carbazole-1,3(2H)-dione), MK 4827 ((S)-2-(4-(piperidin-3-yl)phenyl)-2H-indazole-7-carboxamide), and BGB-290.

137. The method of claim 124 wherein the method further comprises administration of a therapeutically effective quantity of an agent that counters loss of PTEN function.

138. The method of claim 137 wherein the agent that counters loss of PTEN function is selected from the group consisting of temsirolimus, everolimus, AZD6482 ((R)-2-(1-(7-methyl-2-morpholino-4-oxo-4H-pyrido[1,2-a]pyrimidin-9-yl)ethylamino)benzoic acid), MK-2206 (8-(4-(1-aminocyclobutyl)phenyl)-9-phenyl-[1,2,4]triazolo[3,4-f][1,6]naphthyridin-3(2H)-one), and 17-AAG ([(3S,5S,6R,7S,8E,10R,11S,12E,14E)-21-(allylamino)-6-hydroxy-5,11-dimethoxy-3,7,9,15-tetramethyl-16,20,22-trioxo-17-azabicyclo[16.3.1]docosa-8,12,14,18,21-pentaen-10-yl] carbamate).

139. The method of claim 124 wherein the method further comprises administration of a therapeutically effective quantity of an additional anti-neoplastic agent that damages DNA.

140. The method of claim 135 wherein the additional anti-neoplastic agent that damages DNA is selected from the group consisting of cisplatin, carboplatin, oxaliplatin, picoplatin, nedaplatin, satraplatin, tetraplatin, doxorubicin, daunorubicin, methotrexate, 5-fluorouracil, gemcitabine, podophyllotoxin, etoposide, teniposide, cyclophosphamide, chlorambucil, melphalan, carmustine, lomustine, estramustine, semustine, bendamustine, prednamustine, uramustine, chlornaphazine, dacarbazine, altretamine, temozolomide, mitomycin C, streptozotocin, chlorozotocin, capecitabine, floxuridine, 6-mercaptopurine, 8-azaguanine, azathiopurine, 5-ethynyluracil, thioguanine, fludarabine, cytarabine, cladribine, 2-fluoro-arabinosyl-adenine, aminopterin, pemetrexed, ralitrexed, camptothecin, epirubicin, idarubicin, methylnitronitrosoguanidine, topotecan, irinotecan, mechlorethamine, ifosfamide, trofosfamide, busulfan, procarbazine, mitoxantrone, actinomycin, calicheamicin, Tegafur (R,S-1-(tetrahydro-2-furanyl)-5-fluorouracil), 2′,2′-difluoro-2′-deoxycytidine, bischloroethylsulfide, thiotepa, aziridinylbenzoquinone, BCNU, CCNU, 4-methyl CCNU, ACNU, rebeccamycin, bleomycin, pepleomycin, ethylmethanesulfonate, methylmethanesulfonate, dimethylnitrosamine, dimethyl sulfate, and N′-[2-[2-(4-methoxyphenyl)ethenyl]-4-quinazolinyl]-N,N-dimethyl-1,3-propanediamine dihydrochloride.

141. The method of claim 124 wherein the method further comprises administration of a therapeutically effective quantity of an additional agent for treatment of LC.

142. The method of claim 141 wherein the additional agent for treatment of LC is selected from the group consisting of cytarabine, methotrexate, thiotepa, 4-[(3-chloro-2-fluorophenyl)amino]-7-methoxyquinazolin-6-yl(2R)-2,4-dimethylpiperazine-1-carboxylate, microRNA 199b-5p, interleukin-2, a pyridine STAT3/STAT5 modulator, a substituted quinoxaline inhibitor of inhibiting IKKβ and the NFκB and mTOR pathways, rituximab, irinotecan, taurolidine, taurultam, VEGFR-3 fusion proteins, a reaction product of taurultam with glucose, temozolomide, 4-hydroperoxycyclophosphamide, platinum-transferrin, phenylbenzothiazole, stilbene, biphenylalkyne, pyridine derivatives, 7-benzyl-10-(2-methylbenzyl)-2,6,7,8,9,10-hexahydroimidazo[1,2-a]pyrido[4,3-d]pyrimidin-5(3H)-one, 4-iodo-3-nitrobenzamide, interferon-α, interferon-β, a STAT3 inhibitor, coenzyme Q10, arabino-2′-O-methyl nucleosides and derivatives thereof, ricin mutants, methylol taurinamide, methylol-taurultam, an aminoglycan of taurultam, benzimidazole thiophene compounds, chlorambucil, temozolomide, thalidomide, and lenalidomide.

143. A composition to improve the efficacy and/or reduce the side effects of suboptimally administered drug therapy employing a substituted hexitol derivative for the treatment of leptomeningeal carcinomatosis (LC) comprising an alternative selected from the group consisting of:
(i) a therapeutically effective quantity of a modified substituted hexitol derivative or a derivative, analog, or prodrug of a substituted hexitol derivative or a modified substituted hexitol derivative, wherein the modified substituted hexitol derivative or the derivative, analog or prodrug of the substituted hexitol derivative or modified substituted hexitol derivative possesses increased therapeutic efficacy or reduced side effects for treatment of LC as compared with an unmodified substituted hexitol derivative;
(ii) a composition comprising:
(a) a therapeutically effective quantity of a substituted hexitol derivative, a modified substituted hexitol derivative, or a derivative, analog, or prodrug of a substituted hexitol derivative or a modified substituted hexitol derivative; and
(b) at least one additional therapeutic agent, therapeutic agent subject to chemosensitization, therapeutic agent subject to chemopotentiation, diluent, excipient, solvent system, drug delivery system, agent to counteract myelosuppression, or agent that increases the ability of the substituted hexitol to pass through the blood-brain barrier, wherein the composition possesses increased therapeutic efficacy or reduced side effects for treatment of LC as compared with an unmodified substituted hexitol derivative;
(iii) a therapeutically effective quantity of a substituted hexitol derivative, a modified substituted hexitol derivative or a derivative, analog, or prodrug of a substituted hexitol derivative or a modified substituted hexitol derivative that is incorporated into a dosage form, wherein the substituted hexitol derivative, the modified substituted hexitol derivative or the derivative, analog, or prodrug of a substituted hexitol derivative or a modified substituted hexitol derivative incorporated into the dosage form possesses increased therapeutic efficacy or reduced side effects for treatment of LC as compared with an unmodified substituted hexitol derivative;
(iv) a therapeutically effective quantity of a substituted hexitol derivative, a modified substituted hexitol derivative or a derivative, analog, or prodrug of a substituted hexitol derivative or a modified substituted hexitol derivative that is incorporated into a dosage kit and packaging, wherein the substituted hexitol derivative, the modified substituted hexitol derivative or the derivative, analog, or prodrug of a substituted hexitol derivative or a modified substituted hexitol derivative incorporated into the dosage kit and packaging possesses increased therapeutic efficacy or reduced side effects for treatment of LC as compared with an unmodified substituted hexitol derivative; and
(v) a therapeutically effective quantity of a substituted hexitol derivative, a modified substituted hexitol derivative or a derivative, analog, or prodrug of a substituted hexitol derivative or a modified substituted hexitol derivative that is subjected to a bulk drug product improvement, wherein substituted hexitol derivative, a modified substituted hexitol derivative or a derivative, analog, or prodrug of a substituted hexitol derivative or a modified substituted hexitol derivative subjected to the bulk drug product improvement possesses increased therapeutic efficacy or reduced side effects for treatment of LC as compared with an unmodified substituted hexitol derivative.

144. The composition of claim 143 wherein the composition further comprises a therapeutically effective quantity of an additional therapeutic agent for treatment of leptomeningeal carcinomatosis.

145. The composition of claim 144 wherein the additional therapeutic agent for treatment of leptomeningeal carcinomatosis is selected from the group consisting of cytarabine, methotrexate, thiotepa, 4-[(3-chloro-2-fluorophenyl)amino]-7-methoxyquinazolin-6-yl (2R)-2,4-dimethylpiperazine-1-carboxylate, microRNA 199b-5p, interleukin-2, a pyridine STAT3/STAT5 modulator, a substituted quinoxaline inhibitor of inhibiting IKKβ and the NFκB and mTOR pathways, rituximab, irinotecan, taurolidine, taurultam, VEGFR-3 fusion proteins, a reaction product of taurultam with glucose, temozolomide, 4-hydroperoxycyclophosphamide, platinum-transferrin, phenylbenzothiazole, stilbene, biphenylalkyne, pyridine derivatives, 7-benzyl-10-(2-methylbenzyl)-2,6,7,8,9,10-hexahydroimidazo[1,2-a]pyrido[4,3-d]pyrimidin-5(3H)-one, 4-iodo-3-nitrobenzamide, interferon-α, interferon-β, a STAT3 inhibitor, coenzyme Q10, arabino-2′-O-methyl nucleosides and derivatives thereof, ricin mutants, methylol taurinamide, methylol-taurultam, an aminoglycan of taurultam, benzimidazole thiophene compounds, chlorambucil, temozolomide, thalidomide, and lenalidomide.
